(19) United States
(12) Patent Application Publication
(10) Pub. No.: US 2024/0360104 A1
(43) Pub. Date: Oct. 31, 2024

(54) TRANSCRIPTIONAL ENHANCED ASSOCIATED DOMAIN (TEAD) TRANSCRIPTION FACTOR INHIBITORS AND USES THEREOF

(71) Applicant: Dana-Farber Cancer Institute, Inc., Boston, MA (US)

(72) Inventors: Nathanael S. Gray, Stanford, CA (US); Tinghu Zhang, Brookline, MA (US); Mengyang Fan, Allston, MA (US); Jianwei Che, Sharon, MA (US); Wenchao Lu, Brookline, MA (US); Sirano Dhe-Paganon, Holiston, MA (US); Nicholas Paul Kwiatkowski, Brookline, MA (US)

(21) Appl. No.: 18/553,988

(22) PCT Filed: Apr. 26, 2022
(86) PCT No.: PCT/US2022/026268

§ 371 Date: Oct. 4, 2023

Related U.S. Application Data
(60) Provisional application No. 63/291,845, filed on Dec. 20, 2021.
(60) Provisional application No. 63/180,418, filed on Apr. 27, 2021.

(51) Int. Cl. C07D 403/12; A61K 31/40; A61K 31/4155; A61K 31/4178; A61K 31/4192; A61K 31/422; A61K 31/4439; A61K 31/444; A61K 31/454; A61K 31/497; A61K 31/501; A61K 31/506; A61K 31/5377; A61K 31/551; C07D 207/14; C07D 401/12; C07D 401/14; C07D 403/04; C07D 403/14; C07D 405/14; C07D 495/04

(52) U.S. Cl. CPC C07D 403/12; A61K 31/40; A61K 31/4155; A61K 31/4178; A61K 31/4192; A61K 31/422; A61K 31/4439; A61K 31/444; A61K 31/454; A61K 31/497; A61K 31/501; A61K 31/506; A61K 31/5377; A61K 31/551; C07D 207/14; C07D 401/12; C07D 401/14; C07D 403/04; C07D 403/14; C07D 405/14; C07D 495/04

(57) ABSTRACT

Provided herein are compounds of Formula (I′), Formula (II), Formula (III), Formula (IV), Formula (V), Formula (VI), Formula (VII), or Formula (VIII) and pharmaceutically acceptable salts, solvates, hydrates, polymorphs, co-crystals, tautomers, stereoisomers, isotopically labeled derivatives, or prodrugs thereof. Also provided are methods and kits involving the inventive compounds or compositions for treating and/or preventing diseases (e.g., proliferative diseases (e.g., cancers, such as carcinoma, sarcoma, lung cancer, thyroid cancer, skin cancer, ovarian cancer, colorectal cancer, prostate cancer, pancreatic cancer, esophageal cancer, liver cancer, breast cancer)) in a subject. Provided are methods of inhibiting a TEAD transcription factors (e.g., TEAD1, TEAD2, TEAD3, TEAD4) in a subject.

### RELATED APPLICATIONS

The present application claims priority under 35 U.S.C. § 119(e) to U.S. provisional application, U.S. Ser. No. 63/180,418, filed Apr. 27, 2021, and U.S. provisional application, U.S. Ser. No. 63/291,845, filed Dec. 20, 2021, each of which is incorporated herein by reference.

### BACKGROUND OF THE INVENTION

Tr Transcriptional enhanced associate domain (TEAD) transcription factors bind to the co-activator YAP/TAZ, and regulate the transcriptional output of Hippo pathway. The hippo signaling pathway has key roles in organ size control and tumor suppression. Signal transduction in the hippo signaling pathway involves a core kinase cascade, leading to YAP/TAZ phosphorylation. Physiological or pathological inactivation of the hippo signaling pathway leads to dephosphorylation and nuclear accumulation. Nuclear YAP/TAZ binds to transcriptional enhanced associate domains (TEADs) to mediate target gene expression. The TEAD-YAP complex regulates organ development and amplification of oncogenic factors in many cancers (e.g., sarcoma, lung cancer, thyroid cancer, skin cancer, ovarian cancer, colorectal cancer, prostate cancer, pancreatic cancer, esophageal cancer, liver cancer, breast cancer). Several genes in the hippo signaling pathway (e.g., YAP1) have been identified as tumor suppressors, and mutations in these genes have been associated with different human cancers. Additionally, elevated YAP levels have been associated with certain human cancers.

Palmitoylation of TEADs is required for TEAD stability and function in the hippo signaling pathway. The attachment of fatty acid palmitate to cysteine residues regulates protein trafficking, membrane localization, and signaling activities. TEAD transcription factors have been found to possess intrinsic palmitoylating enzyme-like activity and undergo autopalmitoylation.

### SUMMARY OF THE INVENTION

Several small-molecule TEAD inhibitors were recently published in International Application Publication WO 2020/081572, published Apr. 23, 2020, which is incorporated herein by reference. Described herein are TEAD inhibitors of Formula (I′), Formula (I), Formula (II), Formula (III), Formula (IV), Formula (V), Formula (VI), Formula (VII), or Formula (VIII), and pharmaceutically acceptable salts, solvates, hydrates, polymorphs, co-crystals, tautomers, stereoisomers, isotopically labeled derivatives, or prodrugs thereof. The compounds of Formula (I′), Formula (I), Formula (II), Formula (III), Formula (IV), Formula (V), Formula (VI), Formula (VII), or Formula (VIII), and pharmaceutically acceptable salts, solvates, hydrates, polymorphs, co-crystals, tautomers, stereoisomers, isotopically labeled derivatives, prodrugs, compositions, and mixtures thereof, may inhibit the activity of a transcription factor (e.g., a TEAD family transcription factor) in a cell, biological sample, tissue, or subject. Disclosed herein are compounds that bind to a cysteine located in the central pocket of a YAP-binding domain (YBD) on a transcription factor (e.g. a TEAD transcription factor). Methods of using the disclosed compounds, and pharmaceutically acceptable salts, solvates, hydrates, polymorphs, co-crystals, tautomers, stereoisomers, isotopically labeled derivatives, prodrugs, compositions, and mixtures thereof, to study the inhibition of a transcription factor (e.g., TEAD1, TEAD2, TEAD3, TEAD4) are also described. The compounds described herein may be useful as therapeutics for the prevention and/or treatment of diseases associated with the overexpression and/or aberrant (e.g., increased or unwanted) activity of a transcription factor (e.g., TEAD1, TEAD2, TEAD3, or TEAD4). The compounds described herein may be useful in treating and/or preventing a disease or condition, e.g., a proliferative disease (e.g., cancers), in a subject in need thereof. Also provided are uses, methods of use, pharmaceutical compositions, and kits including a compound described herein.

In one aspect, the present disclosure provides compounds of Formula (I′):

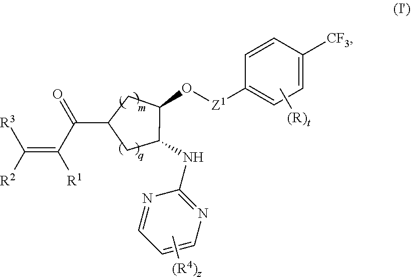

or a pharmaceutically acceptable salt, solvate, hydrate, polymorph, co-crystal, tautomer, stereoisomer, isotopically labeled derivative, prodrug, composition, or mixture thereof, wherein:
R, and R⁴ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
Z¹ is optionally substituted alkylene, optionally substituted alkenylene, optionally substituted alkynylene, optionally substituted carbocyclylene, optionally substituted heterocyclylene, optionally substituted arylene, or optionally substituted heteroarylene;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{a}, when present, can be joined together with the heteroatom to which they are attached to form an optionally substituted heterocyclic ring
   m is 1 or 2;
   q is 1 or 2;
   t is 0, 1, 2, 3, or 4;
   z is 0, 1, 2, or 3.

Exemplary compounds of Formula (I′), include, but are not limited to:

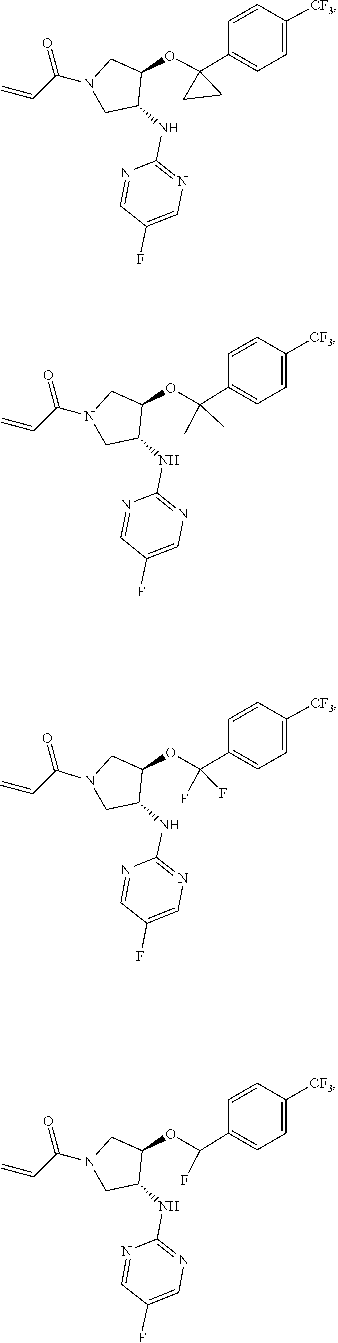 [SMILES: C=CC(=O)N1C[C@@H](Nc2ncc(F)cn2)[C@H](OC2(c3ccc(C(F)(F)F)cc3)CC2)C1 ; C=CC(=O)N1C[C@@H](Nc2ncc(F)cn2)[C@H](OC(C)(C)c2ccc(C(F)(F)F)cc2)C1 ; C=CC(=O)N1C[C@@H](Nc2ncc(F)cn2)[C@H](OC(F)(F)c2ccc(C(F)(F)F)cc2)C1 ; C=CC(=O)N1C[C@@H](Nc2ncc(F)cn2)[C@H](OC(F)c2ccc(C(F)(F)F)cc2)C1]

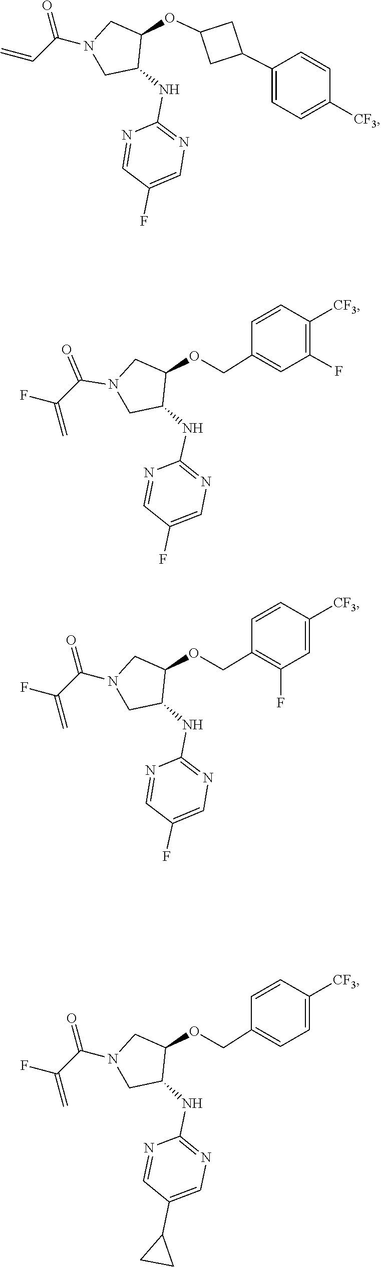 [SMILES: C=CC(=O)N1C[C@@H](Nc2ncc(F)cn2)[C@H](OC2CC(c3ccc(C(F)(F)F)cc3)C2)C1 ; C=C(F)C(=O)N1C[C@@H](Nc2ncc(F)cn2)[C@H](OCc2ccc(C(F)(F)F)c(F)c2)C1 ; C=C(F)C(=O)N1C[C@@H](Nc2ncc(F)cn2)[C@H](OCc2ccc(C(F)(F)F)cc2F)C1 ; C=C(F)C(=O)N1C[C@@H](Nc2ncc(C3CC3)cn2)[C@H](OCc2ccc(C(F)(F)F)cc2)C1]

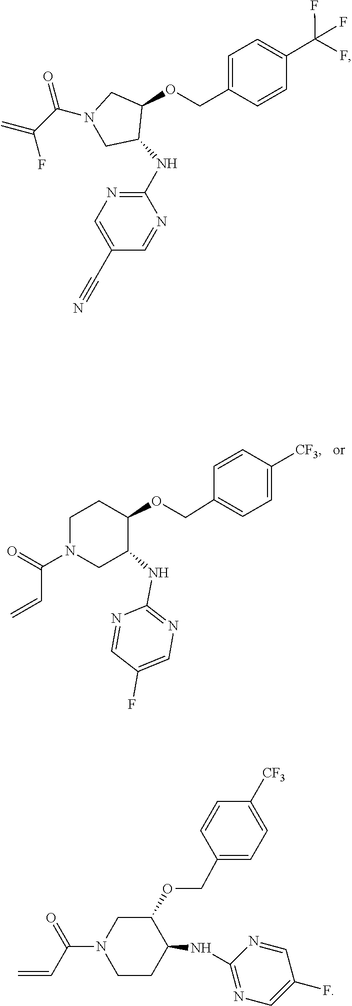

In another aspect, the present disclosure provides compounds of Formula (I):

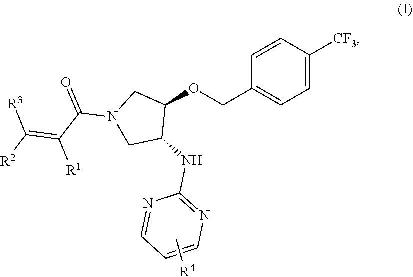

or a pharmaceutically acceptable salt, solvate, hydrate, polymorph, co-crystal, tautomer, stereoisomer, isotopically labeled derivative, prodrug, composition, or mixture thereof, wherein:
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
R⁴ is selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{a}, when present, can be joined together with the heteroatom to which they are attached to form an optionally substituted heterocyclic ring.

Exemplary compounds of Formula (I), include, but are not limited to:

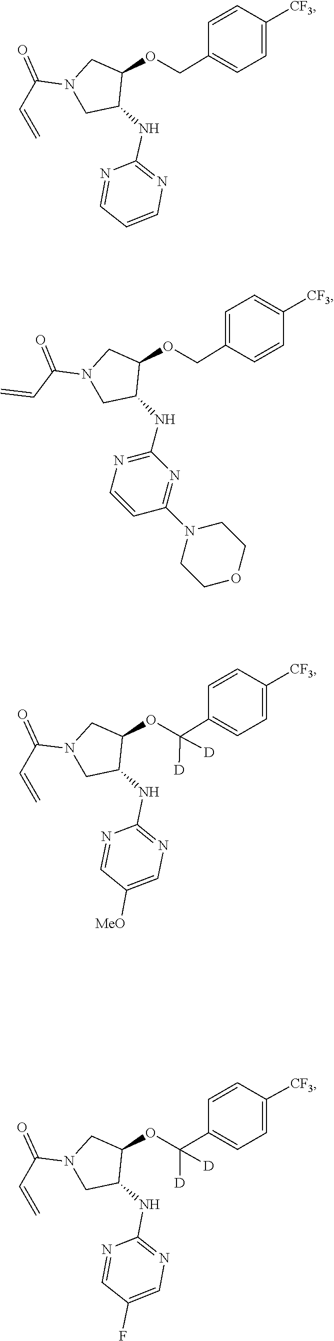 [SMILES: C=CC(=O)N1C[C@@H](Nc2ncccn2)[C@H](OCc2ccc(C(F)(F)F)cc2)C1 ; C=CC(=O)N1C[C@@H](Nc2nccc(N3CCOCC3)n2)[C@H](OCc2ccc(C(F)(F)F)cc2)C1 ; [2H]C([2H])(O[C@@H]1CN(C(=O)C=C)C[C@H]1Nc1ncc(OC)cn1)c1ccc(C(F)(F)F)cc1 ; [2H]C([2H])(O[C@@H]1CN(C(=O)C=C)C[C@H]1Nc1ncc(F)cn1)c1ccc(C(F)(F)F)cc1]

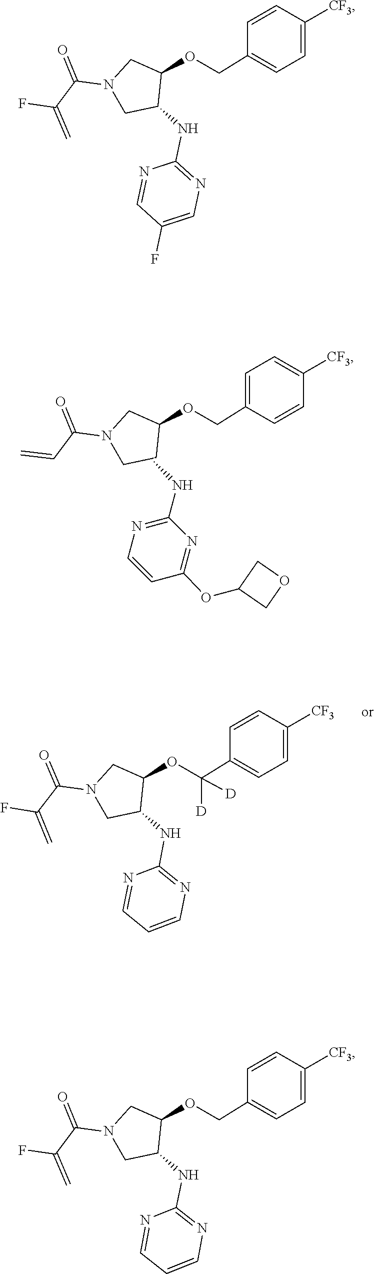

or a pharmaceutically acceptable salt, solvate, hydrate, polymorph, co-crystal, tautomer, stereoisomer, isotopically labeled derivative, prodrug, composition, or mixture thereof.

In another aspect, the present disclosure provides compounds of Formula (II):

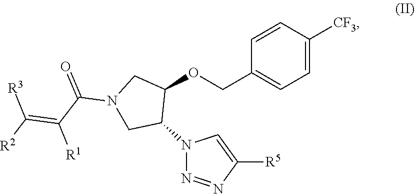

or a pharmaceutically acceptable salt, solvate, hydrate, polymorph, co-crystal, tautomer, stereoisomer, isotopically labeled derivative, prodrug, composition, or mixture thereof, wherein:
R¹, R², and R³, are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}, —C(═O)R^{b}, —C(═O)OR^{b}, —C(═O)N(R^{b})₂, —OC(═O)R^{b}, —N(R^{b})C(═O)R^{b}, —CN, and —NO₂;
R⁵ is selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}, —C(═O)R^{b}, —C(═O)OR^{b}, —C(═O)N(R^{b})₂, —OC(═O)R^{b}, —N(R^{b})C(═O)R^{b}, —CN, and —NO₂;
each occurrence of R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{b}, when present, can be joined together with the heteroatom to which they are attached to form an optionally substituted heterocyclic ring;
provided that the compound is not of the formula:

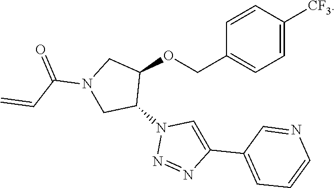 [SMILES: C=CC(=O)N1C[C@@H](n2cc(-c3cccnc3)nn2)[C@H](OCc2ccc(C(F)(F)F)cc2)C1]

Exemplary compounds of Formula (II), include, but are not limited to:

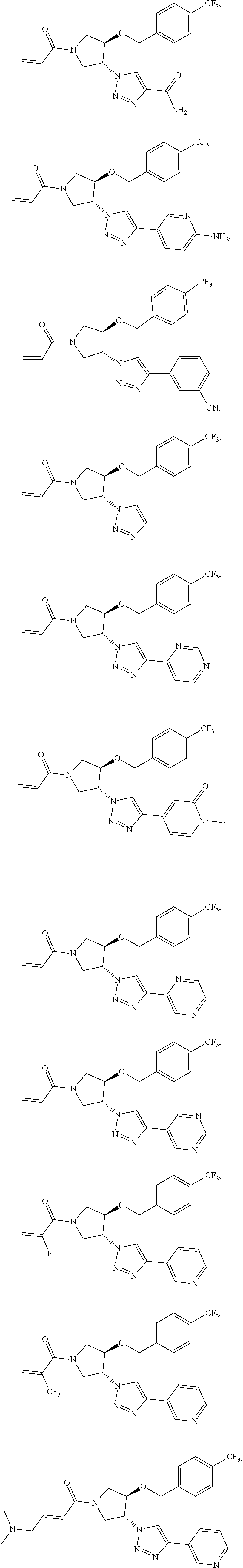

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (III):

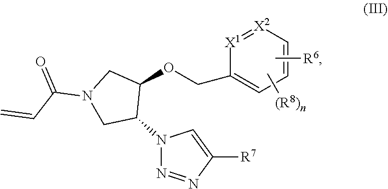

or a pharmaceutically acceptable salt, solvate, hydrate, polymorph, co-crystal, tautomer, stereoisomer, isotopically labeled derivative, prodrug, composition, or mixture thereof, wherein:
R⁶ is —CF₃ or —C≡C—H;
R⁷ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
each instance of R⁸ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
each occurrence of R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of Re, when present, can be joined together with the heteroatom to which they are attached to form an optionally substituted heterocyclic ring;
X¹ and X² are each independently —N═ or —C(H)═; and
n is 0, 1, or 2.

Exemplary compounds of Formula (III), include, but are not limited to:

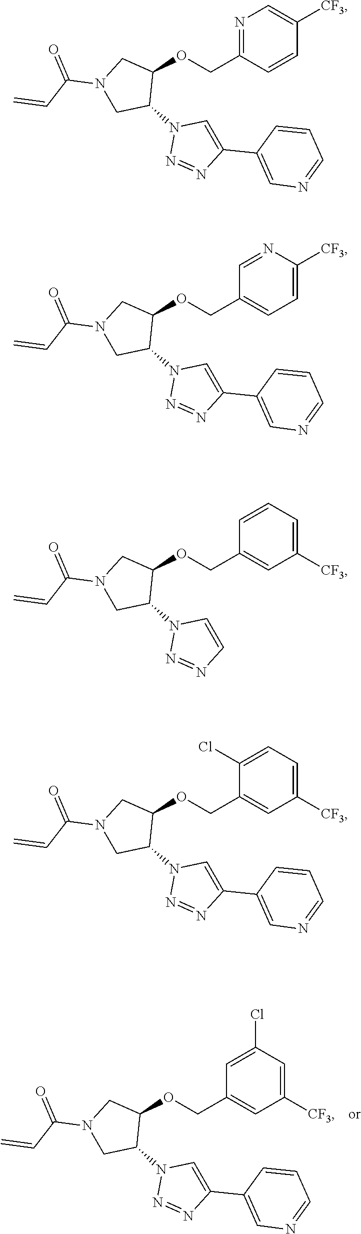 [SMILES: C=CC(=O)N1C[C@@H](n2cc(-c3cccnc3)nn2)[C@H](OCc2ccc(C(F)(F)F)cn2)C1 ; C=CC(=O)N1C[C@@H](n2cc(-c3cccnc3)nn2)[C@H](OCc2ccc(C(F)(F)F)nc2)C1 ; C=CC(=O)N1C[C@@H](n2ccnn2)[C@H](OCc2cccc(C(F)(F)F)c2)C1 ; C=CC(=O)N1C[C@@H](n2cc(-c3cccnc3)nn2)[C@H](OCc2cc(C(F)(F)F)ccc2Cl)C1 ; C=CC(=O)N1C[C@@H](n2cc(-c3cccnc3)nn2)[C@H](OCc2cc(Cl)cc(C(F)(F)F)c2)C1]

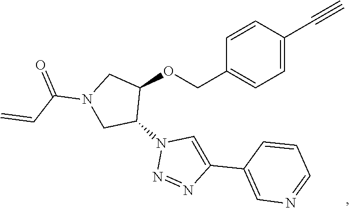 [SMILES: C#Cc1ccc(CO[C@@H]2CN(C(=O)C=C)C[C@H]2n2cc(-c3cccnc3)nn2)cc1]

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (IV):

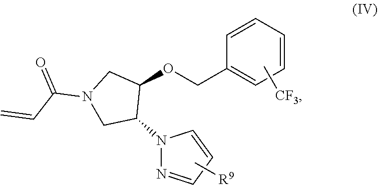

or a pharmaceutically acceptable salt, solvate, hydrate, polymorph, co-crystal, tautomer, stereoisomer, isotopically labeled derivative, prodrug, composition, or mixture thereof, wherein:
R⁹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{d}, —N(R^{d})₂, —SR^{d}, —C(═O)R^{d}, —C(═O)OR^{d}, —C(═O)N(R^{d})₂, —OC(═O)R^{d}, —N(R^{d})C(═O)R^{d}, —CN, or —NO₂;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{d}, when present, can be joined together with the heteroatom to which they are attached to form an optionally substituted heterocyclic ring.

Exemplary compounds of Formula (IV), include, but are not limited to:

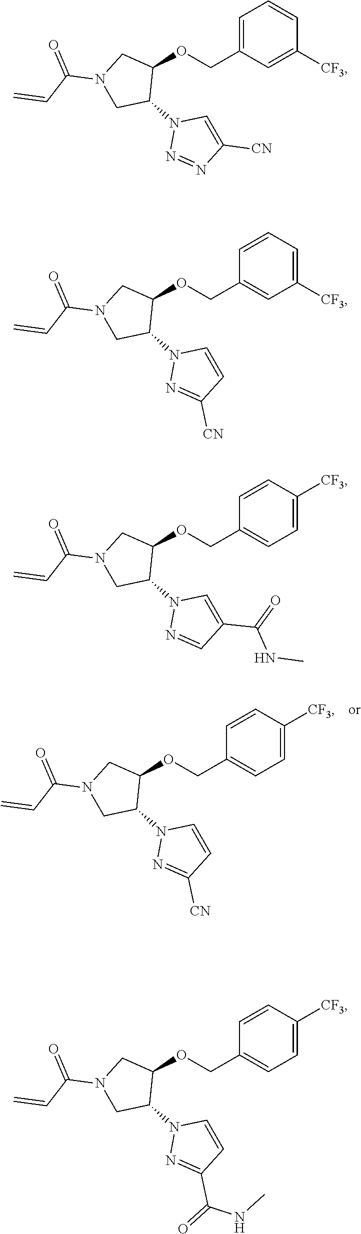 [SMILES: C=CC(=O)N1C[C@@H](n2cc(C#N)nn2)[C@H](OCc2cccc(C(F)(F)F)c2)C1 ; C=CC(=O)N1C[C@@H](n2ccc(C#N)n2)[C@H](OCc2cccc(C(F)(F)F)c2)C1 ; C=CC(=O)N1C[C@@H](n2cc(C(=O)NC)cn2)[C@H](OCc2ccc(C(F)(F)F)cc2)C1 ; C=CC(=O)N1C[C@@H](n2ccc(C#N)n2)[C@H](OCc2ccc(C(F)(F)F)cc2)C1 ; C=CC(=O)N1C[C@@H](n2ccc(C(=O)NC)n2)[C@H](OCc2ccc(C(F)(F)F)cc2)C1]

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (V):

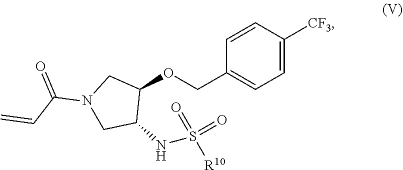

or a pharmaceutically acceptable salt, solvate, hydrate, polymorph, co-crystal, tautomer, stereoisomer, isotopically labeled derivative, prodrug, composition, or mixture thereof, wherein:
R¹⁰ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{e}, —N(R^{e})₂, —SR^{e}, —C(═O)R^{e}, —C(═O)OR^{e}, —C(═O)N(R^{e})₂, —OC(═O)R^{e}, —N(R^{e})C(═O)R^{e}, —CN, or —NO₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{e}, when present, can be joined together with the heteroatom to which they are attached to form an optionally substituted heterocyclic ring.

Exemplary compounds of Formula (V), include, but are not limited to:

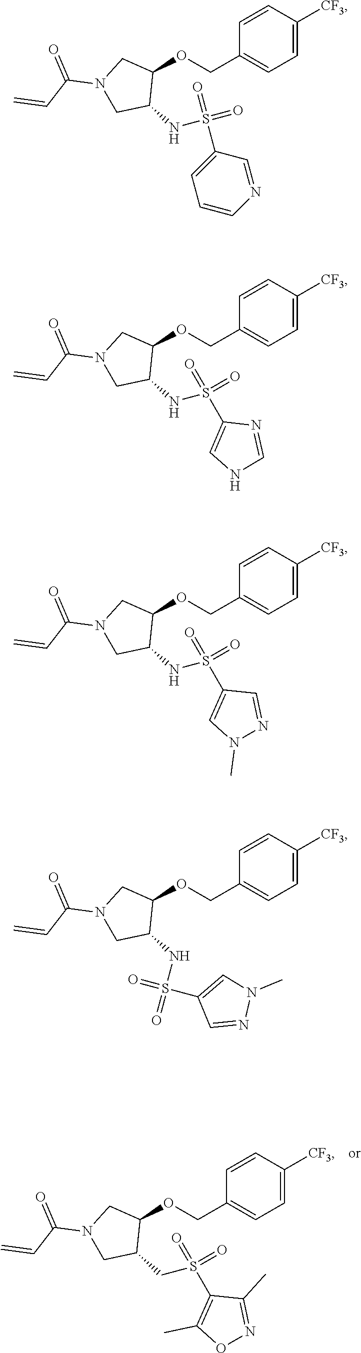 [SMILES: C=CC(=O)N1C[C@@H](NS(=O)(=O)c2cccnc2)[C@H](OCc2ccc(C(F)(F)F)cc2)C1 ; C=CC(=O)N1C[C@@H](NS(=O)(=O)c2c[nH]cn2)[C@H](OCc2ccc(C(F)(F)F)cc2)C1 ; C=CC(=O)N1C[C@@H](NS(=O)(=O)c2cnn(C)c2)[C@H](OCc2ccc(C(F)(F)F)cc2)C1 ; C=CC(=O)N1C[C@@H](NS(=O)(=O)c2cnn(C)c2)[C@H](OCc2ccc(C(F)(F)F)cc2)C1 ; C=CC(=O)N1C[C@@H](CS(=O)(=O)c2c(C)noc2C)[C@H](OCc2ccc(C(F)(F)F)cc2)C1]

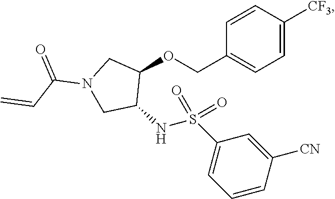 [SMILES: C=CC(=O)N1C[C@@H](NS(=O)(=O)c2cccc(C#N)c2)[C@H](OCc2ccc(C(F)(F)F)cc2)C1]

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (VI):

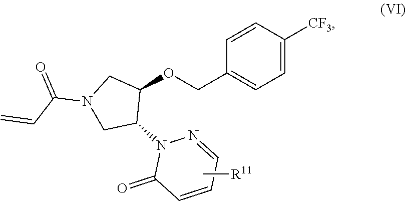

or a pharmaceutically acceptable salt, solvate, hydrate, polymorph, co-crystal, tautomer, stereoisomer, isotopically labeled derivative, prodrug, composition, or mixture thereof, wherein:
R¹¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{f}, —N(R^{f})₂, —SR^{f}, —C(═O)R^{f}, —C(═O)OR^{f}, —C(═O)N(R^{f})₂, —OC(═O)R^{f}, —N(R^{f})C(═O)R^{f}, —CN, or —NO₂;
each occurrence of R^{f} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{f}, when present, can be joined together with the heteroatom to which they are attached to form an optionally substituted heterocyclic ring.

Exemplary compounds of Formula (VI), include, but are not limited to:

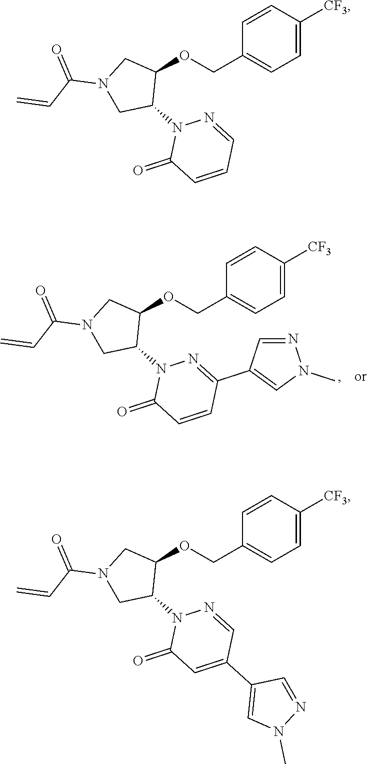

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (VII):

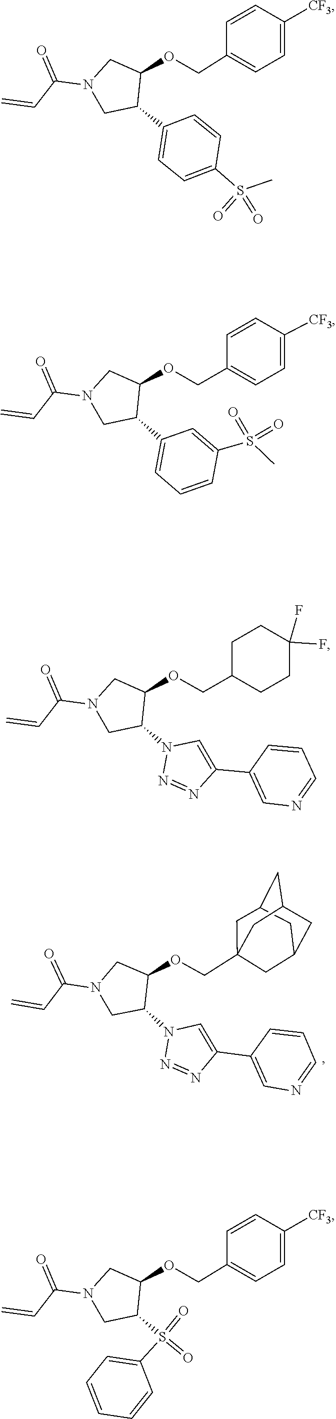 [SMILES: C=CC(=O)N1C[C@@H](OCc2ccc(C(F)(F)F)cc2)[C@H](c2ccc(S(C)(=O)=O)cc2)C1 ; C=CC(=O)N1C[C@@H](OCc2ccc(C(F)(F)F)cc2)[C@H](c2cccc(S(C)(=O)=O)c2)C1 ; C=CC(=O)N1C[C@@H](n2cc(-c3cccnc3)nn2)[C@H](OCC2CCC(F)(F)CC2)C1 ; C=CC(=O)N1C[C@@H](n2cc(-c3cccnc3)nn2)[C@H](OCC23CC4CC(CC(C4)C2)C3)C1 ; C=CC(=O)N1C[C@@H](S(=O)(=O)c2ccccc2)[C@H](OCc2ccc(C(F)(F)F)cc2)C1]

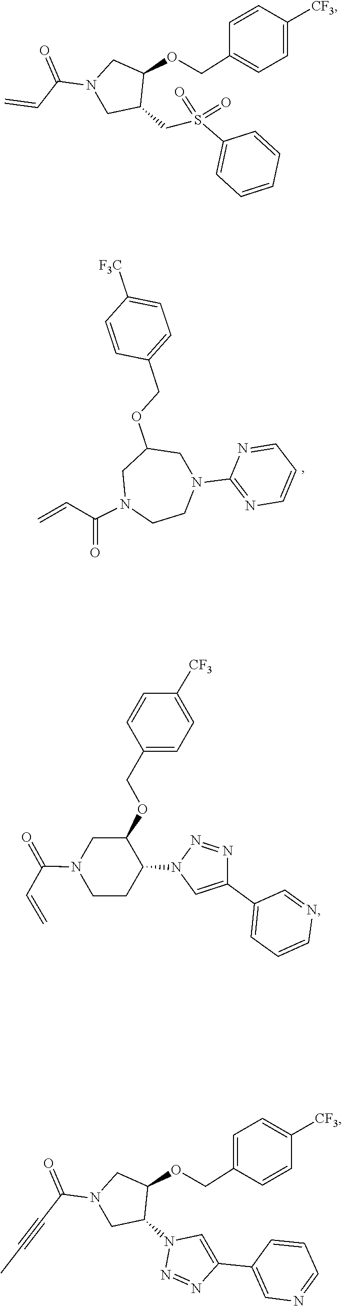 [SMILES: C=CC(=O)N1C[C@@H](CS(=O)(=O)c2ccccc2)[C@H](OCc2ccc(C(F)(F)F)cc2)C1 ; C=CC(=O)N1CCN(c2ncccn2)CC(OCc2ccc(C(F)(F)F)cc2)C1 ; C=CC(=O)N1CC[C@@H](n2cc(-c3cccnc3)nn2)[C@H](OCc2ccc(C(F)(F)F)cc2)C1 ; CC#CC(=O)N1C[C@@H](n2cc(-c3cccnc3)nn2)[C@H](OCc2ccc(C(F)(F)F)cc2)C1]

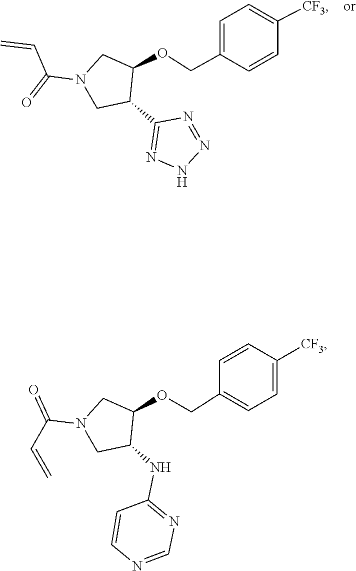

or a pharmaceutically acceptable salt, solvate, hydrate, polymorph, co-crystal, tautomer, stereoisomer, isotopically labeled derivative, prodrug, composition, or mixture thereof.

In one aspect, the present disclosure provides compounds of Formula (VIII):

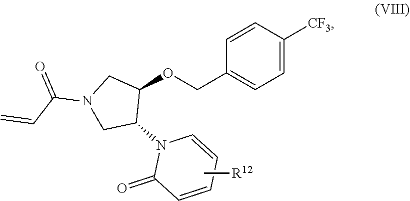

or a pharmaceutically acceptable salt, solvate, hydrate, polymorph, co-crystal, tautomer, stereoisomer, isotopically labeled derivative, prodrug, composition, or mixture thereof, wherein:
R¹² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{h}, —N(R^{h})₂, —SR^{h}, —C(═O)R^{h}, —C(═O)OR^{h}, —C(═O)N(R^{h})₂, —OC(═O)R^{h}, —N(R^{h})C(═O)R^{h}, —CN, or —NO₂;
each occurrence of R^{h} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{h}, when present, can be joined together with the heteroatom to which they are attached to form an optionally substituted heterocyclic ring.

Exemplary compounds of Formula (VIII), include, but are not limited to:

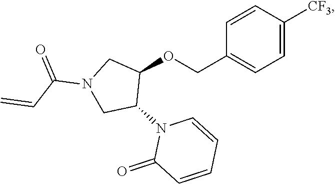 [SMILES: C=CC(=O)N1C[C@@H](n2ccccc2=O)[C@H](OCc2ccc(C(F)(F)F)cc2)C1]

or a pharmaceutically acceptable salt, solvate, hydrate, polymorph, co-crystal, tautomer, stereoisomer, isotopically labeled derivative, prodrug, composition, or mixture thereof.

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating and/or preventing a disease (e.g., a proliferative disease) in a subject in need thereof. The pharmaceutical composition may be useful for inhibiting the activity of TEAD family transcription factor (e.g., TEAD1, TEAD2, TEAD3, or TEAD4) in a subject, biological sample, tissue, or cell. The compounds described herein may be useful in treating and/or preventing a disease or condition, e.g., a proliferative disease (e.g., cancers (e.g., sarcoma, carcinoma, lung cancer, thyroid cancer, skin cancer, ovarian cancer, colorectal cancer, prostate cancer, pancreatic cancer, esophageal cancer, liver cancer, breast cancer)).

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating a disease (e.g., a proliferative disease) in a subject in need thereof, or inhibiting the activity of a TEAD family transcription factor (e.g., TEAD1, TEAD2, TEAD3, or TEAD4) in a subject, biological sample, tissue, or cell. In certain embodiments, the disease is a proliferative disease (e.g., cancer (e.g., carcinoma, sarcoma, carcinoma, lung cancer, thyroid cancer, skin cancer, ovarian cancer, colorectal cancer, prostate cancer, pancreatic cancer, esophageal cancer, liver cancer, breast cancer)).

In another aspect, described herein are methods for treating and/or preventing a disease (e.g., a proliferative disease). Exemplary proliferative diseases which may be treated include diseases associated with the overexpression, unwanted, or increased activity of a TEAD transcription factor (e.g., cancer (e.g., carcinoma, sarcoma, lung cancer, thyroid cancer, skin cancer, ovarian cancer, colorectal cancer, prostate cancer, pancreatic cancer, esophageal cancer, liver cancer, breast cancer)).

Another aspect relates to methods of inhibiting the activity of a transcription factor (e.g., TEAD (e.g., TEAD1, TEAD2, TEAD3, or TEAD4)) using a compound described herein in a biological sample (e.g., cell, tissue). In another aspect, described herein are methods of inhibiting the activity of a transcription factor (e.g., TEAD (e.g., TEAD1, TEAD2, TEAD3, or TEAD4)) using a compound described herein in a subject. In certain embodiments, the method involves the inhibition of TEAD1, TEAD2, TEAD3, or TEAD4.

Described herein are methods for treating cancer in a subject comprising administering to a subject in need thereof an effective amount of a compound, or pharmaceutical composition thereof, as described herein. In certain embodiments, a method described herein further includes administering to the subject an additional pharmaceutical agent. Also described herein are methods for inhibiting a cancer cell in a cell comprising contacting a cell with an effective amount of a compound, or pharmaceutical composition thereof, as described herein. In certain embodiments, a method described herein further includes contacting a cell with an additional pharmaceutical agent (e.g., an anti-proliferative agent).

In yet another aspect, the present disclosure provides compounds of Formula (I), Formula (II), Formula (III), Formula (IV), Formula (V), Formula (VI), and Formula (VII), and pharmaceutically acceptable salts, solvates, hydrates, polymorphs, co-crystals, tautomers, stereoisomers, isotopically labeled derivatives, prodrugs, compositions, and mixtures thereof, for use in the treatment of a disease (e.g., a proliferative disease, such as cancer) in a subject.

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g., prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diasteromers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, —Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₁₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C≡C double bond for which the stereochemistry is not specified (e.g., —CH═CHCH₃ or

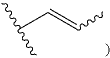

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thienyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[c][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 □ electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. In certain embodiments, the heteroaryl group is thiophene, benzothiophene, furan, isobenzofuran, pyrrole, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, triazole, tetrazole, oxazole, isoxazole, thiazole, oxazole, or the like.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepanyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{a}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{a}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR_{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal Rad substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl. 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl) (C₁₋₆ alkyl), —N(OH) (C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH) OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S) SC₁₋₆ alkyl, —SC(═S) SC₁₋₆ alkyl, —P(═O) (OC₁₋₆ alkyl)₂, —P(═O)(C₁₋₆ alkyl)₂, —OP(═O)(C₁₋₆ alkyl)₂, —OP(═O) (OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two Rec groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo)fluoroenylmethyl carbamate, 2,7-di-t-4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumcoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate. [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di (4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethylencamine, N-ferrocenylmethylamino (Fcm), N-2-picolylamino N′-oxide, N-1,1-dimethylthiomethyleneamine, N-benzylidencamine, N-p-methoxybenzylideneamine, N-diphenylmethylencamine, N-[(2-pyridyl) mesityl]methyleneamine, N—(N′,N′-dimethylaminomethylene)amine, N,N′-isopropylidenediamine, N-p-nitrobenzylideneamine, N-salicylidencamine, N-5-chlorosalicylidencamine, N-(5-chloro-2-hydroxyphenyl)phenylmethylencamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfonamide (Nps), 2,4-dinitrobenzenesulfonamide, pentachlorobenzenesulfenamide, 2-nitro-4-methoxybenzenesulfonamide, triphenylmethylsulfenamide, and 3-nitropyridinesulfenamide (Npys).

In certain embodiments, the substituent present on an oxygen atom is an oxygen protecting group (also referred to herein as a “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis. T. W. Greene and P. G. M. Wuts, 3rd edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis (2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di (p-methoxyphenyl) phenylmethyl, tri (p-methoxyphenyl)methyl, 4-(4′-bromophenacyloxyphenyl)diphenylmethyl, 4,4′,4″-tris (4,5-dichlorophthalimidophenyl)methyl, 4,4′,4″-tris (levulinoyloxyphenyl)methyl, 4,4′,4″-tris (benzoyloxyphenyl)methyl, 3-(imidazol-1-yl)bis (4′,4″-dimethoxyphenyl)methyl, 1,1-bis (4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis (1,1-dimethylpropyl) phenoxyacetate, chlorodiphenylacetate, isobutyrate, monosuccinate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X₋, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)⁻). Exemplary counterions which may be multivalent include CO₃²⁻, HPO₄²⁻, PO₄³⁻, B₄O₇²⁻. SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6^{th} ed. (501-502). Exemplary leaving groups include, but are not limited to, halo (e.g., chloro, bromo, iodo) and activated substituted hydroxyl groups (e.g., —OC(═O)SR^{aa}. —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

The term “small molecule” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The “molecular weight” of a monovalent moiety —R is calculated by subtracting 1 from the molecular weight of the compound R—H. The “molecular weight” of a divalent moiety -L- is calculated by subtracting 2 from the molecular weight of the compound H-L-H.

A “hydrocarbon chain” refers to a substituted or unsubstituted divalent alkyl, alkenyl, or alkynyl group. A hydrocarbon chain includes (1) one or more chains of carbon atoms immediately between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes one chain atom C^{A}, one hydrogen atom on C^{A}, and non-chain substituent —(C^{B}H₂C^{C}H₃). The term “Cₓ hydrocarbon chain,” wherein x is a positive integer, refers to a hydrocarbon chain that includes x number of chain atom(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

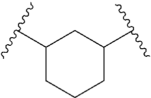 [SMILES: *C1CCCC(*)C1]

is a C₃ hydrocarbon chain. When a range of values is used, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of an unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

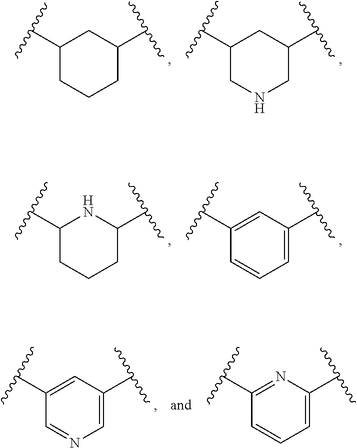 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments,

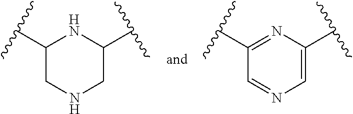 [SMILES: *C1CNCC(*)N1 ; *c1cncc(*)n1]

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

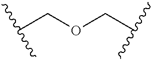 [SMILES: *COC*]

is a C₃ hydrocarbon chain wherein one chain atom is replaced with an oxygen atom.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diasteromers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers, respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering.” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibition of a TEAD transcription factor. In certain embodiments, a therapeutically effective amount is an amount sufficient for treating a proliferative disease.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibition of a TEAD transcription factor. In certain embodiments, a prophylactically effective amount is an amount sufficient for treating a proliferative disease.

As used herein the term “inhibit” or “inhibition” in the context of proteins, for example, in the context of TEAD transcription factors, refers to a reduction in the activity of the transcription factor. In some embodiments, the term refers to a reduction of the level of activity, e.g., TEAD1, TEAD2, TEAD3, or TEAD4 activity, to a level that is statistically significantly lower than an initial level, which may, for example, be a baseline level of activity. In some embodiments, the term refers to a reduction of the level of enzyme activity, e.g., TEAD1, TEAD2, TEAD3, or TEAD4 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of transcription factor activity.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. Sec, e.g., Stedman's Medical Dictionary, 25^{th} ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “transcription factor” is a type of protein that is involved in the process of transcribing DNA into RNA. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. These exemplary human transcription factors include, but are not limited to, AC008770.3. AC023509.3, AC092835.1. AC138696.1. ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1. CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLI1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERF1, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, and ZZZ3.

The term “inhibition,” “inhibiting.” “inhibit,” or “inhibitor” refer to the ability of a compound to reduce, slow, halt, or prevent activity of a particular biological process (e.g., a transcription factor) in a cell relative to vehicle.

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel, bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, raltitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbazine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

### BRIEF DESCRIPTION OF THE DRAWINGS

FIG. 1 shows a graph of the percent inhibition verse the log of the concentration of a representative compound of Formula (I).

FIG. 2 shows a graph of the percent inhibition verse the log of the concentration of representative compounds of Formula (II).

FIG. 3 shows a graph of the percent inhibition verse the log of the concentration of a representative compound of Formula (IV).

FIG. 4 shows a graph of the percent inhibition verse the log of the concentration of representative compounds of Formula (V).

FIG. 5 shows a graph of the percent inhibition verse the log of the concentration of representative compounds of Formula (VII).

FIG. 6 shows an antiproliferation assay on NCI-H226 cells (200 cells/well, 384 plate, 5-day treatment) for representative compounds of the disclosure.

FIG. 7 shows an antiproliferation assay on NCI-H226 cells (200 cells/well, 384 plate, 5-day treatment) for representative compounds of the disclosure.

FIGS. 8-13 show the results of a gel-based anti-palmitoylation assay for various compound of this disclosure.

### DETAILED DESCRIPTION OF CERTAIN EMBODIMENTS OF THE INVENTION

The present disclosure provides compounds of Formula (I′), Formula (I), Formula (II), Formula (III), Formula (IV), Formula (V), Formula (VI), Formula (VII), or Formula (VIII), or a pharmaceutically acceptable salt thereof, and optionally a pharmaceutically acceptable excipient. In certain embodiments, the pharmaceutical composition described herein comprises a compound of Formula (I′), Formula (I), Formula (II), Formula (III), Formula (IV), Formula (V), Formula (VI), Formula (VII), or Formula (VIII), or a pharmaceutically acceptable salt thereof, and a pharmaceutically acceptable excipient. The compounds and compositions as described herein may be useful for modulating (e.g., inhibiting or increasing) the activity (e.g., aberrant or undesired activity, such as increased or decreased activity) of a transcription factor (e.g., TEAD (e.g., TEAD1, TEAD2, TEAD3, or TEAD4)) in a subject, biological sample, tissue, or cell. The compounds may also be useful for the treatment of a wide range of diseases, such as diseases associated with the aberrant activity (e.g., increased activity) of a transcription factor (e.g., TEAD (e.g., TEAD1, TEAD2, TEAD3, or TEAD4)), e.g., proliferative diseases (e.g., cancers (e.g., carcinoma, sarcoma, lung cancer, thyroid cancer, skin cancer, ovarian cancer, colorectal cancer, prostate cancer, pancreatic cancer, esophageal cancer, liver cancer, breast cancer)).

### Compounds

In certain embodiments, a compound described herein is of Formula (I′):

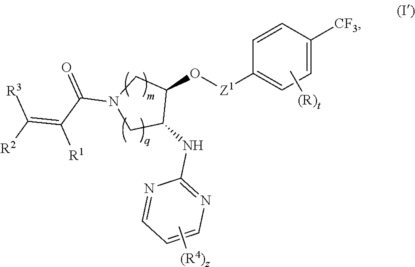

or a pharmaceutically acceptable salt, solvate, hydrate, polymorph, co-crystal, tautomer, stereoisomer, isotopically labeled derivative, prodrug, composition, or mixture thereof, wherein:
R, and R⁴ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
Z¹ is optionally substituted alkylene, optionally substituted alkenylene, optionally substituted alkynylene, optionally substituted carbocyclylene, optionally substituted heterocyclylene, optionally substituted arylene, or optionally substituted heteroarylene;
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{a}, when present, can be joined together with the heteroatom to which they are attached to form an optionally substituted heterocyclic ring
m is 1 or 2;
q is 1 or 2;
tis 0, 1, 2, 3, or 4;
z is 0, 1, 2, or 3.

In certain embodiments, the compound Formula (I′) is of the formula:

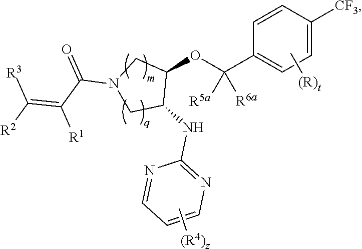

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

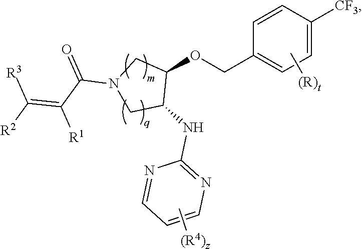

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

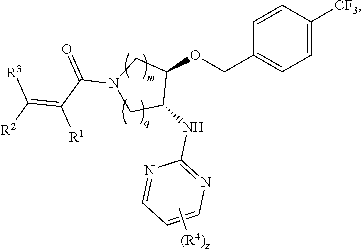

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

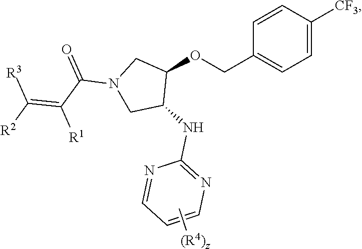

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

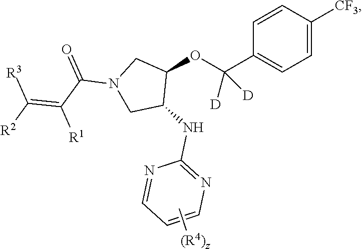

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

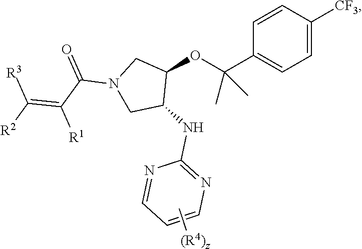

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

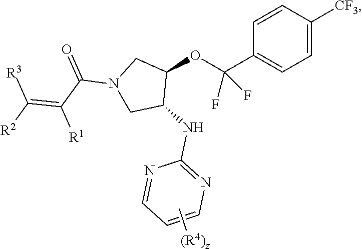

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

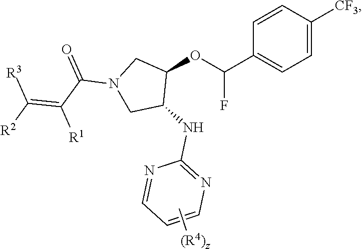

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

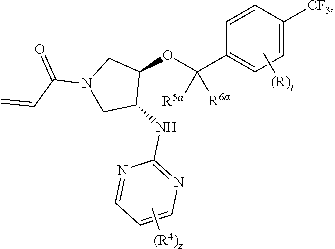

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

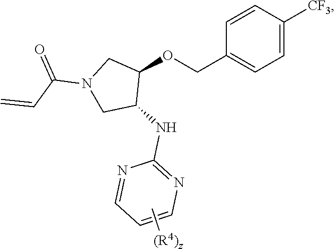

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

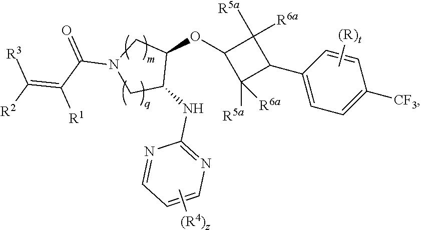

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

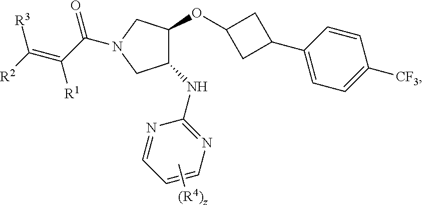

or a pharmaceutically acceptable salt, solvate, hydrate, polymorph, co-crystal, tautomer, stereoisomer, isotopically labeled derivative, prodrug, composition, or mixture thereof.

In certain embodiments, the compound Formula (I′) is of the formula:

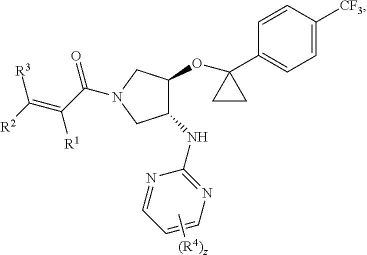

or a pharmaceutically acceptable salt, solvate, hydrate, polymorph, co-crystal, tautomer, stereoisomer, isotopically labeled derivative, prodrug, composition, or mixture thereof.

The compound of Formula (I′) contains the substituent Z¹. In certain embodiments, Z¹ is optionally substituted carbocyclylene. In certain embodiments, Z¹ is of the formula:

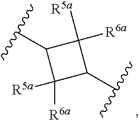

wherein R^{5a} and R^{6a} are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, and —N(R^{a})C(═O)R^{a}; or optionally, R^{5a} and R^{6a} are joined together to form a carbocyclic or heterocyclic ring. In certain embodiments, Z¹ is optionally substituted alkylene. In certain embodiments, Z¹ is optionally substituted C₁-C₆ alkylene. In certain embodiments, Z¹ is of the formula:

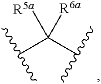

wherein R^{5a} and R^{6a} are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, and —N(R^{a})C(═O)R^{a}; or optionally, R^{5a} and R^{6a} are joined together to form a carbocyclic or heterocyclic ring. In certain embodiments, Z¹ is of the formula:

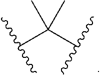 [SMILES: *C(*)(C)C]

In certain embodiments, Z¹ is of the formula:

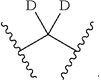 [SMILES: *C(*)([2H])[2H]]

In certain embodiments, Z¹ is of the formula:

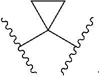 [SMILES: *C1(*)CC1]

In certain embodiments, Z¹ is of the formula:

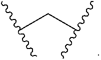 [SMILES: *C*]

In certain embodiments, the compound of Formula (I′) is of the formula:

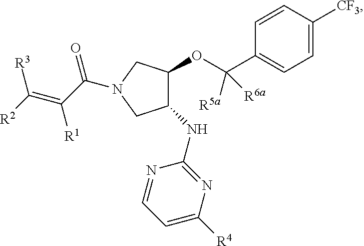

or a pharmaceutically acceptable salt, solvate, hydrate, polymorph, co-crystal, tautomer, stereoisomer, isotopically labeled derivative, or prodrug thereof.

In certain embodiments, the compound of Formula (I′) is of the formula:

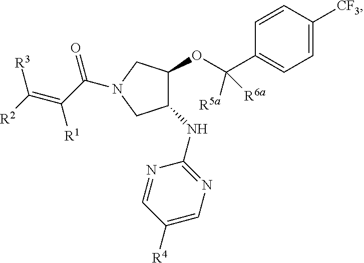

or a pharmaceutically acceptable salt, solvate, hydrate, polymorph, co-crystal, tautomer, stereoisomer, isotopically labeled derivative, or prodrug thereof.

In certain embodiments, the substituent Z¹ contains the substituents R^{5a} and R^{6a}. In certain embodiments, R^{5a} and R^{6a} are each independently selected from hydrogen, halogen, and optionally substituted alkyl; or optionally, R^{5a} and R^{6a} are joined together to form a carbocyclic or heterocyclic ring. In certain embodiments, R^{5a} is hydrogen. In certain embodiments, R^{5a} is halogen. In certain embodiments, R^{5a} is optionally substituted alkyl. In certain embodiments, R^{5a} is optionally substituted methyl. In certain embodiments, R^{5a} is optionally substituted C₁-C₆ alkyl. In certain embodiments, R^{5a} is optionally substituted methyl. In certain embodiments, R^{5a} is unsubstituted methyl. In certain embodiments, R^{5a} is optionally substituted alkenyl. In certain embodiments, R^{5a} is optionally substituted alkynyl. In certain embodiments, R^{5a} is optionally substituted carbocyclyl. In certain embodiments, R^{5a} is optionally substituted heterocyclyl. In certain embodiments, R^{5a} is optionally substituted aryl. In certain embodiments, R^{5a} is optionally substituted heteroaryl. In certain embodiments, R^{5a} is —OR^{a}. In certain embodiments, R^{5a} is —N(R^{a})₂. In certain embodiments, R^{5a} is —SR^{a}. In certain embodiments, R^{5a} is —C(═O)R^{a}. In certain embodiments, R^{5a} is —C(═O)OR^{a}. In certain embodiments. R^{5a} is —C(═O)N(R^{a})₂. In certain embodiments, R^{5a} is —OC(═O)R^{a}. In certain embodiments, R^{5a} is —N(R^{a})C(═O)R^{a}.

In certain embodiments, R^{6a} is hydrogen. In certain embodiments, R^{6a} is halogen. In certain embodiments, R^{6a} is optionally substituted alkyl. In certain embodiments, R^{6a} is optionally substituted C₁-C₆ alkyl. In certain embodiments, R^{6a} is optionally substituted methyl. In certain embodiments, R^{6a} is unsubstituted methyl. In certain embodiments, R^{6a} is optionally substituted alkenyl. In certain embodiments, R^{6a} is optionally substituted alkynyl. In certain embodiments. R^{6a} is optionally substituted carbocyclyl. In certain embodiments, R^{6a} is optionally substituted heterocyclyl. In certain embodiments, R^{6a} is optionally substituted aryl. In certain embodiments, R^{6a} is optionally substituted heteroaryl. In certain embodiments, R^{6a} is —OR^{a}. In certain embodiments, R^{6a} is —N(R^{a})₂. In certain embodiments, R^{6a} is —SR^{a}. In certain embodiments, R^{6a} is —C(═O)R^{a}. In certain embodiments, R^{6a} is —C(═O)OR^{a}. In certain embodiments, R^{6a} is —C(═O)N(R^{a})₂. In certain embodiments, R^{6a} is —OC(═O)R^{a}. In certain embodiments. R^{6a} is —N(R^{a})C(═O)R^{a}.

In certain embodiments, R^{5a} is hydrogen, and R^{6a} is hydrogen. In certain embodiments, R^{5a} is optionally substituted alkyl, and R^{6a} is optionally substituted alkyl. In certain embodiments, R^{5a} is methyl, and R^{6a} is methyl. In certain embodiments, R^{5a} is halogen, and R^{6a} is halogen. In certain embodiments, R^{5a} is fluoro, and R^{6a} is fluoro. In certain embodiments, R^{5a} is hydrogen, and R^{6a} is fluoro. In certain embodiments, R^{5a} and R^{6a} are joined together to form an optionally substituted carbocyclic ring. In certain embodiments. R^{5a} and R^{6a} are joined together to form an optionally substituted cyclopropyl ring. In certain embodiments, R^{5a} and R^{6a} are joined together to form an optionally substituted heterocyclic ring.

In certain embodiments, a compound described herein is of Formula (I):

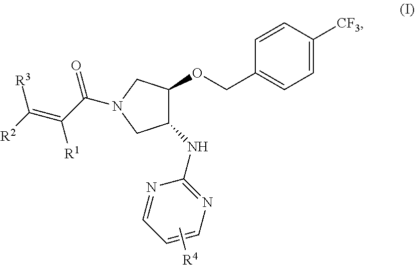

or a pharmaceutically acceptable salt, solvate, hydrate, polymorph, co-crystal, tautomer, stereoisomer, isotopically labeled derivative, prodrug, composition, or mixture thereof, wherein:
R¹, R², R³, and R⁴ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂
each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{a}, when present, can be joined together with the heteroatom to which they are attached to form an optionally substituted heterocyclic ring.

In certain embodiments, the compound of Formula (I) is of the formula:

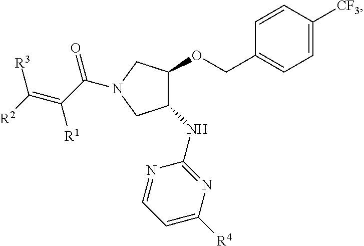

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (I) is of the formula:

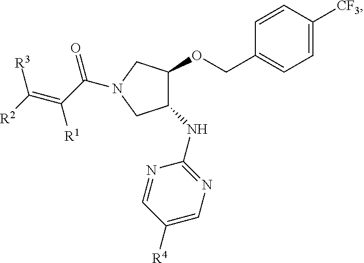

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (I) is of the formula:

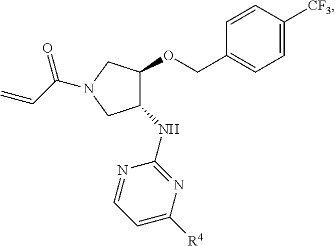

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (I) is of the formula:

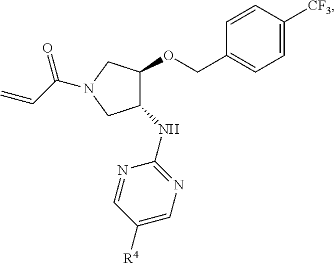

or a pharmaceutically acceptable salt, solvate, hydrate, polymorph, co-crystal, tautomer, stereoisomer, isotopically labeled derivative, prodrug, composition, or mixture thereof.

In certain embodiments, R¹, R², and R³, are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂.

In certain embodiments, R⁴ is each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂.

In certain embodiments. R is hydrogen. In certain embodiments, R is halogen. In certain embodiments, R is fluoro. In certain embodiments, R is optionally substituted alkyl (e.g., -Me, -Et, -i-Pr). In certain embodiments, R is haloalkyl. In certain embodiments, R is optionally substituted —CF₃. In certain embodiments, R is optionally substituted alkenyl. In certain embodiments. R is optionally substituted alkynyl. In certain embodiments, R is optionally substituted carbocyclyl. In certain embodiments, R is optionally substituted heterocyclyl. In certain embodiments. R is optionally substituted aryl. In certain embodiments, R is optionally substituted heteroaryl. In certain embodiments, R is —OR^{a}. In certain embodiments, R is —N(R^{a})₂. In certain embodiments, R is —SR^{a}. In certain embodiments, R is —C(═O)R^{a}. In certain embodiments, R is —C(═O)OR^{a}. In certain embodiments, R is —C(═O)N(R^{a})₂. In certain embodiments, R is —OC(═O)R^{a}. In certain embodiments, R¹ is —N(R^{a})C(═O)R^{a}. In certain embodiments, R is —CN. In certain embodiments, R is —NO₂.

In certain embodiments, R¹ is hydrogen. In certain embodiments, R¹ is halogen. In certain embodiments, R¹ is fluoro. In certain embodiments, R¹ is optionally substituted alkyl (e.g., -Me, -Et, -i-Pr). In certain embodiments, R¹ is haloalkyl. In certain embodiments, R¹ is optionally substituted —CF₃. In certain embodiments, R¹ is optionally substituted alkenyl. In certain embodiments, R¹ is optionally substituted alkynyl. In certain embodiments, R¹ is optionally substituted carbocyclyl. In certain embodiments, R¹ is optionally substituted heterocyclyl. In certain embodiments. R¹ is optionally substituted aryl. In certain embodiments, R¹ is optionally substituted heteroaryl. In certain embodiments, R¹ is —OR^{a}. In certain embodiments, R¹ is —N(R^{a})₂. In certain embodiments, R¹ is —SR^{a}. In certain embodiments, R¹ is —C(═O)R^{a}. In certain embodiments, R′ is —C(═O)OR^{a}. In certain embodiments, R¹ is —C(═O)N(R^{a})₂. In certain embodiments, R¹ is —OC(═O)R^{a}. In certain embodiments, R¹ is —N(R^{a})C(═O)R^{a}. In certain embodiments, R¹ is —CN. In certain embodiments, R¹ is —NO₂.

In certain embodiments, R² is hydrogen. In certain embodiments, R² is halogen. In certain embodiments, R² is optionally substituted alkyl (e.g., -Me, -Et, -i-Pr). In certain embodiments, R² is optionally substituted alkenyl. In certain embodiments, R² is optionally substituted alkynyl. In certain embodiments, R² is optionally substituted carbocyclyl. In certain embodiments, R² is optionally substituted heterocyclyl. In certain embodiments, R² is optionally substituted aryl. In certain embodiments, R² is optionally substituted heteroaryl. In certain embodiments, R² is —OR^{a}. In certain embodiments, R² is —N(R^{a})₂. In certain embodiments, R² is —SR^{a}. In certain embodiments, R² is —C(═O)R^{a}. In certain embodiments, R² is —C(═O)OR^{a}. In certain embodiments, R² is —C(═O)N(R^{a})₂. In certain embodiments, R² is —OC(═O)R^{a}. In certain embodiments, R² is —N(R^{a})C(═O)R^{a}. In certain embodiments, R² is —CN. In certain embodiments, R² is —NO₂. In certain embodiments, R² is of the formula:

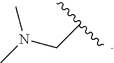 [SMILES: *CN(C)C]

In certain embodiments, R² is of the formula:

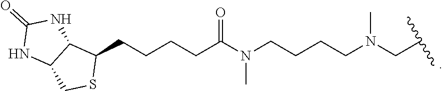 [SMILES: *CN(C)CCCCN(C)C(=O)CCCC[C@H]1SC[C@@H]2NC(=O)N[C@@H]21]

In certain embodiments, R² is of the formula:

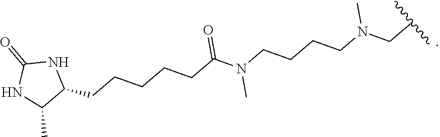 [SMILES: *CN(C)CCCCN(C)C(=O)CCCCC[C@H]1NC(=O)N[C@H]1C]

In certain embodiments, R² is of the formula:

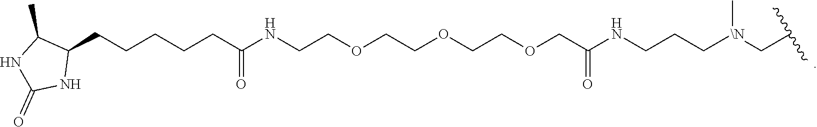

In certain embodiments, R³ is hydrogen. In certain embodiments, R³ is halogen. In certain embodiments, R³ is optionally substituted alkyl (e.g., -Me, -Et, -i-Pr). In certain embodiments, R³ is optionally substituted alkenyl. In certain embodiments, R³ is optionally substituted alkynyl. In certain embodiments, R³ is optionally substituted carbocyclyl. In certain embodiments, R³ is optionally substituted heterocyclyl. In certain embodiments, R³ is optionally substituted aryl. In certain embodiments, R³ is optionally substituted heteroaryl. In certain embodiments, R³ is —OR^{a}. In certain embodiments, R³ is —N(R^{a})₂. In certain embodiments, R³ is —SR^{a}. In certain embodiments, R³ is —C(═O)R^{a}. In certain embodiments, R³ is —C(═O)OR^{a}. In certain embodiments, R³ is —C(═O)N(R^{a})₂. In certain embodiments, R³ is —OC(═O)R^{a}. In certain embodiments, R³ is —N(R^{a})C(═O)R^{a}. In certain embodiments, R³ is —CN. In certain embodiments, R³ is —NO₂.

In certain embodiments, R¹ is H, R² is H, and R³ is H. In certain embodiments, R¹ is halogen, R² is H, and R³ is H. In certain embodiments, R¹ is fluoro, R² is H, and R³ is H. In certain embodiments, R¹ is H, R² is of the formula:

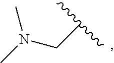 [SMILES: *CN(C)C]

and R³ is H. In certain embodiments, R¹ is H, R² is of the formula:

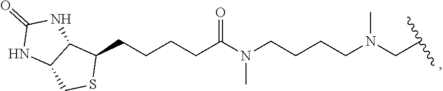 [SMILES: *CN(C)CCCCN(C)C(=O)CCCC[C@H]1SC[C@@H]2NC(=O)N[C@@H]21]

and R³ is H. In certain embodiments, R¹ is H, R² is of the formula:

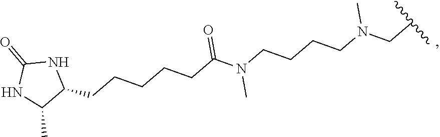 [SMILES: *CN(C)CCCCN(C)C(=O)CCCCC[C@H]1NC(=O)N[C@H]1C]

and R³ is H. In certain embodiments, R¹ is H, R² is of the formula:

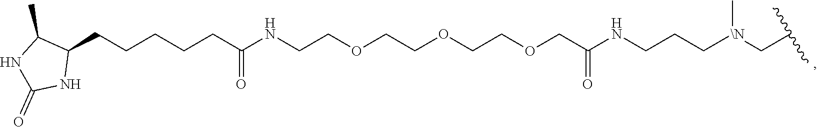

### and R³ is H.

In certain embodiments, R⁴ is hydrogen. In certain embodiments, R⁴ is halogen. In certain embodiments, R⁴ is fluoro. In certain embodiments, R⁴ is halogen. In certain embodiments, R⁴ is optionally substituted alkyl (e.g., -Me, -Et, -i-Pr). In certain embodiments, R⁴ is optionally substituted alkenyl. In certain embodiments, R⁴ is optionally substituted alkynyl. In certain embodiments, R⁴ is optionally substituted carbocyclyl. In certain embodiments, R⁴ is optionally substituted heterocyclyl. In certain embodiments, R⁴ is

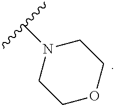 [SMILES: *N1CCOCC1]

In certain embodiments, R⁴ is

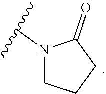 [SMILES: *N1CCCC1=O]

In certain embodiments, R⁴ is

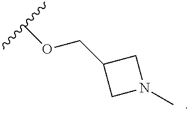 [SMILES: *OCC1CN(C)C1]

In certain embodiments, R⁴ is optionally substituted aryl. In certain embodiments, R⁴ is optionally substituted heteroaryl. In certain embodiments, R⁴ is optionally substituted pyrrolidine. In certain embodiments, R⁴ is optionally substituted

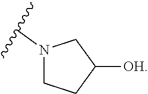 [SMILES: *N1CCC(O)C1]

In certain embodiments, R⁴ is —OR^{a}. In certain embodiments, R⁴ is —OMe. In certain embodiments, R⁴ is

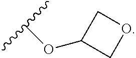 [SMILES: *OC1COC1]

In certain embodiments, R⁴ is —N(R^{a})₂. In certain embodiments, R⁴ is —SR^{a}. In certain embodiments, R⁴ is —C(═O)R^{a}. In certain embodiments, R⁴ is —C(═O)OR^{a}. In certain embodiments, R⁴ is —C(═O)N(R^{a})₂. In certain embodiments, R⁴ is —OC(═O)R^{a}. In certain embodiments, R⁴ is —N(R^{a})C(═O)R^{a}. In certain embodiments, R⁴ is —CN. In certain embodiments, R⁴ is —NO₂.

The substituents R¹, R², R³, and R⁴ contain the substituent R^{a}. In certain embodiments, R^{a} is hydrogen. In certain embodiments, R^{a} is optionally substituted alkyl (e.g., -Me, -Et, -i-Pr). In certain embodiments, R^{a} is -Me. In certain embodiments, R^{a} is optionally substituted alkenyl. In certain embodiments, R^{a} is optionally substituted alkynyl. In certain embodiments, R^{a} is optionally substituted carbocyclyl. In certain embodiments, R^{a} is optionally substituted heterocyclyl. In certain embodiments, R^{a} is optionally substituted aryl. In certain embodiments, R^{a} is optionally substituted heteroaryl. In certain embodiments, R^{a} is a nitrogen protecting group. In certain embodiments, two instances of R^{a}, when present, can be joined together with the heteroatom to which they are attached to form an optionally substituted heterocyclic ring.

In certain embodiments, m is 1. In certain embodiments, m is 2.

In certain embodiments, q is 1. In certain embodiments, q is 2.

In certain embodiments, t is 0. In certain embodiments, t is 1. In certain embodiments, t is 2. In certain embodiments, t is 3. In certain embodiments, t is 4.

In certain embodiments, z is 0. In certain embodiments, z is 1. In certain embodiments, z is 2. In certain embodiments, z is 3.

In certain embodiments, the compound of Formula (I) is of one of the formulae in Table 1 below:

TABLE 1
Compounds of Formula (I′)

| Compound No. | Chemical Structure |
|---|---|
| I-1 | 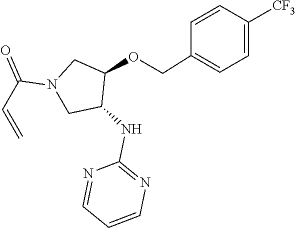 [SMILES: C=CC(=O)N1C[C@@H](Nc2ncccn2)[C@H](OCc2ccc(C(F)(F)F)cc2)C1] |
| I-2 | 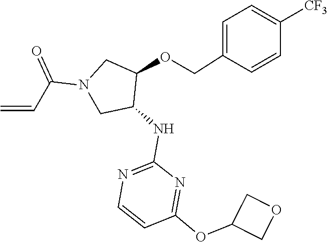 [SMILES: C=CC(=O)N1C[C@@H](Nc2nccc(OC3COC3)n2)[C@H](OCc2ccc(C(F)(F)F)cc2)C1] |
| I-3 | 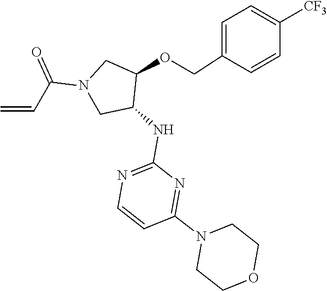 [SMILES: C=CC(=O)N1C[C@@H](Nc2nccc(N3CCOCC3)n2)[C@H](OCc2ccc(C(F)(F)F)cc2)C1] |
| I-4 | 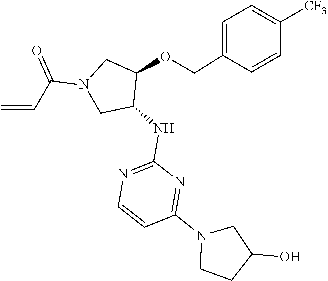 [SMILES: C=CC(=O)N1C[C@@H](Nc2nccc(N3CCC(O)C3)n2)[C@H](OCc2ccc(C(F)(F)F)cc2)C1] |
| I-5 | 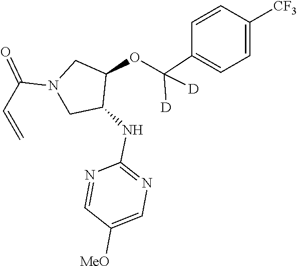 [SMILES: [2H]C([2H])(O[C@@H]1CN(C(=O)C=C)C[C@H]1Nc1ncc(OC)cn1)c1ccc(C(F)(F)F)cc1] |
| I-6 | 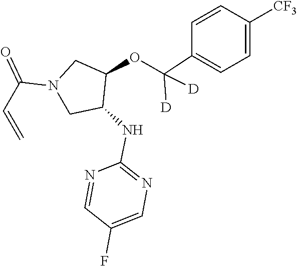 [SMILES: [2H]C([2H])(O[C@@H]1CN(C(=O)C=C)C[C@H]1Nc1ncc(F)cn1)c1ccc(C(F)(F)F)cc1] |
| I-7 | 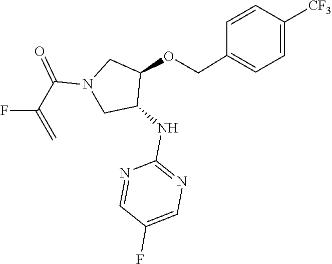 [SMILES: C=C(F)C(=O)N1C[C@@H](Nc2ncc(F)cn2)[C@H](OCc2ccc(C(F)(F)F)cc2)C1] |
| I-8 | 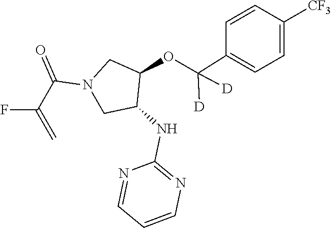 [SMILES: [2H]C([2H])(O[C@@H]1CN(C(=O)C(=C)F)C[C@H]1Nc1ncccn1)c1ccc(C(F)(F)F)cc1] |
| I-9 | 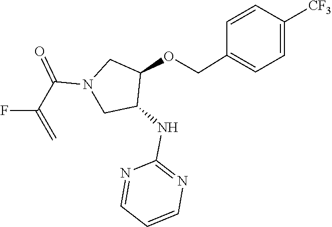 [SMILES: C=C(F)C(=O)N1C[C@@H](Nc2ncccn2)[C@H](OCc2ccc(C(F)(F)F)cc2)C1] |
| I-10 | 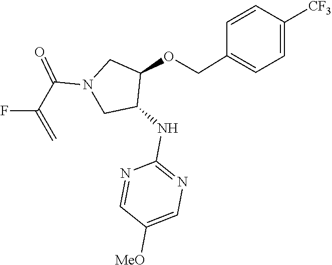 [SMILES: C=C(F)C(=O)N1C[C@@H](Nc2ncc(OC)cn2)[C@H](OCc2ccc(C(F)(F)F)cc2)C1] |
| I-11 | 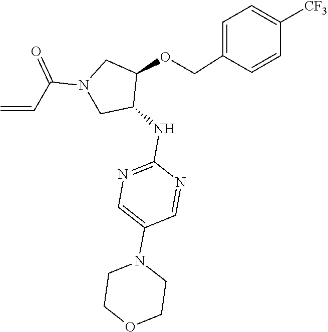 [SMILES: C=CC(=O)N1C[C@@H](Nc2ncc(N3CCOCC3)cn2)[C@H](OCc2ccc(C(F)(F)F)cc2)C1] |
| I-12 | 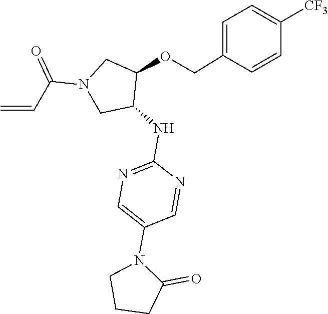 [SMILES: C=CC(=O)N1C[C@@H](Nc2ncc(N3CCCC3=O)cn2)[C@H](OCc2ccc(C(F)(F)F)cc2)C1] |
| I-13 | 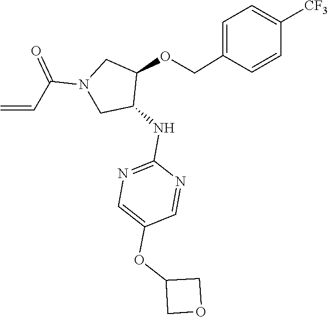 [SMILES: C=CC(=O)N1C[C@@H](Nc2ncc(OC3COC3)cn2)[C@H](OCc2ccc(C(F)(F)F)cc2)C1] |
| I-14 | 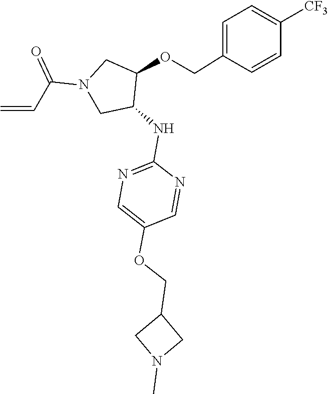 [SMILES: C=CC(=O)N1C[C@@H](Nc2ncc(OCC3CN(C)C3)cn2)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, the compound of Formula (I′) is of one of the formulae in Table 1A below:

TABLE 1A
Compounds of Formula (I′)

| | |
|---|---|
| I-15 | 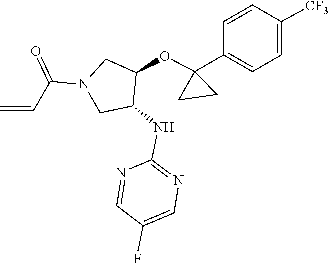 [SMILES: C=CC(=O)N1C[C@@H](Nc2ncc(F)cn2)[C@H](OC2(c3ccc(C(F)(F)F)cc3)CC2)C1] |
| I-16 | 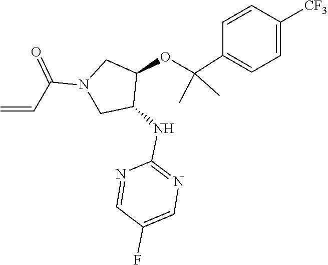 [SMILES: C=CC(=O)N1C[C@@H](Nc2ncc(F)cn2)[C@H](OC(C)(C)c2ccc(C(F)(F)F)cc2)C1] |
| I-17 | 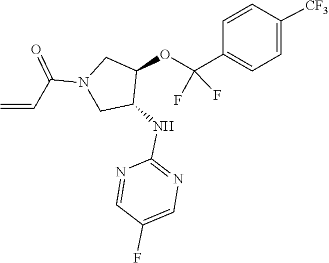 [SMILES: C=CC(=O)N1C[C@@H](Nc2ncc(F)cn2)[C@H](OC(F)(F)c2ccc(C(F)(F)F)cc2)C1] |
| I-18 | 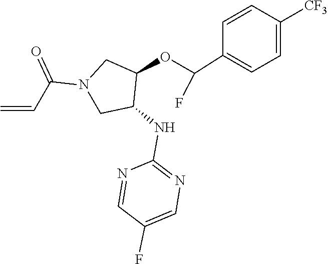 [SMILES: C=CC(=O)N1C[C@@H](Nc2ncc(F)cn2)[C@H](OC(F)c2ccc(C(F)(F)F)cc2)C1] |
| I-19 | 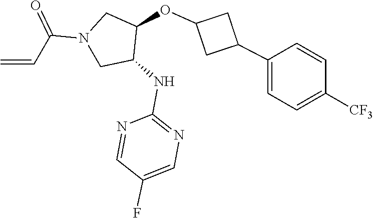 [SMILES: C=CC(=O)N1C[C@@H](Nc2ncc(F)cn2)[C@H](OC2CC(c3ccc(C(F)(F)F)cc3)C2)C1] |
| I-20 | 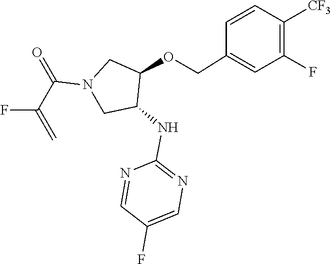 [SMILES: C=C(F)C(=O)N1C[C@@H](Nc2ncc(F)cn2)[C@H](OCc2ccc(C(F)(F)F)c(F)c2)C1] |
| I-21 | 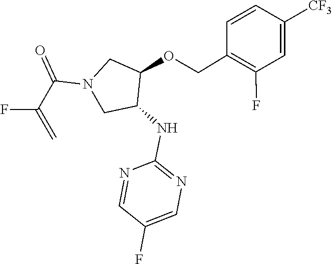 [SMILES: C=C(F)C(=O)N1C[C@@H](Nc2ncc(F)cn2)[C@H](OCc2ccc(C(F)(F)F)cc2F)C1] |
| I-22 | 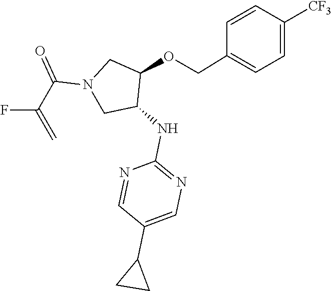 [SMILES: C=C(F)C(=O)N1C[C@@H](Nc2ncc(C3CC3)cn2)[C@H](OCc2ccc(C(F)(F)F)cc2)C1] |
| I-23 | 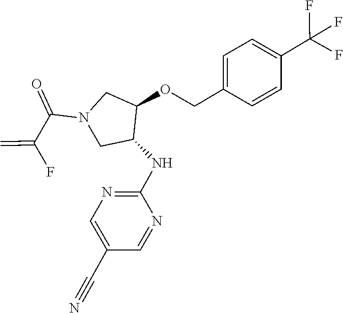 [SMILES: C=C(F)C(=O)N1C[C@@H](Nc2ncc(C#N)cn2)[C@H](OCc2ccc(C(F)(F)F)cc2)C1] |
| I-24 | 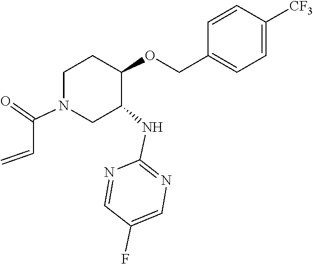 [SMILES: C=CC(=O)N1CC[C@@H](OCc2ccc(C(F)(F)F)cc2)[C@H](Nc2ncc(F)cn2)C1] |
| I-25 | 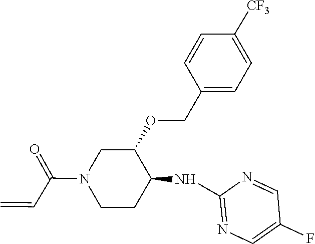 [SMILES: C=CC(=O)N1CC[C@H](Nc2ncc(F)cn2)[C@@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, the compound of Formula (I) is of the formula:

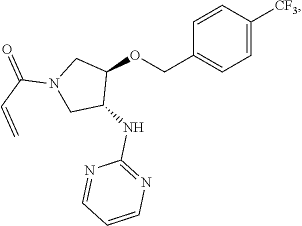 [SMILES: C=CC(=O)N1C[C@@H](Nc2ncccn2)[C@H](OCc2ccc(C(F)(F)F)cc2)C1]

or a pharmaceutically acceptable salt, solvate, hydrate, polymorph, co-crystal, tautomer, stereoisomer, isotopically labeled derivative, prodrug, composition, or mixture thereof. In certain embodiments, the compound of Formula (I) is of the formula

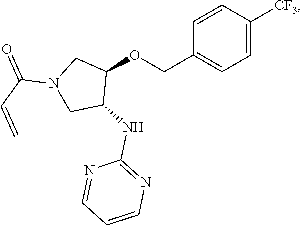 [SMILES: C=CC(=O)N1C[C@@H](Nc2ncccn2)[C@H](OCc2ccc(C(F)(F)F)cc2)C1]

or a pharmaceutically acceptable salt thereof.

In certain embodiments, a compound described herein is a compound of Formula (I), or a pharmaceutically acceptable salt, solvate, hydrate, polymorph, co-crystal, tautomer, stereoisomer, isotopically labeled derivative, prodrug, composition, or mixture thereof. In certain embodiments, a compound described herein is a compound of Formula (I), or a pharmaceutically acceptable salt thereof.

In certain embodiments, a compound described herein is of Formula (II):

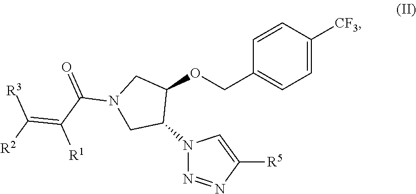

or a pharmaceutically acceptable salt, solvate, hydrate, polymorph, co-crystal, tautomer, stereoisomer, isotopically labeled derivative, prodrug, composition, or mixture thereof, wherein:
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}, —C(═O)R^{b}, —C(═O)OR^{b}, —C(═O)N(R^{b})₂, —OC(═O)R^{b}, —N(R^{b})C(═O)R^{b}, —CN, and —NO₂;
R⁵ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}, —C(═O)R^{b}, —C(═O)OR^{b}, —C(═O)N(R^{b})₂, —OC(═O)R^{b}, —N(R^{b})C(═O)R^{b}, —CN, and —NO₂;
each occurrence of R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{b}, when present, can be joined together with the heteroatom to which they are attached to form an optionally substituted heterocyclic ring
provided that the compound is not of the formula:

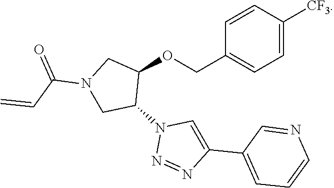 [SMILES: C=CC(=O)N1C[C@@H](n2cc(-c3cccnc3)nn2)[C@H](OCc2ccc(C(F)(F)F)cc2)C1]

In certain embodiments, R⁵ is hydrogen. In certain embodiments, R⁵ is halogen. In certain embodiments, R⁵ is optionally substituted alkyl (e.g., -Me, -Et, -i-Pr). In certain embodiments, R⁵ is optionally substituted C₁-C₆ alkyl. In certain embodiments, R⁵ is CH₂N(R^{b})₂. In certain embodiments, R⁵ is optionally substituted alkenyl. In certain embodiments, R⁵ is optionally substituted alkynyl. In certain embodiments, R⁵ is optionally substituted carbocyclyl. In certain embodiments, R⁵ is optionally substituted heterocyclyl. In certain embodiments, R⁵ is optionally substituted aryl. In certain embodiments, R⁵ is optionally substituted phenyl. In certain embodiments, R⁵ is monosubstituted phenyl. In certain embodiments, R⁵ is of the formula:

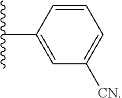 [SMILES: *c1cccc(C#N)c1]

In certain embodiments, R⁵ is of the formula:

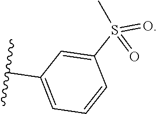 [SMILES: *c1cccc(S(C)(=O)=O)c1]

In certain embodiments, R⁵ is disubstituted phenyl. In certain embodiments, R⁵ is optionally substituted heteroaryl. In certain embodiments, R⁵ is optionally substituted pyridine. In certain embodiments, R⁵ is optionally substituted pyrimidine. In certain embodiments, R⁵ is optionally substituted pyridazine. In certain embodiments, R⁵ is optionally substituted pyrazine. In certain embodiments, R⁵ is

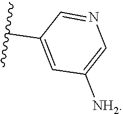 [SMILES: *c1cncc(N)c1]

In certain embodiments, R⁵ is

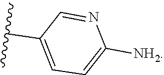 [SMILES: *c1ccc(N)nc1]

In certain embodiments, R⁵ is

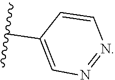 [SMILES: *c1ccnnc1]

In certain embodiments, R⁵ is

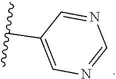 [SMILES: *c1cncnc1]

In certain embodiments, R⁵ is

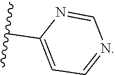 [SMILES: *c1ccncn1]

In certain embodiments, R⁵ is

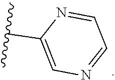 [SMILES: *c1cnccn1]

In certain embodiments, R⁵ is —OR^{b}. In certain embodiments, R⁵ is —N(R^{b})₂. In certain embodiments, R⁵ is —SR^{b}. In certain embodiments, R⁵ is —C(═O)R^{b}. In certain embodiments, R⁵ is —C(═O)OR^{b}. In certain embodiments, R⁵ is —C(═O)N(R^{b})₂. In certain embodiments, R⁵ is —C(═O)N(H)₂. In certain embodiments, R⁵ is —C(═O)N(H)Me. In certain embodiments, R⁵ is —C(═O)N(Me)₂. In certain embodiments, R⁵ is —OC(═O)R^{b}. In certain embodiments, R⁵ is —N(R^{b})C(═O)R^{b}. In certain embodiments, R⁵ is —CN. In certain embodiments, R⁵ is —NO₂.

In certain embodiments, at least one instance of R^{b} is hydrogen. In certain embodiments, two instances of R^{b} are hydrogen. In certain embodiments, one instance of R^{b} is hydrogen, and one instance of R^{b} is methyl. In certain embodiments, two instances of R^{b} are methyl. In certain embodiments, R^{b} is optionally substituted alkyl (e.g., -Me, -Et, -i-Pr). In certain embodiments, R^{b} is -Me. In certain embodiments, R^{b} is optionally substituted alkenyl. In certain embodiments, R^{b} is optionally substituted alkynyl. In certain embodiments, R^{b} is optionally substituted carbocyclyl. In certain embodiments, R^{b} is optionally substituted heterocyclyl. In certain embodiments, R^{b} is optionally substituted aryl. In certain embodiments, R^{b} is optionally substituted heteroaryl. In certain embodiments, R^{b} is a nitrogen protecting group. In certain embodiments, two instances of R^{b}, when present, can be joined together with the heteroatom to which they are attached to form an optionally substituted heterocyclic ring.

In certain embodiments, the compound of Formula (II) is not of the formula:

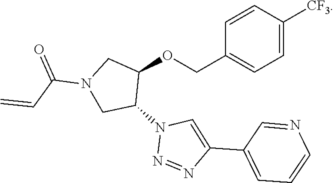 [SMILES: C=CC(=O)N1C[C@@H](n2cc(-c3cccnc3)nn2)[C@H](OCc2ccc(C(F)(F)F)cc2)C1]

In certain embodiments, the compound of Formula (II) is of the formula:

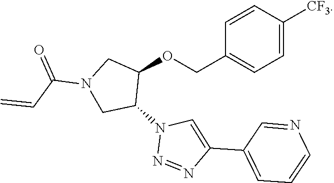 [SMILES: C=CC(=O)N1C[C@@H](n2cc(-c3cccnc3)nn2)[C@H](OCc2ccc(C(F)(F)F)cc2)C1]

In certain embodiments, the compound of Formula (II) is of the formula:

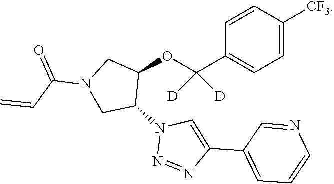 [SMILES: [2H]C([2H])(O[C@@H]1CN(C(=O)C=C)C[C@H]1n1cc(-c2cccnc2)nn1)c1ccc(C(F)(F)F)cc1]

In certain embodiments, the compound of Formula (II) is of one of the formulae in Table 2 below:

TABLE 2
Compounds of Formula (II)

| Com- | |
|---|---|
| pound | |
| No. | Chemical Structure |
| II-1 | 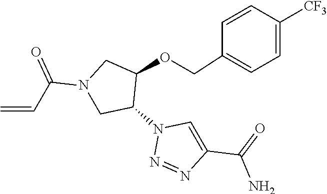 [SMILES: C=CC(=O)N1C[C@@H](n2cc(C(N)=O)nn2)[C@H](OCc2ccc(C(F)(F)F)cc2)C1] |
| II-2 | 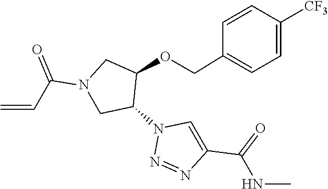 [SMILES: C=CC(=O)N1C[C@@H](n2cc(C(=O)NC)nn2)[C@H](OCc2ccc(C(F)(F)F)cc2)C1] |
| II-3 | 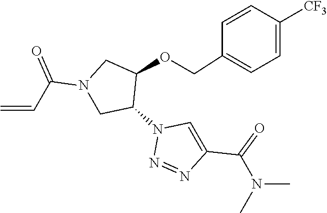 [SMILES: C=CC(=O)N1C[C@@H](n2cc(C(=O)N(C)C)nn2)[C@H](OCc2ccc(C(F)(F)F)cc2)C1] |
| II-4 | 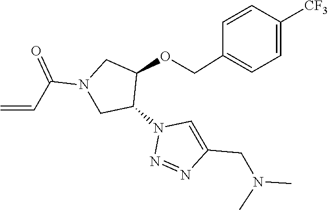 [SMILES: C=CC(=O)N1C[C@@H](n2cc(CN(C)C)nn2)[C@H](OCc2ccc(C(F)(F)F)cc2)C1] |
| II-5 | 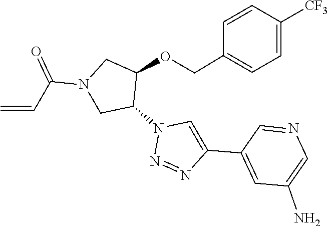 [SMILES: C=CC(=O)N1C[C@@H](n2cc(-c3cncc(N)c3)nn2)[C@H](OCc2ccc(C(F)(F)F)cc2)C1] |
| II-6 | 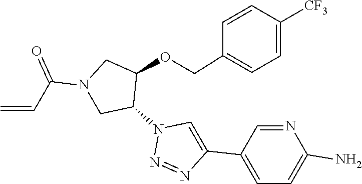 [SMILES: C=CC(=O)N1C[C@@H](n2cc(-c3ccc(N)nc3)nn2)[C@H](OCc2ccc(C(F)(F)F)cc2)C1] |
| II-7 | 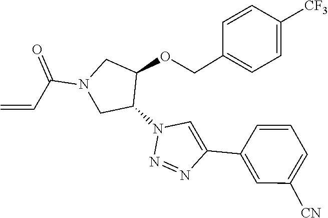 [SMILES: C=CC(=O)N1C[C@@H](n2cc(-c3cccc(C#N)c3)nn2)[C@H](OCc2ccc(C(F)(F)F)cc2)C1] |
| II-8 | 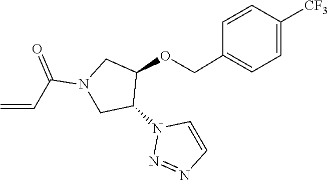 [SMILES: C=CC(=O)N1C[C@@H](n2ccnn2)[C@H](OCc2ccc(C(F)(F)F)cc2)C1] |
| II-9 | 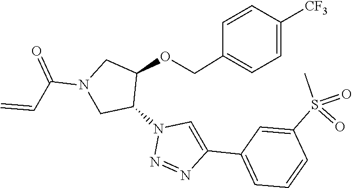 [SMILES: C=CC(=O)N1C[C@@H](n2cc(-c3cccc(S(C)(=O)=O)c3)nn2)[C@H](OCc2ccc(C(F)(F)F)cc2)C1] |
| II-10 | 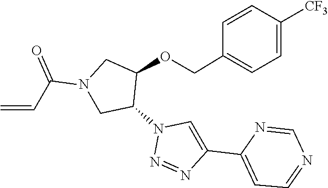 [SMILES: C=CC(=O)N1C[C@@H](n2cc(-c3ccncn3)nn2)[C@H](OCc2ccc(C(F)(F)F)cc2)C1] |
| II-11 | 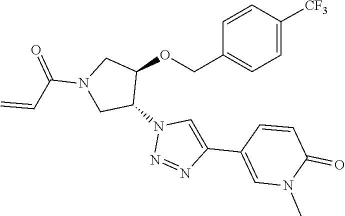 [SMILES: C=CC(=O)N1C[C@@H](n2cc(-c3ccc(=O)n(C)c3)nn2)[C@H](OCc2ccc(C(F)(F)F)cc2)C1] |
| II-12 | 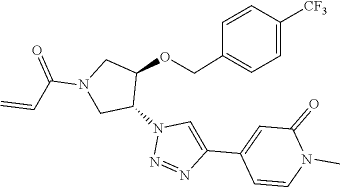 [SMILES: C=CC(=O)N1C[C@@H](n2cc(-c3ccn(C)c(=O)c3)nn2)[C@H](OCc2ccc(C(F)(F)F)cc2)C1] |
| II-13 | 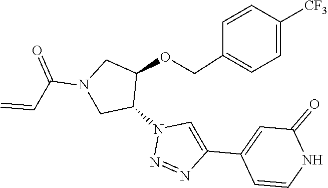 [SMILES: C=CC(=O)N1C[C@@H](n2cc(-c3cc[nH]c(=O)c3)nn2)[C@H](OCc2ccc(C(F)(F)F)cc2)C1] |
| II-14 | 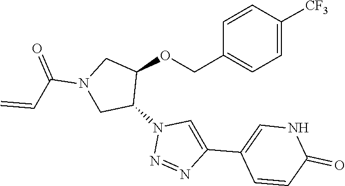 [SMILES: C=CC(=O)N1C[C@@H](n2cc(-c3ccc(=O)[nH]c3)nn2)[C@H](OCc2ccc(C(F)(F)F)cc2)C1] |
| II-15 | 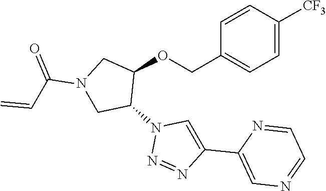 [SMILES: C=CC(=O)N1C[C@@H](n2cc(-c3cnccn3)nn2)[C@H](OCc2ccc(C(F)(F)F)cc2)C1] |
| II-16 | 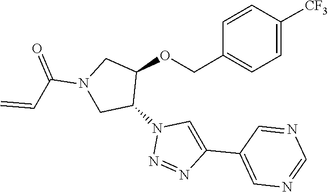 [SMILES: C=CC(=O)N1C[C@@H](n2cc(-c3cncnc3)nn2)[C@H](OCc2ccc(C(F)(F)F)cc2)C1] |
| II-17 | 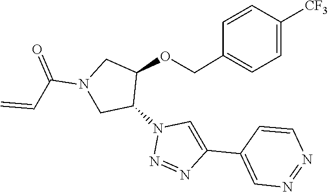 [SMILES: C=CC(=O)N1C[C@@H](n2cc(-c3ccnnc3)nn2)[C@H](OCc2ccc(C(F)(F)F)cc2)C1] |
| II-18 | 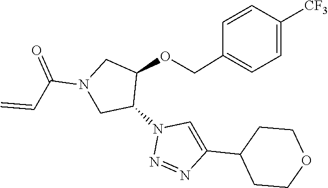 [SMILES: C=CC(=O)N1C[C@@H](n2cc(C3CCOCC3)nn2)[C@H](OCc2ccc(C(F)(F)F)cc2)C1] |
| II-19 | 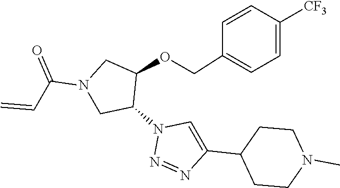 [SMILES: C=CC(=O)N1C[C@@H](n2cc(C3CCN(C)CC3)nn2)[C@H](OCc2ccc(C(F)(F)F)cc2)C1] |
| II-20 | 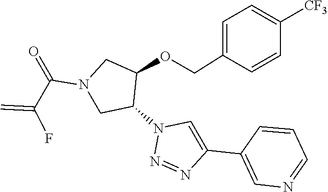 [SMILES: C=C(F)C(=O)N1C[C@@H](n2cc(-c3cccnc3)nn2)[C@H](OCc2ccc(C(F)(F)F)cc2)C1] |
| II-21 | 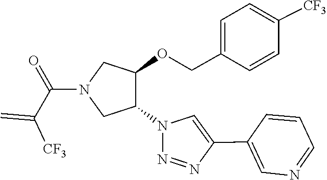 [SMILES: C=C(C(=O)N1C[C@@H](n2cc(-c3cccnc3)nn2)[C@H](OCc2ccc(C(F)(F)F)cc2)C1)C(F)(F)F] |
| II-22 | 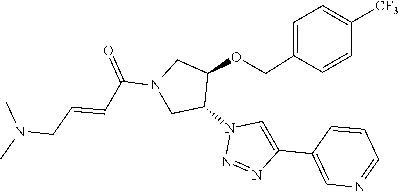 [SMILES: CN(C)C/C=C/C(=O)N1C[C@@H](n2cc(-c3cccnc3)nn2)[C@H](OCc2ccc(C(F)(F)F)cc2)C1] |
| II-23 | 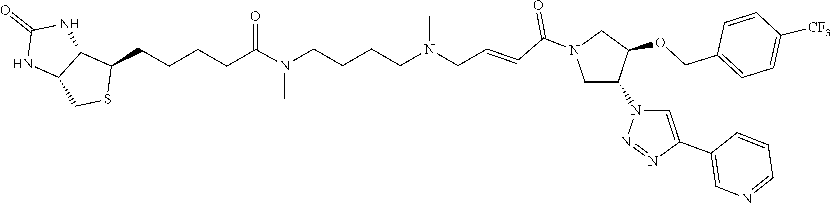 [SMILES: CN(C/C=C/C(=O)N1C[C@@H](n2cc(-c3cccnc3)nn2)[C@H](OCc2ccc(C(F)(F)F)cc2)C1)CCCCN(C)C(=O)CCCC[C@H]1SC[C@@H]2NC(=O)N[C@@H]21] |
| II-24 | 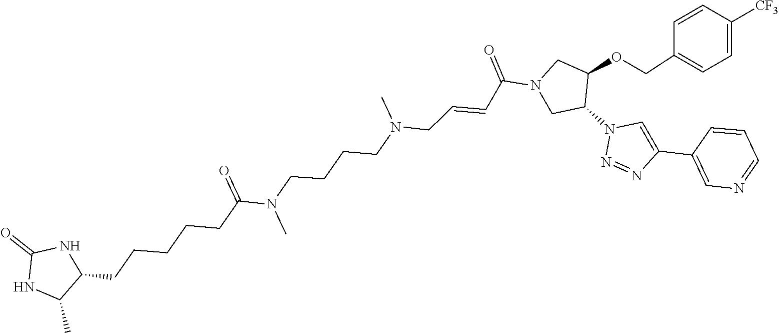 [SMILES: C[C@@H]1NC(=O)N[C@@H]1CCCCCC(=O)N(C)CCCCN(C)C/C=C/C(=O)N1C[C@@H](n2cc(-c3cccnc3)nn2)[C@H](OCc2ccc(C(F)(F)F)cc2)C1] |
| II-25 | 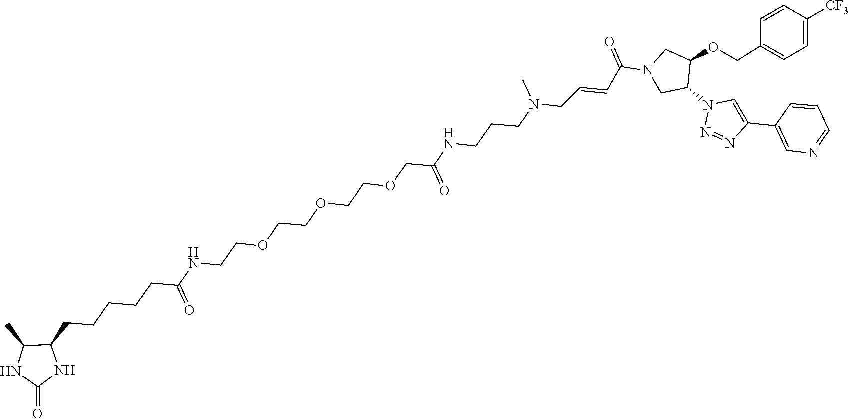 [SMILES: C[C@@H]1NC(=O)N[C@@H]1CCCCCC(=O)NCCOCCOCCOCC(=O)NCCCN(C)C/C=C/C(=O)N1C[C@@H](n2cc(-c3cccnc3)nn2)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound described herein is of Formula (III):

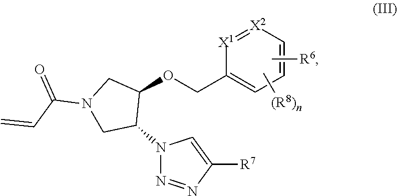

or a pharmaceutically acceptable salt, solvate, hydrate, polymorph, co-crystal, tautomer, stereoisomer, isotopically labeled derivative, prodrug, composition, or mixture thereof, wherein:
R⁶ is —CF₃ or —C≡C—H;
R⁷ and R⁸ are each independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
R⁷ and R⁸ are each independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
each occurrence of Re is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{c}, when present, can be joined together with the heteroatom to which they are attached to form an optionally substituted heterocyclic ring;
X¹ and X² are each independently —N═ or —C(H)═; and
n is 0, 1, or 2.

In certain embodiments, R⁶ is —CF₃ or —C≡C—H. In certain embodiments, R⁶ is —CF₃. In certain embodiments, R⁶ is —C≡C—H.

In certain embodiments, R⁷ or R⁸ is independently hydrogen. In certain embodiments, R⁷ or R⁸ is independently halogen. In certain embodiments, R⁷ or R⁸ is independently optionally substituted alkyl (e.g., -Me, -Et, -i-Pr). In certain embodiments, R⁷ or R⁸ is independently optionally substituted alkenyl. In certain embodiments, R⁷ or R⁸ is independently optionally substituted alkynyl. In certain embodiments, R⁷ or R⁸ is independently optionally substituted carbocyclyl. In certain embodiments, R⁷ or R⁸ is independently optionally substituted heterocyclyl. In certain embodiments, R⁷ or R⁸ is independently optionally substituted aryl. In certain embodiments, R⁷ or R⁸ is independently optionally substituted heteroaryl. In certain embodiments, R⁷ or R⁸ is independently —OR^{c}. In certain embodiments, R⁷ or R⁸ is independently —N(R^{c})₂. In certain embodiments, R⁷ or R⁸ is independently —SR^{c} In certain embodiments, R⁷ or R⁸ is independently —C(═O)R^{c}. In certain embodiments, R⁷ or R⁸ is independently —C(═O)OR^{c}. In certain embodiments, R⁷ or R⁸ is independently —C(═O)N(R^{c})₂. In certain embodiments, R⁷ or R⁸ is independently —OC(═O)R^{c}. In certain embodiments, R⁷ or R⁸ is independently —N(R^{c})C(═O)R^{c}. In certain embodiments, R⁷ or R⁸ is independently —CN. In certain embodiments, R⁷ or R⁸ is independently —NO₂. In certain embodiments, R⁷ is hydrogen. In certain embodiments, R⁷ is optionally substituted heteroaryl. In certain embodiments, R⁷ is optionally substituted pyridine. In certain embodiments, R⁷ is of the formula:

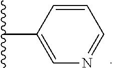 [SMILES: *c1cccnc1]

In certain embodiments, R⁸ is hydrogen. In certain embodiments, R⁸ is halo. In certain embodiments, R⁸ is chloro.

In certain embodiments, n is 0, 1, or 2. In certain embodiments, n is 0. In certain embodiments, n is 1. In certain embodiments, n is 2.

In certain embodiments, a compound of Formula (III) is of Formula (III-a):

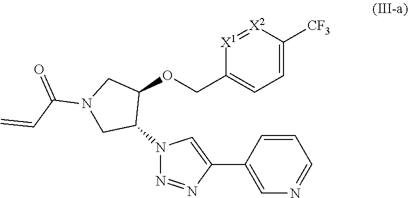

or a pharmaceutically acceptable salt, solvate, hydrate, polymorph, co-crystal, tautomer, stereoisomer, isotopically labeled derivative, prodrug, composition, or mixture thereof.

In certain embodiments, X¹ and X² are each independently —N═ or —C(H)═. In certain embodiments, X¹ is —N═. In certain embodiments, X¹ is —C(H)═. In certain embodiments, X² is —C(H)═. In certain embodiments, X² is —N═. In certain embodiments, X¹ is —N═, and X² is —C(H)═. In certain embodiments, X² is —N═, and X¹ is —C(H)═.

In certain embodiments, the compound of Formula (III) is of Formula (III-b):

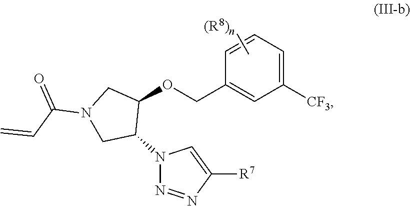

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (III-b) is of Formula (III-b-1):

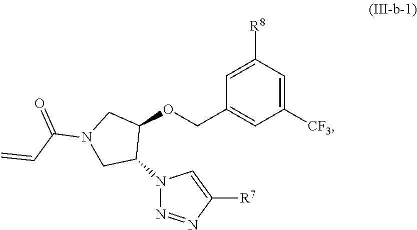

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (III-b) is of Formula (III-b-2):

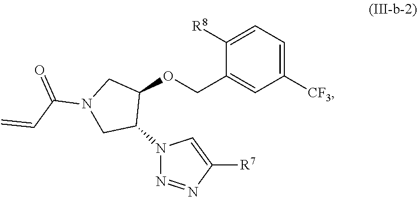

or a pharmaceutically acceptable salt, solvate, hydrate, polymorph, co-crystal, tautomer, stereoisomer, isotopically labeled derivative, prodrug, composition, or mixture thereof.

In some aspects, the substituents R⁷ and R⁸ contain the substituent R^{c}. In certain embodiments, R^{c} is hydrogen. In certain embodiments, R^{c} is optionally substituted alkyl (e.g., -Me, -Et, -i-Pr). In certain embodiments, R^{c} is -Me. In certain embodiments, R^{c} is optionally substituted alkenyl. In certain embodiments, R^{c} is optionally substituted alkynyl. In certain embodiments, R^{c} is optionally substituted carbocyclyl. In certain embodiments, R^{c} is optionally substituted heterocyclyl. In certain embodiments, R^{c} is optionally substituted aryl. In certain embodiments, R^{c} is optionally substituted heteroaryl. In certain embodiments, R^{c} is a nitrogen protecting group. In certain embodiments, two instances of R^{c}, when present, can be joined together with the heteroatom to which they are attached to form an optionally substituted heterocyclic ring.

In certain embodiments, the compound of Formula (III) is of one of the formulae in Table 3 below:

TABLE 3
Compounds of Formula (III)

| Compound | |
|---|---|
| No. | Chemical Structure |
| III-1 | 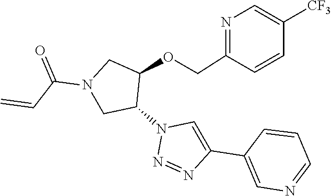 [SMILES: C=CC(=O)N1C[C@@H](n2cc(-c3cccnc3)nn2)[C@H](OCc2ccc(C(F)(F)F)cn2)C1] |
| III-2 | 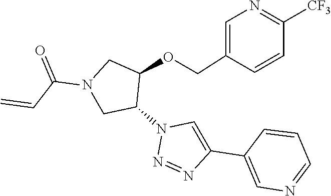 [SMILES: C=CC(=O)N1C[C@@H](n2cc(-c3cccnc3)nn2)[C@H](OCc2ccc(C(F)(F)F)nc2)C1] |
| III-3 | 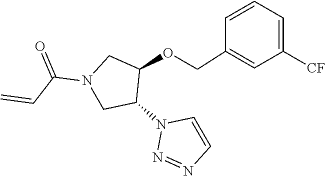 |
| III-4 | 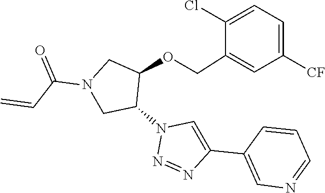 |
| III-5 | 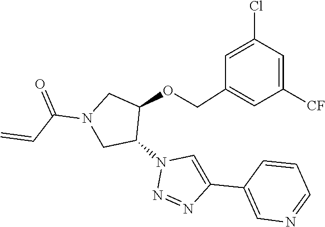 |
| III-6 | 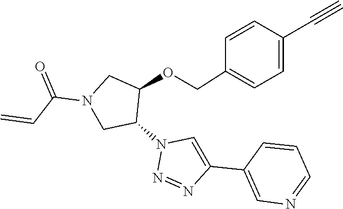 [SMILES: C#Cc1ccc(CO[C@@H]2CN(C(=O)C=C)C[C@H]2n2cc(-c3cccnc3)nn2)cc1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound described herein is of Formula (IV):

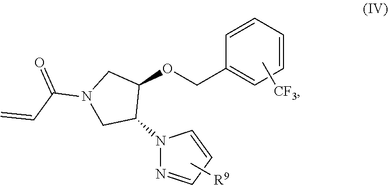

or a pharmaceutically acceptable salt, solvate, hydrate, polymorph, co-crystal, tautomer, stereoisomer, isotopically labeled derivative, prodrug, composition, or mixture thereof, wherein:
R⁹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{d}, —N(R^{d})₂, —SR^{d}, —C(═O)R^{d}, —C(═O)OR^{d}, —C(═O)N(R^{d})₂, —OC(═O)R^{d}, —N(R^{d})C(═O)R^{d}, —CN, or —NO₂;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{d}, when present, can be joined together with the heteroatom to which they are attached to form an optionally substituted heterocyclic ring.

In certain embodiments, the compound of Formula (IV) is a compound of Formula (IV-a):

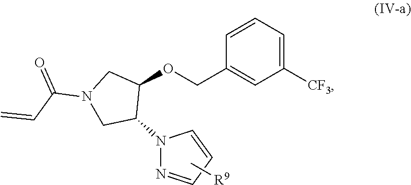

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (IV) or (IV-a) is a compound of the formula:

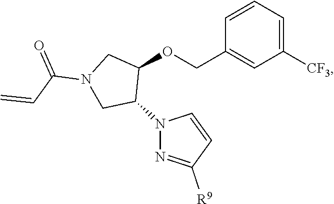

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (IV) or (IV-a) is a compound of the formula:

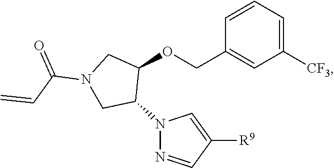

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (IV) is a compound of Formula (IV-b):

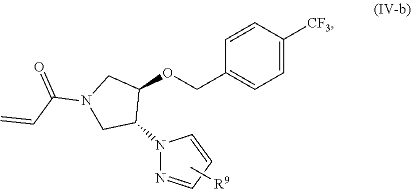

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (IV) or (IV-b) is a compound of the formula:

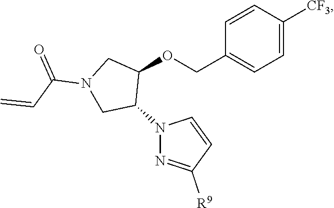

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (IV) or (IV-b) is a compound of the formula:

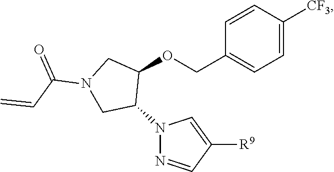

or a pharmaceutically acceptable salt, solvate, hydrate, polymorph, co-crystal, tautomer, stereoisomer, isotopically labeled derivative, prodrug, composition, or mixture thereof.

In certain embodiments, R⁹ is hydrogen. In certain embodiments, R⁹ is halogen. In certain embodiments, R⁹ is optionally substituted alkyl (e.g., -Me, -Et, -i-Pr). In certain embodiments, R⁹ is optionally substituted alkenyl. In certain embodiments, R⁹ is optionally substituted alkynyl. In certain embodiments, R⁹ is optionally substituted carbocyclyl. In certain embodiments, R⁹ is optionally substituted heterocyclyl. In certain embodiments, R⁹ is optionally substituted aryl. In certain embodiments, R⁹ is optionally substituted heteroaryl. In certain embodiments, R⁹ is —OR^{d}. In certain embodiments, R⁹ is —N(R^{d})₂. In certain embodiments, R⁹ is —SR^{d}. In certain embodiments, R⁹ is —C(═O)R^{d}. In certain embodiments, R⁹ is —C(═O)OR^{d}. In certain embodiments, R⁹ is —C(═O)N(Rd)₂. In certain embodiments, R⁹ is —C(═O)N(H)Me. In certain embodiments, R⁹ is —OC(═O)R^{d}. In certain embodiments, R⁹ is —N(R^{d})C(═O)R^{d}. In certain embodiments, R⁹ is —CN. In certain embodiments, R⁹ is —NO₂.

In certain embodiments, R^{d} is hydrogen. In certain embodiments, R^{d} is optionally substituted alkyl (e.g., -Me, -Et, -i-Pr). In certain embodiments, at least on instance of R^{d} is -Me. In certain embodiments, R^{d} is optionally substituted alkenyl. In certain embodiments, R^{d} is optionally substituted alkynyl. In certain embodiments, R^{d} is optionally substituted carbocyclyl. In certain embodiments, R^{d} is optionally substituted heterocyclyl. In certain embodiments, R^{a} is optionally substituted aryl. In certain embodiments, R^{d} is optionally substituted heteroaryl. In certain embodiments, R^{d} is a nitrogen protecting group. In certain embodiments, two instances of R^{d}, when present, can be joined together with the heteroatom to which they are attached to form an optionally substituted heterocyclic ring. In certain embodiments, one instance of R^{d} is methyl and one instance of R^{d} is hydrogen.

In certain embodiments, the compound of Formula (IV) is of one of the formulae in Table 4 below:

TABLE 4
Compounds of Formula (IV)

| Compound | |
|---|---|
| No. | Chemical Structure |
| IV-1 | 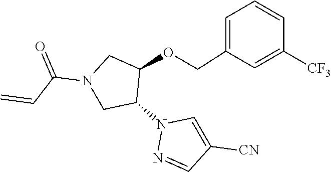 [SMILES: C=CC(=O)N1C[C@@H](n2cc(C#N)cn2)[C@H](OCc2cccc(C(F)(F)F)c2)C1] |
| IV-2 | 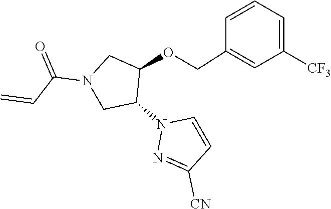 [SMILES: C=CC(=O)N1C[C@@H](n2ccc(C#N)n2)[C@H](OCc2cccc(C(F)(F)F)c2)C1] |
| IV-3 | 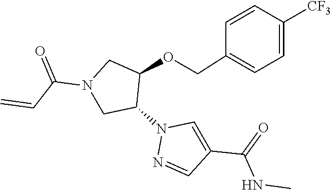 [SMILES: C=CC(=O)N1C[C@@H](n2cc(C(=O)NC)cn2)[C@H](OCc2ccc(C(F)(F)F)cc2)C1] |
| IV-4 | 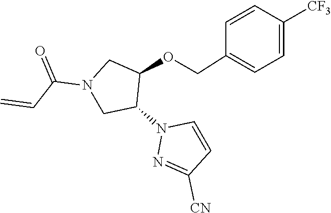 [SMILES: C=CC(=O)N1C[C@@H](n2ccc(C#N)n2)[C@H](OCc2ccc(C(F)(F)F)cc2)C1] |
| IV-5 | 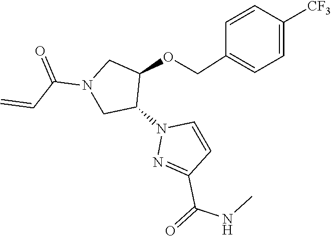 [SMILES: C=CC(=O)N1C[C@@H](n2ccc(C(=O)NC)n2)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound described herein is of Formula (V):

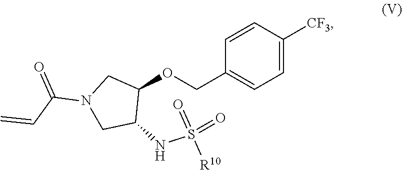

or a pharmaceutically acceptable salt, solvate, hydrate, polymorph, co-crystal, tautomer, stereoisomer, isotopically labeled derivative, prodrug, composition, or mixture thereof, wherein:
R¹⁰ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{e}, —N(R^{e})₂, —SR^{e}, —C(═O)R^{e}, —C(═O)OR^{e}, —C(═O)N(R^{e})₂, —OC(═O)R^{e}, —N(R^{e})C(═O)R^{e}, —CN, or —NO₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of Re, when present, can be joined together with the heteroatom to which they are attached to form an optionally substituted heterocyclic ring.

In certain embodiments, R¹⁰ is hydrogen. In certain embodiments, R¹⁰ is halogen. In certain embodiments, R¹⁰ is optionally substituted alkyl (e.g., -Me, -Et, -i-Pr). In certain embodiments, R¹⁰ is optionally substituted alkenyl. In certain embodiments, R¹⁰ is optionally substituted alkynyl. In certain embodiments, R¹⁰ is optionally substituted carbocyclyl. In certain embodiments, R¹⁰ is optionally substituted heterocyclyl. In certain embodiments, R¹⁰ is optionally substituted aryl. In certain embodiments, R¹⁰ is optionally substituted phenyl. In certain embodiments, R¹⁰ is of the formula:

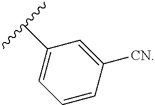 [SMILES: *c1cccc(C#N)c1]

In certain embodiments, R¹⁰ is optionally substituted heteroaryl. In certain embodiments, R¹⁰ is optionally substituted pyridine. In certain embodiments, R¹⁰ is optionally substituted pyrimidine. In certain embodiments, R¹⁰ is optionally substituted pyridazine. In certain embodiments, R¹⁰ is optionally substituted pyrazine. In certain embodiments, R¹⁰ is optionally substituted pyrazole. In certain embodiments, R¹⁰ is optionally substituted imidazole. In certain embodiments, R¹⁰ is optionally substituted oxazole. In certain embodiments, R¹⁰ is of the formula:

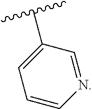 [SMILES: *c1cccnc1]

In certain embodiments, R¹⁰ is of the formula:

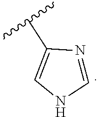 [SMILES: *c1c[nH]cn1]

In certain embodiments, R¹⁰ is of the formula:

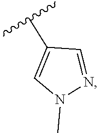 [SMILES: *c1cnn(C)c1]

In certain embodiments, R¹⁰ is of the formula:

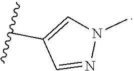 [SMILES: *c1cnn(C)c1]

In certain embodiments, R¹⁰ is of the formula:

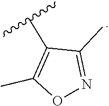

In certain embodiments, R¹⁰ is of the formula:

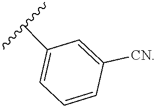 [SMILES: *c1cccc(C#N)c1]

In certain embodiments, R¹⁰ is —OR^{e}. In certain embodiments, R¹⁰ is —N(R^{e})₂. In certain embodiments, R¹⁰ is —SR^{e}. In certain embodiments, R¹⁰ is —C(═O)R^{e}. In certain embodiments, R¹⁰ is —C(═O)OR^{e}. In certain embodiments, R¹⁰ is —C(═O)N(R^{e})₂. In certain embodiments, R¹⁰ is —OC(═O)R^{e}. In certain embodiments, R¹⁰ is —N(R^{e})C(═O)R^{e}. In certain embodiments, R¹⁰ is —CN. In certain embodiments, R¹⁰ is —NO₂.

In some aspects, the substituent R¹⁰ contains the substituent Re. In certain embodiments, R^{e} is hydrogen. In certain embodiments, R^{e} is optionally substituted alkyl (e.g., -Me, -Et, -i-Pr). In certain embodiments, R^{e} is -Me. In certain embodiments, R^{e} is optionally substituted alkenyl. In certain embodiments, R^{e} is optionally substituted alkynyl. In certain embodiments, R^{e} is optionally substituted carbocyclyl. In certain embodiments, R^{e} is optionally substituted heterocyclyl. In certain embodiments, R^{e} is optionally substituted aryl. In certain embodiments, R^{e} is optionally substituted heteroaryl. In certain embodiments, R^{e} is a nitrogen protecting group. In certain embodiments, two instances of Re, when present, can be joined together with the heteroatom to which they are attached to form an optionally substituted heterocyclic ring.

In certain embodiments, the compound of Formula (V) is of one of the formulae in Table 5 below:

TABLE 5
Compounds of Formula (V)

| Compound | |
|---|---|
| No. | Chemical Structure |
| V-1 | 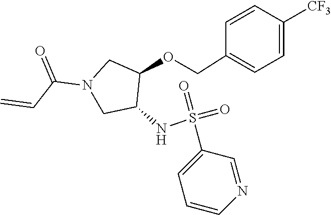 [SMILES: C=CC(=O)N1C[C@@H](NS(=O)(=O)c2cccnc2)[C@H](OCc2ccc(C(F)(F)F)cc2)C1] |
| V-2 | 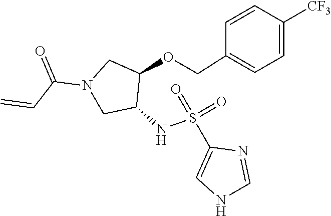 [SMILES: C=CC(=O)N1C[C@@H](NS(=O)(=O)c2c[nH]cn2)[C@H](OCc2ccc(C(F)(F)F)cc2)C1] |
| V-3 | 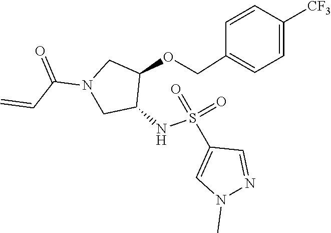 [SMILES: C=CC(=O)N1C[C@@H](NS(=O)(=O)c2cnn(C)c2)[C@H](OCc2ccc(C(F)(F)F)cc2)C1] |
| V-4 | 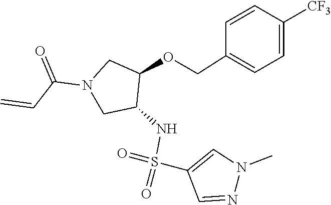 [SMILES: C=CC(=O)N1C[C@@H](NS(=O)(=O)c2cnn(C)c2)[C@H](OCc2ccc(C(F)(F)F)cc2)C1] |
| V-5 | 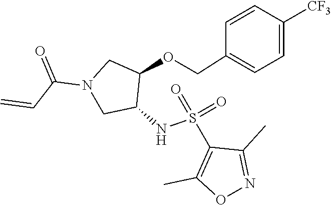 [SMILES: C=CC(=O)N1C[C@@H](NS(=O)(=O)c2c(C)noc2C)[C@H](OCc2ccc(C(F)(F)F)cc2)C1] |
| V-6 | 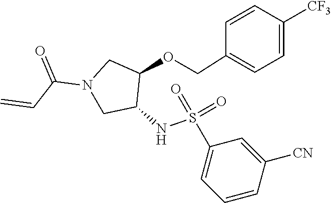 [SMILES: C=CC(=O)N1C[C@@H](NS(=O)(=O)c2cccc(C#N)c2)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound described herein is of Formula (VI):

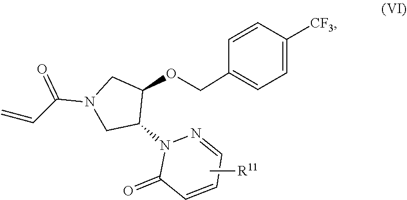

or a pharmaceutically acceptable salt, solvate, hydrate, polymorph, co-crystal, tautomer, stereoisomer, isotopically labeled derivative, prodrug, composition, or mixture thereof, wherein:
R¹¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{f}, —N(R^{f})₂, —SR^{f}, —C(═O)R^{f}, —C(═O)OR^{f}, —C(═O)N(R^{f})₂, —OC(═O)R^{f}, —N(R^{f})C(═O)R^{f}, —CN, or —NO₂;
each occurrence of R^{f} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{f}, when present, can be joined together with the heteroatom to which they are attached to form an optionally substituted heterocyclic ring.

In certain embodiments, the compound of Formula (VI) is a compound of the formula:

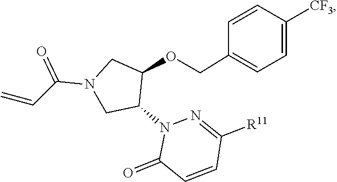

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (VI) is a compound of the formula:

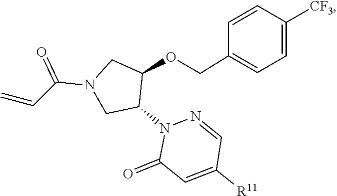

or a pharmaceutically acceptable salt, solvate, hydrate, polymorph, co-crystal, tautomer, stereoisomer, isotopically labeled derivative, prodrug, composition, or mixture thereof.

In certain embodiments, R¹¹ is hydrogen. In certain embodiments, R¹¹ is halogen. In certain embodiments, R¹¹ is optionally substituted alkyl (e.g., -Me, -Et, -i-Pr). In certain embodiments, R¹¹ is optionally substituted alkenyl. In certain embodiments, R¹¹ is optionally substituted alkynyl. In certain embodiments, R¹¹ is optionally substituted carbocyclyl. In certain embodiments, R¹¹ is optionally substituted heterocyclyl. In certain embodiments, R¹¹ is optionally substituted aryl. In certain embodiments, R¹¹ is optionally substituted heteroaryl. In certain embodiments, R¹¹ is optionally substituted imidazole. In certain embodiments, R¹¹ is of the formula:

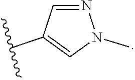 [SMILES: *c1cnn(C)c1]

In certain embodiments, R¹¹ is —OR^{f}. In certain embodiments, R¹¹ is —N(R^{f})₂. In certain embodiments, R¹¹ is —SR^{f}. In certain embodiments, R¹¹ is —C(═O)R^{f}. In certain embodiments, R¹¹ is —C(═O)OR^{f}. In certain embodiments, R¹¹ is —C(═O)N(R^{f})₂. In certain embodiments, R¹¹ is —OC(═O)R^{f}. In certain embodiments, R¹¹ is —N(R^{f})C(═O)R^{f}. In certain embodiments, R¹¹ is —CN. In certain embodiments, R¹¹ is —NO₂.

In some aspects, the substituent R¹¹ contains the substituent R^{f}. In certain embodiments, R′ is hydrogen. In certain embodiments, R^{f} is optionally substituted alkyl (e.g., -Me, -Et, -i-Pr). In certain embodiments, R^{f} is -Me. In certain embodiments, R^{f} is optionally substituted alkenyl. In certain embodiments, R^{f} is optionally substituted alkynyl. In certain embodiments, R^{f} is optionally substituted carbocyclyl. In certain embodiments, R^{f} is optionally substituted heterocyclyl. In certain embodiments, R^{f} is optionally substituted aryl. In certain embodiments, R^{f} is optionally substituted heteroaryl. In certain embodiments, R^{f} is a nitrogen protecting group. In certain embodiments, two instances of R^{f}, when present, can be joined together with the heteroatom to which they are attached to form an optionally substituted heterocyclic ring.

In certain embodiments, the compound of Formula (VI) is of one of the formulae in Table 6 below:

TABLE 6
Compounds of Formula (VI)

| Compound | |
|---|---|
| No. | Chemical Structure |
| VI-1 | 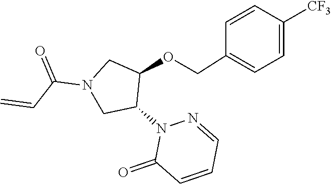 [SMILES: C=CC(=O)N1C[C@@H](n2ncccc2=O)[C@H](OCc2ccc(C(F)(F)F)cc2)C1] |
| VI-2 | 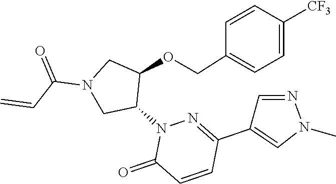 [SMILES: C=CC(=O)N1C[C@@H](n2nc(-c3cnn(C)c3)ccc2=O)[C@H](OCc2ccc(C(F)(F)F)cc2)C1] |
| VI-3 | 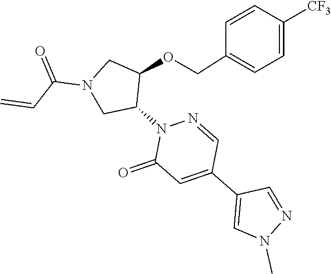 [SMILES: C=CC(=O)N1C[C@@H](n2ncc(-c3cnn(C)c3)cc2=O)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound of Formula (VII) as described herein is of one of the formulae in Table 7 below:

TABLE 7
Compounds of the disclosure

| Compound | |
|---|---|
| No. | Chemical Structure |
| VII-1 | 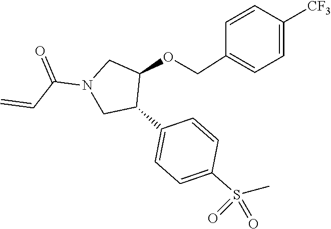 [SMILES: C=CC(=O)N1C[C@@H](OCc2ccc(C(F)(F)F)cc2)[C@H](c2ccc(S(C)(=O)=O)cc2)C1] |
| VII-2 | 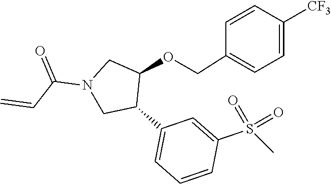 [SMILES: C=CC(=O)N1C[C@@H](OCc2ccc(C(F)(F)F)cc2)[C@H](c2cccc(S(C)(=O)=O)c2)C1] |
| VII-3 | 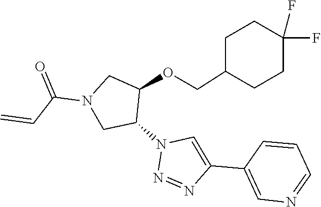 [SMILES: C=CC(=O)N1C[C@@H](n2cc(-c3cccnc3)nn2)[C@H](OCC2CCC(F)(F)CC2)C1] |
| VII-4 | 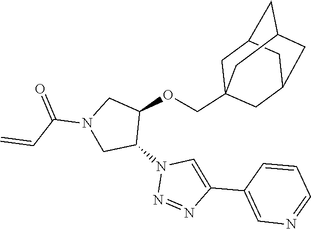 [SMILES: C=CC(=O)N1C[C@@H](n2cc(-c3cccnc3)nn2)[C@H](OCC23CC4CC(CC(C4)C2)C3)C1] |
| VII-5 | 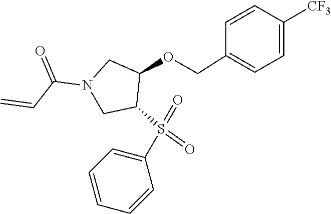 [SMILES: C=CC(=O)N1C[C@@H](S(=O)(=O)c2ccccc2)[C@H](OCc2ccc(C(F)(F)F)cc2)C1] |
| VII-6 | 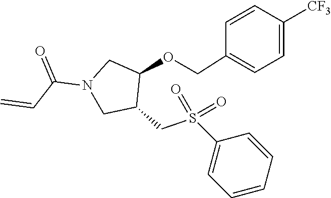 [SMILES: C=CC(=O)N1C[C@@H](CS(=O)(=O)c2ccccc2)[C@H](OCc2ccc(C(F)(F)F)cc2)C1] |
| VII-7 | 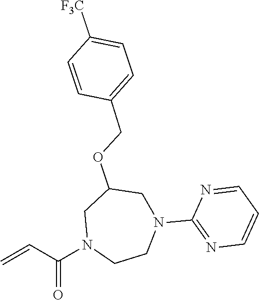 [SMILES: C=CC(=O)N1CCN(c2ncccn2)CC(OCc2ccc(C(F)(F)F)cc2)C1] |
| VII-8 | 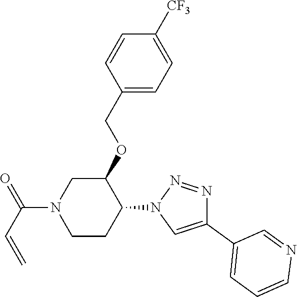 [SMILES: C=CC(=O)N1CC[C@@H](n2cc(-c3cccnc3)nn2)[C@H](OCc2ccc(C(F)(F)F)cc2)C1] |
| VII-9 | 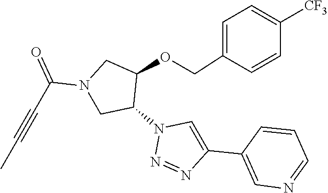 [SMILES: CC#CC(=O)N1C[C@@H](n2cc(-c3cccnc3)nn2)[C@H](OCc2ccc(C(F)(F)F)cc2)C1] |
| VII-10 | 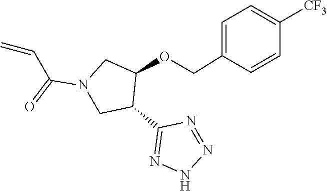 [SMILES: C=CC(=O)N1C[C@@H](OCc2ccc(C(F)(F)F)cc2)[C@H](c2nn[nH]n2)C1] |
| VII-11 | 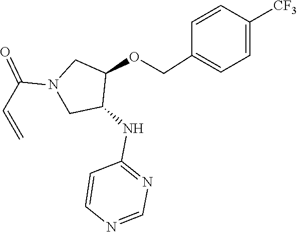 [SMILES: C=CC(=O)N1C[C@@H](Nc2ccncn2)[C@H](OCc2ccc(C(F)(F)F)cc2)C1] |
| VII-12 | 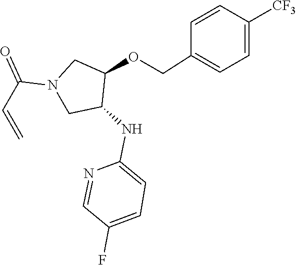 [SMILES: C=CC(=O)N1C[C@@H](Nc2ccc(F)cn2)[C@H](OCc2ccc(C(F)(F)F)cc2)C1] |
| VII-13 | 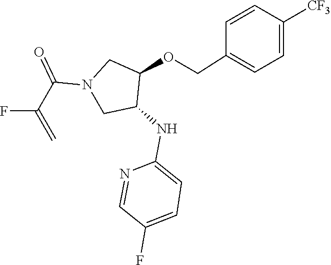 [SMILES: C=C(F)C(=O)N1C[C@@H](Nc2ccc(F)cn2)[C@H](OCc2ccc(C(F)(F)F)cc2)C1] |

and pharmaceutically acceptable salts, solvates, hydrates, polymorphs, co-crystals, tautomers, stereoisomers, isotopically labeled derivatives, prodrugs, compositions, and mixtures thereof.

In certain embodiments, a compound described herein is a compound of Formula (VIII):

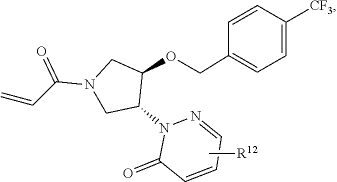

or a pharmaceutically acceptable salt, solvate, hydrate, polymorph, co-crystal, tautomer, stereoisomer, isotopically labeled derivative, prodrug, composition, or mixture thereof, wherein:
R¹² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{h}, —N(R^{h})₂, —SR^{h}, —C(═O)R^{h}, —C(═O)OR^{h}, —C(═O)N(R^{h})₂, —OC(═O)R^{h}, —N(R^{h})C(═O)R^{h}, —CN, or —NO₂;
each occurrence of R^{h} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{h}, when present, can be joined together with the heteroatom to which they are attached to form an optionally substituted heterocyclic ring.

In certain embodiments, the compound of Formula (VIII) is a compound of the formula:

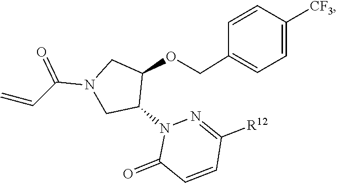

or a pharmaceutically acceptable salt, solvate, hydrate, polymorph, co-crystal, tautomer, stereoisomer, isotopically labeled derivative, prodrug, composition, or mixture thereof.

In certain embodiments, the compound of Formula (VIII) is a compound of the formula:

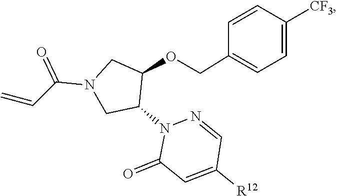

or a pharmaceutically acceptable salt, solvate, hydrate, polymorph, co-crystal, tautomer, stereoisomer, isotopically labeled derivative, prodrug, composition, or mixture thereof.

In certain embodiments, R¹² is hydrogen. In certain embodiments, R¹² is halogen. In certain embodiments, R¹² is optionally substituted alkyl (e.g., -Me, -Et, -i-Pr). In certain embodiments, R¹² is optionally substituted alkenyl. In certain embodiments, R¹² is optionally substituted alkynyl. In certain embodiments, R¹² is optionally substituted carbocyclyl. In certain embodiments, R¹² is optionally substituted heterocyclyl. In certain embodiments, R¹² is optionally substituted aryl. In certain embodiments, R¹² is optionally substituted heteroaryl. In certain embodiments, R¹² is optionally substituted imidazole. In certain embodiments, R¹² is of the formula:

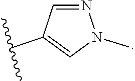 [SMILES: *c1cnn(C)c1]

In certain embodiments, R¹² is —OR^{h}. In certain embodiments, R¹² is —N(R^{h})₂. In certain embodiments, R¹² is —SR^{h}. In certain embodiments, R¹² is —C(═O)R^{h}. In certain embodiments, R¹² is —C(═O)OR^{h}. In certain embodiments, R¹² is —C(═O)N(R^{h})₂. In certain embodiments, R¹² is —OC(═O)R^{h}. In certain embodiments, R¹² is —N(R^{h})C(═O)R^{h}. In certain embodiments, R¹² is —CN. In certain embodiments, R¹² is —NO₂.

In some aspects, the substituent R¹² contains the substituent R^{h}. In certain embodiments, R^{h} is hydrogen. In certain embodiments, R^{h} is optionally substituted alkyl (e.g., -Me, -Et, -i-Pr). In certain embodiments, R^{h} is -Me. In certain embodiments, R^{h} is optionally substituted alkenyl. In certain embodiments, R^{h} is optionally substituted alkynyl. In certain embodiments, R^{h} is optionally substituted carbocyclyl. In certain embodiments, R^{h} is optionally substituted heterocyclyl. In certain embodiments, R^{h} is optionally substituted aryl. In certain embodiments, R^{h} is optionally substituted heteroaryl. In certain embodiments, R^{h} is a nitrogen protecting group. In certain embodiments, two instances of R^{h}, when present, can be joined together with the heteroatom to which they are attached to form an optionally substituted heterocyclic ring.

In certain embodiments, the compounds of Formula (VIII) are of the formula (VIII-1):

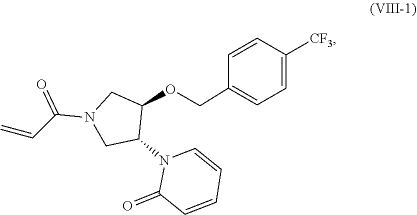 [SMILES: C=CC(=O)N1C[C@@H](n2ccccc2=O)[C@H](OCc2ccc(C(F)(F)F)cc2)C1]

or a pharmaceutically acceptable salt, solvate, hydrate, polymorph, co-crystal, tautomer, stereoisomer, isotopically labeled derivative, prodrug, composition, or mixture thereof.

In certain embodiments, the compounds described herein are provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for reducing the risk of developing a disease (e.g., proliferative disease) in a subject in need thereof. In certain embodiments, the effective amount is an amount effective for inhibiting the activity (e.g., aberrant activity, such as increased activity or undesired activity) of a transcription factor (e.g., TEAD) in a subject or cell. In certain embodiments, the effective amount is an amount effective for modulating the activity of the hippo signaling pathway in a subject or cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

In certain embodiments, the effective amount is an amount effective for inhibiting the activity of a transcription factor by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of a TEAD family transcription factor by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of a TEAD family transcription factor by a range between a percentage described in this paragraph and another percentage described in this paragraph, inclusive.

In certain embodiments, the transcription factor is a TEAD family transcription factor. In certain embodiments, the TEAD is TEAD1. In certain embodiments, the TEAD is TEAD2. In certain embodiments, the TEAD is TEAD3. In certain embodiments, the TEAD is TEAD4. In certain embodiments, the present disclosure provides inhibitors of the TEAD family of transcription factors (e.g., TEAD1, TEAD2, TEAD3, TEAD4). In certain embodiments, the inventive compounds inhibit the activity of a TEAD. In certain embodiments, the inhibitor is selective for the TEAD family of transcription factors.

The present disclosure provides methods of using the compounds described herein, e.g., as biological probes to study the hippo signaling pathway, or the inhibition of the activity of a transcription factor (e.g., TEAD (e.g., TEAD1, TEAD2, TEAD3, TEAD4)), and as therapeutics, e.g., in the treatment and/or prevention of diseases associated with the overexpression and/or aberrant activity of the hippo signaling pathway or a transcription factor (e.g., TEAD (e.g., TEAD1, TEAD2, TEAD3, TEAD4,)). In certain embodiments, the compound covalently binds TEADs (e.g., TEAD1). In certain embodiments, the diseases treated and/or prevented include, but are not limited to, proliferative diseases. The proliferative diseases include, but are not limited to, cancer (e.g., carcinoma, sarcoma, lung cancer, thyroid cancer, skin cancer, ovarian cancer, colorectal cancer, prostate cancer, pancreatic cancer, esophageal cancer, liver cancer, breast cancer). In certain embodiments, the cancer is a sarcoma. In certain embodiments, the cancer is Kaposi's sarcoma. In certain embodiments, the cancer is associated with the overexpression and/or aberrant activity of a transcription factor (e.g., TEAD (e.g., TEAD1, TEAD2, TEAD3, TEAD4)). Also provided by the present disclosure are mixtures, compositions, pharmaceutical compositions, kits, methods, and uses of a compound of Formula (I′), Formula (I), Formula (II), Formula (III), Formula (IV), Formula (V), Formula (VI), Formula (VII), or Formula (VIII), as described herein.

The compounds described herein may be useful in treating and/or preventing diseases (e.g., proliferative diseases (e.g., cancers)) or diseases associated with the activity of a transcription factor (e.g., TEAD1, TEAD2, TEAD3, TEAD4) in a subject, or inhibiting the activity of a transcription factor (e.g., TEAD1, TEAD2, TEAD3, TEAD4) in a subject or biological sample. In certain embodiments, a compound described herein is a compound of Formula (I′), Formula (I), Formula (II), Formula (III), Formula (IV), Formula (V), Formula (VI), Formula (VII), or Formula (VIII), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I′), Formula (I), Formula (II), Formula (III), Formula (IV), Formula (V), Formula (VI), Formula (VII), or Formula (VIII), or a pharmaceutically acceptable salt thereof. In certain embodiments, a compound described herein is a compound of Formula (I′), Formula (I), Formula (II), Formula (III), Formula (IV), Formula (V), Formula (VI), Formula (VII), or Formula (VIII), or a composition thereof.

Certain compounds described herein bind, covalently modify, and/or inhibit a transcription factor. In certain embodiments, the compounds described herein irreversibly inhibit a transcription factor. In certain embodiments, the compounds described herein reversibly inhibit a transcription factor. In certain embodiments, the transcription factor is a transcription enhancer factor. In certain embodiments, the transcription factor is a TEAD family transcription factor. In certain embodiments, the transcription factor is TEAD1. In certain embodiments, the transcription factor is TEAD2. In certain embodiments, the transcription factor is TEAD3. In certain embodiments, the transcription factor is TEAD4. In certain embodiments, the compounds described herein covalently bind to the transcription factor (e.g., TEAD (e.g., TEAD1, TEAD2, TEAD3, or TEAD4)). In certain embodiments, the compounds described herein reversibly bind to the transcription factor (e.g., TEAD (e.g., TEAD1, TEAD2, TEAD3, or TEAD4)). In certain embodiments, the compounds described herein non-reversibly bind to the transcription factor (e.g., TEAD (e.g., TEAD1, TEAD2, TEAD3, or TEAD4)). In certain embodiments, the compounds described herein modulate the activity of a transcription factor (e.g., TEAD (e.g., TEAD1, TEAD2, TEAD3, or TEAD4)). In certain embodiments, the compounds described herein inhibit the activity of a transcription factor (e.g., a TEAD family transcription factor (e.g., TEAD (e.g., TEAD1, TEAD2, TEAD3, or TEAD4)). In certain embodiments, the compounds described herein reversibly inhibit the activity of a transcription factor (e.g., TEAD (e.g., TEAD1, TEAD2, TEAD3, or TEAD4)). In certain embodiments, the compounds described herein irreversibly inhibit the activity of a transcription factor (e.g., TEAD (e.g., TEAD1, TEAD2, TEAD3, or TEAD4)). In certain embodiments, the compounds described herein covalently inhibit the activity of a transcription factor (e.g., TEAD (e.g., TEAD1, TEAD2, TEAD3, or TEAD4)).

The binding affinity of a compound described herein to a transcription factor (e.g., TEAD (e.g., TEAD1, TEAD2, TEAD3, or TEAD4)) may be measured by the dissociation constant (K_{d}) value of an adduct of the compound and the transcription factor (e.g., TEAD (e.g., TEAD1, TEAD2, TEAD3, or TEAD4)) using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the activity of a transcription factor (e.g., TEAD (e.g., TEAD1, TEAD2, TEAD3, or TEAD4)) is inhibited by a compound described herein. The inhibition of the activity of a transcription factor (e.g., a TEAD family transcription factor (e.g., TEAD (e.g., TEAD1, TEAD2, TEAD3, or TEAD4)) by a compound described herein may be measured by determining the half maximal inhibitory concentration (IC₅₀) of the compound when the compound, or a pharmaceutical composition thereof, is contacted with the transcription factor (e.g., TEAD (e.g., TEAD1, TEAD2, TEAD3, or TEAD4)). The IC₅₀ values may be obtained using methods known in the art (e.g., by a competition binding assay). In certain embodiments, the IC₅₀ value of a compound described herein is not more than about 1 mM, not more than about 100 μM, not more than about 10 μM, not more than about 1 μM, not more than about 100 nM, not more than about 10 nM, or not more than about 1 nM.

The compounds described herein may selectively modulate the activity of a transcription factor (e.g., TEAD (e.g., TEAD1, TEAD2, TEAD3, or TEAD4)). In certain embodiments, the compounds selectively increase the activity of a transcription factor (e.g., TEAD (e.g., TEAD1, TEAD2, TEAD3, or TEAD4)) In certain embodiments, the compounds selectively inhibit the activity of a transcription factor (e.g., TEAD (e.g., TEAD1, TEAD2, TEAD3, or TEAD4)). In certain embodiments, the compounds inhibit the activity of two or more protein transcription factors (e.g., TEAD (e.g., TEAD1, TEAD2, TEAD3, or TEAD4)) to the same extent. In certain embodiments, the compounds increase the activity of two or more transcription factors (e.g., TEAD (e.g., TEAD1, TEAD2, TEAD3, or TEAD4)) to the same extent.

The selectivity of a compound described herein in inhibiting the activity of a first transcription factor (e.g., TEAD1, TEAD2, TEAD3, or TEAD4) over a second transcription factor may be measured by the quotient of the IC₅₀ value of the compound in inhibiting the activity of the second transcription factor (e.g., TEAD1, TEAD2, TEAD3, or TEAD4) over the IC₅₀ value of the compound in inhibiting the activity of the first transcription factor (e.g., TEAD1, TEAD2, TEAD3, or TEAD4). The selectivity of a compound described herein in modulating the activity of a first transcription factor (e.g., TEAD1, TEAD2, TEAD3, or TEAD4) over a second transcription factor may also be measured by the quotient of the K_{d} value of an adduct of the compound and the second transcription factor over the K_{d} value of an adduct of the compound and the first transcription factor (e.g., TEAD1, TEAD2, TEAD3, or TEAD4). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold.

### Pharmaceutical Compositions, Kits, and Administration

The present disclosure also provides pharmaceutical compositions comprising a compound as described herein and optionally a pharmaceutically acceptable excipient. In certain embodiments, a compound described herein is a compound of Formula (I′), Formula (I), Formula (II), Formula (III), Formula (IV), Formula (V), Formula (VI), Formula (VII), or Formula (VIII), or a pharmaceutically acceptable salt, solvate, hydrate, polymorph, co-crystal, tautomer, stereoisomer, isotopically labeled derivative, or prodrug thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for inhibiting the activity of a transcription factor (e.g., TEAD (e.g., TEAD1, TEAD2, TEAD3, or TEAD4)). In certain embodiments, a therapeutically effective amount is an amount effective for treating a disease (e.g., a disease associated with aberrant activity of a TEAD (e.g., proliferative disease). In certain embodiments, a therapeutically effective amount is an amount effective for inhibiting the activity of a TEAD (e.g., TEAD1, TEAD2, TEAD3, or TEAD4)) and treating a disease (e.g., a disease associated with aberrant activity of a transcription factor (e.g., TEAD (e.g., TEAD1, TEAD2, TEAD3, or TEAD4)) (e.g., proliferative disease). In certain embodiments, a therapeutically effective amount is an amount effective for inducing apoptosis of a cell (e.g., cell in vivo or in vitro). In certain embodiments, a prophylactically effective amount is an amount effective for inhibiting the activity of a protein (e.g., TEAD (e.g., TEAD1, TEAD2, TEAD3, or TEAD4)). In certain embodiments, a prophylactically effective amount is an amount effective for preventing or keeping a subject in need thereof in remission of a disease (e.g., a disease associated with the activity of a TEAD (e.g., proliferative disease, such as cancer). In certain embodiments, a prophylactically effective amount is an amount effective for inhibiting the activity of a TEAD, and preventing or keeping a subject in need thereof in remission of a disease (e.g., a disease associated with activity of a TEAD (e.g., proliferative disease, such as cancer).

In certain embodiments, the effective amount is an amount effective for inhibiting the activity of a transcription factor (e.g., TEAD (e.g., TEAD1, TEAD2, TEAD3, or TEAD4)) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a TEAD (e.g., TEAD1, TEAD2, TEAD3, or TEAD4)) by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or composition described herein is in vitro. In certain embodiments, the cell being contacted with a compound or composition described herein is in vivo.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine I 131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is ibrutinib. In certain embodiments, the additional pharmaceutical agent is a transcription factor inhibitor (e.g., a TEAD family transcription factor inhibitor). In certain embodiments, the additional pharmaceutical agent is a binder or inhibitor of a TEAD (e.g., TEAD1, TEAD2, TEAD3, or TEAD4)). In certain embodiments, the additional pharmaceutical agent is a binder or inhibitor of a TEAD. In certain embodiments, the additional pharmaceutical agent is a binder or inhibitor of TEAD1. In certain embodiments, the additional pharmaceutical agent is a binder or inhibitor of TEAD2. In certain embodiments, the additional pharmaceutical agent is a binder or inhibitor of TEAD3. In certain embodiments, the additional pharmaceutical agent is a binder or inhibitor of TEAD4. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., transcription factor inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy, including, but not limited to, surgery, radiation therapy, transplantation (e.g., stem cell transplantation, bone marrow transplantation), immunotherapy, and chemotherapy.

Thus, in one aspect, provided are kits including a first container comprising a compound or pharmaceutical composition described herein. In certain embodiments, the kits are useful for treating a disease (e.g., proliferative disease) in a subject in need thereof. In certain embodiments, the kits are useful for preventing a disease (e.g., proliferative disease) in a subject in need thereof. In certain embodiments, the kits are useful for inhibiting the activity (e.g., aberrant or unwanted activity, such as increased activity) of a transcription factor (e.g., TEAD1, TEAD2, TEAD3, or TEAD4) in a subject, biological sample, tissue, or cell. In certain embodiments, the kits are useful for inducing apoptosis of a cell (e.g., cell in vivo or in vitro).

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease) in a subject in need thereof. In certain embodiments, the kits and instructions provide for preventing a disease (e.g., proliferative disease) in a subject in need thereof. In certain embodiments, the kits and instructions provide for modulating (e.g., inhibiting) the activity (e.g., aberrant activity, such as increased activity) of a transcription factor (e.g., TEAD1, TEAD2, TEAD3, TEAD4) in a subject, biological sample, tissue, or cell. In certain embodiments, the kits and instructions provide for inducing apoptosis of a cell. A kit described herein may include one or more additional pharmaceutical agents described herein as a separate composition.

### Methods of Treatment and Uses

The present disclosure provides methods of modulating (e.g., inhibiting or increasing) the activity (e.g., aberrant or undesired activity, such as increased or decreased activity) of a transcription factor (e.g., TEAD (e.g., TEAD1, TEAD2, TEAD3, or TEAD4)). The present disclosure provides methods of modulating (e.g., inhibiting or increasing) the activity (e.g., aberrant activity, such as increased or decreased activity) of a TEAD (e.g., TEAD1, TEAD2, TEAD3, or TEAD4) in a subject, biological sample, tissue, or cell. The present disclosure also provides methods for the treatment of a wide range of diseases, such as diseases associated with the aberrant activity (e.g., increased activity) of a transcription factor, e.g., proliferative diseases, in a subject in need thereof. The present disclosure provides methods for the treatment and/or prevention of a proliferative disease (e.g., cancers (e.g., carcinoma, sarcoma, lung cancer, thyroid cancer, skin cancer, ovarian cancer, colorectal cancer, prostate cancer, pancreatic cancer, esophageal cancer, liver cancer, breast cancer)).

The present disclosure also provides a compound of Formula (I′), Formula (I), Formula (II), Formula (III), Formula (IV), Formula (V), Formula (VI), Formula (VII), or Formula (VIII), or a pharmaceutically acceptable salt, co-crystal, tautomer, stereoisomer, solvate, hydrate, polymorph, isotopically enriched derivative, or prodrug thereof, for use in the treatment of diseases, such as proliferative diseases, in a subject in need thereof.

The present disclosure also provides uses of a compound of Formula (I′), Formula (I), Formula (II), Formula (III), Formula (IV), Formula (V), Formula (VI), Formula (VII), or Formula (VIII), or a pharmaceutically acceptable salt, co-crystal, tautomer, stereoisomer, solvate, hydrate, polymorph, isotopically enriched derivative, or prodrug thereof, in the manufacture of a medicament for the treatment of diseases, such as proliferative diseases, in a subject in need thereof.

In another aspect, the present disclosure provides methods of modulating the activity of a transcription factor (e.g., TEAD (e.g., TEAD1, TEAD2, TEAD3, or TEAD4)) in a subject, biological sample, tissue, or cell. In certain embodiments, provided are methods of inhibiting the activity of a transcription factor in a subject. In certain embodiments, provided are methods of inhibiting the activity of a transcription factor in a cell. In certain embodiments, provided are methods of increasing the activity of a transcription factor (e.g., TEAD (e.g., TEAD1, TEAD2, TEAD3, or TEAD4)) in a subject. The compounds described herein may exhibit transcription factor inhibitory activity; the ability to inhibit a transcription enhancer factor; the ability to inhibit a TEAD family transcription factor; the ability to inhibit TEAD; the ability to inhibit TEAD1, without inhibiting another transcription factor (e.g., TEAD2, TEAD3, or TEAD4); the ability to inhibit TEAD2, without inhibiting another transcription factor (e.g., TEAD1, TEAD3, or TEAD4); the ability to inhibit TEAD3, without inhibiting another transcription factor (e.g., TEAD1, TEAD2, or TEAD4); the ability to inhibit TEAD4, without inhibiting another transcription factor (e.g., TEAD2, TEAD3, or TEAD4); a therapeutic effect and/or preventative effect in the treatment of cancers; a therapeutic effect and/or preventative effect in the treatment of proliferative diseases; and/or a therapeutic profile (e.g., optimum safety and curative effect) that is superior to existing chemotherapeutic agents.

In certain embodiments, provided are methods of decreasing the activity of a transcription factor (e.g., TEAD (e.g., TEAD1, TEAD2, TEAD3, or TEAD4)) in a subject or biological sample (e.g., cell, tissue) by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a transcription factor (e.g., TEAD (e.g., TEAD1, TEAD2, TEAD3, or TEAD4)) in a subject or cell is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a transcription factor (e.g., TEAD1 (e.g., TEAD2, TEAD3, or TEAD4)) in a subject or cell is selectively inhibited by the method. In some embodiments, the activity of a transcription factor (e.g., TEAD (e.g., TEAD1, TEAD2, TEAD3, or TEAD4)) in a subject or cell is selectively decreased by the method.

Without wishing to be bound by any particular theory, the compounds described herein are able to bind (e.g., covalently modify) the transcription factor being inhibited. In certain embodiments, a compound described herein is able to bind (e.g., covalently modify) the transcription factor. In certain embodiments, the compound described herein is able to covalently bind a cysteine residue of the transcription factor. In certain embodiments, the compound is capable of covalently binding the central pocket of the YAP/TAZ domain of a TEAD family transcription factor. In certain embodiments, the compound is capable of covalently binding TEAD1, TEAD2, TEAD3, or TEAD4. In certain embodiments, the compound is capable of covalently modifying TEAD1, TEAD2, TEAD3, or TEAD4. In certain embodiments, the compound is capable of covalently modifying YAP-binding domain of a TEAD transcription factor. In certain embodiments, the compound is capable of covalently modifying TEAD1. In certain embodiments, the compound is capable of covalently modifying TEAD2. In certain embodiments, the compound is capable of covalently modifying TEAD3. In certain embodiments, the compound is capable of covalently modifying TEAD4.

In another aspect, the present disclosure provides methods of inhibiting the activity of a transcription factor in a subject by administering to the subject an effective amount (e.g., therapeutically effective amount) of a compound, or pharmaceutical composition thereof, as described herein. In another aspect, the present disclosure provides methods of inhibiting the activity of a transcription factor (e.g., TEAD (e.g., TEAD1, TEAD2, TEAD3, or TEAD4)) in a biological sample by contacting the biological sample with an effective amount of a compound, or pharmaceutical composition thereof, as described herein. In another aspect, the present disclosure provides methods of inhibiting the activity of a transcription factor in a tissue or cell comprising contacting the tissue or cell with an effective amount of a compound, or pharmaceutical composition thereof, as described herein.

In another aspect, the present disclosure provides methods of inhibiting the activity of a transcription factor (e.g., TEAD (e.g., TEAD1, TEAD2, TEAD3, or TEAD4)) in a cell by contacting the cell with an effective amount of a compound, or pharmaceutical composition thereof, as described herein.

In certain embodiments, the biological sample being contacted with the compound or composition is breast tissue, bone marrow, lymph node, lymph tissue, spleen, or blood. In certain embodiments, the biological sample being contacted with the compound or composition is a tumor or cancerous tissue. In certain embodiments, the biological sample being contacted with the compound or composition is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In certain embodiments, the cell or tissue being contacted with the compound or composition is present in vitro. In certain embodiments, the cell or tissue being contacted with the compound or composition is present in vivo. In certain embodiments, the cell or tissue being contacted with the compound or composition is present ex vivo. In certain embodiments, the cell or tissue being contacted with the compound or composition is a malignant cell.

The disease (e.g., proliferative disease) to be treated or prevented using the compounds described herein may be associated with increased activity of a transcription factor, such as a TEAD (e.g., TEAD1, TEAD2, TEAD3, or TEAD4). The disease (e.g., proliferative disease) to be treated or prevented using the compounds described herein may be associated with the overexpression of a transcription factor, such as a TEAD (e.g., TEAD1, TEAD2, TEAD3, or TEAD4).

In certain embodiments, the disease (e.g., proliferative disease) to be treated or prevented using the compounds described herein may be associated with the overexpression of a TEAD (e.g., TEAD1, TEAD2, TEAD3, or TEAD4). A disease (e.g., proliferative disease) may be associated with aberrant activity of a TEAD (e.g., TEAD1, TEAD2, TEAD3, or TEAD4). Aberrant activity of a TEAD (e.g., TEAD1, TEAD2, TEAD3, TEAD4) may be elevated and/or inappropriate or undesired activity of the TEAD. The compounds described herein, and pharmaceutically acceptable salts, solvates, hydrates, polymorphs, co-crystals, tautomers, stereoisomers, isotopically labeled derivatives, or prodrugs thereof, may inhibit the activity of a TEAD (e.g., TEAD1, TEAD2, TEAD3, or TEAD4) and be useful in treating and/or preventing diseases (e.g., proliferative diseases). The compounds described herein, and pharmaceutically acceptable salts, solvates, hydrates, polymorphs, co-crystals, tautomers, stereoisomers, isotopically labeled derivatives, and prodrugs thereof, may inhibit the activity of a TEAD and be useful in treating and/or preventing diseases (e.g., proliferative diseases). The compounds described herein, and pharmaceutically acceptable salts, solvates, hydrates, polymorphs, co-crystals, tautomers, stereoisomers, isotopically labeled derivatives, and prodrugs thereof, may inhibit the activity of a TEAD and be useful in treating and/or preventing diseases (e.g., proliferative diseases).

All types of biological samples described herein or known in the art are contemplated as being within the scope of the invention. In certain embodiments, the disease (e.g., proliferative disease) to be treated or prevented using the compounds described herein is cancer. All types of cancers disclosed herein or known in the art are contemplated as being within the scope of the invention. In certain embodiments, the proliferative disease is carcinoma. In certain embodiments, the proliferative disease is fallopian tube carcinoma. In certain embodiments, the proliferative disease is carcinoma. In certain embodiments, the proliferative disease is Kaposi's carcinoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2⁺ breast cancer. In certain embodiments, the proliferative disease is HER2 breast cancer. In certain embodiments, the proliferative disease is triple-negative breast cancer (TNBC). In certain embodiments, the proliferative disease is lung cancer. In certain embodiments, the proliferative disease is thyroid cancer. In certain embodiments, the proliferative disease is skin cancer. In certain embodiments, the proliferative disease is ovarian cancer. In certain embodiments, the proliferative disease is prostate cancer. In certain embodiments, the proliferative disease is pancreatic cancer. In certain embodiments, the proliferative disease is esophageal cancer. In certain embodiments, the proliferative disease is liver cancer. In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention.

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein.

The inventive compounds or compositions may synergistically augment inhibition of TEAD induced by the additional pharmaceutical agent(s) in the biological sample or subject. Thus, the combination of the inventive compounds or compositions and the additional pharmaceutical agent(s) may be useful in treating proliferative diseases resistant to a treatment using the additional pharmaceutical agent(s) without the inventive compounds or compositions.

In certain embodiments, a kit described herein includes a first container comprising a compound or pharmaceutical composition described herein. In certain embodiments, a kit described herein is useful in treating and/or preventing a disease, such as a proliferative disease (e.g., cancers (e.g., sarcoma, carcinoma, lung cancer, thyroid cancer, skin cancer, ovarian cancer, colorectal cancer, prostate cancer, pancreatic cancer, esophageal cancer, liver cancer, breast cancer)), in a subject in need thereof, inhibiting the activity of a transcription factor (e.g., a TEAD (e.g., TEAD1, TEAD2, TEAD3, or TEAD4)) in a subject, biological sample, tissue, or cell, and/or inducing apoptosis in a cell.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. Antiproliferation Assay on NCI-H226 Cells (200 Cells/Well, 384 Plate, 5-Day Treatment)

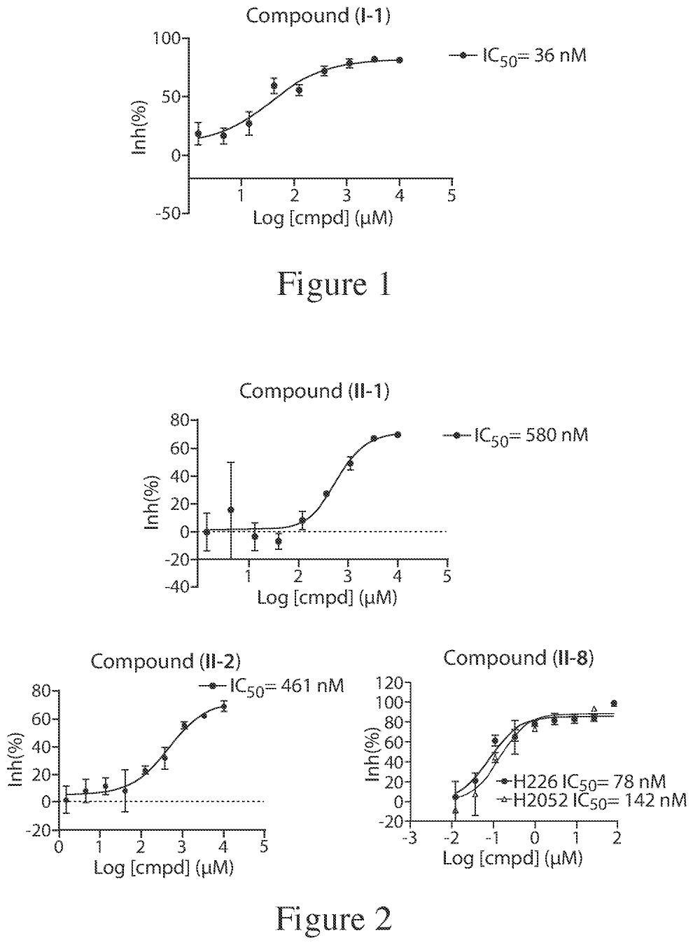

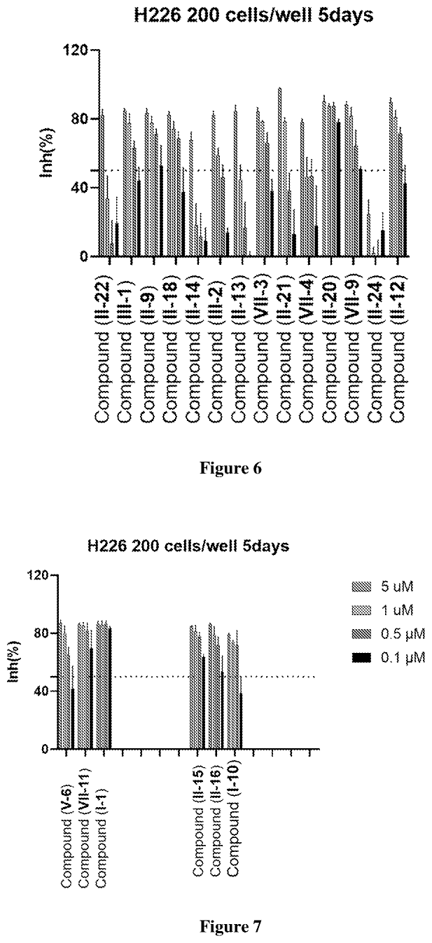

The compounds of the disclosure were tested for their ability in an antiproliferation assay using NCI-H226 cells (FIG. 6 and FIG. 7). The following table summarizes the IC₅₀ values calculated from the graph of the percent inhibition versus the log of the concentration of representative compounds of the disclosure. Compound (II-8) also showed an IC₅₀ of 142 nM using H2052 cells (FIG. 2).

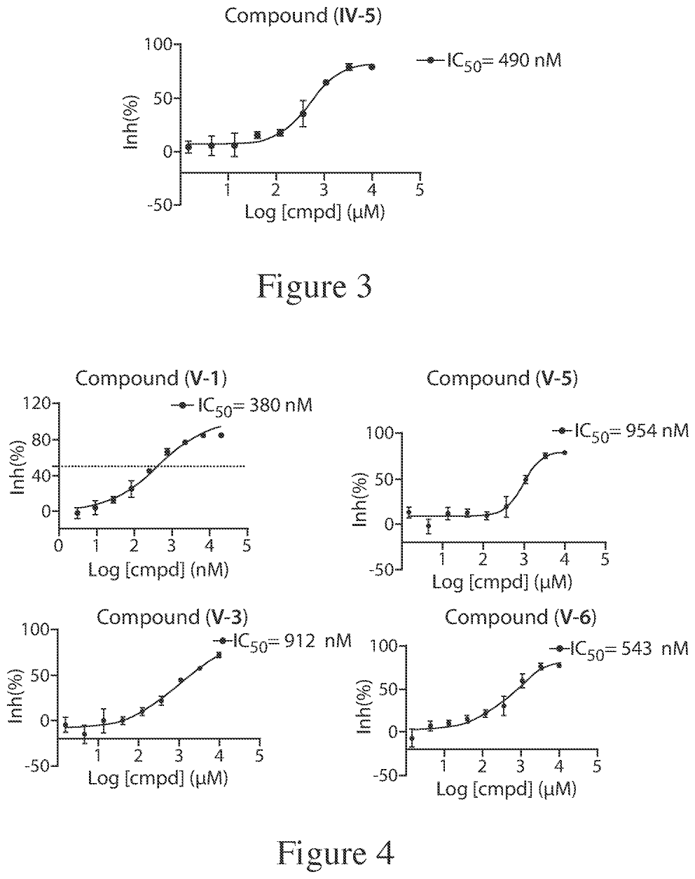

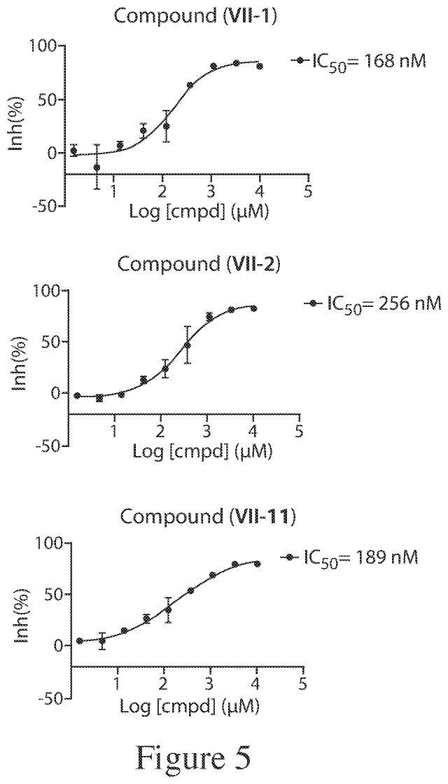

For 2D adherent cell viability experiment, the cells were seeded at 384-well plate (Corning, no. 3570) at the density of 200 cells/well (FIGS. 1-7). The next day, compounds were added using Janus workstation (PerkinElmer). After 5 days treatment, the cell viability was measured by CellTiter-Glo kit (Promega, no. G7570) as the manufacturer recommended.

TABLE 8
IC₅₀ values for representative compounds

| Compound No. | Chemical Structure | IC50 (nM) |
|---|---|---|
| I-1 | 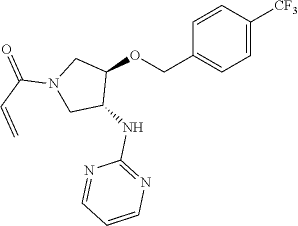 [SMILES: C=CC(=O)N1C[C@@H](Nc2ncccn2)[C@H](OCc2ccc(C(F)(F)F)cc2)C1] | 36 |
| I-9 | 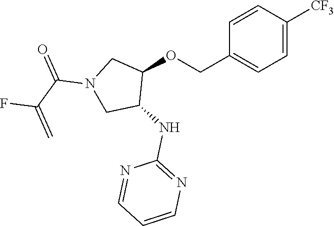 [SMILES: C=C(F)C(=O)N1C[C@@H](Nc2ncccn2)[C@H](OCc2ccc(C(F)(F)F)cc2)C1] | 23 |
| II-1 | 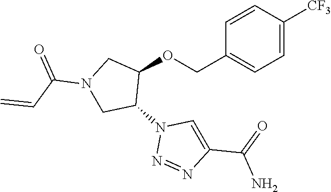 [SMILES: C=CC(=O)N1C[C@@H](n2cc(C(N)=O)nn2)[C@H](OCc2ccc(C(F)(F)F)cc2)C1] | 580 |
| II-2 | 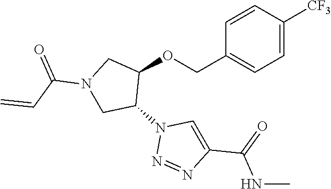 [SMILES: C=CC(=O)N1C[C@@H](n2cc(C(=O)NC)nn2)[C@H](OCc2ccc(C(F)(F)F)cc2)C1] | 461 |
| II-5 | 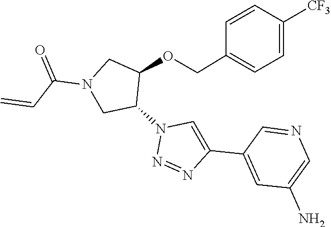 [SMILES: C=CC(=O)N1C[C@@H](n2cc(-c3cncc(N)c3)nn2)[C@H](OCc2ccc(C(F)(F)F)cc2)C1] | 172 |
| II-6 | 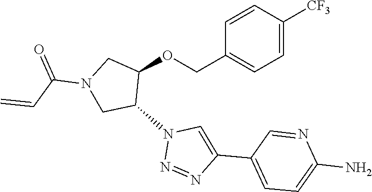 [SMILES: C=CC(=O)N1C[C@@H](n2cc(-c3ccc(N)nc3)nn2)[C@H](OCc2ccc(C(F)(F)F)cc2)C1] | 112 |
| II-7 | 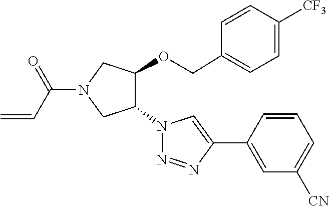 [SMILES: C=CC(=O)N1C[C@@H](n2cc(-c3cccc(C#N)c3)nn2)[C@H](OCc2ccc(C(F)(F)F)cc2)C1] | 377 |
| II-8 | 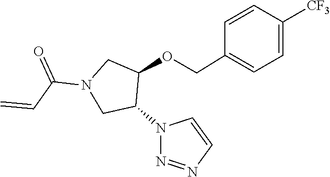 [SMILES: C=CC(=O)N1C[C@@H](n2ccnn2)[C@H](OCc2ccc(C(F)(F)F)cc2)C1] | 78 |
| II-19 | 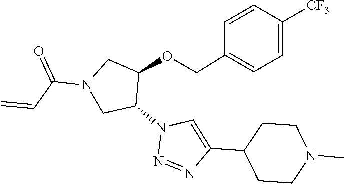 [SMILES: C=CC(=O)N1C[C@@H](n2cc(C3CCN(C)CC3)nn2)[C@H](OCc2ccc(C(F)(F)F)cc2)C1] | 193 |
| III-6 | 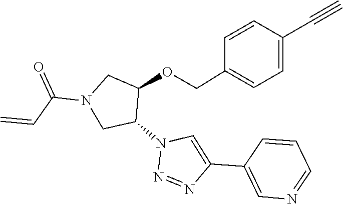 [SMILES: C#Cc1ccc(CO[C@@H]2CN(C(=O)C=C)C[C@H]2n2cc(-c3cccnc3)nn2)cc1] | 256 |
| IV-5 | 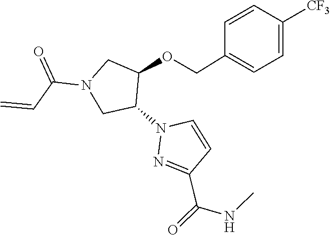 [SMILES: C=CC(=O)N1C[C@@H](n2ccc(C(=O)NC)n2)[C@H](OCc2ccc(C(F)(F)F)cc2)C1] | 490 |
| V-1 | 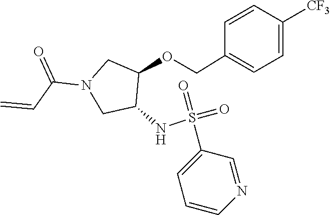 [SMILES: C=CC(=O)N1C[C@@H](NS(=O)(=O)c2cccnc2)[C@H](OCc2ccc(C(F)(F)F)cc2)C1] | 380 |
| V-3 | 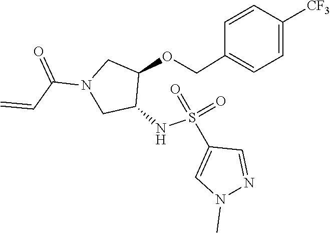 [SMILES: C=CC(=O)N1C[C@@H](NS(=O)(=O)c2cnn(C)c2)[C@H](OCc2ccc(C(F)(F)F)cc2)C1] | 912 |
| V-5 | 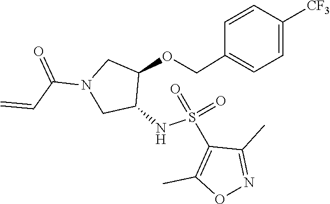 [SMILES: C=CC(=O)N1C[C@@H](NS(=O)(=O)c2c(C)noc2C)[C@H](OCc2ccc(C(F)(F)F)cc2)C1] | 954 |
| V-6 | 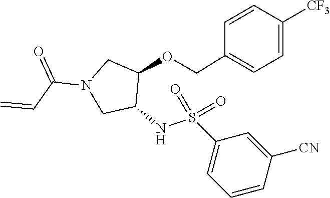 [SMILES: C=CC(=O)N1C[C@@H](NS(=O)(=O)c2cccc(C#N)c2)[C@H](OCc2ccc(C(F)(F)F)cc2)C1] | 543 |
| VI-2 | 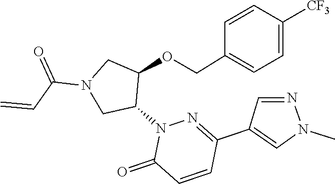 [SMILES: C=CC(=O)N1C[C@@H](n2nc(-c3cnn(C)c3)ccc2=O)[C@H](OCc2ccc(C(F)(F)F)cc2)C1] | 99 |
| VI-3 | 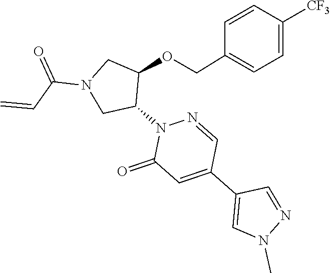 [SMILES: C=CC(=O)N1C[C@@H](n2ncc(-c3cnn(C)c3)cc2=O)[C@H](OCc2ccc(C(F)(F)F)cc2)C1] | 41 |
| VII-1 | 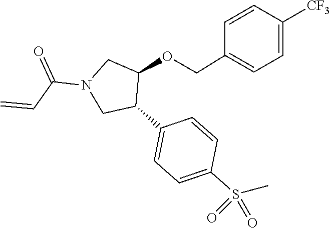 [SMILES: C=CC(=O)N1C[C@@H](OCc2ccc(C(F)(F)F)cc2)[C@H](c2ccc(S(C)(=O)=O)cc2)C1] | 168 |
| VII-2 | 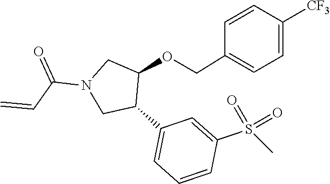 [SMILES: C=CC(=O)N1C[C@@H](OCc2ccc(C(F)(F)F)cc2)[C@H](c2cccc(S(C)(=O)=O)c2)C1] | 256 |
| VII-11 | 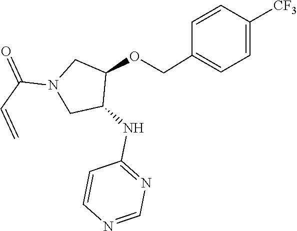 [SMILES: C=CC(=O)N1C[C@@H](Nc2ccncn2)[C@H](OCc2ccc(C(F)(F)F)cc2)C1] | 189 |

### Example 2. Gel-Based Anti-Palmitoylation Assay

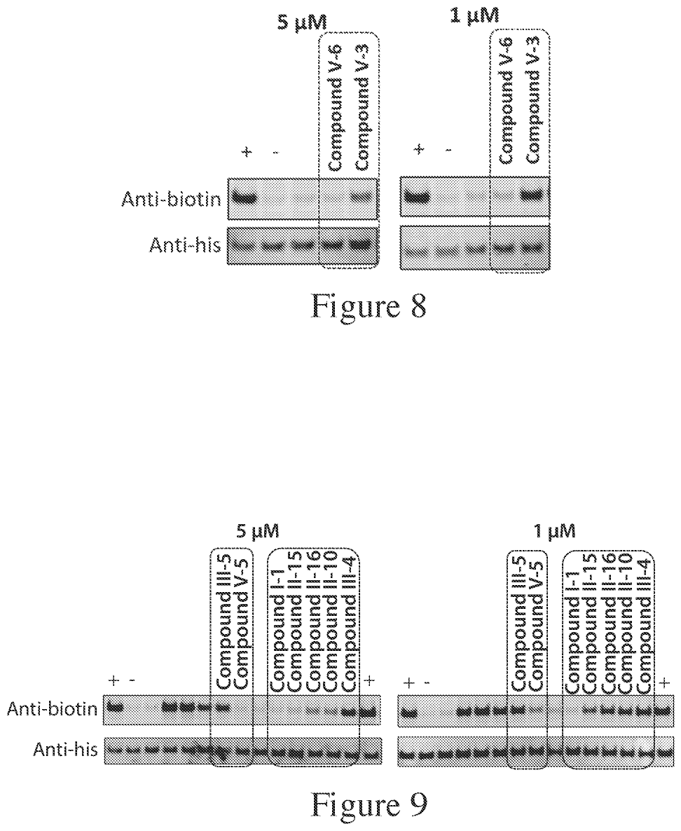

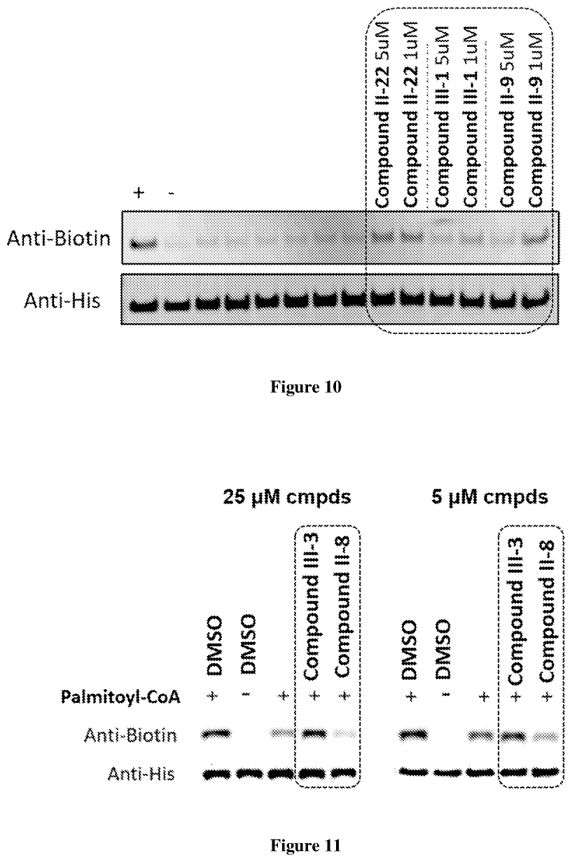

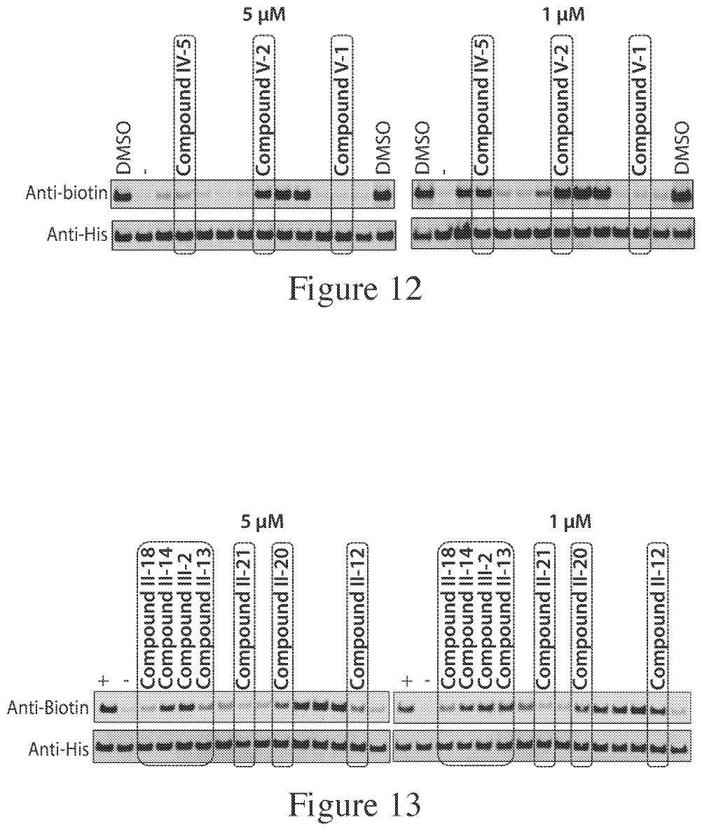

A 1 μM His-tag TEAD-YBD recombinant protein was incubated with inhibitors at the indicated concentrations at 37° C. for 2 h followed by the addition of palmitoyl alkyne-coenzyme A (Cayman chemical, no. 15968) in a total volume of 50 μL (See, FIGS. 8-13). After 30 min reaction, 5 μL 10% SDS were added and 5 μL click reagents were added to start click reaction. After another 1 h, 4× loading buffer were added to the reaction mixture and the samples subjected for western blot analysis. IRDye 800CW Streptavidin (LI-COR, no. 92632230) and His-Tag Mouse mAb (Cell Signaling, no. 2366S) was used for biotin detection and His-tag detection. The blots were imaged on Odyssey CLx Imager (LI-COR).

### Example 3. Synthesis of Compounds

Compounds IV-2, IV-3, IV-4, IV-5, VI-1, VI-2, VI-3, VII-1, VII-2, and VIII-1 were synthesized according to general synthesis route I shown in Scheme 1 and using the specific reagents as detailed in Table 9. Scheme 2 shows the synthesis of Compound IV-1.

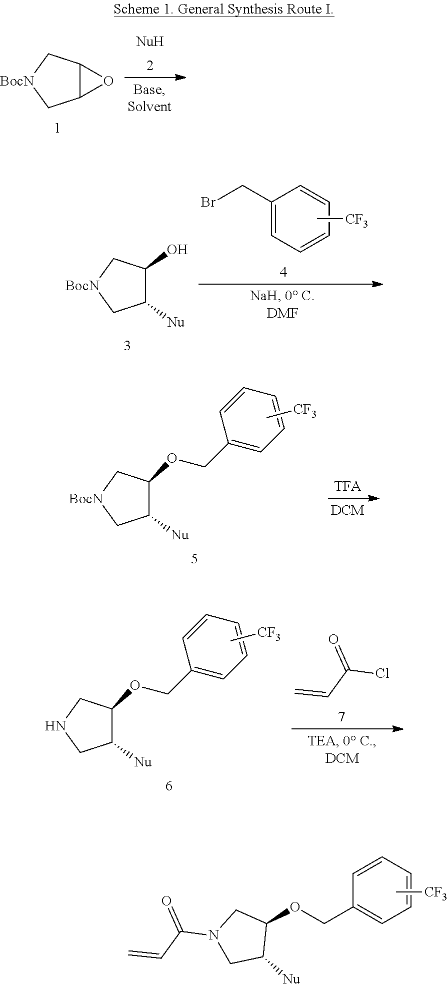

TABLE 9

| | | Base, | |
|---|---|---|---|
| Compound | 2 (NuH) | Solvent | 4 |
| 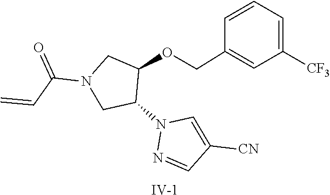 | 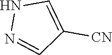 | Cs₂CO₃, DMF | 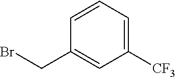 [SMILES: FC(F)(F)c1cccc(CBr)c1] |
| 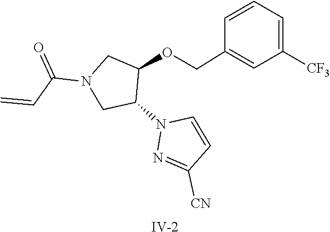 [SMILES: C=CC(=O)N1C[C@@H](n2ccc(C#N)n2)[C@H](OCc2cccc(C(F)(F)F)c2)C1 ; "IV-2"] | 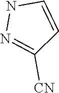 | Cs₂CO₃, DMF | 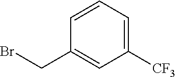 [SMILES: FC(F)(F)c1cccc(CBr)c1] |
| 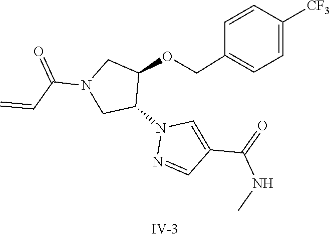 [SMILES: C=CC(=O)N1C[C@@H](n2cc(C(=O)NC)cn2)[C@H](OCc2ccc(C(F)(F)F)cc2)C1 ; "IV-3"] | 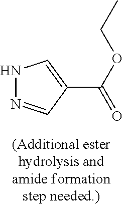 | Cs₂CO₃, DMF | 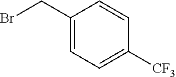 [SMILES: FC(F)(F)c1ccc(CBr)cc1] |
| 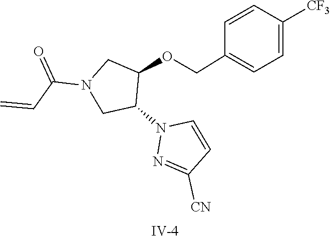 [SMILES: C=CC(=O)N1C[C@@H](n2ccc(C#N)n2)[C@H](OCc2ccc(C(F)(F)F)cc2)C1 ; "IV-4"] | 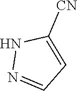 | Cs₂CO₃, DMF | 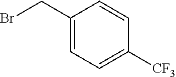 [SMILES: FC(F)(F)c1ccc(CBr)cc1] |
| 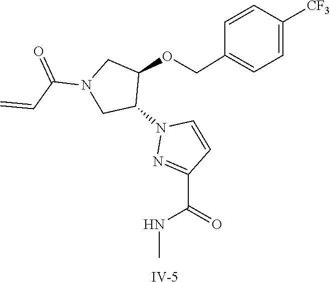 | 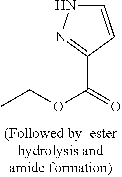 | Cs₂CO₃, DMF | 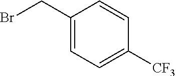 [SMILES: FC(F)(F)c1ccc(CBr)cc1] |
| 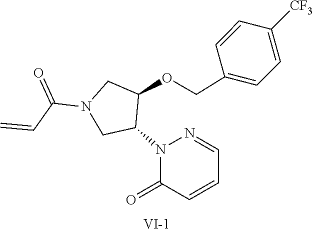 | 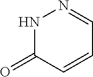 | NaH, DMF | 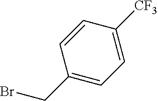 [SMILES: FC(F)(F)c1ccc(CBr)cc1] |
| 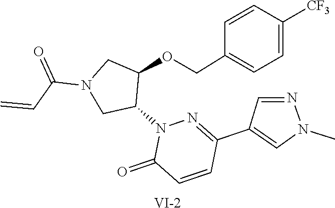 | 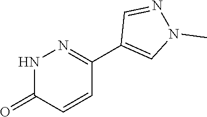 | Cs₂CO₃, EtOH, 80° C. | 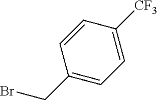 [SMILES: FC(F)(F)c1ccc(CBr)cc1] |
| 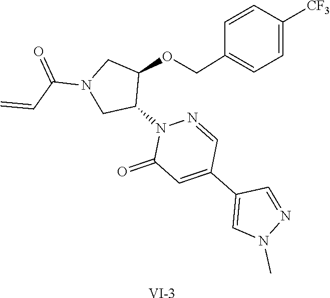 [SMILES: C=CC(=O)N1C[C@@H](n2ncc(-c3cnn(C)c3)cc2=O)[C@H](OCc2ccc(C(F)(F)F)cc2)C1 ; "VI-3"] | 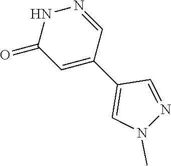 | Cs₂CO₃, EtOH, 80° C. | 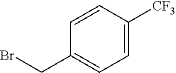 [SMILES: FC(F)(F)c1ccc(CBr)cc1] |
| 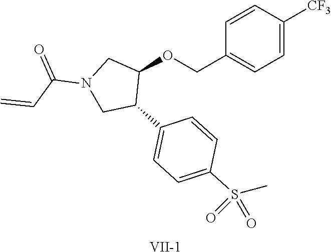 [SMILES: C=CC(=O)N1C[C@@H](OCc2ccc(C(F)(F)F)cc2)[C@H](c2ccc(S(C)(=O)=O)cc2)C1 ; "VII-1"] | 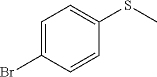 | 1, Mg, CuI THF 2, mCPBA DCM | 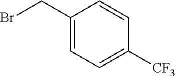 [SMILES: FC(F)(F)c1ccc(CBr)cc1] |
| 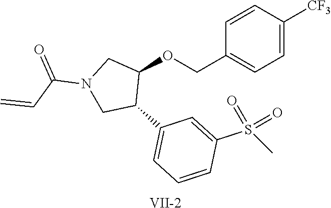 | 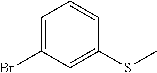 | 1, Mg, CuI THF 2, mCPBA DCM | 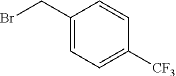 [SMILES: FC(F)(F)c1ccc(CBr)cc1] |

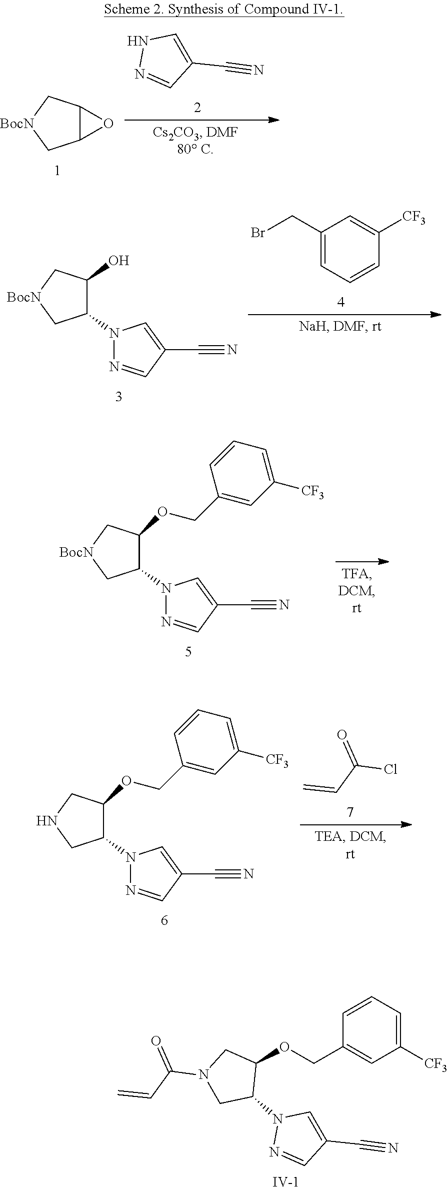

### Step 1: Synthesis of Tert-Butyl trans-3-(4-cyano-1H-pyrazol-1-yl)-4-hydroxypyrrolidine-1-carboxylate (Compound 3)

A mixture of tert-butyl 6-oxa-3-azabicyclo[3.1.0]hexane-3-carboxylate (200 mg, 1.08 mmol), 1H-pyrazole-4-carbonitrile (100 mg, 1.08 mmol) and Cs₂CO₃ (351 mg, 1.08 mmol) in DMF (10 mL) was stirred at 80° C. under N₂ overnight. The reaction mixture was cooled down to rt, diluted with water (50 mL) and extracted with ethyl acetate (50 mL×2), the combined organic was washed with H₂O (50 mL), dried over anhydrous Na₂SO₄, concentrated and purified by flash column chromatography on silica gel (ethyl acetate in petroleum ether=20% v/v) to get compound 3 as yellow oil (200 mg, yield 66%). LC-MS (ESI) m/z: 179 [M+H−100]⁺.

### Step 2: Synthesis of Tert-Butyl trans-3-(4-cyano-1H-pyrazol-1-yl)-4-(3-(trifluoromethyl)benzyloxy)pyrrolidine-1-carboxylate (Compound 5)

A mixture of tert-butyl trans-3-(4-cyano-1H-pyrazol-1-yl)-4-hydroxypyrrolidine-1-carboxylate (20 mg, 0.07 mmol), 1-(bromomethyl)-3-(trifluoromethyl)benzene (17 mg, 0.07 mmol) and NaH (3 mg, 0.07 mmol) in DMF (5 mL) was stirred at rt under N₂ for 2 hours. The reaction mixture was diluted with water (50 mL) and extracted with ethyl acetate (50 mL×2), the combined organic was washed with H₂O (50 mL), dried over anhydrous Na₂SO₄, concentrated and purified by flash column chromatography on silica gel (ethyl acetate in petroleum ether=40% v/v) to obtain the crude compound 5 as yellow oil (36 mg, yield 98%). LC-MS (ESI) m/z: 437 [M+H−100]⁺.

### Step 3: Synthesis of 1-((trans-4-(3-(trifluoromethyl)benzyloxy)pyrrolidin-3-yl)-1H-pyrazole-4-carbonitrile (Compound 6)

A mixture of compound 5 (36 mg, 0.08 mmol) and TFA (1 mL) in DCM (3 mL) was stirred at rt under N₂ for 2 hours. The mixture was concentrated to leave the crude compound 6 as yellow oil (26 mg, yield 96%), which was used directly for next step. LC-MS (ESI) m/z: 337 [M+H]⁺.

### Step 4: Synthesis of 1-(trans-1-acryloyl-4-(3-(trifluoromethyl)benzyloxy)pyrrolidin-3-yl)-1H-pyrazole-4-carbonitrile (IV-1)

To a mixture of compound 6 (30 mg, 0.089 mmol) and TEA (18 mg, 0.178 mmol) in DCM (5 mL) was added acryloyl chloride (8 mg, 0.089 mmol), the mixture was stirred at rt under N₂ for 2 hours. The mixture was concentrated and purified by prep-HPLC (MeCN/H2O/TFA) to obtain the target product as yellow oil (6 mg, yield 18%). LC-MS (ESI) m/z: 391 [M+H]⁺. ¹H-NMR (400 MHz, CD₃OD) δ (ppm): 8.48 (d, J=6.5 Hz, 1H), 8.00 (s, 1H), 7.75-7.56 (m, 4H), 6.69 (ddd, J=16.8, 10.4, 8.4 Hz, 1H), 6.47-6.31 (m, 1H), 5.86 (ddd, J=10.4, 5.8, 1.9 Hz, 1H), 5.36-5.18 (m, 1H), 4.78 (t, J=3.5 Hz, 2H), 4.58 (dd, J=18.5, 5.7 Hz, 1H), 4.40-3.65 (m, 4H).

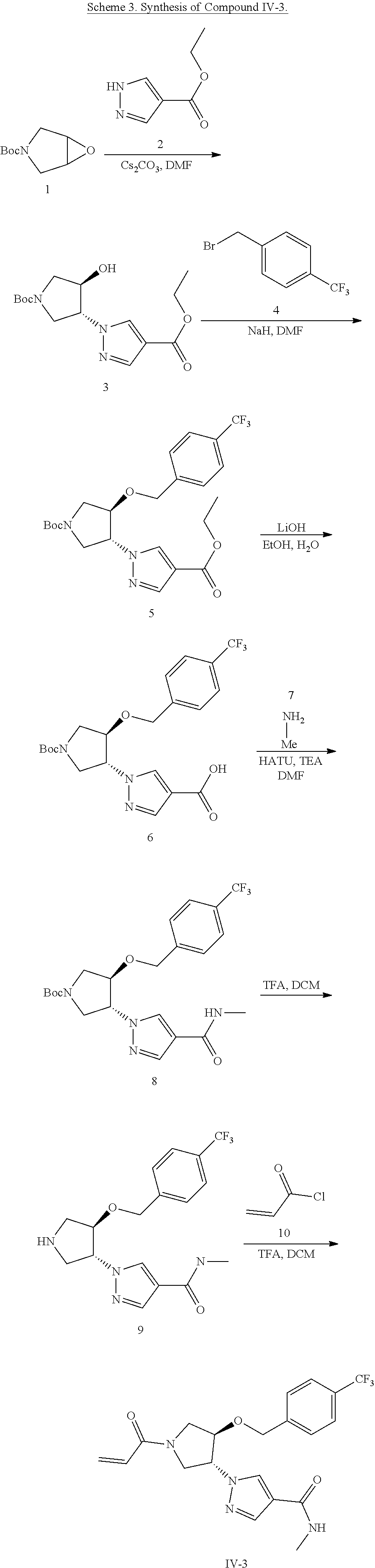

### Step 1: Synthesis of ethyl 1-(trans-1-(tert-butoxycarbonyl)-4-hydroxypyrrolidin-3-yl)-1H-pyrazole-4-carboxylate (Compound 3)

A mixture of tert-butyl 6-oxa-3-azabicyclo[3.1.0]hexane-3-carboxylate (647 mg, 3.5 mmol), ethyl 1H-pyrazole-4-carboxylate (500 mg, 3.5 mmol) and Cs₂CO₃ (1160 mg, 3.5 mmol) in DMF (10 mL) was stirred at 80° C. at rt under N₂ overnight. The reaction mixture was diluted with water (100 mL) and extracted with ethyl acetate (100 mL), the organic was washed with water (100 mL), dried over anhydrous Na₂SO₄, concentrated and purified by column chromatography on silica gel (ethyl acetate in petroleum ether=50% v/v) to obtain compound 3 as white oil (900 mg, yield 79%). LC-MS (ESI) m/z: 348 [M+23]⁺.

### Step 2: Synthesis of ethyl 1-(trans-1-(tert-butoxycarbonyl)-4-(4-(trifluoromethyl)benzyloxy)pyrrolidin-3-yl)-1H-pyrazole-4-carboxylate (Compound 5)

A mixture of compound 3 (370 mg, 1.1 mmol), 1-(bromomethyl)-4-(trifluoromethyl)benzene (272 mg, 1.1 mmol) and NaH (44 mg, 1.1 mmol) in DMF (10 mL) was stirred at rt under N₂ for 2 hours. The reaction mixture was monitored by LCMS. The mixture was extracted with ethyl acetate (100 mL), washed with water (50 mL), dried over anhydrous Na₂SO₄, filtered and concentrated to leave crude compound 5 as yellow oil (500 mg, yield 94%). LC-MS (ESI) m/z: 428 [M+H−56]⁺.

### Step 3: Synthesis of 1-(trans-1-(tert-butoxycarbonyl)-4-(4-(trifluoromethyl)benzyloxy)pyrrolidin-3-yl)-1H-pyrazole-4-carboxylic acid (Compound 6)

A mixture of compound 5 (200 mg, 0.41 mmol) and LiOH (50 mg. 2 mmol) in H₂O (4 mL) and EtOH (10 mL) was stirred at rt overnight. The reaction mixture was concentrated to leave the crude compound 6 as yellow oil (150 mg, yield 80%). LC-MS (ESI) m/z: 400 [M+H−56]⁺.

### Step 4: Synthesis of tert-butyl-trans-3-(4-(methylcarbamoyl)-1H-pyrazol-1-yl)-4-((4-(trifluoromethyl)benzyl)oxy)pyrrolidine-1-carboxylate (Compound 8)

A mixture of compound 6 (150 mg, 0.32 mmol), methylamine (10 mg, 0.32 mmol), HATU (182 mg. 0.48 mmol) and TEA (97 mg, 0.64 mmol) in DMF (4 mL) was stirred at rt overnight. The mixture was purified directly by prep-HPLC to obtain compound 8 as oil (65 mg, yield 43%). LC-MS (ESI) m/z: 413 [M+H−56]⁺.

### Synthesis of N-methyl-1-(trans-4-((4-(trifluoromethyl)benzyl)oxy)pyrrolidin-3-yl)-1H-pyrazole-4-carboxamide (Compound 9)

A mixture of compound 8 (65 mg. 0.13 mmol) and TFA (3 mL) in DCM (5 mL) was stirred at rt under N₂ for 2 hours. The mixture was concentrated to leave the crude compound 9 as yellow oil (45 mg, yield 93%). LC-MS (ESI) m/z: 369 [M+H]⁺.

### Step 4: Synthesis of 1-(trans-1-acryloyl-4-(4-(trifluoromethyl)benzyloxy)pyrrolidin-3-yl)-N-methyl-1H-pyrazole-4-carboxamide (IV-3)

A mixture of compound 9 (50 mg, 0.13 mmol), acryloyl chloride (12 mg. 0.13 mmol) and TEA (26 mg, 0.26 mmol) in DCM (5 mL) was stirred at rt under N₂ for 2 hours. The mixture was concentrated and purified by prep-HPLC to obtain the target compound IV-3 as white solid (16 mg, yield 29%). LC-MS (ESI) m/z: 423 [M+H]⁺. ¹H NMR (400 MHz, DMSO-d₆): δ (ppm) 8.28 (d, J=7.3 Hz, 1H), 8.09 (d, J=3.0 Hz, 1H), 7.89 (d, J=3.5 Hz, 1H), 7.70 (d, J=8.1 Hz, 2H), 7.51 (d, J=8.1 Hz, 2H), 6.62 (ddd, J=16.7, 10.3, 4.0 Hz, 1H), 6.18 (dt, J=16.8, 2.3 Hz, 1H), 5.71 (ddd, J=10.2, 5.9, 2.3 Hz, 1H), 5.31-5.04 (m, 1H), 4.69 (d, J=4.9 Hz, 2H), 4.39 (dd, J=23.0, 5.3 Hz, 1H), 4.25-3.49 (m, 4H), 2.71 (d, J=4.5 Hz, 3H).

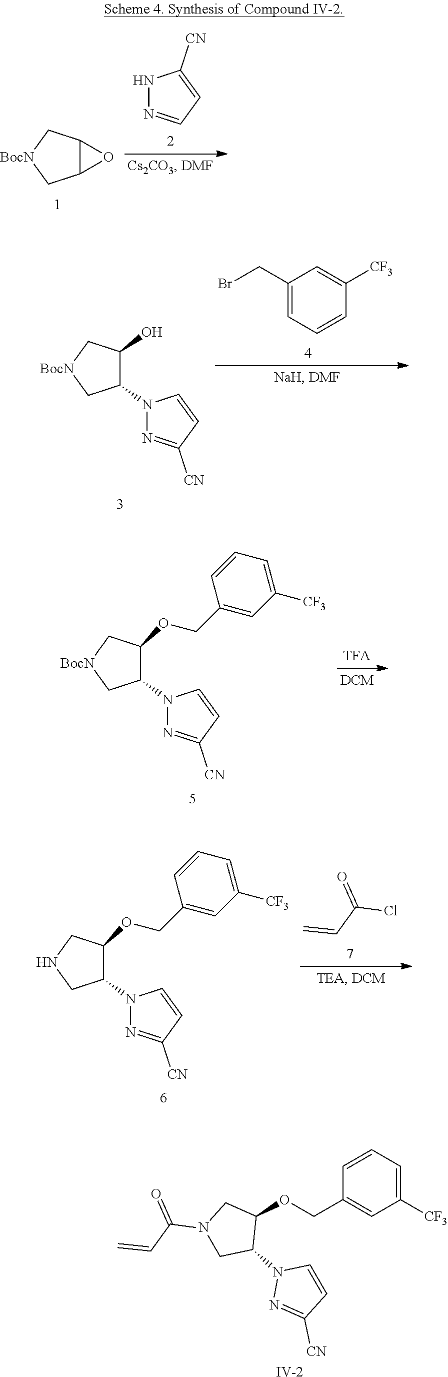

### Step 1: Synthesis of tert-butyl trans-3-(3-cyano-1H-pyrazol-1-yl)-4-hydroxypyrrolidine-1-carboxylate (Compound 3)

A mixture of tert-butyl 6-oxa-3-azabicyclo[3.1.0]hexane-3-carboxylate (397 mg, 2.15 mmol), 1H-pyrazole-5-carbonitrile (200 mg, 2.15 mmol) and Cs₂CO₃ (698 mg, 2.15 mmol) in DMF (5 mL) was stirred at 80° C. under N₂ overnight. The reaction mixture was monitored by LCMS. The mixture was diluted with ethyl acetate (50 mL), washed with H₂O (50 mL), dried over anhydrous Na₂SO₄, concentrated and purified by column chromatography on silica gel (ethyl acetate in petroleum ether=50% v/v) to obtain compound 3 as clear oil (145 mg, yield 38%). LC-MS (ESI) m/z: 179 [M+H−100]⁺.

### Step 2: Synthesis of Tert-Butyl trans-3-(3-cyano-1H-pyrazol-1-yl)-4-(3-(trifluoromethyl)benzyloxy)pyrrolidine-1-carboxylate (Compound 5)

To a mixture of compound 3 (200 mg, 0.71 mmol) in DMF (10 mL) was added NaH (29 mg, 0.71 mmol), the mixture was stirred at rt for 10 minutes, and then 1-(bromomethyl)-3-(trifluoromethyl)benzene (170 mg, 0.71 mmol) was added, the mixture was stirred at rt under N₂ for 2 hours. The reaction mixture was monitored by LCMS. The mixture was diluted with ethyl acetate (100 mL), washed with H₂O (50 mL), dried over anhydrous Na₂SO₄, filtered and concentrated to leave the crude compound 5 as yellow oil (260 mg, yield 85%). LC-MS (ESI) m/z: 437 [M+H−100]⁺.

### Step 3: Synthesis of 1-(trans-4-(3-(trifluoromethyl)benzyloxy)pyrrolidin-3-yl)-1H-pyrazole-3-carbonitrile (Compound 6)

A mixture of compound 5 (100 mg, 0.23 mmol) and TFA (3 mL) in DCM (10 mL) was stirred at rt under N₂ for 2 hours. The mixture was concentrated to leave the crude compound 6 as yellow oil (70 mg, yield 90%). LC-MS (ESI) m/z: 337 [M+H]⁺.

### Step 4: Synthesis of 1-(trans-1-acryloyl-4-(3-(trifluoromethyl)benzyloxy)pyrrolidin-3-yl)-1H-pyrazole-3-carbonitrile (IV-2)

A mixture of compound 6 (80 mg, 0.24 mmol), acryloyl chloride (21 mg, 0.24 mmol) and TEA (48 mg, 0.24 mmol) in DCM (5 mL) was stirred at rt under N₂ for 2 hours. The mixture was concentrated and purified by prep-HPLC (MeCN/H₂O/TFA) to obtain IV-2 as yellow oil (35 mg, yield 37%). LC-MS (ESI) m/z: 391 [M+H]⁺. ¹H NMR (400 MHz, CD₃OD): δ (ppm) 7.96 (dd, J=4.9, 2.5 Hz, 1H), 7.69-7.47 (m, 4H), 6.84 (t, J=2.5 Hz, 1H), 6.64 (ddd, J=16.8, 10.4, 8.9 Hz, 1H), 6.34 (d, J=16.7 Hz, 1H), 5.81 (ddd, J=10.4, 6.0, 1.9 Hz, 1H), 5.34-5.11 (m, 1H), 4.71 (t, J=3.9 Hz, 2H), 4.52 (dd, J=19.2, 5.7 Hz, 1H), 4.34-3.65 (m, 4H).

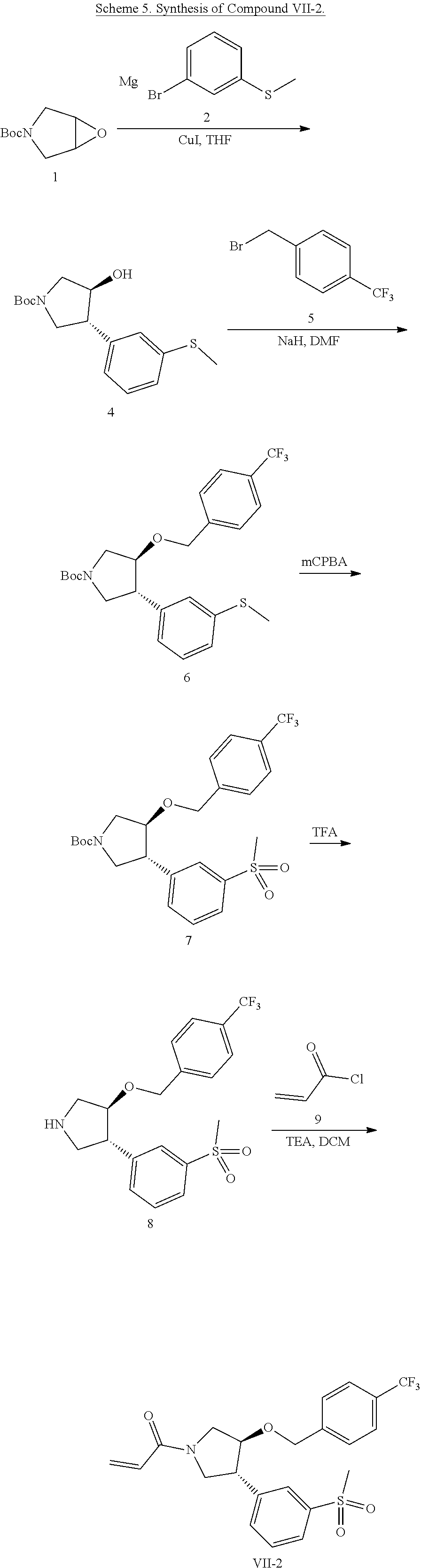

### Step 1: Synthesis of Tert-Butyl trans-3-hydroxy-4-(3-(methylthio)phenyl)pyrrolidine-1-carboxylate (Compound 4)

A mixture of (3-bromophenyl) (methyl) sulfane (1085 mg, 5.4 mmol) and Mg (129 mg, 5.4 mmol) in THF (10 mL) was stirred at reflux under N₂ for 2 hours, then the mixture was cooled down to 0° C., tert-butyl 6-oxa-3-azabicyclo[3.1.0]hexane-3-carboxylate (1000 mg. 5.4 mmol) and CuI (102 mg, 0.54 mmol) in THF (5 mL) was added, the resulting mixture was stirred at 50° C. overnight. The mixture was cooled down to rt, diluted with water (50 mL) and extracted with ethyl acetate (100 mL). The organic layer was dried over anhydrous Na₂SO₄, concentrated and purified by column chromatography on silica gel (ethyl acetate in petroleum ether=30% v/v) to obtain the product 4 as white solid (850 mg, yield 51%). LC-MS (ESI) m/z: 254 [M+H−56]⁺.

### Step 2: Synthesis of Tert-Butyl trans-3-(3-(methylthio)phenyl)-4-(4-(trifluoromethyl)benzyloxy)pyrrolidine-1-carboxylate (Compound 6)

To a mixture of tert-butyl trans-3-hydroxy-4-(3-(methylthio)phenyl)pyrrolidine-1-carboxylate (200 mg. 0.64 mmol) and NaH (26 mg, 0.64 mmol) in DMF (5 mL) was added 1-(bromomethyl)-4-(trifluoromethyl)benzene (154 mg, 0.54 mmol), the mixture was stirred at rt under N₂ for 2 hours, diluted with water (50 mL) and extracted with ethyl acetate (100 mL). The organic layer was dried over anhydrous Na₂SO₄, filtered and concentrated to leave crude product 6 as yellow oil (250 mg, yield 83%), which was used directly for next step. LC-MS (ESI) m/z: 368 [M+H−100]⁺.

### Step 3: Synthesis of Tert-Butyl trans-3-(3-(methylsulfonyl)phenyl)-4-(4-(trifluoromethyl)benzyloxy)pyrrolidine-1-carboxylate (Compound 7)

A mixture of tert-butyl trans-3-(3-(methylthio)phenyl)-4-((4-(trifluoromethyl)benzyl)oxy)pyrrolidine-1-carboxylate (260 mg, 0.55 mmol) and mCPBA (239 mg, 1.4 mmol) in DCM (10 mL) was stirred at rt under N₂ overnight. The mixture was concentrated and purified by flash column chromatography on silica gel (ethyl acetate in petroleum ether=30% v/v) to obtain the product 7 as yellow oil (30 mg, yield 11%). LC-MS (ESI) m/z: 522 [M+23]⁺.

### Step 4: Synthesis of trans-3-(3-(methylsulfonyl)phenyl)-4-(4-(trifluoromethyl)benzyloxy)pyrrolidine (Compound 8)

A mixture of tert-butyl trans-3-(3-(methylsulfonyl)phenyl)-4-((4-(trifluoromethyl)benzyl)oxy)pyrrolidine-1-carboxylate (30 mg, 0.06 mmol) and TFA (3 mL) in DCM (5 mL) was stirred at rt for 2 hours. The mixture was concentrated to leave crude product 8 as yellow oil (23 mg, yield 100%). LC-MS (ESI) m/z: 400 [M+H]⁺.

### Step 5: Synthesis of trans-3-(3-(methylsulfonyl)phenyl)-4-(4-(trifluoromethyl)benzyloxy)pyrrolidin-1-yl)prop-2-en-1-one (VII-2)

A mixture of trans-3-(3-(methylsulfonyl)phenyl)-4-(4-(trifluoromethyl)benzyloxy)pyrrolidine (25 mg, 0.06 mmol), acryloyl chloride (6 mg, 0.06 mmol) and TEA (12 mg, 0.12 mmol) in DCM (5 mL) was stirred at rt for 2 hours. The mixture was concentrated and purified by prep-HPLC (MeCN/H₂O/TFA) to obtain the target VII-2 as white solid (7 mg yield 26%). LC-MS (ESI) m/z: 454 [M+H]⁺. ¹H-NMR (400 MHz, CD₃OD) δ (ppm) 7.98-7.88 (m, 2H), 7.71-7.59 (m, 4H), 7.47 (d, J=8.0 Hz, 2H), 6.67 (td, J=16.3, 10.5 Hz, 1H), 6.35 (dd, J=16.8, 1.8 Hz, 1H), 5.81 (dd, J=10.4, 2.2 Hz, 1H), 4.68 (q, J=12.7 Hz, 2H), 4.42-3.95 (m, 3H), 3.94-3.53 (m, 3H), 3.14 (s, 3H).

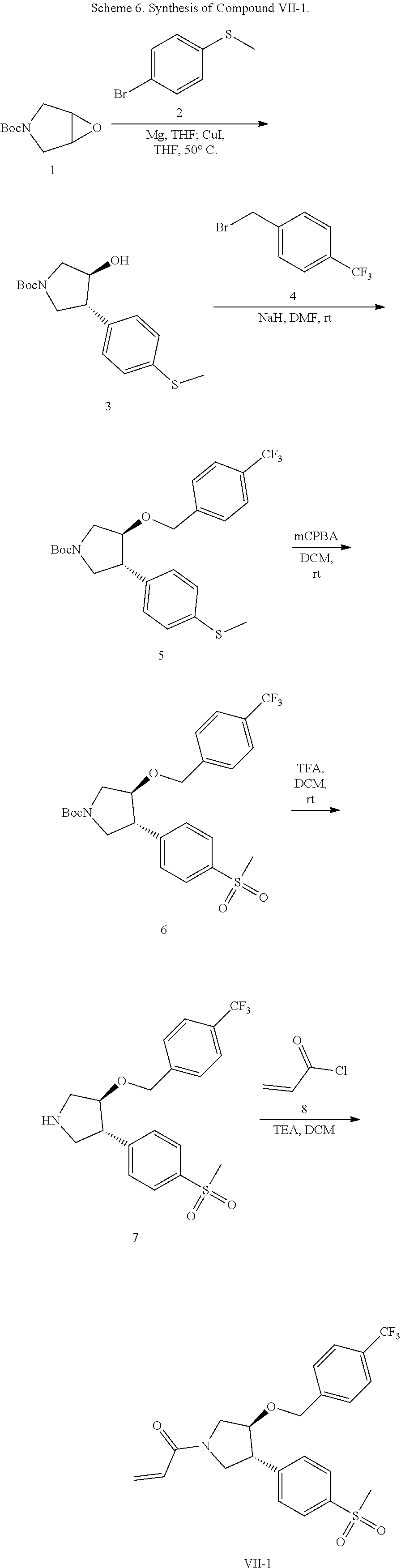

### Step 1: Synthesis of tert-butyl trans-3-hydroxy-4-(4-(methylthio)phenyl)pyrrolidine-1-carboxylate (Compound 3)

A mixture of (4-bromophenyl) (methyl) sulfane (2190 mg, 10.8 mmol) and Mg (259 mg, 10.8 mmol) in THF (200 mL) was stirred at refluxed under N₂ for 2 hours. Then the reaction mixture was cooled down to 0° C., a mixture of tert-butyl 6-oxa-3-azabicyclo[3.1.0]hexane-3-carboxylate (2000 mg, 10.8 mmol) and CuI (205 mg, 1.08 mmol) in THF (10 mL) was added, the resulting mixture was stirred at 50° C. overnight. The mixture was diluted with water (50 mL) and extracted with ethyl acetate (50 mL×2), the combined organic was washed with water (100 mL), dried over anhydrous Na₂SO₄, concentrated and purified by flash column chromatography on silica gel (ethyl acetate in petroleum ether=30% v/v) to obtain the target compound 3 as white solid (3 g, yield 90%). LC-MS (ESI) m/z: 254 [M+H−56]⁺.

### Step 2: Synthesis of Tert-Butyl trans-3-(4-(methylthio)phenyl)-4-(4-(trifluoromethyl)benzyloxy)pyrrolidine-1-carboxylate (Compound 5)

To a solution of compound 3 (500 mg, 1.6 mmol) in DMF (10 mL) was added NaH (64 mg, 1.6 mmol), the mixture was stirred at 0° C. for 10 minutes, and then 1-(bromomethyl)-4-(trifluoromethyl)benzene (386 mg, 1.6 mmol) was added, the reaction mixture was stirred at rt under N₂ for 2 hours, diluted with water (50 mL) and extracted with ethyl acetate (50 mL×2), the combined organic was washed with water (100 mL), dried over anhydrous Na₂SO₄, concentrated and purified by flash column chromatography on silica gel (ethyl acetate in petroleum ether=40% v/v) to obtain compound 5 as yellow oil (660 mg, yield 88%). LC-MS (ESI) m/z: 412 [M+H−56]⁺.

### Step 3: Synthesis of Tert-Butyl trans-3-(4-(methylsulfonyl)phenyl)-4-(4-(trifluoromethyl)benzyloxy)pyrrolidine-1-carboxylate (Compound 6)

A mixture of compound 5 (300 mg, 0.64 mmol) and mCPBA (276 mg, 1.6 mmol) in DCM (10 mL) was stirred at rt under N₂ overnight. The mixture was concentrated and purified by flash column chromatography on silica gel (ethyl acetate in petroleum ether=40% v/v) to obtain compound 6 as yellow oil (260 mg, yield 81%). LC-MS (ESI) m/z: 522 [M+23]⁺.

### Step 4: Synthesis of trans-3-(4-(methylsulfonyl)phenyl)-4-(4-(trifluoromethyl)benzyloxy)pyrrolidine (Compound 7)

A mixture of compound 6 (100 mg. 0.2 mmol) and TFA (3 mL) in DCM (5 mL) was stirred at rt under N₂ for 2 hours. The mixture was concentrated to leave crude compound 7 as yellow oil (80 mg, yield 100%). LC-MS (ESI) m/z: 400 [M+H]⁺.

### Step 5: Synthesis of 1-(trans-3-(4-(methylsulfonyl)phenyl)-4-(4-(trifluoromethyl)benzyloxy)pyrrolidin-1-yl)prop-2-en-1-one (VII-1)

To a mixture of compound 7 (90 mg, 0.2 mmol) and TEA (44 mg. 0.4 mmol) in DCM (5 mL) was added acryloyl chloride (19 mg, 0.2 mmol), the mixture was stirred at rt under N₂ for 2 hours. The mixture was concentrated and purified by prep-HPLC (MeCN/H₂O/TFA) to obtain the target compound VII-1 as a white solid (20 mg, yield 18%). LC-MS (ESI) m/z: 454 [M+H]⁺. ¹H NMR (400 MHz, DMSO-d₆) δ (ppm) 7.90 (dd, J=8.3, 2.6 Hz, 2H), 7.69 (d, J=8.3, 2.6 Hz, 2H), 7.60 (d, J=8.4 Hz, 2H), 7.48 (d, J=8.0 Hz, 2H), 6.69-6.58 (m, 1H), 6.22-6.15 (m, 1H), 5.75-5.67 (m, 1H), 4.66 (d, J=3.4 Hz, 2H), 4.33-4.22 (m, 1H), 4.13-3.99 (m, 1H), 3.95-3.40 (m, 4H), 3.21 (s, 3H).

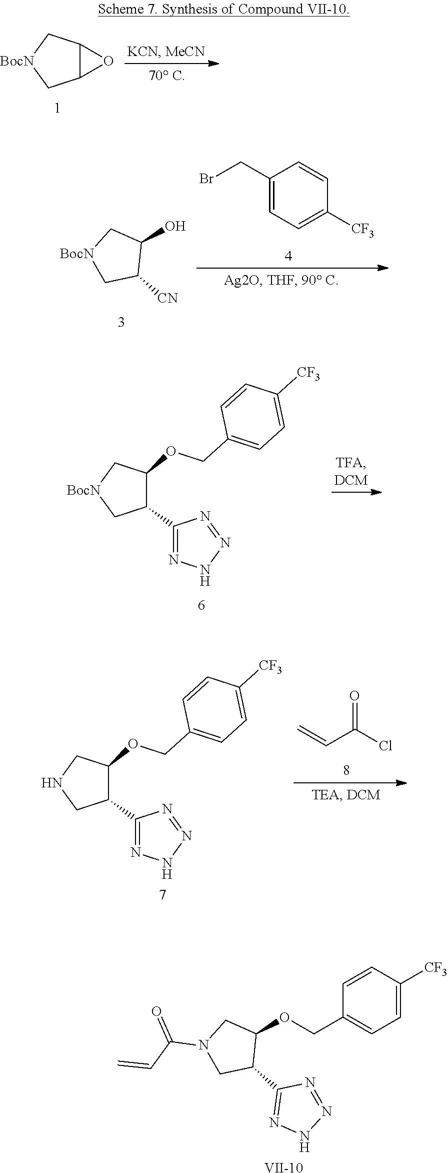

### Step 1: Synthesis of Tert-Butyl trans-3-cyano-4-hydroxypyrrolidine-1-carboxylate (Compound 3)

A mixture of tert-butyl 6-oxa-3-azabicyclo[3.1.0]hexane-3-carboxylate (1200 mg, 6.48 mmol), KCN (840 mg, 12.96 mmol) and LiClO₄ (1032 mg, 9.72 mmol) in CH₃CN (20 mL) was stirred at 70° C. under N₂ for 2 days n. The mixture was cooled down to rt, diluted with water (100 mL) and extracted with ethyl acetate (100 mL×2), the combined organic was washed with H₂O (50 mL), dried over anhydrous Na₂SO₄, concentrated and purified by prep-HPLC to obtain compound 3 as clear oil (1 g, yield 72%). LC-MS (ESI) m/z: 235 [M+23]⁺.

### Step 2: Synthesis of Tert-Butyl trans-3-cyano-4-(4-(trifluoromethyl)benzyloxy)pyrrolidine-1-carboxylate (Compound 5)

A mixture of compound 3 (300 mg, 1.41 mmol), 1-(bromomethyl)-4-(trifluoromethyl)benzene (360 mg, 1.41 mmol) and Ag₂O (1620 mg, 9.2 mmol) in THF (25 mL) was stirred at 90° C. under N₂ overnight. The reaction mixture was diluted with water (50 mL) and extracted with ethyl acetate (60 mL), the organic was washed with water (40 mL), dried over anhydrous Na₂SO₄, concentrated and purified by flash column chromatography on silica gel (ethyl acetate in petroleum ether=40% v/v) to obtain the crude compound 5 as yellow oil (300 mg). LC-MS (ESI) m/z: 315 [M+H−56]⁺.

### Step 3: Synthesis of Tert-Butyl trans-3-(2H-tetrazol-5-yl)-4-(4-(trifluoromethyl)benzyloxy)pyrrolidine-1-carboxylate (Compound 6)

A mixture of compound 5 (300 mg, 0.81 mmol), NaN₃ (90 mg, 1.2 mmol) and NH₄Cl (72 mg, 4.02 mmol) in DMF (5 mL) was stirred at 130° C. under N₂ overnight. The mixture was monitored by LCMS. The mixture was diluted with water (50 mL) and extracted with ethyl acetate (50 mL), the organic was washed with water (40 mL), dried over anhydrous Na₂SO₄, concentrated and purified by prep-HPLC to obtain compound 6 as yellow solid (60 mg, yield 24%). LC-MS (ESI) m/z: 314 [M+H−100]⁺.

### Step 4: Synthesis of 5-(trans-4-(4-(trifluoromethyl)benzyloxy)pyrrolidin-3-yl)-2H-tetrazole (Compound 7)

A mixture of compound 6 (50 mg, 0.1 mmol) and TFA (1 mL) in DCM (3 mL) was stirred at rt under N₂ for 2 hours. The mixture was concentrated to leave crude compound 7 as yellow oil (34 mg, yield 100%). LC-MS (ESI) m/z: 314 [M+H]⁺.

### Step 5: Synthesis of 1-(trans-3-(2H-tetrazol-5-yl)-4-(4-(trifluoromethyl)benzyloxy)pyrrolidin-1-yl)prop-2-en-1-one (VII-10)

A mixture of compound 7 (50 mg, 0.16 mmol), acryloyl chloride (20 mg, 0.16 mmol) and TEA (40 mg, 0.32 mmol) in DCM (5 mL) was stirred at rt for 2 hours. The mixture was concentrated and purified by prep-HPLC to obtain compound VII-10 as white solid (15 mg, yield 25%). LC-MS (ESI) m/z: 368 [M+H]⁺. ¹H NMR (400 MHz, CD₃OD) δ (ppm) 7.66 (d, J=8.1 Hz, 2H), 7.54 (d, J=6.8 Hz, 2H), 6.69-6.57 (m, 1H), 6.36-6.28 (m, 1H), 5.83-5.75 (m, 1H), 4.75 (s, 2H), 4.57-4.44 (m, 1H), 4.29-3.93 (m, 4H), 3.90-3.69 (m, 2H).

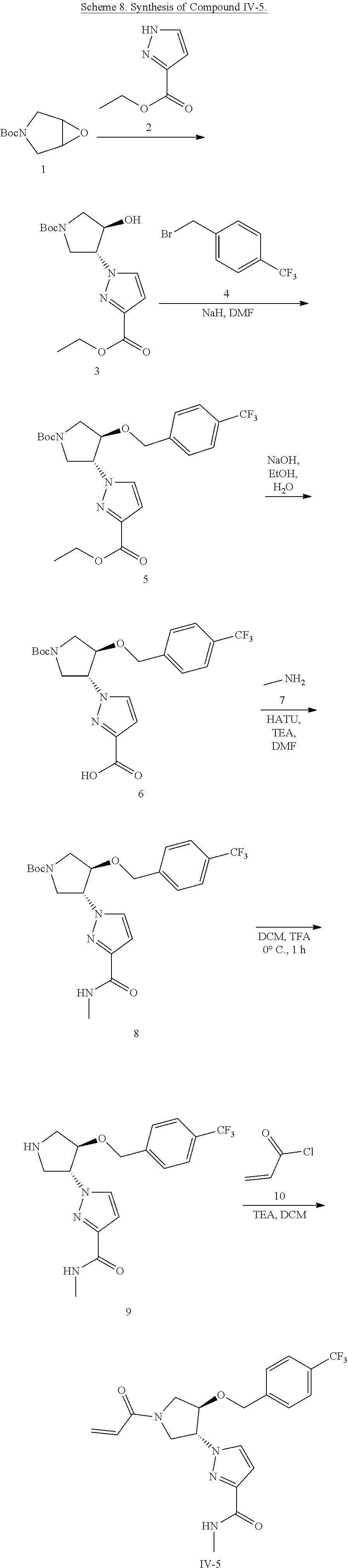

### Step 1: Synthesis of ethyl 1-(trans-1-(tert-butoxycarbonyl)-4-hydroxypyrrolidin-3-yl)-1H-pyrazole-3-carboxylate (Compound 3)

To the solution of tert-butyl 6-oxa-3-azabicyclo[3.1.0]hexane-3-carboxylate (1320 mg, 7.13 mmol) in dry DMF (20 mL) was added ethyl 1H-pyrazole-3-carboxylate (1000 mg, 7.13 mmol). The mixture was stirred at 50° C. overnight. The mixture was diluted with water (100 mL) and extracted with EtOAc (100 mL×3), the combined organics were washed with brine (200 mL), dried over anhydrous Na₂SO₄, concentrated and purified by column chromatography on silica gel (eluting with petroleum ether/ethyl acetate) to obtain the target compound 3 as oil (800 mg, yield 34.5%). LC-MS (ESI) m/z: 348 [M+Na]⁺.

### Step 2: Synthesis of ethyl 1-(trans-1-(tert-butoxycarbonyl)-4-(4-(trifluoromethyl)benzyloxy)pyrrolidin-3-yl)-1H-pyrazole-3-carboxylate (Compound 5)

To a solution of ethyl 1-(trans-1-(tert-butoxycarbonyl)-4-hydroxypyrrolidin-3-yl)-1H-pyrazole-3-carboxylate (700 mg, 2.15 mmol) in DMF (20 mL) was added NaH (60%, 103 mg, 2.58 mmol), the mixture was stirred at 0° C. under N₂ for 0.5 hour, and then 1-(bromomethyl)-4-(trifluoromethyl)benzene (512 mg, 2.15 mmol) was added, the resulting mixture was stirred at rt overnight. The mixture was diluted with water (100 mL) and extracted with EtOAc (100 mL×3), the combined organics were washed with brine (200 mL), dried over anhydrous Na₂SO₄, concentrated and purified by column chromatography on silica gel (eluting with petroleum ether/ethyl acetate) to obtain the target compound 5 as oil (700 mg, yield 67.4%). LC-MS (ESI) m/z: 506 [M+Na]⁺.

### Step 3: Synthesis of 1-(trans-1-(tert-butoxycarbonyl)-4-(4-(trifluoromethyl)benzyloxy)pyrrolidin-3-yl)-1H-pyrazole-3-carboxylic Acid (Compound 6)

To a solution of ethyl 1-(trans-1-(tert-butoxycarbonyl)-4-(4-(trifluoromethyl)benzyloxy)pyrrolidin-3-yl)-1H-pyrazole-3-carboxylate (400 mg. 0.83 mmol) in EtOH (15 mL) was added the solution of NaOH (132 mg. 3.31 mmol) in H₂O (15 mL). The mixture was stirred at 80° C. for 2 hours. The mixture was poured into aqueous HCl solution (1N, 40 mL) and extracted with EtOAc (100 mL×3), the combined organics were washed with brine (200 mL), dried over anhydrous Na₂SO₄, filtered and concentrated to leave the crude compound 6 as oil (400 mg, crude). LC-MS (ESI) m/z: 400 [M−55]⁺.

### Step 4: Synthesis of Tert-Butyl trans-3-(3-(methylcarbamoyl)-1H-pyrazol-1-yl)-4-(4-(trifluoromethyl)benzyloxy)pyrrolidine-1-carboxylate (Compound 8)

To a solution of 1-(trans-1-(tert-butoxycarbonyl)-4-(4-(trifluoromethyl)benzyloxy)-pyrrolidin-3-yl)-1H-pyrazole-3-carboxylic acid (400 mg. 0.88 mmol) in DMF (15 mL) was added methanamine solution in THF (0.4 mL, 2 M, 0.88 mmol), HATU (400 mg, 1.04 mmol) and Et₃N (177.6 mg, 3.12 mmol). The mixture was stirred at rt for 3 hours, diluted with water (100 mL) and extracted with EtOAc (50 mL×3), the combined organics were washed with brine (100 mL), dried over anhydrous Na₂SO₄, concentrated and purified by column chromatography on silica gel (eluting with DCM/MeOH) to obtain the target compound 8 as oil (340 mg, yield 82.7%). LC-MS (ESI) m/z: 491 [M+Na]⁺.

### Step 5: Synthesis of N-methyl-1-(trans-4-(4-(trifluoromethyl)benzyloxy)pyrrolidin-3-yl)-1H-pyrazole-3-carboxamide (Compound 9)

To the solution of compound 8 (290 mg. 0.62 mmol) in DCM (10 mL) was added TFA (2 mL), the mixture was stirred at 0° C. for 1 hour, and then concentrated to leave crude compound 9 as oil (300 mg, crude). LC-MS (ESI) m/z: 369 [M+H]⁺.

### Step 6: Synthesis of 1-(trans-1-acryloyl-4-(4-(trifluoromethyl)benzyloxy)pyrrolidin-3-yl)-N-methyl-1H-pyrazole-3-carboxamide (Compound IV-5)

To the solution of compound 9 (250 mg. 0.74 mmol) in DCM (15 mL) was added acryloyl chloride (61 mg. 0.74 mmol) and Et₃N (137 mg, 1.47 mmol). The mixture was stirred at 0° C. for 1 hour, and then diluted with water (100 mL) and extracted with EtOAc (50 mL×2), the combined organics were washed with brine (100 mL), dried over anhydrous Na₂SO₄, concentrated and purified by prep-HPLC to obtain the target compound IV-5 as solid (50 mg, yield 16%). LC-MS (ESI) m/z: 423 [M+H]⁺. ¹H NMR (400 MHz, CD₃OD) δ (ppm) 7.87-7.78 (m, 1H), 7.62 (d, J=8.0 Hz, 2H), 7.46 (d, J=8.0 Hz, 2H), 6.76 (d, J=2.4 Hz, 1H), 6.64 (ddd, J=17.4, 10.4, 7.4 Hz, 1H), 6.34 (dd, J=16.8, 1.6 Hz, 1H), 5.85-5.77 (m, 1H), 5.20-5.08 (m, 1H), 4.72-4.67 (m, 2H), 4.60-4.49 (m, 1H), 4.33-4.19 (m, 1H), 4.17-3.96 (m, 2H), 3.85-3.65 (m, 1H), 2.90 (s, 3H).

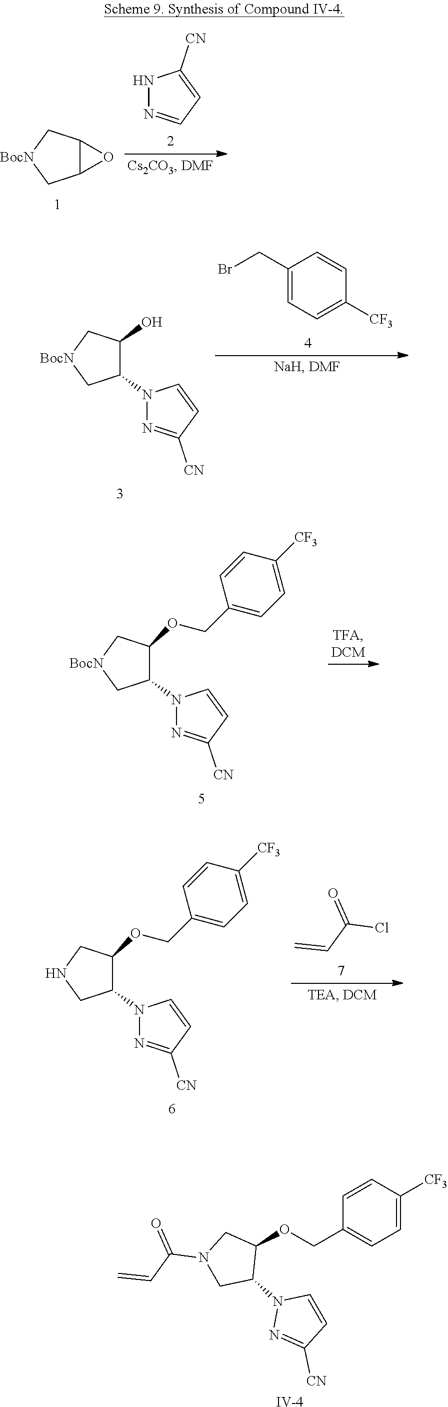

### Step 1: Synthesis of tert-butyl trans-3-(3-cyano-1H-pyrazol-1-yl)-4-hydroxypyrrolidine-1-carboxylate (Compound 3)

A mixture of tert-butyl 6-oxa-3-azabicyclo[3.1.0]hexane-3-carboxylate (397 mg, 2.15 mmol), 1H-pyrazole-5-carbonitrile (200 mg, 2.15 mmol) and Cs₂CO₃ (698 mg, 2.15 mmol) in DMF (5 mL) was stirred at 80° C. overnight. The mixture was diluted with water (100 mL) and extracted with ethyl acetate (100 mL), the organic was washed with H₂O (100 mL), dried over anhydrous Na₂SO₄, concentrated and purified by column chromatography on silica gel (ethyl acetate in petroleum ether=50% v/v) to obtain compound 3 as white oil (200 mg, yield 33%). LC-MS (ESI) m/z: 279 [M+H]⁺.

### Step 2: Synthesis of tert-butyl trans-3-(3-cyano-1H-pyrazol-1-yl)-4-(4-(trifluoromethyl)benzyloxy)pyrrolidine-1-carboxylate (Compound 5)

To the solution of compound 3 (200 mg, 0.71 mmol) in DMF (10 mL) was added NaH (28 mg, 0.71 mmol), the mixture was stirred at rt for 10 minutes, and then 1-(bromomethyl)-4-(trifluoromethyl)benzene (172 mg, 0.71 mmol) was added, the resulting mixture was stirred at rt under N₂ for 2 hours, diluted with water (100 mL) and extracted with ethyl acetate (100 mL), the organic was washed with H₂O (100 mL), dried over anhydrous Na₂SO₄, filtered and concentrated to leave the crude compound 5 as yellow oil (300 mg, yield 96%), which was used directly for next step. LC-MS (ESI) m/z: 337 [M+H−100]⁺.

### Step 3: Synthesis of 1-(trans-4-(4-(trifluoromethyl)benzyloxy)pyrrolidin-3-yl)-1H-pyrazole-3-carbonitrile (Compound 6)

A mixture of compound 5 (200 mg, 0.46 mmol) and TFA (3 mL) in DCM (10 mL) was stirred at rt for 2 hours. The mixture was concentrated to leave the crude compound 6 as yellow oil (100 mg yield 64%), which was used directly for next step. LC-MS (ESI) m/z: 337 [M+H]⁺.

### Step 4: Synthesis of 1-(trans-1-acryloyl-4-(4-(trifluoromethyl)benzyloxy)pyrrolidin-3-yl)-1H-pyrazole-3-carbonitrile (IV-4)

A mixture of compound 6 (100 mg, 0.29 mmol), acryloyl chloride (26 mg, 0.29 mmol) and TEA (58 mg, 0.58 mmol) in DCM (5 mL) was stirred at rt for 2 hours. The mixture was concentrated and purified by prep-HPLC to obtain the target compound IV-4 as yellow oil (16 mg yield 14%). LC-MS (ESI) m/z: 391 [M+H]⁺. ¹H NMR (400 MHz, CD₃OD) δ (ppm) 7.96 (dd, J=5.1, 2.5 Hz, 1H), 7.65 (d, J=8.2 Hz, 2H), 7.50 (d, J=8.1 Hz, 2H), 6.85 (t, J=2.7 Hz, 1H), 6.69-6.55 (m, 1H), 6.34 (dd, J=16.8, 1.9 Hz, 1H), 5.81 (ddd, J=10.4, 4.5, 1.9 Hz, 1H), 5.34-5.11 (m, 1H), 4.72 (d, J=4.8 Hz, 2H), 4.58-3.68 (m, 5H).

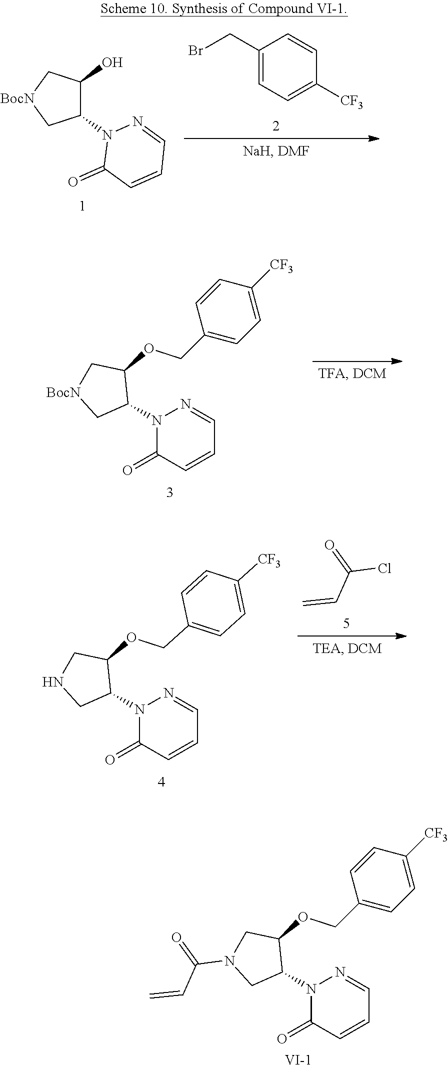

### Step 1: Synthesis of Tert-Butyl trans-3-(6-oxopyridazin-1(6H)-yl)-4-(4-(trifluoromethyl)benzyloxy)pyrrolidine-1-carboxylate (Compound 3)

To a solution of tert-butyl trans-3-hydroxy-4-(6-oxopyridazin-1(6H)-yl)pyrrolidine-1-carboxylate (200 mg, 0.71 mmol) in DMF (10 mL) was added NaH (60%, 32.74 mg, 0.85 mmol), the mixture was stirred at 0° C. under N₂ for 0.5 hour, and then 1-(bromomethyl)-4-(trifluoromethyl)benzene (169.4 mg, 0.71 mmol) was added, the resulting mixture was stirred at rt for 2 hours, diluted with water (100 mL) and extracted with EtOAc (500 mL×2), the combined organics were washed with brine (50 mL), dried over anhydrous Na₂SO₄, concentrated to leave crude compound 3 as oil (300 mg, crude). LC-MS (ESI) m/z: 439 [M+H]⁺.

### Step 2: Synthesis of 2-(trans-4-(4-(trifluoromethyl)benzyloxy)pyrrolidin-3-yl)pyridazin-3(2H)-one (Compound 4)

The mixture of tert-butyl trans-3-(6-oxopyridazin-1(6H)-yl)-4-(4-(trifluoromethyl)benzyloxy)pyrrolidine-1-carboxylate (300 mg, 0.68 mmol) and TFA (3 mL) in DCM (10 mL) was stirred at 0° C. for 1 hour, and then concentrated to leave crude compound 4 as oil (240 mg, crude). LC-MS (ESI) m/z: 339 [M+H]⁺.

### Step 3: Synthesis of 2-(trans-1-acryloyl-4-(4-(trifluoromethyl)benzyloxy)pyrrolidin-3-yl)pyridazin-3(2H)-one (Compound VI-1)

To a solution of 2-(trans-4-(4-(trifluoromethyl)benzyloxy)pyrrolidin-3-yl)pyridazin-3(2H)-one (240 mg, 0.71 mmol) in DCM (10 mL) was added acryloyl chloride (64 mg, 0.71 mmol) and Et₃N (143 mg, 1.42 mmol). The mixture was stirred at 0° C. for 1 hour, and then concentrated and purified by prep-HPLC to obtain the target compound VI-1 as solid (61 mg, yield 22%). LC-MS (ESI) m/z: 394 [M+H]⁺. ¹H NMR (400 MHz, CD₃OD) δ (ppm) 7.94 (dd, J=3.8, 1.6 Hz, 1H), 7.65 (d, J=8.0 Hz, 2H), 7.57 (d, J=7.9 Hz, 2H), 7.50-7.40 (m, 1H), 7.02 (dt, J=9.5, 1.6 Hz, 1H), 6.64 (ddd, J=16.8, 12.6, 10.5 Hz, 1H), 6.32 (dd, J=16.8, 1.5 Hz, 1H), 5.84-5.76 (m, 1H), 5.69-5.59 (m, 1H), 4.82 (dd, J=19.8, 7.6 Hz, 2H), 4.42-4.29 (m, 1H), 4.24-4.09 (m, 1H), 4.05-3.99 (m, 1H), 3.99-3.83 (m, 1H), 3.77 (d, J=3.7 Hz, 1H).

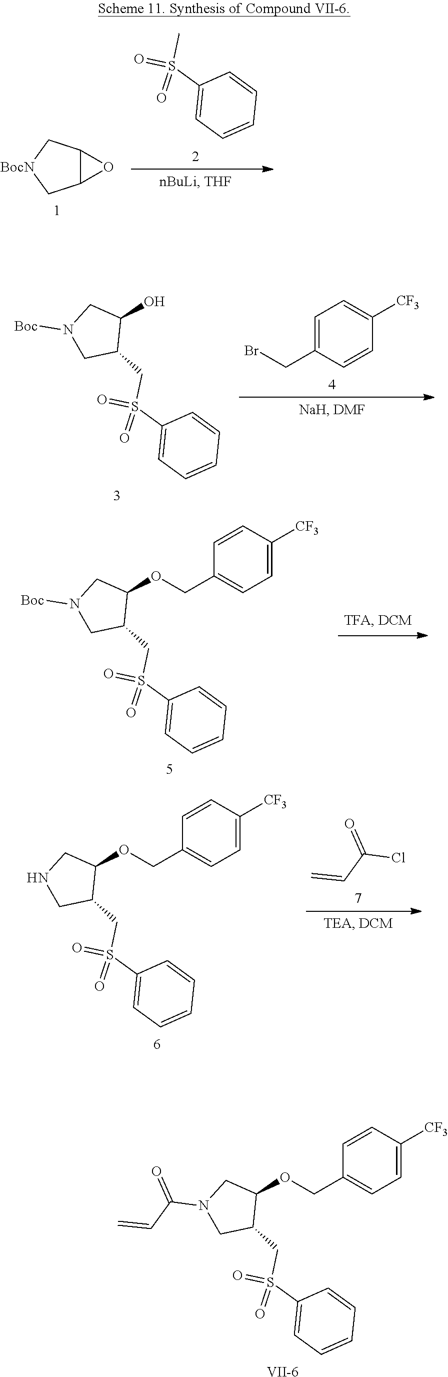

### Step 1: Synthesis of Tert-Butyl trans-3-hydroxy-4-(phenylsulfonylmethyl)pyrrolidine-1-carboxylate (Compound 3)

To a solution of (methylsulfonyl)benzene (421 mg, 2.7 mmol) in THF (10 mL) was added nBuLi (2.5 M in THF, 1.3 mL, 3.2 mmol), the mixture was stirred at −78° C. for 10 minutes, and then tert-butyl 6-oxa-3-azabicyclo[3.1.0]hexane-3-carboxylate (500 mg, 2.7 mmol) was added, the mixture was stirred at −78° C. under N₂ for 2 hours, and warmed gradually to rt. The mixture was diluted with water (50 mL) and extracted with ethyl acetate (50 mL), the organic was washed with water (50 mL), dried over anhydrous Na₂SO₄, concentrated and purified by prep-HPLC to obtain compound 3 as clear oil (660 mg, yield 52%). LC-MS (ESI) m/z: 286 [M−55]⁺.

### Step 2: Synthesis of Tert-Butyl trans-3-(phenylsulfonylmethyl)-4-(4-(trifluoromethyl)benzyloxy)pyrrolidine-1-carboxylate (Compound 5)

To a solution of compound 3 (100 mg, 0.29 mmol) in DMF (5 mL) was added NaH (12 mg, 0.29 mmol), the mixture was stirred at rt for 10 minutes, and then 1-(bromomethyl)-4-(trifluoromethyl)benzene (70 mg, 0.29 mmol) was added, the mixture was stirred at rt under N₂ for 2 hours, quenched with water (50 mL) and extracted with ethyl acetate (50 mL), the organic was washed with water (50 mL), dried over anhydrous Na₂SO₄, concentrated and purified by flash column chromatography on silica gel (ethyl acetate in petroleum ether=30% v/v) to obtain compound 5 as yellow oil (130 mg, yield 90%). LC-MS (ESI) m/z: 400 [M+H−100]⁺.

### Step 3: Synthesis of trans-3-(phenylsulfonylmethyl)-4-(4-(trifluoromethyl)benzyloxy)pyrrolidine (Compound 6)

A mixture of compound 5 (130 mg, 0.26 mmol) and TFA (3 mL) in DCM (5 mL) was stirred at rt for 2 hours, the mixture was concentrated to leave crude compound 6 as yellow oil (80 mg, yield 77%), which was used directly for next step. LC-MS (ESI) m/z: 400 [M+H]⁺.

### Step 4: Synthesis of 1-(trans-3-(phenylsulfonylmethyl)-4-(4-(trifluoromethyl)benzyloxy)pyrrolidin-1-yl)prop-2-en-1-one (Compound VII-6)

A mixture of compound 6 (90 mg, 0.2 mmol), acryloyl chloride (19 mg, 0.2 mmol) and TEA (44 mg, 0.4 mmol) in DCM (5 mL) was stirred at rt for 2 hours. The mixture was concentrated and purified by prep-HPLC to obtain compound VII-6 as white solid (20 mg, yield 22%). LC-MS (ESI) m/z: 454 [M+H]⁺. ¹H NMR (400 MHz, DMSO-d6) δ (ppm) 7.95 (dd, J=7.2, 5.1 Hz, 2H), 7.73 (dq, J=22.2, 7.5 Hz, 5H), 7.52 (d, J=8.0 Hz, 2H), 6.59-6.41 (m, 1H), 6.13 (dd, J=16.8, 2.2 Hz, 1H), 5.75-5.53 (m, 1H), 4.58 (d, J=6.8 Hz, 2H), 4.08-3.36 (m, 7H), 2.71-2.52 (m, 1H).

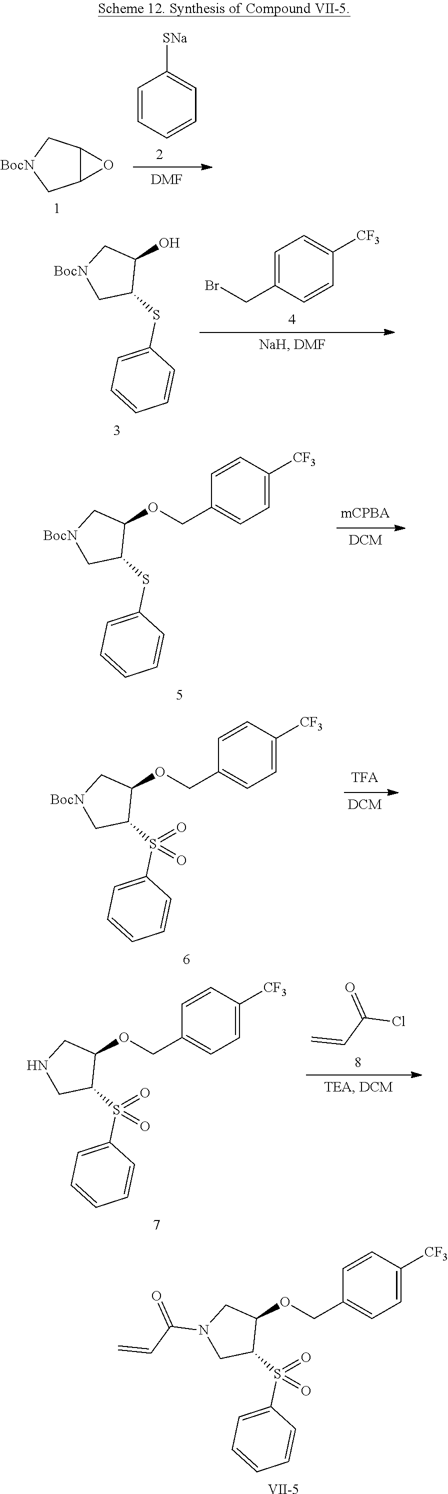

### Step 1: Synthesis of Tert-Butyl trans-3-hydroxy-4-(phenylthio)pyrrolidine-1-carboxylate (Compound 3)

A mixture of tert-butyl 6-oxa-3-azabicyclo[3.1.0]hexane-3-carboxylate (420 mg, 2.2 mmol) and sodium benzenethiolate (300 mg, 2.27 mmol) in DMF (5 mL) was stirred at rt under N₂ overnight. The mixture was diluted with water (50 mL) and extracted with ethyl acetate (100 mL), the organic was washed with water (50 mL), dried over anhydrous Na₂SO₄, concentrated and purified by column chromatography on silica gel (ethyl acetate in petroleum ether=30% v/v) to obtain compound 3 as oil (500 mg, yield 76%). LC-MS (ESI) m/z: 196 [M+H−100]⁺.

### Step 2: Synthesis of Tert-Butyl trans-3-(phenylthio)-4-(4-(trifluoromethyl)benzyloxy)pyrrolidine-1-carboxylate (Compound 5)

To a solution of compound 3 (500 mg, 0.84 mmol) in DMF (5 mL) was added NaH (34 mg, 0.84 mmol), the mixture was stirred at rt for 10 minutes, and then 1-(bromomethyl)-4-(trifluoromethyl)benzene (202 mg, 0.84 mmol) was added, the mixture was stirred at rt under N₂ for 2 hours, diluted with water (50 mL) and extracted with ethyl acetate (100 mL), the organic was washed with water (50 mL), dried over anhydrous Na₂SO₄, filtered and concentrated to leave crude compound 5 as yellow oil (300 mg, yield 78%), which was used directly for next step. LC-MS (ESI) m/z: 354 [M+H−100]⁺.

### Step 3: Synthesis of Tert-Butyl trans-3-(phenylsulfonyl)-4-(4-(trifluoromethyl)benzyloxy)pyrrolidine-1-carboxylate (Compound 6)

A mixture of compound 5 (3000 mg, 0.66 mmol) and mCPBA (284 mg, 1.65 mmol) in DCM (10 mL) was stirred at rt overnight. The mixture was concentrated and purified by flash column chromatography on silica gel (ethyl acetate in petroleum ether=30% v/v) to obtain compound 6 as yellow oil (230 mg, yield 72%). LC-MS (ESI) m/z: 386 [M+H−100]⁺.

### Step 4: Synthesis of trans-3-(phenylsulfonyl)-4-(4-(trifluoromethyl)benzyloxy)-pyrrolidine (Compound 7)

A mixture of compound 6 (100 mg, 0.2 mmol) and TFA (3 mL) in DCM (5 mL) was stirred at rt for 2 hours. The mixture was concentrated to leave crude compound 7 as yellow oil (80 mg yield 100%). LC-MS (ESI) m/z: 386 [M+H]⁺.

### Step 5: Synthesis of 1-(trans-3-(phenylsulfonyl)-4-(4-(trifluoromethyl)benzyloxy)pyrrolidin-1-yl)prop-2-en-1-one (VII-5)

A mixture of compound 7 (80 mg, 0.2 mmol), acryloyl chloride (19 mg, 0.2 mmol) and TEA (44 mg, 0.4 mmol) in DCM (5 mL) was stirred at rt for 2 hours. The mixture was concentrated and purified by prep-HPLC to obtain compound VII-5 as white solid (12 mg, yield 13%). LC-MS (ESI) m/z: 440 [M+H]⁺. ¹H NMR (400 MHz, CD₃OD) δ (ppm) 7.85 (d, J=7.7 Hz, 2H), 7.67 (t, J=7.4 Hz, 1H), 7.51 (dt, J=27.0, 6.9 Hz, 4H), 7.25 (dd, J=14.3, 8.1 Hz, 2H), 6.58-6.37 (m, 1H), 6.19 (dd, J=16.8, 1.6 Hz, 1H), 5.67 (dd, J=18.1, 7.4 Hz, 1H), 4.55-4.31 (m, 3H), 4.22-3.83 (m, 3H), 3.79-3.52 (m, 2H).

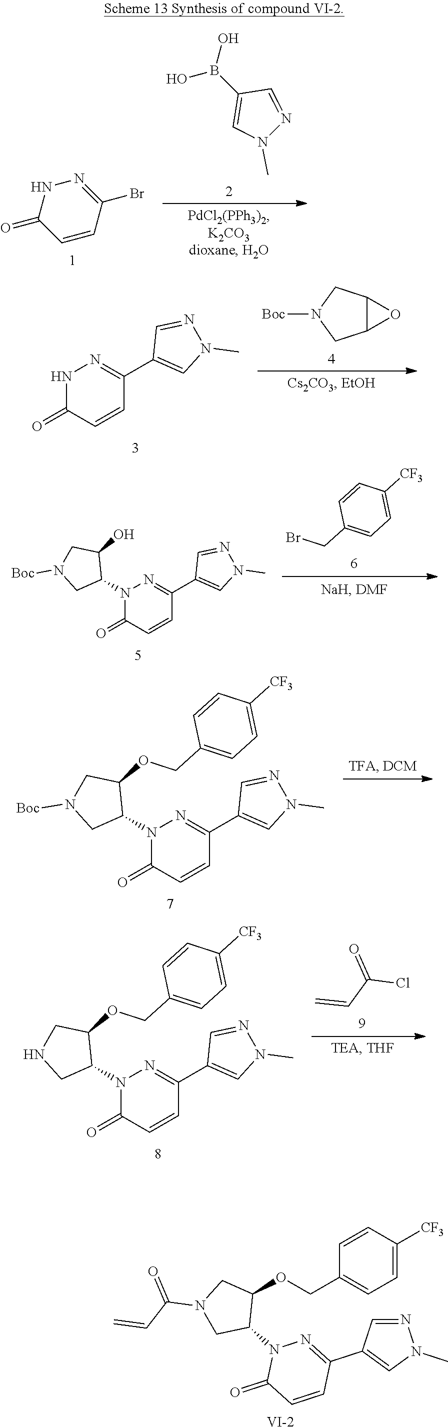

### Step 1: Synthesis of 6-(1-methyl-1H-pyrazol-4-yl)pyridazin-3(2H)-one (Compound 3)

To a solution of 6-bromopyridazin-3(2H)-one (1 g, 5.75 mmol) in 1,4-dioxane (32 mL) and H₂O (8 mL) was added 1-methyl-1H-pyrazol-4-ylboronic acid (0.794 g, 6.3 mmol), K₂CO₃ (1.58 g, 11.5 mmol) and PdCl₂(PPh₃)₂ (0.121 mg. 0.17 mmol). The mixture was stirred at 110° C. for 16 h. The mixture was diluted with water (100 mL) and extracted with EtOAc (3×50 mL), the combined organics were washed with brine (100 mL), dried over anhydrous Na₂SO₄, purified by column chromatography on silica gel (ethyl acetate in petroleum ether=50% v/v) to obtain the target compound 3 as solid (900 mg, yield 88.5%). LC-MS (ESI) m/z: 177 [M+H]⁺.

### Step 2: Synthesis of Tert-Butyl trans-3-hydroxy-4-(3-(1-methyl-1H-pyrazol-4-yl)-6-oxopyridazin-1(6H)-yl)pyrrolidine-1-carboxylate (Compound 5)

To a solution of 6-(1-methyl-1H-pyrazol-4-yl)pyridazin-3(2H)-one (800 mg, 4.55 mmol) in EtOH (40 mL) was added tert-butyl 6-oxa-3-azabicyclo[3.1.0]hexane-3-carboxylate (1681 mg. 9.10 mmol) and Cs₂CO₃ (4436 mg, 13.65 mmol). The mixture was stirred at 80° C. for 16 h., diluted with water (100 mL) and extracted with EtOAc (3×50 mL), the combined organics were washed with brine (100 mL), dried over anhydrous Na₂SO₄, concentrated and purified by column chromatography on silica gel (ethyl acetate in petroleum ether=50% v/v) to obtain the target compound 5 as oil (500 mg, yield 30.5%). LC-MS (ESI) m/z: 362 [M+H]⁺.

### Step 3: Synthesis of Tert-Butyl trans-3-(3-(1-methyl-1H-pyrazol-4-yl)-6-oxopyridazin-1(6H)-yl)-4-(4-(trifluoromethyl)benzyloxy)pyrrolidine-1-carboxylate (Compound 7)

To a solution of tert-butyl trans-3-hydroxy-4-(3-(1-methyl-1H-pyrazol-4-yl)-6-oxopyridazin-1(6H)-yl)pyrrolidine-1-carboxylate (450 mg, 1.25 mmol) in DMF (10 mL) was added NaH (60%, 60 mg, 2.50 mmol), the mixture was stirred at 0° C. under N₂ for 0.5 h., and then 1-(bromomethyl)-4-(trifluoromethyl)benzene (326 mg, 1.37 mmol) was added. The resulting mixture was stirred at rt for 16 h., diluted with water (100 mL) and extracted with EtOAc (3×50 mL), the combined organics were washed with brine (100 mL), dried over anhydrous Na₂SO₄, concentrated and purified by column chromatography on silica gel (ethyl acetate in petroleum ether=50% v/v) to obtain the target compound 7 as solid (300 mg, yield 46.3%). LC-MS (ESI) m/z: 520 [M+H]⁺.

### Step 4: Synthesis of 6-(1-methyl-1H-pyrazol-4-yl)-2-(trans-4-(4-(trifluoromethyl)benzyloxy)pyrrolidin-3-yl)pyridazin-3(2H)-one (Compound 8)

A mixture of tert-butyl trans-3-(3-(1-methyl-1H-pyrazol-4-yl)-6-oxopyridazin-1(6H)-yl)-4-(4-(trifluoromethyl)benzyloxy)pyrrolidine-1-carboxylate (250 mg, 0.2 mmol) and TFA (2 mL) in DCM (10 mL) was stirred at RT for 2 h. The mixture was basified to pH˜8 with NaHCO₃ solution and extracted with EtOAc (3×50 mL), the combined organics were washed with brine (100 mL), dried over anhydrous Na₂SO₄, filtered and concentrated to leave crude compound 8 as oil (200 mg, crude). LC-MS (ESI) m/z: 420 [M+H]⁺.

### Step 5: Synthesis of 2-(trans-1-acryloyl-4-(4-(trifluoromethyl)benzyloxy)pyrrolidin-3-yl)-6-(1-methyl-1H-pyrazol-4-yl)pyridazin-3(2H)-one (Compound VI-2)

To s solution of 6-(1-methyl-1H-pyrazol-4-yl)-2-(trans-4-(4-(trifluoromethyl)benzyloxy)pyrrolidin-3-yl)pyridazin-3(2H)-one (180 mg, 0.43 mmol) in THF (10 mL) was added acryloyl chloride (39 mg, 0.43 mmol) and Et₃N (87 mg, 0.86 mmol). The mixture was stirred at 0° C. for 1 h., concentrated and purified by preparative HPLC to obtain the target compound VI-2 as solid (127 mg, yield 62.6%). LC-MS (ESI) m/z: 474 [M+H]⁺. ¹H NMR (400 MHz, DMSO) δ 8.11 (d, J=4.9 Hz, 1H), 7.84-7.73 (m, 2H), 7.68 (d, J=6.9 Hz, 2H), 7.55 (d, J=8.1 Hz, 2H), 7.03 (dd, J=9.7, 3.0 Hz, 1H), 6.66 (dd, J=16.7, 10.3 Hz, 1H), 6.21 (ddd, J=16.8, 5.5, 2.3 Hz, 1H), 5.72 (ddd, J=10.2, 6.5, 2.3 Hz, 1H), 5.56-5.44 (m, 1H), 4.76 (qd, J=12.9, 4.0 Hz, 2H), 4.44-4.30 (m, 1H), 4.14-3.90 (m, 2H), 3.87-3.50 (m, 5H).

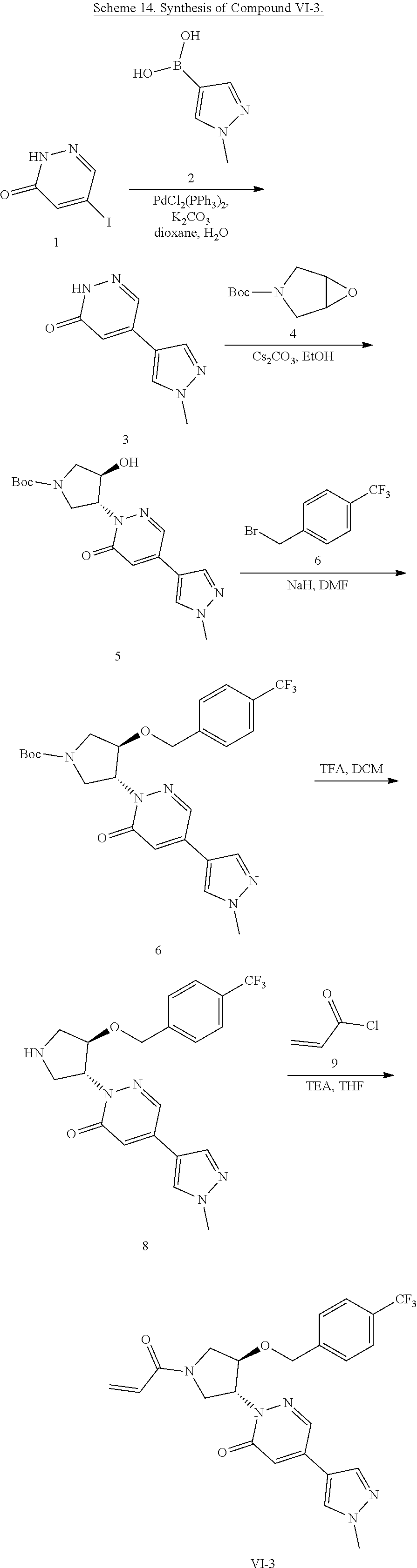

### Step 1: Synthesis of 5-(1-methyl-1H-pyrazol-4-yl)pyridazin-3(2H)-one (Compound 3)

To a solution of 5-iodopyridazine-3(2H)-one (1 g, 4.5 mmol) in dioxane (32 mL) and H₂O (8 mL) was added (1-methyl-1H-pyrazol-4-yl) boronic acid (0.63 g, 5.0 mmol), K₂CO₃ (1.24 g, 9.0 mmol) and PdCl₂(PPh₃)₂ (95 mg, 0.135 mmol). The mixture was stirred at 110° C. under N₂ for 14 h. The mixture was diluted with water (100 mL) and extracted with EtOAc (3×50 mL), the combined organics were washed with brine (100 mL), dried over anhydrous Na₂SO₄, purified by column chromatography on silica gel (eluting with DCM/MeOH=10:1) to obtain the target compound 3 as solid (500 mg, yield 63%). LC-MS (ESI) m/z: 177 [M+H]⁺.

### Step 2: Synthesis of Tert-Butyl trans-3-hydroxy-4-(4-(1-methyl-1H-pyrazol-4-yl)-6-oxopyridazin-1(6H)-yl)pyrrolidine-1-carboxylate (Compound 5)

To a solution of 5-(1-methyl-1H-pyrazol-4-yl)pyridazin-3(2H)-one (500 mg, 2.84 mmol) in EtOH (40 mL) was added tert-butyl 6-oxa-3-azabicyclo[3.1.0]hexane-3-carboxylate (1050 mg, 5.68 mmol) and Cs₂CO₃ (2769 mg, 8.52 mmol). The mixture was stirred at 80° C. for 16 h., diluted with water (100 mL) and extracted with EtOAc (3×50 mL), the combined organics were washed with brine (100 mL), dried over anhydrous Na₂SO₄, concentrated and purified by column chromatography on silica gel (eluting with DCM/MeOH=10:1) to obtain the target compound 5 as oil (500 mg, yield 48.7%). LC-MS (ESI) m/z: 362 [M+H]⁺.

### Step 3: Synthesis of Tert-Butyl trans-3-(4-(1-methyl-1H-pyrazol-4-yl)-6-oxopyridazin-1(6H)-yl)-4-((4-(trifluoromethyl)benzyl)oxy)pyrrolidine-1-carboxylate (Compound 7)

To a solution of tert-butyl trans-3-hydroxy-4-(4-(1-methyl-1H-pyrazol-4-yl)-6-oxopyridazin-1(6H)-yl)pyrrolidine-1-carboxylate (450 mg, 1.25 mmol) in DMF (10 mL) was added NaH (60 mg, 2.50 mmol), the mixture was stirred at 0° C. under N₂ for 0.5 h., and then 1-(bromomethyl)-4-(trifluoromethyl)benzene (326 mg, 1.37 mmol) was added. The resulting mixture was stirred at rt for 16 h., diluted with water (100 mL) and extracted with EtOAc (3×50 mL), the combined organics were washed with brine (100 mL), dried over anhydrous Na₂SO₄, concentrated and purified by column chromatography on silica gel (eluting with EA/PE=1:1) to obtain the target compound 7 as solid (300 mg, yield 46.3%). LC-MS (ESI) m/z: 420 [M−100]⁺.

### Step 4: Synthesis of 5-(1-methyl-1H-pyrazol-4-yl)-2-(trans-4-((4-(trifluoromethyl)benzyl)oxy)pyrrolidin-3-yl)pyridazin-3(2H)-one (Compound 8)

A mixture of compound 7 (250 mg, 0.5 mmol) and TFA (2 mL) in DCM (10 mL) was stirred at RT for 2 h. The mixture was basified to pH˜8 with NaHCO₃ solution and extracted with EtOAc (3×50 mL), the combined organics were washed with brine (100 mL), dried over anhydrous Na₂SO₄, filtered and concentrated to leave crude compound 8 as oil (200 mg, crude). LC-MS (ESI) m/z: 420 [M+H]⁺.

### Step 5: Synthesis of 2-(trans-1-acryloyl-4-((4-(trifluoromethyl)benzyl)oxy)pyrrolidin-3-yl)-5-(1-methyl-1H-pyrazol-4-yl)pyridazin-3(2H)-one (Compound VI-3)

To a solution of compound 8 (180 mg, 0.43 mmol) in THF (10 mL) was added acryloyl chloride (39 mg, 0.43 mmol) and Et₃N (87 mg, 0.86 mmol). The mixture was stirred at 0° C. for 1 h., concentrated and purified by preparative HPLC to obtain the target compound VI-3 as solid (19 mg, yield 9.4%). LC-MS (ESI) m/z: 474 [M+H]⁺. ¹H NMR (400 MHz, DMSO) δ 8.43 (s, 1H), 8.31 (dd, J=4.5, 2.2 Hz, 1H), 8.13 (s, 1H), 7.69 (d, J=8.1 Hz, 2H), 7.56 (d, J=8.0 Hz, 2H), 7.12 (t, J=2.1 Hz, 1H), 6.61 (ddd, J=16.7, 10.3, 2.2 Hz, 1H), 6.18 (d, J=16.5 Hz, 1H), 5.70 (d, J=11.6 Hz, 1H), 5.51 (dd, J=20.2, 5.1 Hz, 1H), 4.86-4.66 (m, 2H), 4.29 (d, J=20.6 Hz, 1H), 4.13-3.94 (m, 1H), 3.94-3.76 (m, 5H), 3.74-3.54 (m, 1H).

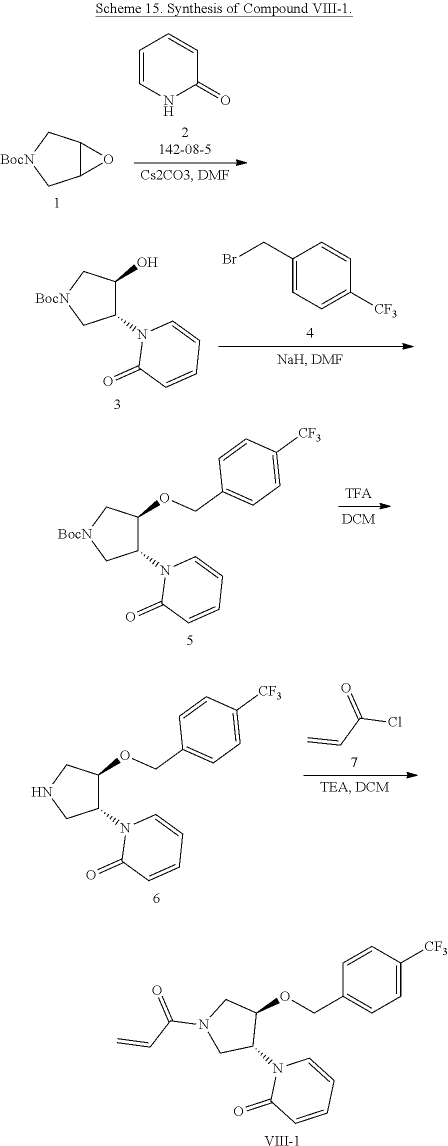

### Step 1: Synthesis of trans-tert-butyl 3-hydroxy-4-(pyridin-2-yloxy)pyrrolidine-1-carboxylate (Compound 3)

A mixture of tert-butyl 6-oxa-3-azabicyclo[3.1.0]hexane-3-carboxylate (973 mg, 5.26 mmol), pyridin-2(1H)-one (500 mg, 5.26 mmol) and Cs₂CO₃ (1710 mg, 5.26 mmol) in DMF (10 mL) was stirred at 80° C. under N₂ overnight. The mixture was diluted with water (100 mL) and extracted with ethyl acetate (50 mL), the organic was washed with brine (50 mL), dried over anhydrous Na₂SO₄, concentrated and purified by flash column chromatography on silica gel (ethyl acetate in petroleum ether=50% v/v) to obtain compound 3 as yellow oil (500 mg, yield 52.5%). LC-MS (ESI) m/z: 182 [M+H]⁺.

### Step 2: Synthesis of trans-tert-butyl 3-(pyridin-2-yloxy)-4-(4-(trifluoromethyl)benzyloxy)pyrrolidine-1-carboxylate (Compound 5)

To a mixture of compound 3 (140 mg, 0.5 mmol) and NaH (20 mg, 0.5 mmol) in DMF (5 mL) was added 1-(bromomethyl)-4-(trifluoromethyl)benzene (119 mg, 0.5 mmol), the mixture was stirred at rt under N₂ for 2 hours, quenched with water (100 mL) and extracted with ethyl acetate (50 mL), the organic was washed with brine (50 mL), dried over anhydrous Na₂SO₄, filtered and concentrated to leave the crude compound 5 as yellow oil (160 mg, yield 73%), which was used directly in the next step. LC-MS (ESI) m/z: 383 [M+H−56]⁺.

### Step 3: Synthesis of 2-(trans-4-(4-(trifluoromethyl)benzyloxy)pyrrolidin-3-yloxy)pyridine (Compound 6)

A mixture of compound 5 (160 mg, 0.36 mmol) and TFA (3 mL) in DCM (10 mL) was stirred at rt for 2 hours, the mixture was concentrated to leave the crude compound 6 as yellow oil (130 mg, yield 106%). LC-MS (ESI) m/z: 339 [M+H]⁺.

### Step 4: Synthesis of 1-(trans-3-(pyridin-2-yloxy)-4-(4-(trifluoromethyl)benzyloxy)pyrrolidin-1-yl)prop-2-en-1-one (VIII-1))

A mixture of compound 6 (130 mg, 0.36 mmol), acryloyl chloride (32 mg, 0.36 mmol) and TEA (107 mg, 1.06 mmol) in DCM (10 mL) was stirred at rt under N₂ for 2 hours. The mixture was concentrated and the residue was purified by prep-HPLC (MeCN/H₂O/TFA) to obtain product as yellow oil (42 mg, yield 29%). LC-MS (ESI) m/z: 393 [M+H]⁺. ¹H-NMR (400 MHz, CD₃OD): δ (ppm) 7.63 (d, J=7.3 Hz, 2H), 7.58-7.42 (m, 4H), 6.72-6.52 (m, 2H), 6.47-6.25 (m, 2H), 5.82 (dt, J=10.4, 1.9 Hz, 1H), 5.45-5.24 (m, 1H), 4.85-4.65 (m, 2H), 4.21 (dddd, J=21.2, 17.5, 10.6, 5.6 Hz, 3H), 4.01-3.87 (m, 1H), 3.75 (ddd, J=17.2, 12.6, 3.6 Hz, 1H).

Compounds I-1, I-6, VII-11 were synthesized according to general synthesis route II shown below in Scheme 16 and using the specific reagents as detailed in Table 10. The synthesis of Compound I-9 is shown in Scheme 17.

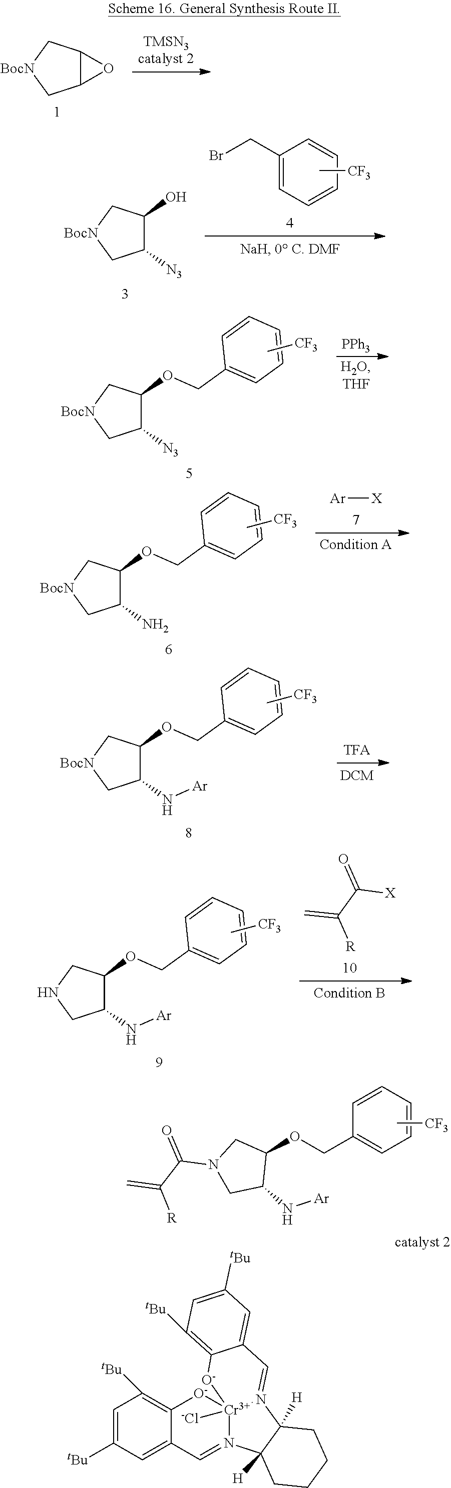

TABLE 10

| | | 7 | 10 |
|---|---|---|---|
| Compound | 4 | Condition A | Condition B |
| 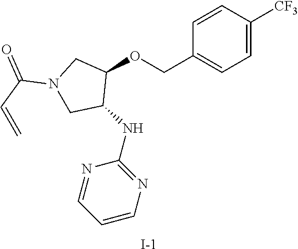 [SMILES: C=CC(=O)N1C[C@@H](Nc2ncccn2)[C@H](OCc2ccc(C(F)(F)F)cc2)C1 ; [I-]] | 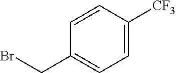 [SMILES: FC(F)(F)c1ccc(CBr)cc1] | 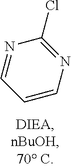 | 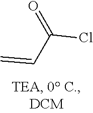 |
| 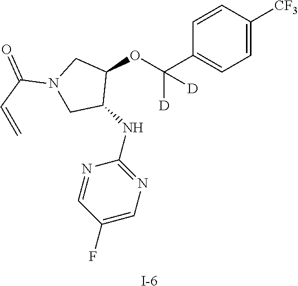 | 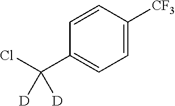 [SMILES: [2H]C([2H])(Cl)c1ccc(C(F)(F)F)cc1] | 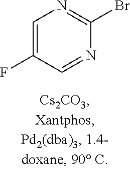 | 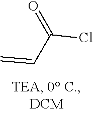 |
| 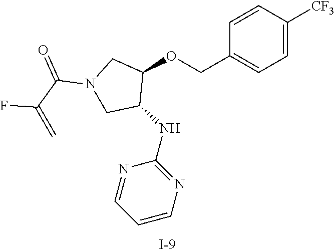 | 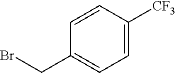 [SMILES: FC(F)(F)c1ccc(CBr)cc1] | 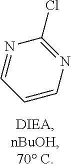 | 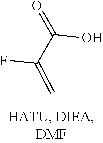 |
| 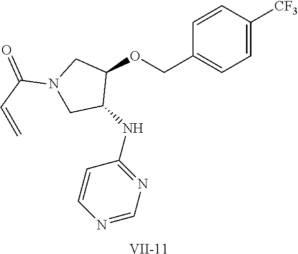 | 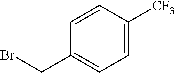 [SMILES: FC(F)(F)c1ccc(CBr)cc1] | 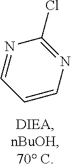 | 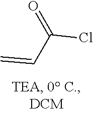 |

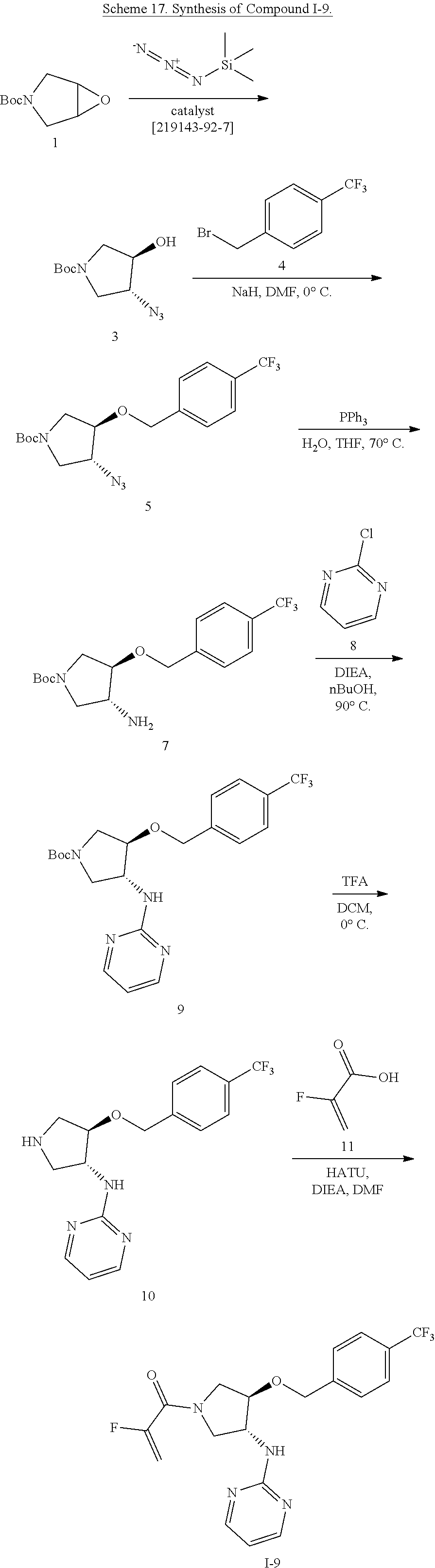

### Step 1: Synthesis of (3R, 4R)-tert-butyl 3-azido-4-hydroxypyrrolidine-1-carboxylate (Compound 3)

A mixture of tert-butyl 6-oxa-3-azabicyclo[3.1.0]hexane-3-carboxylate (4 g, 21.6 mmol), TMSN₃ (2.664 g, 23.2 mmol) and chiral catalyst (1S,2S)-(−)-[1,2-cyclohexanediamino-N,N′-bis (3,5-di-t-butylsalicylidene)]chromium (III) chloride (328 mg, 0.42 mmol) was stirred at rt under N₂ overnight. The reaction mixture was treated with MeOH (60 mL) and K₂CO₃ (1.788 g, 12.8 mmol) and continued to stir at rt for 5 hours. The reaction mixture was diluted with ethyl acetate (300 mL), washed with water (300 mL×2), dried over anhydrous Na₂SO₄, concentrated and purified by flash column chromatography on silica gel (ethyl acetate in petroleum ether=30% v/v) to obtain the compound 3 as clear oil (3.5 g, 96% e.e., yield 71%). LC-MS (ESI) m/z: 129 [M+H−100]⁺.

### Step 2: Synthesis of (3R, 4R)-tert-butyl 3-azido-4-(4-(trifluoromethyl)benzyloxy)-pyrrolidine-1-carboxylate (5)

A mixture of compound 3 (3 g, 13.1 mmol), 1-(bromomethyl)-4-(trifluoromethyl)benzene (3.1 g, 13.1 mmol) and 60% NaH (0.6 g, 15.7 mmol) in DMF (20 mL) was stirred at 0° C. under N₂ for 6 hours. The reaction mixture was diluted with water (200 mL) and extracted with ethyl acetate (200 mL), the organic was washed with water (100 mL), dried over anhydrous Na₂SO₄, concentrated and purified by flash column chromatography on silica gel (ethyl acetate in petroleum ether=20% v/v) to obtain compound 5 as oil (3.8 g, yield 75%). LC-MS (ESI) m/z: 287 [M+H−100]⁺.

### Step 3: Synthesis of (3R, 4R)-tert-butyl 3-amino-4-(4-(trifluoromethyl)benzyloxy)-pyrrolidine-1-carboxylate (Compound 7)

A mixture of compound 5 (1000 mg, 2.58 mmol), PPh₃ (814 mg, 3.1 mmol) and H₂O (930 mg, 51.6 mmol) in THF (40 mL) was stirred at 70° C. under N₂ for 5 hours. The reaction mixture was cooled down to rt, diluted with water (100 mL) and extracted with ethyl acetate (300 mL), the organic was washed with water (200 mL), dried over anhydrous Na₂SO₄, concentrated and purified by prep-HPLC to obtain compound 7 as yellow oil (800 mg, yield 86%). LC-MS (ESI) m/z: 261 [M+H−100]⁺.

### Step 4: Synthesis of (3R, 4R)-tert-butyl 3-(pyrimidin-2-ylamino)-4-(4-(trifluoromethyl)benzyloxy)pyrrolidine-1-carboxylate (Compound 9)

A mixture of compound 7 (600 mg, 1.6 mmol), 2-chloropyrimidine (240 mg, 1.84 mmol) and DIPEA (420 mg, 3.24 mmol) in n-BuOH (6 mL) was stirred at 70° C. under N₂ overnight. The reaction mixture was concentrated and purified by prep-HPLC to obtain compound 9 as oil (500 mg, yield 71%). LC-MS (ESI) m/z: 439 [M+H]⁺.

### Step 5: Synthesis of N-((3R, 4R)-4-(4-(trifluoromethyl)benzyloxy)pyrrolidin-3-yl)pyrimidin-2-amine (Compound 10)

A mixture of compound 9 (400 mg, 0.91 mmol) and TFA (1 mL) in DCM (3 mL) was stirred at rt under N₂ for 2 hours. The mixture was concentrated to leave the crude compound 10 as yellow oil (400 mg, yield 97%), which was used directly for next step. LC-MS (ESI) m/z: 339 [M+H]⁺.

### Step 6: Synthesis of 2-fluoro-1-((3R, 4R)-3-(pyrimidin-2-ylamino)-4-(4-(trifluoromethyl)benzyloxy)pyrrolidin-1-yl)prop-2-en-1-one (I-9)

A mixture of compound 10 (200 mg, 0.58 mmol), 2-fluoroacrylic acid (60 mg, 0.69 mmol), HATU (256 mg, 0.69 mmol) and DIEA (224 mg, 1.74 mmol) in DMF (5 mL) was stirred rt under N₂ overnight. The reaction mixture was concentrated and purified by prep-HPLC to obtain the target product as white solid (150 mg, yield 63%). LC-MS (ESI) m/z: 411 [M+H]⁺. ¹H-NMR (400 MHz, CD₃OD) δ (ppm): 8.44 (s, 2H), 7.82-7.50 (m, 4H), 6.83 (dd, J=8.3, 4.9 Hz, 1H), 5.50 (dt, J=47.1, 3.3 Hz, 1H), 5.27 (dt, J=16.5, 3.4 Hz, 1H), 4.82 (dd, J=13.3, 8.8 Hz, 2H), 4.68-4.57 (m, 1H), 4.28-3.67 (m, 5H).

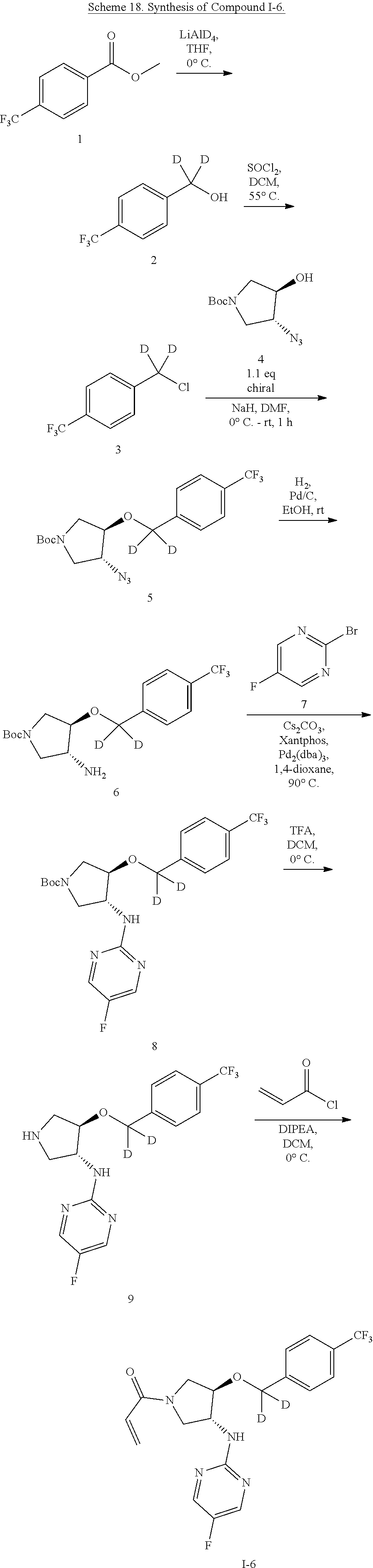

### Step 1: Synthesis of (4-(trifluoromethyl)phenyl)methan-d₂-ol (Compound 2)

To the solution of methyl 4-(trifluoromethyl)benzoate (4.08 g, 20 mmol) in THF (100 mL) was added LiAlD₄ (1.764 g, 42 mmol) in several portions at 0° C. The resulting mixture was stirred at 0° C. and monitored by LC-MS. After the completion of the reaction, saturated aqueous NH₄Cl (150 mL) was added carefully, and the resulting mixture was extracted with EtOAc (3×100 mL). The combined organic layer was dried over anhydrous Na₂SO₄ and concentrate under reduce pressure to afford the desired product as oil (3.418 g, yield 96.0%), which was used directly for the next step. LC-MS (ESI) m/z: 161 [M−17]⁺.

### Step 2: Synthesis of 1-(chloromethyl-d₂)-4-(trifluoromethyl)benzene (Compound 3)

To the solution of compound 2 (3.3 g, 18.54 mmol) in DCM (100.0 mL) was added SOCl₂ (5.84 g, 40.79 mmol). The resulting solution was stirred at 50° C. for 2 hours and monitored by LC-MS. After the completion of the reaction, the mixture was washed with water (150 mL), saturated NaHCO₃ (100 mL×3) and brine (100 mL), dried over anhydrous Na₂SO₄, filtered and concentrated under reduce pressure to leave the crude product (3.578 g, 98.2%) as a colorless liquid, which was used directly without further purification. LC-MS (ESI) m/z: 161 [M−36]⁺.

### Step 3: Synthesis of tert-butyl (3R, 4R)-3-azido-4-((4-(trifluoromethyl)phenyl)methoxy-d₂)pyrrolidine-1-carboxylate (Compound 5)

To the solution of compound 4 (chiral, 2.07 g. 9.08 mmol) in DMF (50.0 mL) was added NaH (346 mg, 8.65 mmol) at 0° C., the mixture was stirred at 0° C. for 0.5 hour, and then compound 3 (1.7 g. 8.65 mmol) was added. The resulting mixture was stirred at 0° C. for 3 hours and monitored by LC-MS. After the completion of the reaction, the mixture was diluted with cold H₂O (100 mL) and extracted with EtOAc (100 mL×3), the combined organic was washed with brine (100 mL×2), dried over anhydrous Na₂SO₄, filtered and concentrated under vacuum to afford the desired product (2.0 g, 59.6%) as a light yellow liquid, which was used directly without further purification. LC-MS (ESI) m/z: 289 [M-100+H]⁺.

### Step 4: Synthesis of tert-butyl (3R, 4R)-3-amino-4-((4-(trifluoromethyl)phenyl)methoxy-d₂)pyrrolidine-1-carboxylate (Compound 6)

A mixture of compound 5 (1.80, 4.64 mmol) and Pd/C (10%, 24.7 mg) in EtOH (100 mL) was stirred at room temperature under H₂ (1 atm) for 16 hours. The reaction mixture was filtered through celite, the filtrate was concentrated under reduced pressure to afford the desired product (1.68 g. 100%) as a light yellow liquid, which was used directly for the next step without further purification. LC-MS (ESI) m/z: 263 [M−100+H]⁺.

### Step 5: Synthesis of tert-butyl (3R, 4R)-3-((5-fluoropyrimidin-2-yl)amino)-4-((4-(trifluoromethyl)phenyl)methoxy-d₂)pyrrolidine-1-carboxylate (Compound 8)

To a three necked flask was added compound 6 (1.149 g. 3.175 mmol) and 1,4-dioxane (30 mL), followed by the addition of 2-bromo-5-fluoropyrimidine (0.843 g, 4.763 mmol), Pd₂(dba)₃ (0.290 g. 0.318 mmol), Xantphos (0.551 g, 0.953 mmol), and Cs₂CO₃ (195.5 mg, 9.525 mmol). The flask was evacuated, and refilled with N₂ for three times. The resulting mixture was stirred at 90° C. for 16 hours. After the completion of the reaction, the solvent was removed under reduce pressure to leave the crude product, which was purified by flash column chromatography on silica gel (eluting with 1% MeOH in DCM) to give the desired compound 8 (0.66 g, 45.4% yield) as a brown solid. LC-MS (ESI) m/z: 359 [M+H−100]⁺.

### Step 6: Synthesis of 5-fluoro-N-((3R, 4R)-4-((4-(trifluoromethyl)phenyl)methoxy-d₂)pyrrolidin-3-yl)pyrimidin-2-amine (Compound 9)

To the solution of compound 8 (0.40 g, 0.873 mmol) in DCM (20.0 mL) was added TFA (1.493 g, 13.10 mmol). The resulting solution was stirred at 0° C. for 3 hours and monitored by LC-MS. After the completion of the reaction, the mixture was washed with H₂O (30 mL×2) and saturated NaHCO₃ aqueous (30 mL×2), dried over anhydrous Na₂SO₄, filtered and concentrated under reduce pressure to afford the desired product 9 (0.30 g, 96.0% yield) as a viscous colorless liquid, which was used directly for the next step without further purification. LC-MS (ESI) m/z: 359 [M+H]⁺.

### Step 7: Synthesis of 5-fluoro-N-((3R, 4R)-4-((4-(trifluoromethyl)phenyl)methoxy-d₂)pyrrolidin-3-yl)pyrimidin-2-amine (I-6)

To the solution of compound 9 (260.0 mg, 0.726 mmol) and DIPEA (516 mg, 4 mmol) in DCM (15.0 mL) was added acryloyl chloride (65.7 mg, 0.726 mmol) dropwise. The resulting solution was stirred at 0° C. for 20 minutes and monitored by LC-MS. After the completion of the reaction, the mixture was concentrated and purified by flash column chromatography on silica gel (eluting with 1% MeOH in DCM) to give the desired product (90 mg, 30.0% yield) as a white solid. LC-MS (ESI) m/z: 413 [M+H]⁺. ¹H NMR (400 MHz, DMSO-d₆) δ (ppm) 8.43 (s, 1H), 7.74-7.58 (m, 5H), 6.62-6.54 (m, 1H), 6.16 (dt, J=16.8, 3.2 Hz, 1H), 5.70-5.65 (m, 1H), 4.75 (d, J=5.6 Hz, 0.29H), 4.38 (dt, J=18.4, 6.0 Hz, 1H), 4.09 (dt, J=21.6, 2.0 Hz, 1H), 3.75-3.55 (m, 3H), ¹⁹F NMR (376 MHz, DMSO-d6) δ (ppm): −60.88, −60.89, −156.10, −156.11.

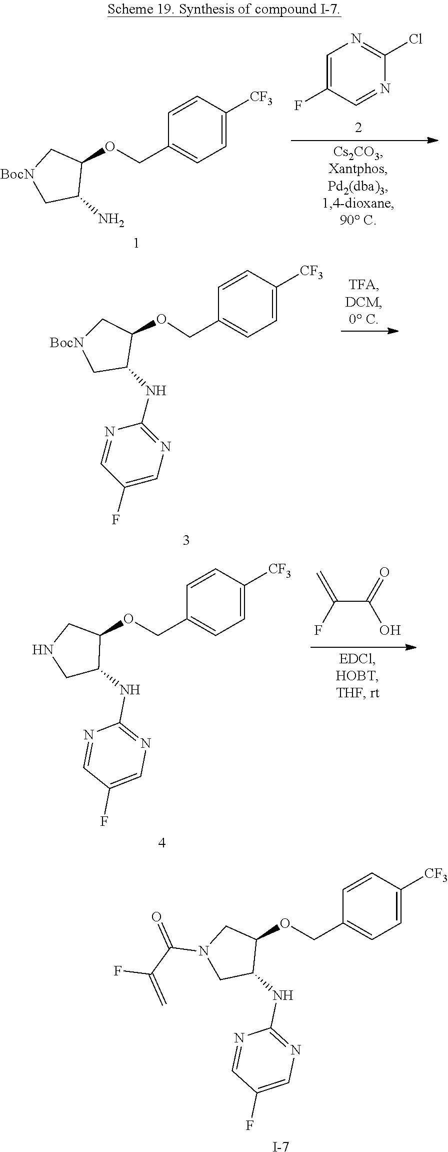

### Step 1: Synthesis of tert-butyl trans-3-((5-fluoropyrimidin-2-yl)amino)-4-((4-(trifluoromethyl)benzyl)oxy)pyrrolidine-1-carboxylate (Compound 3)

A mixture of tert-butyl trans-3-amino-4-((4-(trifluoromethyl)benzyl)oxy)pyrrolidine-1-carboxylate (500 mg, 1.39 mmol), 2-chloro-5-fluoropyrimidine (274.3 mg, 2.07 mmol), Pd₂(dba)₃ (127.3 mg, 0.139 mmol), Xantphos (241.3 mg, 0.417 mmol) and Cs₂CO₃ (905.7 mg. 2.78 mmol) in 1,4-dioxane (15 mL) was stirred at 90° C. under N₂ for 16 h. The mixture was then concentrated and purified by flash column chromatography on silica gel (eluting with 1% DCM in MeOH) to give compound 3 as a brown liquid (450 mg, 71.0% yield). LC-MS (ESI) m/z: 357 [M+H−100]⁺.

### Step 2: Synthesis of 5-fluoro-N-(trans-4-((4-(trifluoromethyl)benzyl)oxy)pyrrolidin-3-yl)pyrimidin-2-amine (Compound 4)

To the solution of compound 3 (0.30 g, 0.66 mmol) in DCM (8.0 mL) was added TFA (2.25 g, 19.8 mmol). The mixture was stirred at 0° C. for 3 h., diluted with DCNM (50 mL), washed with H₂O (30 mL×2) and saturated NaHCO₃ solution (30 mL×2), the DCM layer was concentrated under reduce pressure to leave crude compound 4 as a viscous colorless oil (0.23 g. 97.9% yield), which was used directly for the next step. LC-MS (ESI) m/z: 357 [M+H]⁺.

### Step 3: Synthesis of 2-fluoro-1-(trans-3-((5-fluoropyrimidin-2-yl)amino)-4-((4-(trifluoromethyl)benzyl)oxy)pyrrolidin-1-yl)prop-2-en-1-one (I-7)

A mixture of 2-fluoroacrylic acid (178 mg, 0.5 mmol), EDCI (191.7 mg, 1.0 mmol) and HOBt (135 mg, 1.0 mmol) in THF (10.0 mL) was stirred at room temperature for 0.5 h., and then compound 4 (90.0 mg, 1.0 mmol) was added, the resulting mixture was stirred at room temperature for 16 h. After the completion of the reaction, the mixture was concentrated and purified by prep-HPLC to afford the desired product I-7 as a white solid (80.1 mg. 37.4% yield). LC-MS (ESI) m/z: 429 [M+H]⁺. ¹H NMR (400 MHz, CD₃OD) δ (ppm) 8.27 (s, 2H), 7.65 (d, J=7.6 Hz, 2H), 7.62-7.52 (m, 2H), 5.49 (dt, J=47.2, 3.2 Hz, 1H), 5.25 (dt, J=16.5, 3.2 Hz, 1H), 4.86-4.75 (m, 2H), 4.50 (dd, J=13.3, 5.6 Hz, 1H), 4.18-4.09 (m, 1H), 4.01-3.89 (m, 1H), 3.89-3.75 (m, 2H), 3.68 (t, J=12.0 Hz, 1H). ¹⁹F NMR (376 MHz, DMSO-d₆) δ −63.955, −63.959, −111.50, −111.53, −157.66, −157.68.

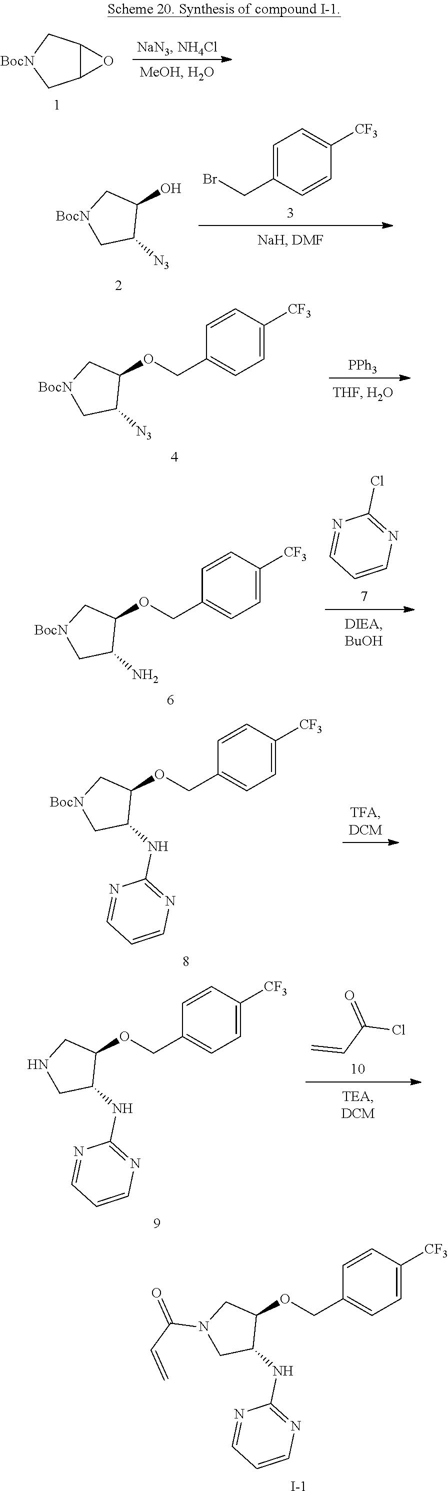

### Step 1: Synthesis of trans-tert-butyl 3-azido-4-hydroxypyrrolidine-1-carboxylate (Compound 2)

A mixture of tert-butyl 6-oxa-3-azabicyclo[3.1.0]hexane-3-carboxylate (10 g, 54 mmol), NaN₃ (7 g, 108 mmol) and NH₄Cl (2.8 g, 54 mmol) in MeOH (120 mL) and H₂O (20 mL) was stirred at 65° C. overnight. The reaction mixture was diluted with water (200 mL) and extracted with ethyl acetate (100 mL×3), the combined organic was washed with water (200 mL×2), dried over anhydrous Na₂SO₄, filtered and concentrated to leave crude compound 2 as oil (11 g, yield 89%). LC-MS (ESI) m/z: 129 [M+H−100]⁺.

### Step 2: Synthesis of tert-butyl trans-3-azido-4-(4-(trifluoromethyl)benzyloxy)pyrrolidine-1-carboxylate (Compound 4)

To a solution of compound 2 (3 g, 13.1 mmol) in THF (50 mL) was added NaH (0.6 g, 15.7 mmol), the mixture was stirred at rt for 10 minutes, and then 1-(bromomethyl)-4-(trifluoromethyl)benzene (3.1 g, 13.1 mmol) was added, the resulting mixture was stirred at rt under N₂ for 6 hours, diluted with water (200 mL) and extracted with ethyl acetate (200 mL), the organic was washed with water (100 mL), dried over anhydrous Na₂SO₄, concentrated and purified by flash column chromatography on silica gel (ethyl acetate in petroleum ether=20% v/v) to obtain compound 4 as yellow oil (4 g, yield 79%). LC-MS (ESI) m/z: 287 [M+H−100]⁺.

### Step 3: Synthesis of tert-butyl trans-3-amino-4-(4-(trifluoromethyl)benzyloxy)pyrrolidine-1-carboxylate (Compound 6)

A mixture of compound 4 (3 g, 7.7 mmol), PPh₃ (2.4 g, 9.3 mmol) and H₂O (2.8 g, 154 mmol) in THF (20 mL) was stirred at 70° C. under N₂ for 5 hours. The mixture was diluted with water (200 mL) and extracted with ethyl acetate (200 mL), the organic was washed with water (200 mL), dried over anhydrous Na₂SO₄, concentrated and purified by prep-HPLC to obtain compound 6 as yellow oil (2.3 g, yield 83%). LC-MS (ESI) m/z: 261 [M+H−100]⁺.

### Step 4: Synthesis of tert-butyl trans-3-(pyrimidin-2-ylamino)-4-(4-(trifluoromethyl)benzyloxy)pyrrolidine-1-carboxylate (Compound 8)

A mixture of compound 6 (300 mg, 0.81 mmol), 2-chloropyrimidine (120 mg, 0.99 mmol) and DIPEA (210 mg, 1.62 mmol) in BuOH (3 mL) was stirred at 70° C. under N₂ overnight. The mixture was concentrated and purified by prep-HPLC to obtain compound 8 as oil (270 mg, yield 76%). LC-MS (ESI) m/z: 439 [M+H]⁺.

### Step 5: Synthesis of N-(trans-4-(4-(trifluoromethyl)benzyloxy)pyrrolidin-3-yl)pyrimidin-2-amine (Compound 9)

A mixture of compound 8 (60 mg, 0.13 mmol) and TFA (1 mL) in DCM (3 mL) was stirred at rt for 2 hours. The mixture was concentrated to leave crude compound 9 as yellow oil (50 mg, yield 95%). LC-MS (ESI) m/z: 339 [M+H]⁺.

### Step 6: Synthesis of 1-(trans-3-(pyrimidin-2-ylamino)-4-(4-(trifluoromethyl)benzyloxy)pyrrolidin-1-yl)prop-2-en-1-one (I-1)

A mixture of compound 9 (50 mg, 0.15 mmol), acryloyl chloride (15 mg, 0.15 mmol), and TEA (30 mg, 0.3 mmol) in DCM (5 mL) was stirred at rt for 2 hours. The mixture was concentrated and purified by prep-HPLC to obtain compound I-1 as white solid (13 mg, yield 22%). LC-MS (ESI) m/z: 393 [M+H]⁺. ¹H NMR (400 MHz, CD₃OD) δ 8.33 (d, J=4.8 Hz, 2H), 7.63 (dd, J=28.8, 8.2 Hz, 4H), 6.74-6.55 (m, 2H), 6.31 (dd, J=16.8, 1.9 Hz, 1H), 5.78 (dd, J=10.4, 1.7 Hz, 1H), 4.85 (t, J=6.6 Hz, 2H), 4.61 (dd, J=20.0, 6.0 Hz, 1H), 4.27-4.17 (m, 1H), 4.11-3.67 (m, 4H).

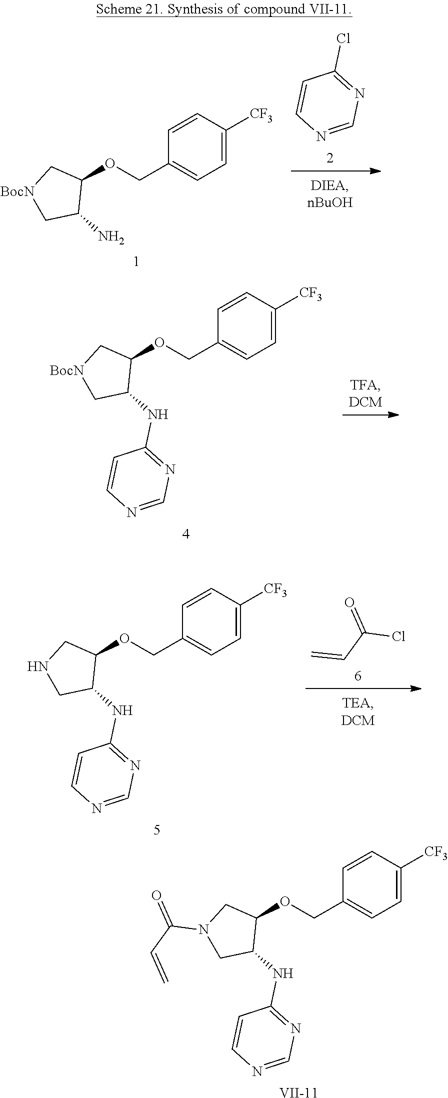

### Step 1: Synthesis of tert-butyl trans-3-(pyrimidin-4-ylamino)-4-(4-(trifluoromethyl)benzyloxy)pyrrolidine-1-carboxylate (Compound 4)

A mixture of tert-butyl trans-3-amino-4-((4-(trifluoromethyl)benzyl)oxy)pyrrolidine-1-carboxylate (300 mg, 0.81 mmol), 4-chloropyrimidine (120 mg, 0.99 mmol) and DIPEA (210 mg, 1.62 mmol) in n-BuOH (3 mL) was stirred at 70° C. under N₂ overnight. The reaction mixture was concentrated and purified by prep-HPLC to obtain compound 4 as oil (60 mg, yield 16%). LC-MS (ESI) m/z: 339 [M+H−100]⁺.

### Step 2: Synthesis of N-(trans-4-(4-(trifluoromethyl)benzyloxy)pyrrolidin-3-yl)pyrimidin-4-amine (Compound 5)

A mixture of compound 4 (60 mg, 0.13 mmol) and TFA (1 mL) in DCM (3 mL) was stirred at rt under N₂ for 2 hours. The mixture was concentrated to leave crude compound 5 as yellow oil (45 mg, yield 100%). LC-MS (ESI) m/z: 339 [M+H]⁺.

### Step 3: Synthesis of 1-(trans-3-(pyrimidin-4-ylamino)-4-(4-(trifluoromethyl)benzyloxy)pyrrolidin-1-yl)prop-2-en-1-one (VII-11)

A mixture of compound 5 (50 mg, 0.15 mmol), acryloyl chloride (15 mg, 0.15 mmol), and TEA (30 mg, 0.3 mmol) in DCM (5 mL) was stirred at rt under N₂ for 2 hours. The mixture was concentrated and purified by prep-HPLC to obtain compound VII-11 as a white solid (10 mg, yield 17%). LC-MS (ESI) m/z: 393 [M+H]⁺. ¹H NMR (400 MHz, CD₃OD) δ 8.50 (s, 1H), 8.04 (dd, J=6.1, 2.0 Hz, 1H), 7.64 (dd, J=28.0, 8.2 Hz, 4H), 6.61 (ddd, J=32.9, 16.4, 6.3 Hz, 2H), 6.33 (dd, J=16.8, 1.9 Hz, 1H), 5.80 (d, J=10.5 Hz, 1H), 4.82 (dd, J=40.8, 35.2 Hz, 3H), 4.27-3.65 (m, 5H).

Compounds V-3, V-5, and V-6 were generated according to General Synthesis Route III shown in Scheme 22 and using the specific reagents as detailed in Table 11. Scheme 23 shows the synthesis of Compound V-2.

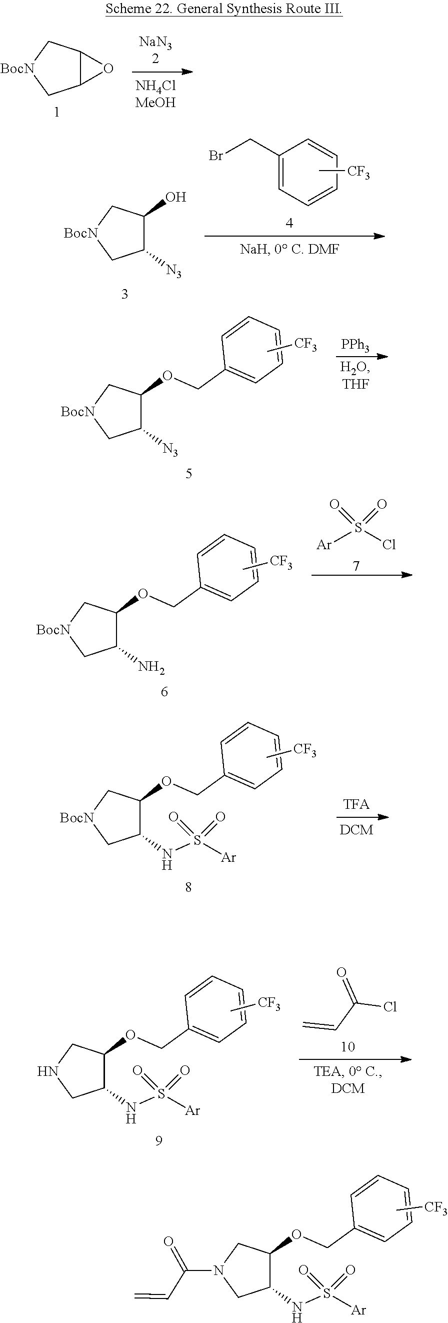

TABLE 11

| Compound | 4 | 7 |
|---|---|---|
| 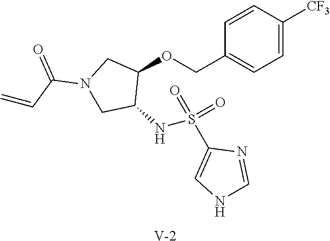 | 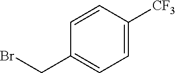 [SMILES: FC(F)(F)c1ccc(CBr)cc1] | 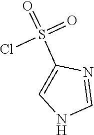 |
| 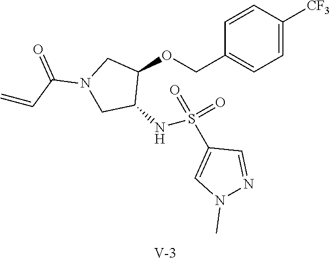 | 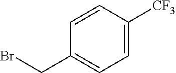 [SMILES: FC(F)(F)c1ccc(CBr)cc1] | 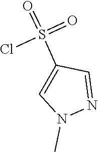 |
| 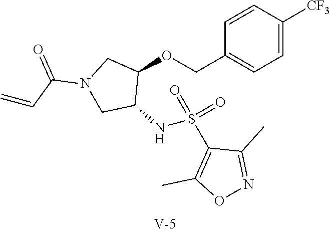 | 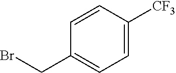 [SMILES: FC(F)(F)c1ccc(CBr)cc1] | 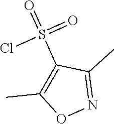 |
| 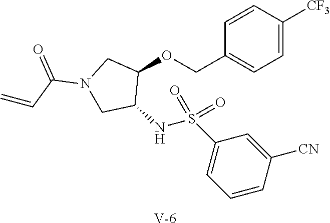 | 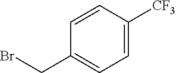 [SMILES: FC(F)(F)c1ccc(CBr)cc1] | 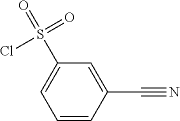 [SMILES: N#Cc1cccc(S(=O)(=O)Cl)c1] |

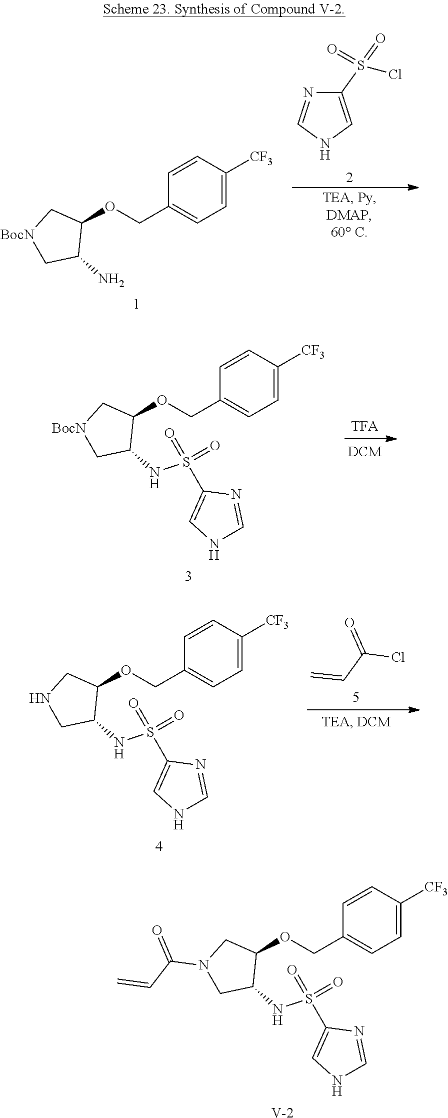

### Step 1: Synthesis of tert-butyl trans-3-(1H-imidazole-4-sulfonamido)-4-(4-(trifluoromethyl)benzyloxy)pyrrolidine-1-carboxylate (Compound 3)

A mixture of tert-butyl trans-3-amino-4-((4-(trifluoromethyl)benzyl)oxy)pyrrolidine-1-carboxylate (100 mg, 0.25 mmol), 1H-imidazole-4-sulfonyl chloride (83 mg, 0.5 mmol), DMAP (10 mg, 0.05 mmol) and TEA (80 mg, 0.75 mmol) in Pyridine (3 mL) was stirred at 60° C. under N₂ for 2 hours. The reaction mixture was cooled down to rt, diluted with water (100 mL) and extracted with ethyl acetate (50 mL), the organic was washed with water (30 mL), dried over anhydrous Na₂SO₄, concentrated and purified by column chromatography on silica gel (ethyl acetate in petroleum ether=40% v/v) to obtain compound 3 as white solid (50 mg, yield 41%). LC-MS (ESI) m/z: 391 [M+H−100]⁺.

### Step 2: Synthesis of N-(trans-4-(4-(trifluoromethyl)benzyloxy)pyrrolidin-3-yl)-1H-imidazole-4-sulfonamide (Compound 4)

A mixture of compound 3 (50 mg, 0.1 mmol) and TFA (1 mL) in DCM (3 mL) was stirred at rt under N₂ for 2 hours. The mixture was concentrated to leave crude compound 4 as yellow oil (35 mg, yield 90%), which was used directly for next step. LC-MS (ESI) m/z: 391 [M+H]⁺.

### Step 3: Synthesis of N-(trans-1-acryloyl-4-(4-(trifluoromethyl)benzyloxy)pyrrolidin-3-yl)-1H-imidazole-4-sulfonamide (V-2)

To a mixture of compound 4 (35 mg, 0.09 mmol) and TEA (18 mg, 0.18 mmol) in DCM (5 mL) was added acryloyl chloride (8 mg, 0.09 mmol), the mixture was stirred at rt under N₂ for 2 hours, concentrated and purified by prep-HPLC to obtain desired compound as white solid (3 mg, yield 7%). LC-MS (ESI) m/z: 445 [M+H]⁺. ¹H-NMR (400 MHz, CD₃OD) δ (ppm): 7.86 (s, 1H), 7.73 (s, 1H), 7.66 (d, J=8.0 Hz, 2H), 7.61-7.48 (m, 2H), 6.55 (d, J=10.6 Hz, 1H), 6.28 (d, J=16.2 Hz, 1H), 5.76 (d, J=10.3 Hz, 1H), 4.77-4.60 (m, 2H), 4.10 (t, J=29.8 Hz, 2H), 3.93-3.84 (m, 1H), 3.75-3.48 (m, 3H).

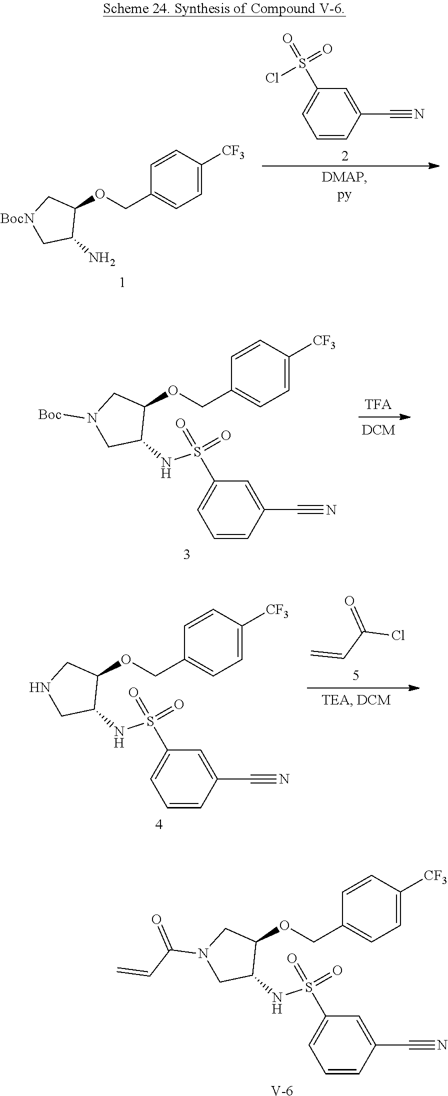

### Step 1: Synthesis of tert-butyl trans-3-(3-cyanophenylsulfonamido)-4-(4-(trifluoromethyl)benzyloxy)pyrrolidine-1-carboxylate (Compound 3)

A mixture of tert-butyl trans-3-amino-4-((4-(trifluoromethyl)benzyl)oxy)pyrrolidine-1-carboxylate (80 mg, 0.2 mmol), 3-cyanobenzenesulfonyl chloride (50 mg, 0.26 mmol) and DMAP (3 mg, 0.02 mmol) in Pyridine (5 mL) was stirred at 60° C. under N₂ for 2 hours. The mixture was concentrated and purified by prep-HPLC to obtain compound 3 as yellow oil (60 mg, yield 38%). LC-MS (ESI) m/z: 426 [M+H−100]⁺.

### Step 2: Synthesis of 3-cyano-N-(trans-4-(4-(trifluoromethyl)benzyloxy)pyrrolidin-3-yl)benzenesulfonamide (Compound 4)

A mixture of compound 3 (50 mg, 0.1 mmol) and TFA (1 mL) in DCM (5 mL) was stirred at rt for 2 hours. The mixture was concentrated to leave crude compound 4 as yellow oil (40 mg, yield 94%), which was used directly for next step. LC-MS (ESI) m/z: 426 [M+H]⁺.

### Step 3: Synthesis of N-(trans-1-acryloyl-4-(4-(trifluoromethyl)benzyloxy)pyrrolidin-3-yl)-3-cyanobenzenesulfonamide (V-6)

A mixture of compound 4 (40 mg, 0.09 mmol), acryloyl chloride (8 mg, 0.09 mmol) and TEA (18 mg, 0.18 mmol) in DCM (3 mL) was stirred at rt for 2 hours. The mixture was concentrated and purified by prep-HPLC to obtain compound V-6 as white solid (6 mg, yield 16%). LC-MS (ESI) m/z: 480 [M+H]⁺. ¹H NMR (400 MHz, CD₃OD) δ (ppm) 8.81-8.36 (m, 1H), 8.30-8.12 (m, 2H), 8.02-7.93 (m, 1H), 7.81-7.63 (m, 3H), 7.47 (dd, J=12.2, 8.1 Hz, 2H), 6.54 (ddd, J=22.7, 16.8, 10.4 Hz, 1H), 6.28 (dd, J=16.8, 1.8 Hz, 1H), 5.77 (dd, J=10.2, 2.2 Hz, 1H), 4.64 (d, J=21.3 Hz, 2H), 4.15-3.87 (m, 3H), 3.79-3.39 (m, 3H).

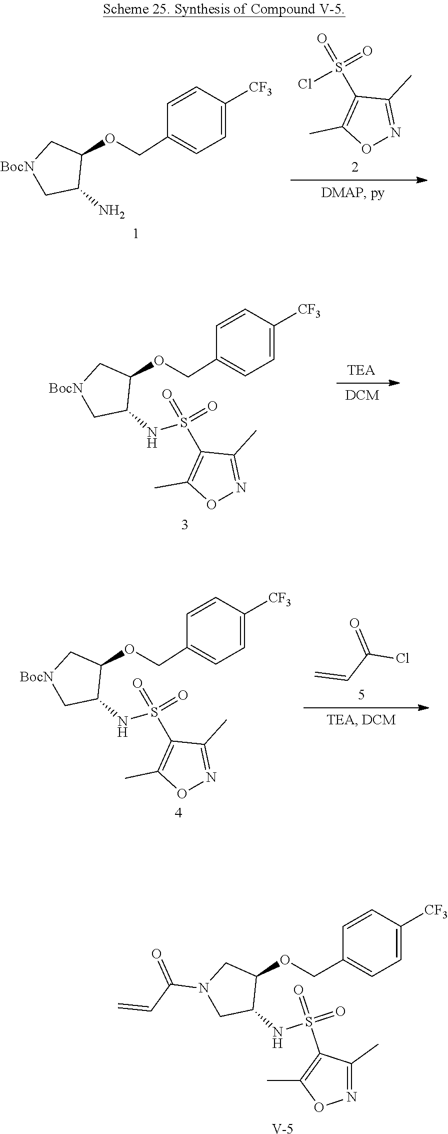

### Step 1: Synthesis of tert-butyl trans-3-(3,5-dimethylisoxazole-4-sulfonamido)-4-(4-(trifluoromethyl)benzyloxy)pyrrolidine-1-carboxylate (Compound 3)

A mixture of tert-butyl trans-3-amino-4-((4-(trifluoromethyl)benzyl)oxy)pyrrolidine-1-carboxylate (100 mg, 0.2 mmol), 3,5-dimethylisoxazole-4-sulfonyl chloride (70 mg, 0.3 mmol) and DMAP (10 mg, 0.02 mmol) in Pyridine (5 mL) was stirred at rt under N₂ for 2 hours. The mixture was concentrated and purified by prep-HPLC to obtain compound 3 as yellow oil (30 mg, yield 36%). LC-MS (ESI) m/z: 420 [M+H−100]⁺.

### Step 2: Synthesis of tert-butyl trans-3-(3,5-dimethylisoxazole-4-sulfonamido)-4-(4-(trifluoromethyl)benzyloxy)pyrrolidine-1-carboxylate (Compound 4)

A mixture of compound 3 (25 mg, 0.05 mmol) and TFA (1 mL) in DCM (3 mL) was stirred at rt for 2 hours. The mixture was concentrated to leave crude compound 4 as yellow oil (20 mg, yield 95%). LC-MS (ESI) m/z: 420 [M+H]⁺.

### Step 3: Synthesis of N-(trans-1-acryloyl-4-(4-(trifluoromethyl)benzyloxy)pyrrolidin-3-yl)-3,5-dimethylisoxazole-4-sulfonamide (V-5)

A mixture of compound 4 (20 mg, 0.05 mmol), acryloyl chloride (5 mg, 0.05 mmol), and TEA (10 mg, 0.1 mmol) in DCM (5 mL) was stirred at rt for 2 hours. The mixture was concentrated and purified by prep-HPLC to obtain compound V-5 as white solid (3 mg, yield 13%). LC-MS (ESI) m/z: 474 [M+H]⁺. ¹H NMR (400 MHz, CD₃OD) δ (ppm) 7.66 (d, J=8.1 Hz, 2H), 7.49 (t, J=7.4 Hz, 2H), 6.57 (ddd, J=16.8, 10.4, 8.6 Hz, 1H), 6.30 (dd, J=16.8, 1.5 Hz, 1H), 5.78 (d, J=10.4 Hz, 1H), 4.66 (d, J=13.2 Hz, 2H), 4.18-3.44 (m, 6H), 2.61 (d, J=1.3 Hz, 3H), 2.37 (s, 3H).

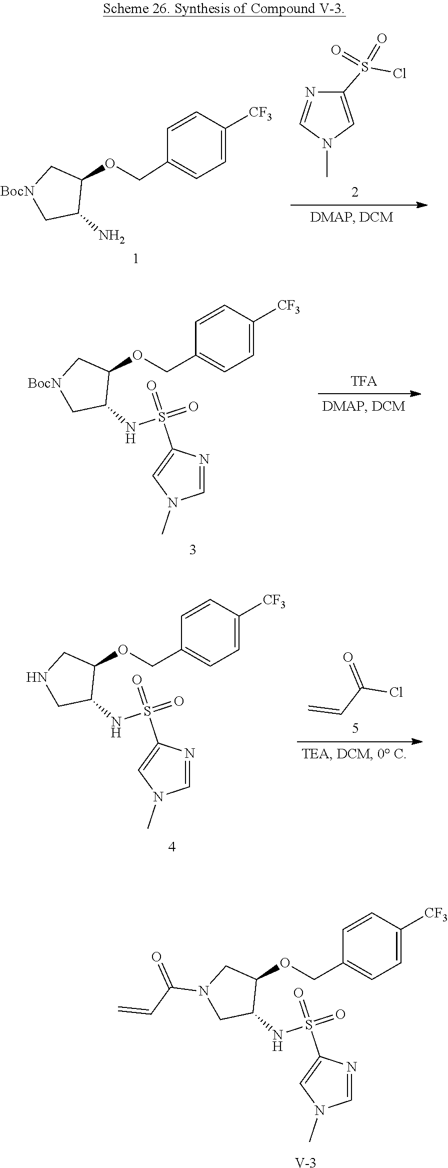

### Step 1: Synthesis of tert-butyl trans-3-(1-methyl-1H-imidazole-4-sulfonamido)-4-(4-(trifluoromethyl)benzyloxy)pyrrolidine-1-carboxylate (Compound 3)

A mixture of compound 1 (50 mg, 0.14 mmol), 1-methyl-1H-imidazole-4-sulfonyl chloride (29 mg, 0.16 mmol) and DMAP (2 mg, 0.014 mmol) in Pyridine (3 mL) was stirred at 60° C. under N₂ for 2 hours. The reaction mixture was concentrated and purified by prep-HPLC to obtain the target compound 3 as yellow solid (20 mg, yield 28%). LC-MS (ESI) m/z: 405 [M+H−100]⁺.

### Step 2: Synthesis of 1-methyl-N-(trans-4-(4-(trifluoromethyl)benzyloxy)pyrrolidin-3-yl)-1H-imidazole-4-sulfonamide (Compound 4)

A mixture of compound 3 (20 mg, 0.04 mmol) and TFA (1 mL) in DCM (5 mL) was stirred at rt under N₂ for 2 hours. The mixture was concentrated to leave the crude compound 4 as yellow oil (15 mg, yield 92%). LC-MS (ESI) m/z: 405 [M+H]⁺.

### Step 3: Synthesis of N-(trans-1-acryloyl-4-(4-(trifluoromethyl)benzyloxy)pyrrolidin-3-yl)-1-methyl-1H-imidazole-4-sulfonamide (V-3)

A mixture of compound 4 (15 mg, 0.037 mmol), acryloyl chloride (4 mg, 0.037 mmol) and TEA (8 mg, 0.074 mmol) in DCM (5 mL) was stirred at rt under N₂ for 2 hours. The mixture was concentrated and purified by prep-HPLC to obtain compound V-3 as white solid (5 mg, yield 36%). LC-MS (ESI) m/z: 459 [M+H]⁺. ¹H NMR (400 MHz, CD₃OD) δ (ppm) 8.14 (s, 1H), 7.80 (d, J=1.8 Hz, 1H), 7.67 (d, J=8.0 Hz, 2H), 7.52 (t, J=7.8 Hz, 2H), 6.66-6.48 (m, 1H), 6.38-6.24 (m, 1H), 5.77 (dd, J=10.4, 1.9 Hz, 1H), 4.68 (dd, J=16.3, 6.6 Hz, 2H), 4.23-4.09 (m, 1H), 3.94-3.46 (m, 8H).

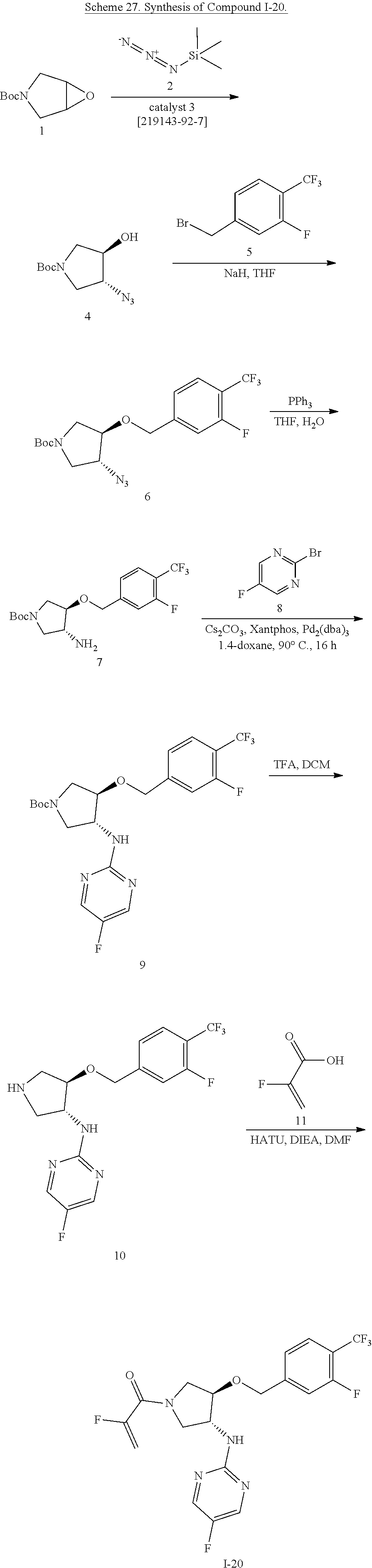

### Step 1: Synthesis of tert-butyl (3R,4R)-3-azido-4-hydroxypyrrolidine-1-carboxylate (Compound 4)

A mixture of tert-butyl 6-oxa-3-azabicyclo[3.1.0]hexane-3-carboxylate (4 g, 21.6 mmol), TMSN₃ (2.664 g, 23.2 mmol) and chiral catalyst (1S,2S)-(−)-[1,2-cyclohexanediamino-n,n′-bis (3,5-di-t-butylsalicylidene)]chromium (iii) chloride (328 mg, 0.42 mmol) was stirred at rt under N₂ overnight. The reaction mixture was treated with MeOH (60 mL) and K₂CO₃ (1.788 mg, 12.8 mmol) and continued to stir at rt for 5 hours. The reaction mixture was diluted with ethyl acetate (300 mL), washed with water (300 mL×2), dried over anhydrous Na₂SO₄, concentrated and purified by flash column chromatography on silica gel (ethyl acetate in petroleum ether=30% v/v) to obtain the target compound 2 as clear oil (3.5 g, 96% e.e., yield 71%). LC-MS (ESI) m/z: 129 [M+H−100]⁺.

### Step 2: Synthesis of tert-butyl (3R, 4R)-3-azido-4-(3-fluoro-4-(trifluoromethyl)benzyloxy)pyrrolidine-1-carboxylate (Compound 6)

To a solution of compound 2 (500 mg, 2.19 mmol) in THF (10 mL) was added NaH (105 mg, 2.62 mmol), the mixture was stirred at rt for 15 minutes, and then 4-(bromomethyl)-2-fluoro-1-(trifluoromethyl)benzene (560 mg, 2.19 mmol) was added. The resulting mixture was stirred at rt under N₂ for 6 hours. The mixture was diluted with water (100 mL) and extracted with ethyl acetate (100 mL), the organic was separated, washed with water (50 mL), dried over anhydrous Na₂SO₄, concentrated and purified by flash column chromatography (ethyl acetate:petroleum ether=5:1) to obtain compound 6 as clear oil (300 mg, yield 34%). LC-MS (ESI) m/z: t_{R}: 2.24 min, m/z: 305 [M+H−100]⁺.

### Step 3: Synthesis of tert-butyl (3R, 4R)-3-amino-4-(3-fluoro-4-(trifluoromethyl)benzyloxy)pyrrolidine-1-carboxylate (Compound 7)

A mixture of compound 6 (300 mg, 0.74 mmol), PPh₃ (233 mg, 0.89 mmol) and H₂O (266 mg, 14.8 mmol) in THF (20 mL) was stirred at 70° C. for 5 hours. The mixture was diluted with water (100 mL) and extracted with ethyl acetate (100 mL), the organic was washed with water (200 mL), dried over anhydrous Na₂SO₄, concentrated and purified by prep-HPLC to obtain compound 7 as yellow oil (200 mg, yield 72%). LC-MS (ESI) m/z: 279 [M+H−100]⁺.

### Step 4: Synthesis of tert-butyl (3R, 4R)-3-(3-fluoro-4-(trifluoromethyl)benzyloxy)-4-(5-fluoropyrimidin-2-ylamino)pyrrolidine-1-carboxylate (Compound 9)

A mixture of compound 7 (150 mg, 0.4 mmol), 2-bromo-5-fluoropyrimidine (84 mg, 0.47 mmol), Cs₂CO₃ (260 mg, 0.8 mmol), Xantphos (69 mg, 0.12 mmol) and Pd₂(dba)₃ (18 mg. 0.02 mmol) in 1,4-dioxane (5 mL) was stirred at 90° C. under N₂ overnight. The mixture was concentrated and purified by prep-HPLC to obtain compound 9 as brown solid (80 mg, yield 42%). LC-MS (ESI) m/z: 419 [M+H−56]⁺.

### Step 5: Synthesis of 5-fluoro-N-((3R,4R)-4-((3-fluoro-4-(trifluoromethyl)benzyl)oxy)pyrrolidin-3-yl)pyrimidin-2-amine (Compound 10)

A mixture of compound 9 (60 mg, 0.13 mmol) and TFA (1 mL) in DCM (3 mL) was at stirred 0° C. for 2 hours. The mixture was concentrated to leave the crude compound 10 as yellow oil (45 mg, yield 92%), which was used directly for next step. LC-MS (ESI) m/z: 375 [M+H]⁺.

### Step 6: Synthesis of 2-fluoro-1-(trans-3-(3-fluoro-4-(trifluoromethyl)benzyloxy)-4-(5-fluoropyrimidin-2-ylamino)pyrrolidin-1-yl)prop-2-en-1-one (Compound I-20)

A mixture of compound 10 (45 mg, 0.12 mmol), 2-fluoroacrylic acid (13 mg, 0.15 mmol), HATU (57 mg, 0.15 mmol) and DIEA (46 mg, 0.36 mol) in DMF (10 mL) was stirred at rt overnight. The mixture was concentrated and purified by prep-HPLC to obtain compound I-20 as white solid (25 mg, yield 47%). LC-MS (ESI) m/z: 447 [M+H]⁺. ¹H NMR (400 MHz, CD₃OD) δ (ppm) 8.26 (s, 2H), 7.65 (t, J=7.8 Hz, 1H), 7.36 (dt, J=11.1, 5.5 Hz, 2H), 5.49 (dd, J=47.2, 2.3 Hz, 1H), 5.25 (d, J=16.5 Hz, 1H), 4.82 (d, J=4.3 Hz, 2H), 4.50 (dd, J=14.6, 5.7 Hz, 1H), 4.20-3.67 (m, 5H).

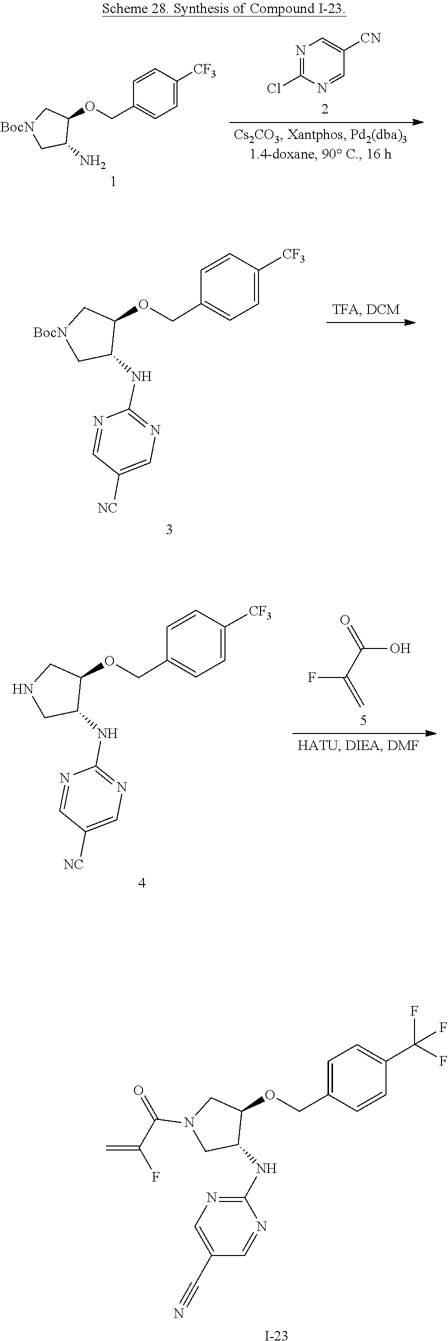

### Step 1: Synthesis of tert-butyl (3R, 4R)-3-(5-cyanopyrimidin-2-ylamino)-4-(4-(trifluoromethyl)benzyloxy)pyrrolidine-1-carboxylate (Compound 3)

A mixture of tert-butyl (3R, 4R)-3-amino-4-((4-(trifluoromethyl)benzyl)oxy)pyrrolidine-1-carboxylate (200 mg, 0.55 mmol), 2-chloropyrimidine-5-carbonitrile (77 mg, 0.55 mmol) and DIEA (142 mg, 1.1 mmol) in DMF (1 mL) was stirred at 110° C. under N₂ overnight. The mixture was concentrated and purified by flash column chromatography on silica gel (ethyl acetate in petroleum ether=60% v/v) to obtain compound 3 as white solid (200 mg, yield 78%). LC-MS (ESI) m/z: 408 [M+H−56]⁺.

### Step 2: Synthesis of 2-((3R, 4R)-4-(4-(trifluoromethyl)benzyloxy)pyrrolidin-3-ylamino)pyrimidine-5-carbonitrile (Compound 4)

A mixture of compound 3 (80 mg, 0.17 mmol) and TFA (1 mL) and in DCM (3 mL) was stirred at 0° C. for 2 hours. The mixture was concentrated to leave the crude compound 4 as yellow oil (60 mg, yield 97%), which was used directly for next step. LC-MS (ESI) m/z: 364 [M+H]⁺.

### Step 3: Synthesis of 2-(trans-1-(2-fluoroacryloyl)-4-(4-(trifluoromethyl)benzyloxy)pyrrolidin-3-ylamino)pyrimidine-5-carbonitrile (I-23)

A mixture of compound 4 (50 mg, 0.14 mmol), 2-fluoroacrylic acid (12 mg, 0.14 mmol), HATU (64 mg, 0.17 mmol) and DIEA (72 mg, 0.56 mmol) in DMF (5 mL) was stirred at rt for 5 hours. The mixture was concentrated and purified by prep-HPLC to obtain compound I-23 as white solid (10 mg, yield 16%). LC-MS (ESI) m/z: 436 [M+H]⁺. ¹H NMR (400 MHz, CD₃OD) δ (ppm) 8.66 (br, 1H), 8.56 (br, 1H), 7.70-7.51 (m, 4H), 5.49 (dt, J=47.2, 3.8 Hz, 1H), 5.26 (dt, J=16.5, 3.5 Hz, 1H), 4.85-4.75 (m, 2H), 4.68-4.60 (m, 1H), 4.21-3.65 (m, 5H).

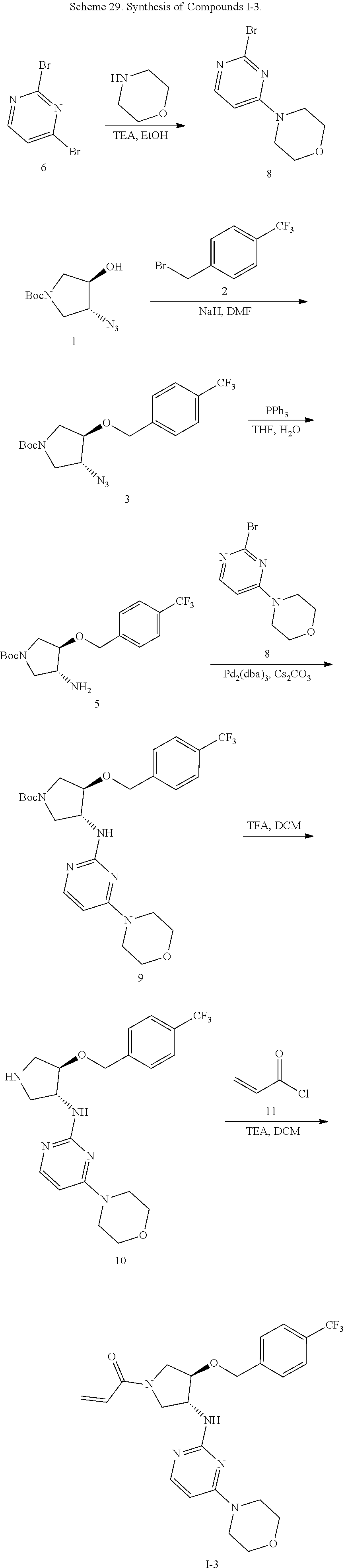

### Step 1: Synthesis of tert-butyl (3R,4R)-3-azido-4-(4-(trifluoromethyl)-benzyloxy)pyrrolidine-1-carboxylate (Compound 3)

To a solution of tert-butyl (3R,4R)-3-azido-4-hydroxypyrrolidine-1-carboxylate (1500 mg, 6.57 mmol) in DMF (80 mL) was added NaH (315 mg, 7.86 mmol), the mixture was stirred at rt for 10 minutes, and then 1-(bromomethyl)-4-(trifluoromethyl)benzene (1570 mg, 6.57 mmol) was added, the resulting mixture was stirred at rt under N₂ for 6 hours. The mixture was quenched with water (200 mL) and extracted with ethyl acetate (100 mL×2), the combined organic was washed with water (200 mL), dried over anhydrous Na₂SO₄, concentrated and purified by flash column chromatography on silica gel (ethyl acetate:petroleum ether=5:1) to obtain compound 3 as clear oil (2.2 g, yield 86%). LC-MS (ESI) m/z: 287 [M+H−100]⁺.

### Step 2: Synthesis of tert-butyl (3R,4R)-3-amino-4-(4-(trifluoromethyl)benzyloxy)pyrrolidine-1-carboxylate (Compound 5)

A mixture of compound 3 (1500 mg, 3.8 mmol), PPh₃ (1221 mg, 4.6 mmol) and H₂O (1386 mg, 72 mmol) in THF (20 mL) was stirred at 70° C. for 5 hours. The mixture was diluted with water (200 mL) and extracted with ethyl acetate (200 mL), the organic was washed with water (200 mL), dried over anhydrous Na₂SO₄, concentrated and purified by prep-HPLC to obtain compound 5 as yellow oil (1.25 g, yield 91%). LC-MS (ESI) m/z: 261 [M+H−100]⁺.

### Step 3: Synthesis of 4-(2-bromopyrimidin-4-yl)morpholine (Compound 8)

A mixture of 2,4-dibromopyrimidine (500 mg, 2.1 mmol), morpholine (182 mg, 2.1 mmol) and TEA (424 mg, 4.2 mmol) in EtOH (5 mL) was stirred at rt overnight. The mixture was concentrated and purified by flash column chromatography on silica gel (ethyl acetate in petroleum ether=30% v/v) to obtain compound 8 as white solid (300 mg, yield 58%). LC-MS (ESI) m/z: 244 [M+H]⁺.

### Step 4: Synthesis of tert-butyl (3R,4R)-3-(4-morpholinopyrimidin-2-ylamino)-4-(4-(trifluoromethyl)benzyloxy)pyrrolidine-1-carboxylate (Compound 9)

A mixture of compound 5 (200 mg, 0.5 mmol), compound 8 (145 mg, 0.6 mmol), Cs₂CO₃ (324 mg, 1.0 mmol), Xantphos (29 mg, 0.05 mmol) and Pd₂(dba)₃ (46 mg, 0.05 mmol) in 1,4-dioxane (5 mL) was stirred at 110° C. under N₂ overnight. The mixture was concentrated and purified by flash column chromatography on silica gel (ethyl acetate in petroleum ether=40% v/v) to obtain compound 9 as white solid (100 mg, yield 38%). LC-MS (ESI) m/z: 524 [M+H]⁺.

### Step 5: Synthesis of 4-morpholino-N-((3R,4R)-4-(4-(trifluoromethyl)benzyloxy)pyrrolidin-3-yl)pyrimidin-2-amine (Compound 10)

A mixture of compound 9 (40 mg, 0.076 mmol) and TFA (1 mL) in DCM (2 mL) was stirred at rt for 2 hours. The mixture was concentrated to leave the crude compound 10 as yellow oil (30 mg, crude), which was used directly for next step. LC-MS (ESI) m/z: 424 [M+H]⁺.

### Step 6: Synthesis of 1-((3R,4R)-3-(4-morpholinopyrimidin-2-ylamino)-4-(4-(trifluoromethyl)benzyloxy)pyrrolidin-1-yl)prop-2-en-1-one (Compound I-3)

A mixture of compound 10 (30 mg, 0.07 mmol), acryloyl chloride (7 mg, 0.07 mmol), and TEA (14 mg, 0.14 mmol) in DCM (5 mL) was stirred at rt for 2 hours. The mixture was concentrated and purified by prep-HPLC to obtain compound I-3 as white solid (6 mg, yield 18%). LC-MS (ESI) m/z: 478 [M+H]⁺. ¹H NMR (400 MHz, CD₃OD) δ (ppm) 7.81 (d, J=6.2 Hz, 1H), 7.63 (d, J=8.1 Hz, 2H), 7.53 (d, J=8.0 Hz, 2H), 6.65-6.55 (m, 1H), 6.28 (dd, J=16.8, 1.9 Hz, 1H), 6.09 (dd, J=6.2, 1.0 Hz, 1H), 5.75 (dd, J=10.4, 1.9 Hz, 1H), 4.83-4.73 (m, 2H), 4.63-4.49 (m, 1H), 4.28-4.16 (m, 1H), 4.10-3.54 (m, 13H).

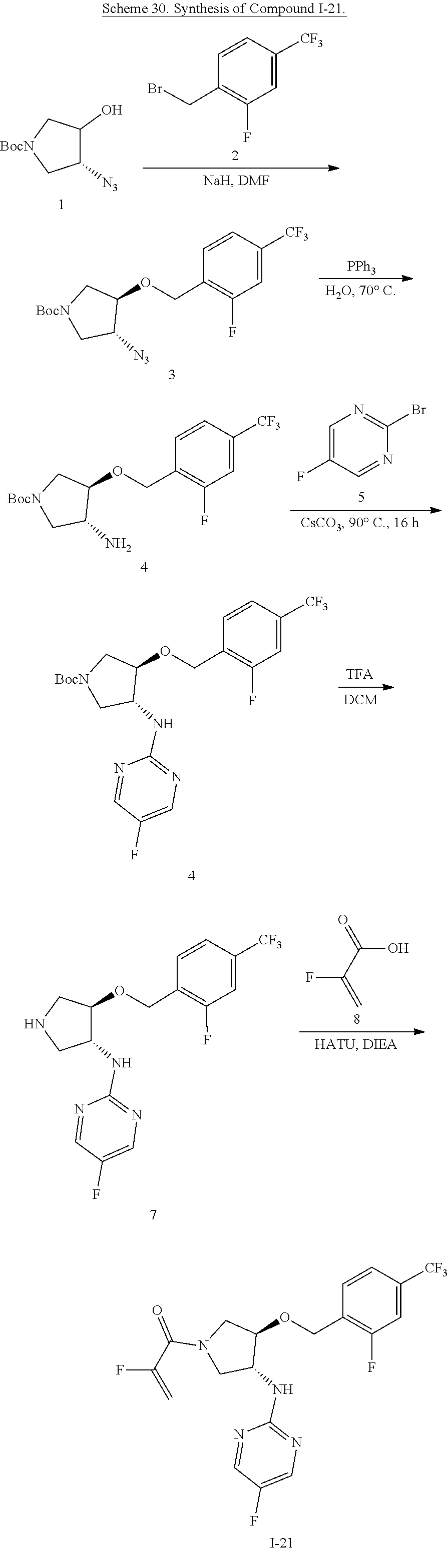

### Step 1: Synthesis of tert-butyl (3R,4R)-3-azido-4-(2-fluoro-4-(trifluoromethyl)benzyloxy)pyrrolidine-1-carboxylate (Compound 3)

To a solution of tert-butyl (3R, 4R)-3-azido-4-hydroxypyrrolidine-1-carboxylate (350 mg, 1.53 mmol) in DMF (20 mL) was added NaH (60%, 73 mg, 1.84 mmol), the mixture was stirred at rt for 10 minutes, and then 1-(bromomethyl)-2-fluoro-4-(trifluoromethyl)benzene (393 mg, 1.53 mmol) was added, the mixture was stirred at rt under N₂ for 6 hours, quenched with water (100 mL) and extracted with ethyl acetate (100 mL), the organic was washed with water (50 mL), dried over anhydrous Na₂SO₄, concentrated and purified by flash column chromatography (ethyl acetate:petroleum ether=5:1) to obtain compound 3 as clear oil (300 mg, yield 48%). LC-MS (ESI) m/z: 305 [M+H−100]⁺.

### Step 2: Synthesis of tert-butyl (3R,4R)-3-amino-4-((2-fluoro-4-(trifluoromethyl)benzyl)oxy)pyrrolidine-1-carboxylate (Compound 4)

A mixture of compound 3 (300 mg, 0.74 mmol). PPh₃ (233 mg, 0.89 mmol) and H₂O (266 mg, 14.8 mmol) in THF (20 mL) was stirred at 70° C. under N₂ for 5 hours. The mixture was diluted with water (100 mL) and extracted with ethyl acetate (100 mL), the extract was washed with water (100 mL), dried over anhydrous Na₂SO₄, concentrated and purified by flash column chromatography on silica gel (MeOH in DCM=10% v/v) to obtain compound 4 as clear oil (200 mg, yield 71%). LC-MS (ESI) m/z: t_{R}: 2.24 min, m/z: 279 [M+H−100]⁺.

### Step 3: Synthesis of tert-butyl (3R,4R)-3-(2-fluoro-4-(trifluoromethyl)benzyloxy)-4-(5-fluoropyrimidin-2-ylamino)pyrrolidine-1-carboxylate (Compound 6)

A mixture of compound 4 (200 mg, 0.53 mmol). 2-bromo-5-fluoropyrimidine (112 mg. 0.63 mmol) and Cs₂CO₃ (344 mg, 1.06 mmol) in DMF (3 mL) was stirred at 100° C. under N₂ overnight. The mixture was concentrated and purified by flash column chromatography on silica gel (ethyl acetate in petroleum ether=50% v/v) to obtain compound 6 as yellow solid (50 mg, yield 20%). LC-MS (ESI) m/z: 419 [M+H−56]⁺.

### Step 4: Synthesis of 5-fluoro-N-((3R,4R)-4-(2-fluoro-4-(trifluoromethyl)benzyloxy)pyrrolidin-3-yl)pyrimidin-2-amine (Compound 7)

A mixture of compound 6 (50 mg, 0.1 mmol) and TFA (1 mL) and in DCM (3 mL) was stirred at 0° C. for 2 hours. The reaction mixture was concentrated to leave the crude compound 7 as yellow oil (40 mg, yield 100%), which was used directly for next step. LC-MS (ESI) m/z: 375 [M+H]⁺.

### Step 5: Synthesis of 2-fluoro-1-((3R,4R)-3-(2-fluoro-4-(trifluoromethyl)benzyloxy)-4-(5-fluoropyrimidin-2-ylamino)pyrrolidin-1-yl)prop-2-en-1-one (I-21)

A mixture of compound 7 (30 mg, 0.08 mmol). 2-fluoroacrylic acid (9 mg, 0.1 mmol). HATU (36 mg, 0.1 mmol) and DIEA (31 mg, 0.24 mmol) in DMF (5 mL) was stirred at rt overnight. The mixture was concentrated and purified by prep-HPLC to obtain compound I-21 as yellow solid (2 mg, yield 6%). LC-MS (ESI) m/z: 447 [M+H]⁺. ¹H NMR (400 MHz, CD₃OD) δ (ppm) 8.26 (s, 2H), 7.70 (q, J=7.9 Hz, 1H), 7.52 (d, J=8.0 Hz, 1H), 7.45 (d, J=10.4 Hz, 1H), 5.48 (d, J=47.1, 2.0 Hz, 1H), 5.25 (dd, J=16.5, 3.3 Hz, 1H), 4.93 (s, 2H), 4.54-4.47 (m, 1H), 4.23-3.79 (m, 4H), 3.74-3.63 (m, 1H).

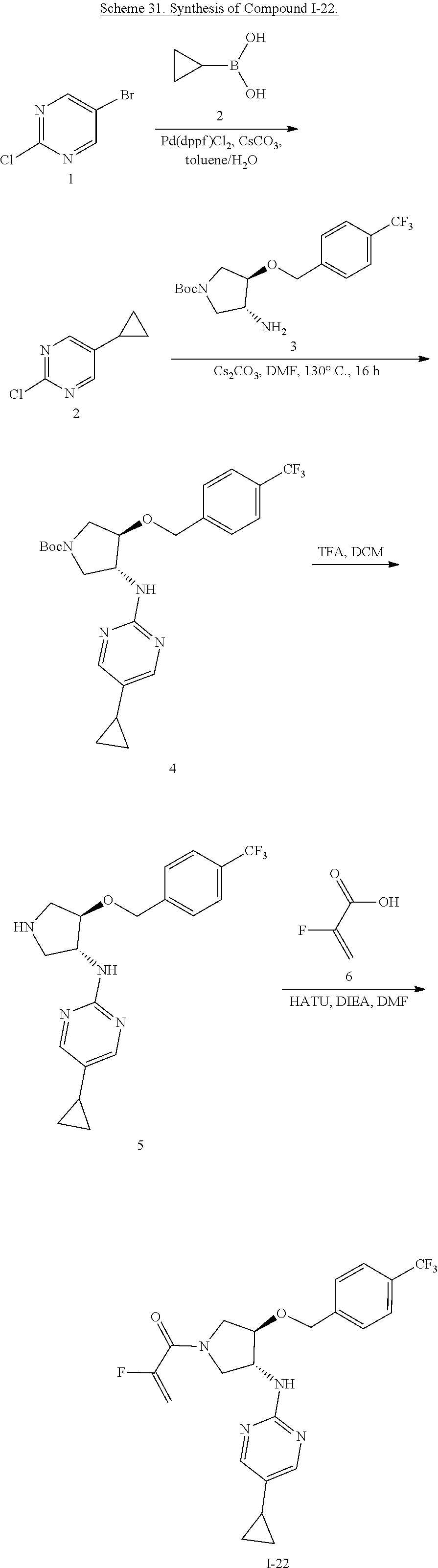

### Step 1: Synthesis of 2-chloro-5-cyclopropylpyrimidine (Compound 2)

A mixture of 5-bromo-2-chloropyrimidine (900 mg, 4.7 mmol), cyclopropylboronic acid (608 mg, 7.1 mmol), Cs₂CO₃ (4582 mg, 14.1 mmol) and Pd(dppf)Cl₂ (41 mg, 0.05 mmol) in toluene (5 mL) and H₂O (0.5 mL) was stirred at 110° C. under N₂ overnight. The mixture was concentrated and purified by flash column chromatography on silica gel (ethyl acetate in petroleum ether=10% v/v) to obtain compound 2 as white solid (450 mg, yield 62%). LC-MS (ESI) m/z: 155 [M+H]⁺.

### Step 2: Synthesis of tert-butyl (3R, 4R)-3-(5-cyclopropylpyrimidin-2-ylamino)-4-(4-(trifluoromethyl)benzyloxy)pyrrolidine-1-carboxylate (Compound 4)

A mixture of compound 2 (171 mg, 1.1 mmol), tert-butyl (3R,4R)-3-amino-4-((4-(trifluoromethyl)benzyl)oxy)pyrrolidine-1-carboxylate (200 mg, 0.55 mmol) and Cs₂CO₃ (358 mg, 1.1 mmol) in DMF (2 mL) was stirred at 130° C. under N₂ overnight. The mixture was concentrated and purified by flash column chromatography on silica gel (ethyl acetate in petroleum ether=70% v/v) to obtain compound 4 as clear oil (90 mg, yield 34%). LC-MS (ESI) m/z: 479 [M+H]⁺.

### Step 3: Synthesis of 5-cyclopropyl-N-((3R,4R)-4-((4-(trifluoromethyl)benzyl)oxy)pyrrolidin-3-yl)pyrimidin-2-amine (Compound 5)

A mixture of compound 4 (40 mg, 0.08 mmol) and TFA (1 mL) in DCM (3 mL) was stirred at 0° C. for 2 hours. The reaction mixture was concentrated to leave the crude compound 5 as yellow oil (30 mg, yield 98%), which was used directly for next step. LC-MS (ESI) m/z: 379 [M+H]⁺.

### Step 4: Synthesis of 1-((3R,4R)-3-(5-cyclopropylpyrimidin-2-ylamino)-4-(4-(trifluoromethyl)benzyloxy)pyrrolidin-1-yl)-2-fluoroprop-2-en-1-one (I-22)

A mixture of compound 5 (40 mg, 0.10 mmol), 2-fluoroacrylic acid (9 mg, 0.10 mmol), HATU (46 mg, 0.12 mmol) and DIEA (52 mg, 0.40 mmol) in DMF (5 mL) was stirred at rt for 5 hours. The mixture was concentrated and purified by prep-HPLC to obtain compound I-22 as white solid (15 mg, yield 33%). LC-MS (ESI) m/z: 451 [M+H]⁺. ¹H NMR (400 MHz, CD₃OD) δ (ppm) 8.12 (s, 2H), 7.64 (d, J=7.1 Hz, 2H), 7.60-7.50 (m, 2H), 5.48 (dt, J=47.2, 3.0 Hz, 1H), 5.25 (dt, J=16.5, 3.2 Hz, 1H), 4.84-4.76 (m, 2H), 4.53 (dd, J=12.4, 6.0 Hz, 1H), 4.20-3.62 (m, 5H), 1.81-1.72 (m, 1H), 0.99-0.83 (m, 2H), 0.67-0.54 (m, 2H).

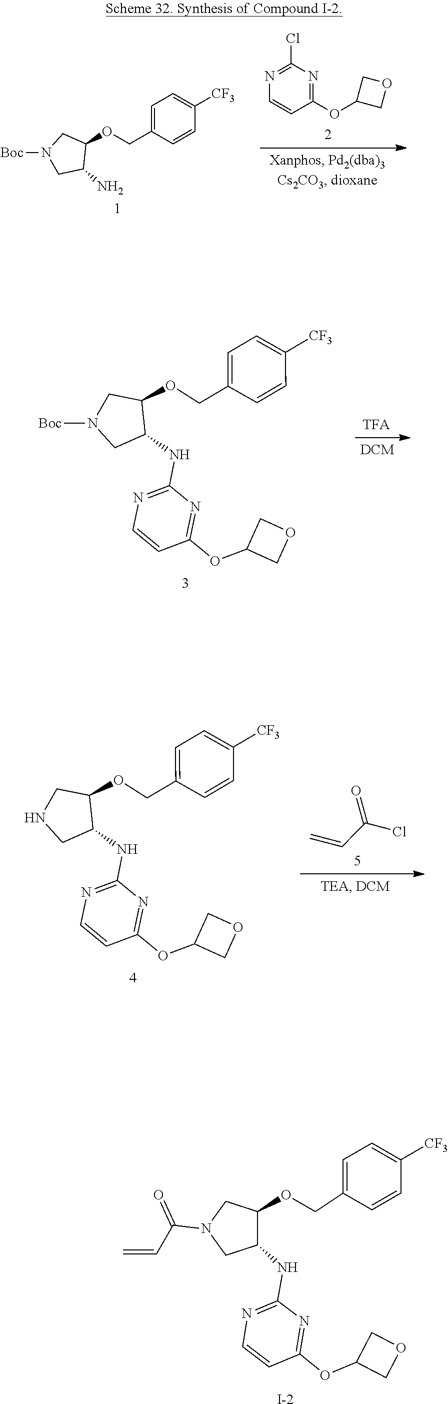

### Step 1: Synthesis of tert-butyl trans-3-(4-(oxetan-3-yloxy)pyrimidin-2-ylamino)-4-(4-(trifluoromethyl)benzyloxy)pyrrolidine-1-carboxylate (Compound 3)

A mixture of tert-butyl trans-3-amino-4-((4-(trifluoromethyl)benzyl)oxy)pyrrolidine-1-carboxylate (103 mg, 0.55 mmol), 2-chloro-4-(oxetan-3-yloxy)pyrimidine (200 mg, 0.55 mmol), Cs₂CO₃ (357 mg, 1.1 mmol), Xantphos (95 mg, 0.17 mmol) and Pd₂(dba)₃ (25 mg, 0.003 mmol) in 1,4-dioxane (6 mL) was stirred at 100° C. under N₂ overnight. The mixture was concentrated and purified by flash column chromatography on silica gel (ethyl acetate in petroleum ether=60% v/v) to obtain compound 3 as yellow solid (50 mg, yield 18%). LC-MS (ESI) m/z: 511 [M+H]⁺.

### Step 2: Synthesis of 4-(oxetan-3-yloxy)-N-(trans-4-(4-(trifluoromethyl)benzyloxy)pyrrolidin-3-yl)pyrimidin-2-amine (Compound 4)

A mixture of compound 3 (45 mg, 0.09 mmol) and TFA (1 mL) and in DCM (3 mL) was stirred 0° C. for 2 h. The reaction mixture was concentrated to leave crude compound 4 as yellow oil (35 mg, crude), which was used directly for next step. LC-MS (ESI) m/z: 411 [M+H]⁺.

### Step 3: Synthesis of 1-(trans-3-(4-(oxetan-3-yloxy)pyrimidin-2-ylamino)-4-(4-(trifluoromethyl)benzyloxy)pyrrolidin-1-yl)prop-2-en-1-one (I-2)

A mixture of compound 4 (35 mg, 0.08 mmol), acryloyl chloride (8 mg, 0.08 mmol) and TEA (10 mg, 0.16 mmol) in DCM (10 mL) was stirred at rt for 2 h. The mixture was concentrated and purified by prep-HPLC to obtain compound I-2 as yellow solid (12 mg, yield 32%). LC-MS (ESI) m/z: 465 [M+H]⁺. ¹H NMR (400 MHz, CD₃OD) δ (ppm) 8.06 (d, J=5.7 Hz, 1H), 7.60 (dd, J=34.2, 8.0 Hz, 4H), 6.67-6.54 (m, 1H), 6.35-6.13 (m, 2H), 5.80-5.53 (m, 2H), 4.99-4.92 (m, 2H), 4.79 (d, J=5.8 Hz, 2H), 4.66 (d, J=7.6 Hz, 2H), 4.52 (s, 1H), 4.25-4.14 (m, 1H), 4.08-3.60 (m, 4H).

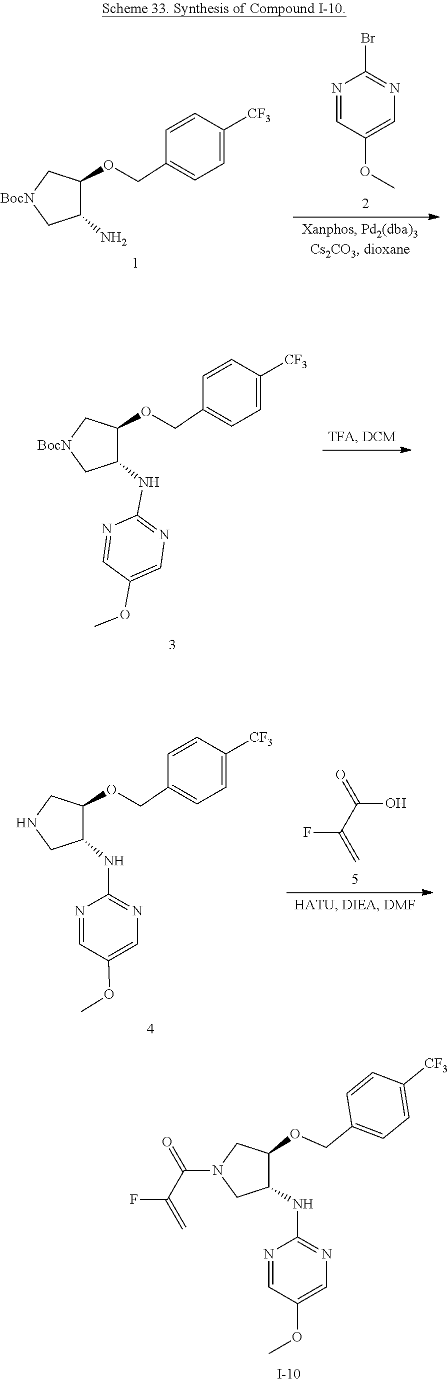

### Step 1: Synthesis of tert-butyl (3R,4R)-3-(5-methoxypyrimidin-2-ylamino)-4-(4-(trifluoromethyl)benzyloxy)pyrrolidine-1-carboxylate (Compound 3)

A mixture of tert-butyl (3R,4R)-3-amino-4-((4-(trifluoromethyl)benzyl)oxy)pyrrolidine-1-carboxylate (100 mg, 0.27 mmol), 2-bromo-5-methoxypyrimidine (78 mg, 0.41 mmol), Xantphos (15 mg, 0.027 mmol), Pd₂(dba)₃ (12 mg, 0.0135 mmol) and Cs₂CO₃ (263 mg, 0.81 mmol) in DMF (5 mL) was stirred at 90° C. for 1 h. The mixture was concentrated and purified by prep-HPLC to obtain compound 3 as white solid (60 mg, yield 47%). LC-MS (ESI) m/z: 469 [M+H]⁺.

### Step 2: Synthesis of 5-methoxy-N-((3R,4R)-4-(4-(trifluoromethyl)benzyloxy)pyrrolidin-3-yl)pyrimidin-2-amine (Compound 4)

A mixture of compound 3 (50 mg, 0.1 mmol) and TFA (1 mL) in DCM (3 mL) was stirred at rt under N₂ for 2 h. The mixture was concentrated leave the crude compound 4 as yellow oil (40 mg, yield 100%). LC-MS (ESI) m/z: 369 [M+H]⁺.

### Step 3: Synthesis of 2-fluoro-1-((3R,4R)-3-(5-methoxypyrimidin-2-ylamino)-4-(4-(trifluoromethyl)benzyloxy)pyrrolidin-1-yl)prop-2-en-1-one (compound I-10)

A mixture of compound 4 (50 mg, 0.13 mmol), 2-fluoroacrylic acid (15 mg, 0.16 mmol), HATU (60 mg, 0.16 mmol) and DIEA (50 mg, 0.39 mmol) in DMF (5 mL) was stirred at rt overnight. The mixture was purified directly by prep-HPLC to obtain I-10 as white solid (20 mg, yield 35%). LC-MS (ESI) m/z: 441 [M+H]⁺. ¹H NMR (400 MHz, CD₃OD) δ (ppm) 8.16 (d, J=2.8 Hz, 2H), 7.71-7.49 (m, 4H), 5.49 (dt, J=47.2, 3.1 Hz, 1H), 5.25 (dt, J=16.6, 3.2 Hz, 1H), 4.87-4.77 (m, 2H), 4.51 (dd, J=12.9, 5.7 Hz, 1H), 4.21-3.66 (m, 8H).

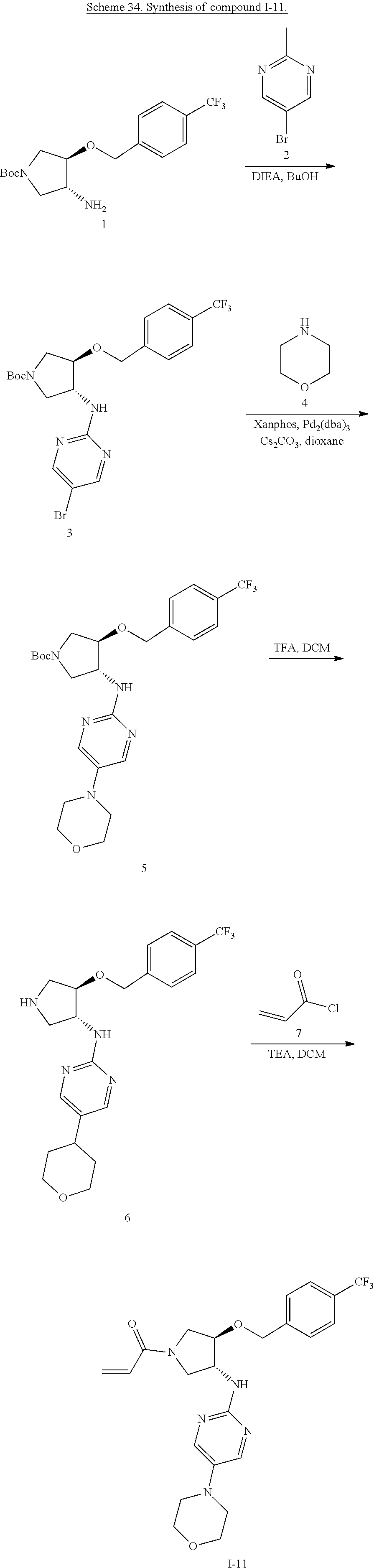

### Step 1: Synthesis of tert-butyl trans-3-(5-bromopyrimidin-2-ylamino)-4-(4-(trifluoromethyl)benzyloxy)pyrrolidine-1-carboxylate (Compound 3)

A mixture of tert-butyl trans-3-amino-4-((4-(trifluoromethyl)benzyl)oxy)pyrrolidine-1-carboxylate (300 mg, 0.8 mmol), 5-bromo-2-iodopyrimidine (284 mg, 0.99 mmol) and DIPEA (206 mg, 1.6 mmol) in BuOH (3 mL) was stirred at 70° C. under N₂ overnight. The mixture was concentrated and purified by flash column chromatography on silica gel (ethyl acetate in petroleum ether=90% v/v) to obtain compound 3 as yellow solid (400 mg, yield 97%). LC-MS (ESI) m/z: 461 [M+H−56]⁺.

### Step 2: Synthesis of tert-butyl trans-3-(5-morpholinopyrimidin-2-ylamino)-4-(4-(trifluoromethyl)benzyloxy)pyrrolidine-1-carboxylate (Compound 5)

A mixture of compound 3 (100 mg, 0.2 mmol), morpholine (20 mg, 0.2 mmol), tBuONa (40 mg, 0.04 mmol), Xantphos (12 mg, 0.02 mmol) and Pd₂(dba)₃ (20 mg, 0.02 mmol) in toluene (6 mL) was stirred at 110° C. under N₂ overnight. The mixture was concentrated and purified by flash column chromatography on silica gel (ethyl acetate in petroleum ether=90% v/v) to obtain compound 5 as yellow solid (35 mg, yield 33%). LC-MS (ESI) m/z: 524 [M+H]⁺.

### Step 3: Synthesis of 5-morpholino-N-(trans-4-(4-(trifluoromethyl)benzyloxy)pyrrolidin-3-yl)pyrimidin-2-amine (Compound 6)

A mixture of compound 5 (20 mg, 0.04 mmol) and TFA (1 mL) in DCM (3 mL) was stirred at rt for 2 h. The mixture was concentrated to leave the crude compound 6 as yellow oil (16 mg, yield 94%). LC-MS (ESI) m/z: 424 [M+H]⁺.

### Step 4: Synthesis of 1-(trans-3-(5-morpholinopyrimidin-2-ylamino)-4-(4-(trifluoromethyl)benzyloxy)pyrrolidin-1-yl)prop-2-en-1-one (I-11)

A mixture of compound 6 (16 mg, 0.04 mmol), acryloyl chloride (4 mg, 0.04 mmol) and TEA (8 mg, 0.08 mmol) in DCM (5 mL) was stirred at rt for 2 h. The mixture was concentrated and purified by prep-HPLC to obtain compound I-11 as white solid (10 mg, yield 52%). LC-MS (ESI) m/z: 478 [M+H]⁺. ¹H NMR (400 MHz, CD₃OD) δ (ppm) 8.13 (s. 2H), 7.64 (d, J=8.2 Hz, 2H), 7.57 (d, J=8.2 Hz, 2H), 6.61 (ddd, J=16.4, 10.4, 5.7 Hz, 1H), 6.29 (dd, J=16.8, 1.9 Hz, 1H), 5.75 (dd, J=10.4, 1.4 Hz, 1H), 4.82 (t, J=6.2 Hz, 2H), 4.55-4.45 (m, 1H), 4.22-4.14 (m, 1H), 4.04-3.66 (m, 8H), 3.01 (dd, J=5.6, 3.8 Hz, 4H).

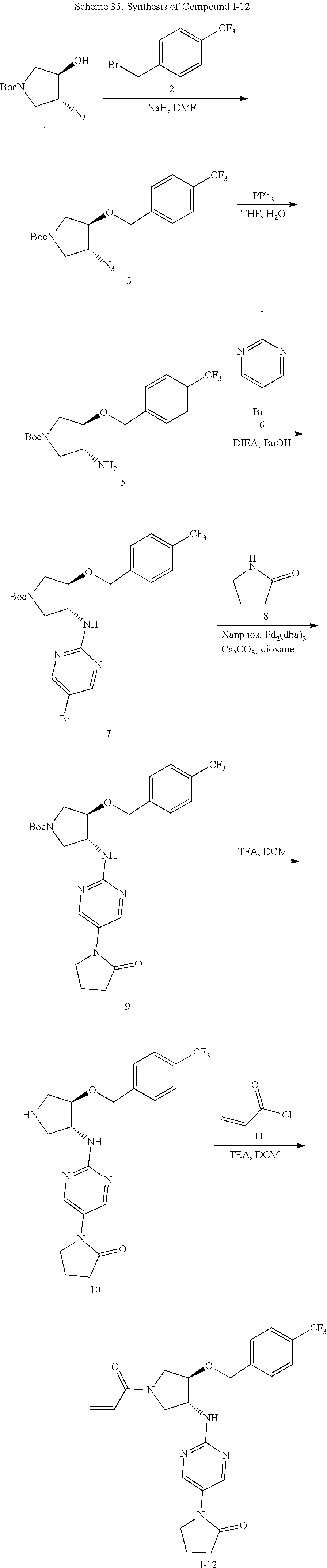

### Step 1: Synthesis of tert-butyl trans-3-azido-4-(4-(trifluoromethyl)benzyloxy)pyrrolidine-1-carboxylate (Compound 3)

A mixture of tert-butyl trans-3-azido-4-hydroxypyrrolidine-1-carboxylate (500 mg, 2.19 mmol), 1-(bromomethyl)-4-(trifluoromethyl)benzene (524 mg, 2.19 mmol) and NaH (105 mg, 2.62 mmol) in THF (10 mL) was stirred at rt under N₂ for 6 h. The mixture was diluted with water (100 mL) and extracted with ethyl acetate (100 mL), the organic was washed with water (50 mL), dried over anhydrous Na₂SO₄, concentrated and purified by flash column chromatography (ethyl acetate:petroleum ether=1:5) to obtain compound 3 as white oil (500 mg, yield 59%). LC-MS (ESI) m/z: 287 [M+H−100]⁺.

### Step 2: Synthesis of tert-butyl trans-3-amino-4-(4-(trifluoromethyl)benzyloxy)pyrrolidine-1-carboxylate (Compound 5)

A mixture of compound 3 (1000 mg, 2.58 mmol), PPh₃ (810 mg, 3.1 mmol) and H₂O (930 mg, 5.16 mmol) in THF (10 mL) was stirred at 70° C. under N₂ for 5 h. The mixture was diluted with water (50 mL) and extracted with ethyl acetate (3×50 mL), the combined organic was washed with water (100 mL), dried over anhydrous Na₂SO₄, concentrated and purified by prep-HPLC to obtain compound 5 as yellow oil (800 mg, yield 86%). LC-MS (ESI) m/z: 261 [M+H−100]⁺.

### Step 3: Synthesis of tert-butyl trans-3-(5-bromopyrimidin-2-ylamino)-4-(4-(trifluoromethyl)benzyloxy)pyrrolidine-1-carboxylate (Compound 7)

A mixture of compound 5 (150 mg, 0.4 mmol), 5-bromo-2-iodopyrimidine (130 mg, 0.46 mmol) and DIPEA (105 mg, 0.81 mmol) in BuOH (3 mL) was stirred at 70° C. under N₂ overnight. The mixture was then concentrated and purified by flash column chromatography on silica gel (ethyl acetate in petroleum ether=50% v/v) to obtain compound 7 as white oil (200 mg, yield 96%). LC-MS (ESI) m/z: 461 [M+H−56]⁺.

### Step 4: Synthesis of tert-butyl trans-3-(5-(2-oxopyrrolidin-1-yl)pyrimidin-2-ylamino)-4-(4-(trifluoromethyl)benzyloxy)pyrrolidine-1-carboxylate (Compound 9)

A mixture of compound 7 (100 mg, 0.2 mmol), pyrrolidin-2-one (20 mg, 0.2 mmol), Cs₂CO₃ (130 mg, 0.4 mmol) and Pd(dppf)Cl₂ (20 mg, 0.02 mmol) in toluene (6 mL) was stirred at 110° C. under N₂ overnight. The mixture was diluted with water (50 mL) and extracted with ethyl acetate (30 mL), the organic was washed with water (20 mL), dried over anhydrous Na₂SO₄, concentrated and purified by flash column chromatography on silica gel (ethyl acetate in petroleum ether=90% v/v) to obtain compound 9 as yellow solid (35 mg, yield 33%). LC-MS (ESI) m/z: 522 [M+H]⁺.

### Step 5: Synthesis of 1-(2-(trans-4-(4-(trifluoromethyl)benzyloxy)pyrrolidin-3-ylamino)pyrimidin-5-yl)pyrrolidin-2-one (Compound 10)

A mixture of compound 9 (30 mg, 0.06 mmol) and TFA (1 mL) in DCM (3 mL) was stirred at rt for 2 h. The mixture was concentrated to leave the crude compound 10 as yellow oil (20 mg, yield 79%). LC-MS (ESI) m/z: 422 [M+H]⁺.

### Step 6: Synthesis of 1-(2-(trans-1-acryloyl-4-(4-(trifluoromethyl)benzyloxy)pyrrolidin-3-ylamino)pyrimidin-5-yl)pyrrolidin-2-one (I-12)

A mixture of compound 10 (30 mg, 0.07 mmol), acryloyl chloride (6 mg, 0.07 mmol) and TEA (14 mg, 0.14 mmol) in DCM (5 mL) was stirred at rt under N₂ for 2 h. The mixture was concentrated and purified to obtain compound I-12 as white solid (10 mg, yield 30%). LC-MS (ESI) m/z: 476 [M+H]⁺. ¹H NMR (400 MHz, CD₃OD) δ (ppm) 8.54 (s, 2H), 7.64 (d, J=8.2 Hz, 2H), 7.57 (d, J=8.2 Hz, 2H), 6.61 (ddd, J=17.0, 10.4, 6.7 Hz, 1H), 6.29 (dd, J=16.8, 1.9 Hz, 1H), 5.76 (dd, J=10.4, 1.3 Hz, 1H), 4.83 (t, J=6.9 Hz, 2H), 4.58 (dd, J=19.8, 6.1 Hz, 1H), 4.25-4.13 (m, 1H), 4.04-3.66 (m, 6H), 2.56 (t, J=8.1 Hz, 2H), 2.26-2.13 (m, 2H).

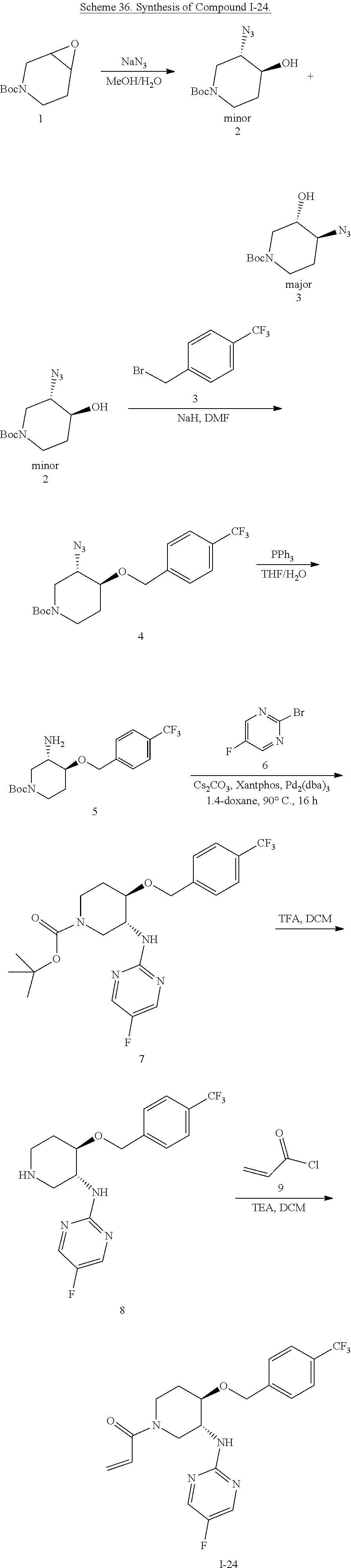

### Step 1: Synthesis of tert-butyl trans-3-azido-4-hydroxypiperidine-1-carboxylate (Compound 2)

A mixture of tert-butyl 7-oxa-3-azabicyclo[4.1.0]heptane-3-carboxylate (1000 mg, 5.0 mmol), NaN₃ (650 mg, 10.0 mmol) and NH₄Cl (270 mg, 5.0 mmol) in MeOH/H₂O (60 mL) was stirred at 65° C. overnight. The reaction mixture was concentrated and purified by flash column chromatography on silica gel (Et₂O in petroleum ether=40% v/v) to obtain compound 2 (200 mg) as yellow oil. LC-MS (ESI) m/z: 143 [M+H−100]⁺.

### Step 2: Synthesis of tert-butyl trans-3-azido-4-(4-(trifluoromethyl)benzyloxy)piperidine-1-carboxylate (Compound 4)

A mixture of compound 2 (200 mg, 0.83 mmol), 1-(bromomethyl)-4-(trifluoromethyl)benzene (198 mg, 0.83 mmol) and NaH (40 mg, 0.99 mmol) in DMF (15 mL) was stirred at 0° C. for 5 hours. The reaction was monitored by LCMS. The mixture was diluted with water (100 mL) and extracted with ethyl acetate (100 mL×2), the combined organic was washed with water (100 mL), dried over anhydrous Na₂SO₄, concentrated and purified by column chromatography on silica gel (ethyl acetate in petroleum ether=20% v/v) to obtain compound 4 as oil (200 mg, yield 60%). LC-MS (ESI) m/z: 301 [M+H−100]⁺.

### Step 3: Synthesis of tert-butyl trans-3-amino-4-(4-(trifluoromethyl)benzyloxy)piperidine-1-carboxylate (Compound 5)

A mixture of compound 4 (100 mg, 0.25 mmol), PPh₃ (79 mg, 0.3 mmol) and H₂O (90 mg, 5.0 mmol) in THF (10 mL) was stirred at 70° C. for 5 hours. The mixture was diluted with water (100 mL) and extracted with ethyl acetate (100 mL), the organic was washed with water (200 mL), dried over anhydrous Na₂SO₄, concentrated and purified by flash column chromatography on silica gel (MeOH in DCM=10% v/v) to obtain compound 5 as oil (75 mg). LC-MS (ESI) m/z: 319 [M−55]⁺.

### Step 4: Synthesis of tert-butyl trans-3-(5-fluoropyrimidin-2-ylamino)-4-(4-(trifluoromethyl)benzyloxy)piperidine-1-carboxylate (Compound 7)

A mixture of compound 5 (130 mg, 0.35 mmol), 2-bromo-5-fluoropyrimidine (61 mg. 0.35 mmol), Cs₂CO₃ (228 mg, 0.70 mmol), Xantphos (58 mg, 0.10 mmol) and Pd₂(dba)₃ (16 mg, 0.02 mmol) in 1,4-dioxane (6 mL) was stirred at 100° C. under N₂ overnight. The mixture was concentrated and purified by flash column chromatography on silica gel (ethyl acetate in petroleum ether=60% v/v) to obtain compound 7 as yellow solid (70 mg). LC-MS (ESI) m/z: 471 [M+H]⁺.

### Step 5: Synthesis of 5-fluoro-N-(trans-4-(4-(trifluoromethyl)benzyloxy)piperidin-3-yl)pyrimidin-2-amine (Compound 8)

A mixture of compound 7 (65 mg, 0.14 mmol) and TFA (1 mL) and in DCM (3 mL) was stirred at 0° C. for 2 h. The mixture was concentrated to leave crude compound 8 (50 mg) as yellow oil. LC-MS (ESI) m/z: 371 [M+H]⁺.

### Step 6: Synthesis of 1-(trans-3-((5-fluoropyrimidin-2-yl)amino)-4-((4-(trifluoromethyl)benzyl)oxy)piperidin-1-yl)prop-2-en-1-one (I-24)

A mixture of compound 8 (50 mg, 0.14 mmol), acryloyl chloride (13 mg, 0.14 mmol) and TEA (28 mg, 0.28 mmol) in DCM (10 mL) was stirred at rt for 2 h. The mixture was concentrated and purified by prep-HPLC to obtain compound I-24 as yellow solid (10 mg). LC-MS (ESI) m/z: 425 [M+H]⁺. ¹H NMR (400 MHz, CD₃OD) δ (ppm) 8.24 (d, J=12.4 Hz, 2H), 7.58-7.53 (m, 4H), 6.87-6.60 (m, 1H), 6.19 (t, J=16.6 Hz, 1H), 5.74-5.68 (m, 1H), 4.79-4.65 (m, 2H), 4.12-4.06 (m, 3H), 3.75 (s, 1H), 3.45 (dd, J=13.3, 7.5 Hz, 2H), 2.17 (s, 1H), 1.68 (s, 1H).

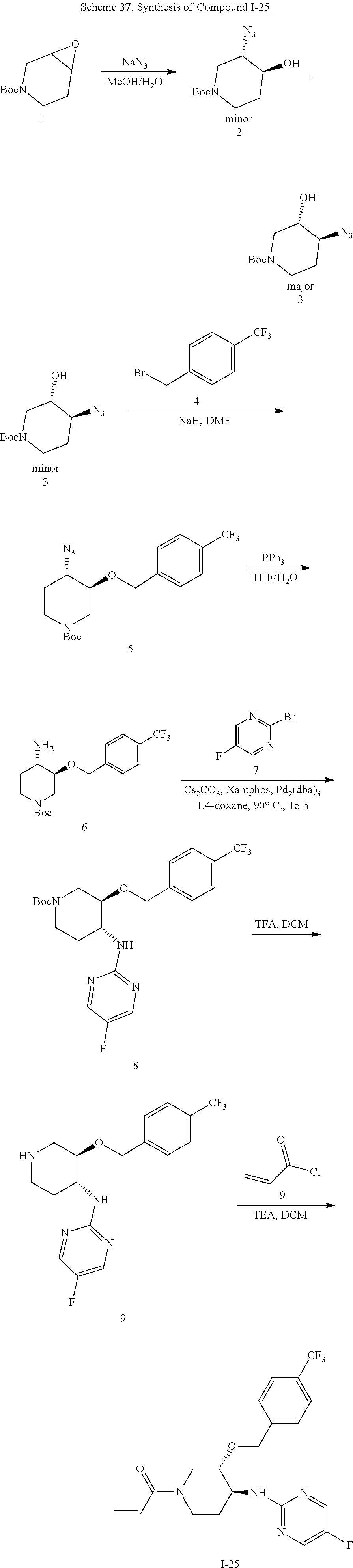

### Step 1: Synthesis of tert-butyl trans-4-azido-3-hydroxypiperidine-1-carboxylate (Compound 3)

A mixture of tert-butyl 7-oxa-3-azabicyclo[4.1.0]heptane-3-carboxylate (1000 mg, 5.0 mmol), NaN₃ (650 mg, 10.0 mmol) and NH₄Cl (270 mg, 5.0 mmol) in MeOH/H₂O (60 mL) was stirred at 65° C. overnight. The reaction mixture was concentrated and purified by flash column chromatography on silica gel (Et₂O in petroleum ether=40% v/v) to obtain compound 3 (700 mg) as yellow oil. LC-MS (ESI) m/z: 143 [M+H−100]⁺.

### Step 2: Synthesis of tert-butyl trans-4-azido-3-(4-(trifluoromethyl)benzyloxy)piperidine-1-carboxylate (Compound 5)

A mixture of compound 3 (300 mg, 1.24 mmol), 1-(bromomethyl)-4-(trifluoromethyl)benzene (295 mg, 1.24 mmol) and NaH (60 mg, 1.50 mmol) in DMF (15 mL) was stirred at 0° C. for 5 hours. The reaction was monitored by LCMS. The mixture was diluted with water (100 mL) and extracted with ethyl acetate (100 mL×2), the combined organic was washed with water (100 mL), dried over anhydrous Na₂SO₄, concentrated and purified by flash column chromatography on silica gel (ethyl acetate in petroleum ether=20% v/v) to obtain compound 5 as oil (350 mg, yield 70%). LC-MS (ESI) m/z: 301 [M+H−100]⁺.

### Step 3: Synthesis of tert-butyl trans-4-amino-3-(4-(trifluoromethyl)benzyloxy)piperidine-1-carboxylate (Compound 6)

A mixture of compound 5 (350 mg, 0.87 mmol), PPh₃ (275 mg, 1.05 mmol) and H₂O (31 mg, 1.74 mmol) in THF (10 mL) was stirred at 70° C. for 5 hours. The mixture was diluted with water (100 mL) and extracted with ethyl acetate (100 mL), the organic was washed with water (200 mL), dried over anhydrous Na₂SO₄, concentrated and purified by flash column chromatography on silica gel (MeOH in DCM=10% v/v) to obtain compound 6 as oil (270 mg). LC-MS (ESI) m/z: 319 [M−55]⁺.

### Step 4: Synthesis of tert-butyl trans-4-(5-fluoropyrimidin-2-ylamino)-3-(4-(trifluoromethyl)benzyloxy)piperidine-1-carboxylate (Compound 8)

A mixture of compound 6 (150 mg, 0.4 mmol), 2-bromo-5-fluoropyrimidine (71 mg, 0.40 mmol), Cs₂CO₃ (260 mg, 0.8 mmol), Xantphos (69 mg, 0.12 mmol) and Pd₂(dba)₃ (18 mg. 0.02 mmol) in 1,4-dioxane (6 mL) was stirred at 100° C. under N₂ overnight. The mixture was concentrated and purified by flash column chromatography on silica gel (ethyl acetate in petroleum ether=60% v/v) to obtain compound 8 as yellow solid (60 mg). LC-MS (ESI) m/z: 471 [M+H]⁺.

### Step 5: Synthesis of 5-fluoro-N-(trans-3-(4-(trifluoromethyl)benzyloxy)piperidin-4-yl)pyrimidin-2-amine (Compound 9)

A mixture of compound 8 (50 mg, 0.11 mmol) and TFA (1 mL) and in DCM (3 mL) was stirred at 0° C. for 2 h. The mixture was concentrated to leave crude compound 9 (40 mg) as yellow oil. LC-MS (ESI) m/z: 371 [M+H]⁺.

### Step 6: Synthesis of 1-(trans-4-(5-fluoropyrimidin-2-ylamino)-3-(4-(trifluoromethyl)benzyloxy)piperidin-1-yl)prop-2-en-1-one (I-25)

A mixture of compound 9 (40 mg, 0.11 mmol), acryloyl chloride (10 mg, 0.11 mmol) and TEA (22 mg, 0.22 mmol) in DCM (10 mL) was stirred at rt for 2 h. The mixture was concentrated and purified by prep-HPLC to obtain compound I-25 as yellow solid. LC-MS (ESI) m/z: 425 [M+H]⁺. ¹H NMR (400 MHz, CD₃OD) δ (ppm) 8.21 (s, 2H), 7.51 (dt, J=15.9, 8.4 Hz, 4H), 6.76 (ddd, J=20.8, 16.8, 10.7 Hz, 1H), 6.19 (t, J=15.5 Hz, 1H), 5.73 (dd, J=14.7, 11.0 Hz, 1H), 4.76-4.55 (m, 2H), 4.41-3.42 (m, 6H), 2.13 (s, 1H), 1.61 (dt, J=14.7, 6.8 Hz, 1H).

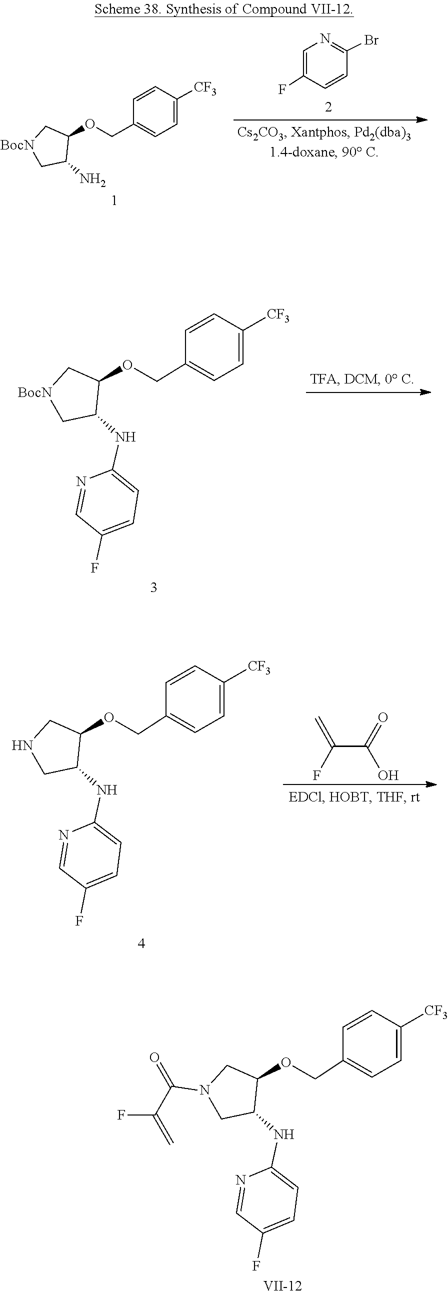

### Step 1: Synthesis of tert-butyl trans-3-((5-fluoropyridin-2-yl)amino)-4-((4-(trifluoromethyl)benzyl)oxy)pyrrolidine-1-carboxylate (Compound 3)

To a three necked flask was added compound 1 (racemic, 500 mg, 1.39 mmol) and 1,4-dioxane (15 mL), followed by the addition of 2-bromo-5-fluoropyridine 2 (274.3 mg, 2.07 mmol), Pd₂(dba)₃ (127.3 mg, 0.139 mmol), Xantphos (241.3 mg, 0.417 mmol) and Cs₂CO₃ (905.7 mg, 2.78 mmol). The flask was evacuated, and refilled with N₂ for three times. The resulting mixture was stirred at 90° C. under N₂ for 16 hours. After the completion of the reaction, the solvent was removed under reduce pressure to leave the crude product, which was purified by flash column chromatography on silica gel (eluting with 1% DCM in MeOH) to afford the desired compound 3 as a brown liquid (350 mg, 55.3% yield). LC-MS (ESI) m/z: 456 [M+H]⁺.

### Step 2: Synthesis of 5-fluoro-N-(trans-4-((4-(trifluoromethyl)benzyl)oxy)pyrrolidin-3-yl)pyridin-2-amine

To the solution of compound 3 (0.30 g, 0.66 mmol) in DCM (8.0 mL) was added TFA (2.25 g, 19.8 mmol). The resulting mixture was stirred at 0° C. for 3 hours. After the completion of the reaction, the mixture was washed with H₂O (30 mL×2) and saturated NaHCO₃ aqueous (30 mL×2), the DCM layer was concentrated under reduce pressure to leave the desired compound 4 as a viscous colorless liquid (0.22 g, 93.9% yield), which was used directly for the next step. LC-MS (ESI) m/z: 356 [M+H]⁺.

### Step 3: Synthesis of 2-fluoro-1-(trans-3-((5-fluoropyridin-2-yl)amino)-4-((4-(trifluoromethyl)benzyl)oxy)pyrrolidin-1-yl)prop-2-en-1-one (VII-12)

A mixture of 2-fluoroacrylic acid (45.0 mg, 0.50 mmol), EDCI (45 mg, 0.50 mmol) and HOBt (95.9 mg, 0.50 mmol) in THF (5.0 mL) was stirred at room temperature for 0.5 hour, and then compound 4 (90 mg, 0.25 mmol) was added, the resulting mixture was stirred at room temperature for 16 hours, concentrated and purified by prep-HPLC to afford the desired product VII-12 as a white solid (58.3 mg, 54.6% yield). LC-MS (ESI) m/z: 428 [M+H]⁺. ¹H NMR (400 MHz, CD₃OD) δ (ppm) 7.94 (d, J=1.8 Hz, 1H), 7.64 (d, J=7.2 Hz, 2H), 7.59-7.46 (m, 3H), 6.79-6.68 (m, 1H), 5.55-5.48 (m, 1H), 5.26 (dt, J=16.5, 3.3 Hz, 1H), 4.85-4.73 (m, 2H), 4.53-4.41 (m, 1H), 4.26-4.08 (m, 2H), 4.04-3.91 (m, 1H), 3.88-3.77 (m, 2H), 3.75-3.60 (m, 1H). Hz, 1H), ¹⁹F NMR (376 MHz, DMSO-d₆) δ (ppm): −63.93, −63.94, −111.53, −111.74, −144.20, −144.35.

Compounds II-1, II-2, II-3, II-4, II-5, II-6, II-7, II-9, II-10, II-11, II-12, II-14, II-15, II-16, II-17, II-19, II-20, II-21, II-22, VII-9, III-1, III-2, III-4, III-6, VII-3, VII-4 were synthesized according to general synthesis route IV shown in Scheme 39 below and using the specific using the specific reagents as detailed in Table 12.

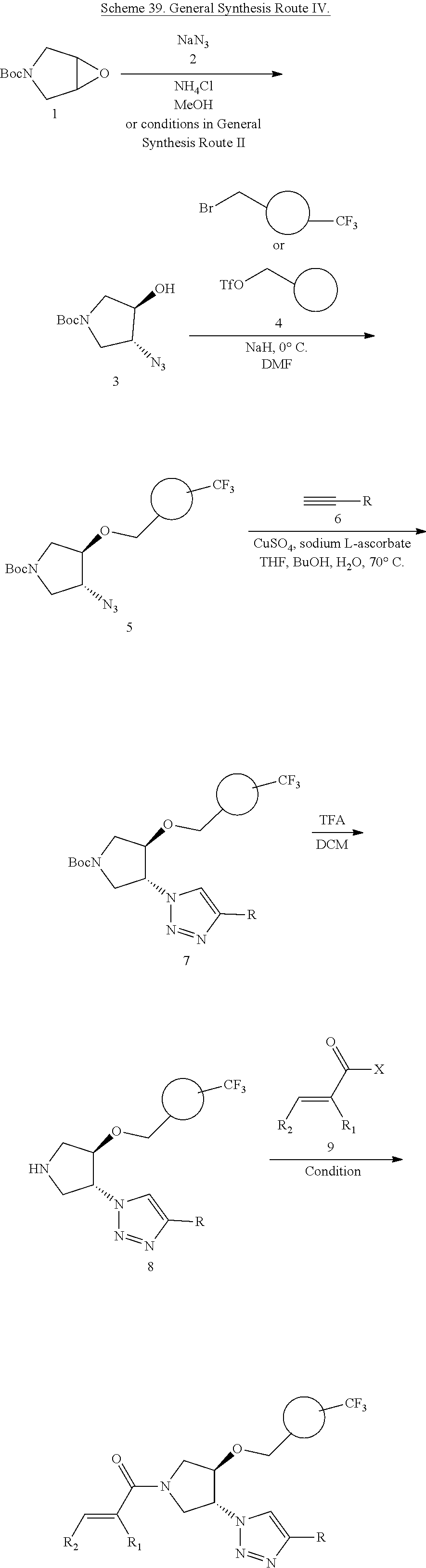

TABLE 12

| Compound | 4 | 6 | 9 (w/Conditions) |
|---|---|---|---|
| 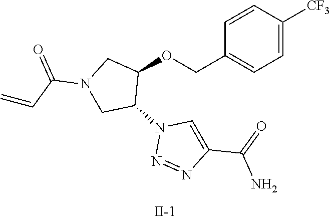 [SMILES: C=CC(=O)N1C[C@@H](n2cc(C(N)=O)nn2)[C@H](OCc2ccc(C(F)(F)F)cc2)C1 ; "II-1"] | 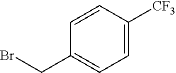 [SMILES: FC(F)(F)c1ccc(CBr)cc1] | 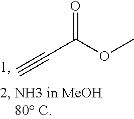 | 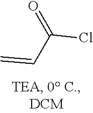 |
| 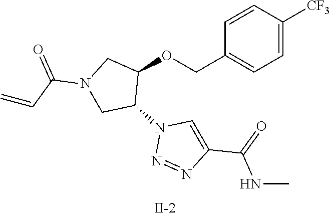 [SMILES: C=CC(=O)N1C[C@@H](n2cc(C(=O)NC)nn2)[C@H](OCc2ccc(C(F)(F)F)cc2)C1 ; "II-2"] | 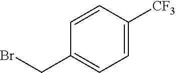 [SMILES: FC(F)(F)c1ccc(CBr)cc1] | 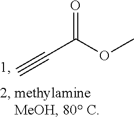 | 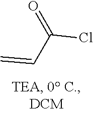 |
| 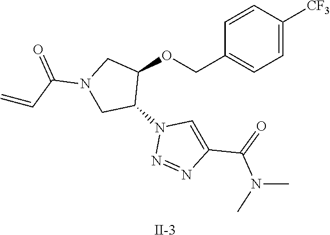 [SMILES: C=CC(=O)N1C[C@@H](n2cc(C(=O)N(C)C)nn2)[C@H](OCc2ccc(C(F)(F)F)cc2)C1 ; "II-3"] | 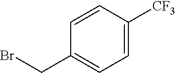 [SMILES: FC(F)(F)c1ccc(CBr)cc1] | 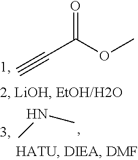 | 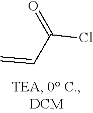 |
| 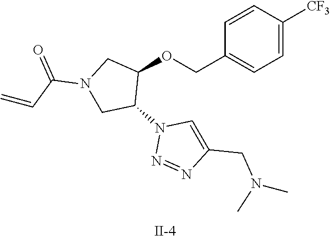 [SMILES: C=CC(=O)N1C[C@@H](n2cc(CN(C)C)nn2)[C@H](OCc2ccc(C(F)(F)F)cc2)C1 ; "II-4"] | 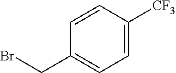 [SMILES: FC(F)(F)c1ccc(CBr)cc1] | 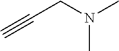 | 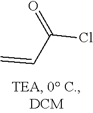 |
| 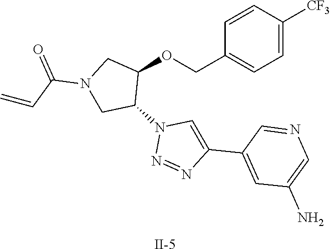 [SMILES: C=CC(=O)N1C[C@@H](n2cc(-c3cncc(N)c3)nn2)[C@H](OCc2ccc(C(F)(F)F)cc2)C1 ; "II-5"] | 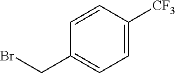 [SMILES: FC(F)(F)c1ccc(CBr)cc1] | 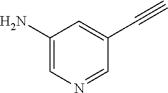 | 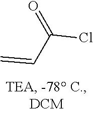 |
| 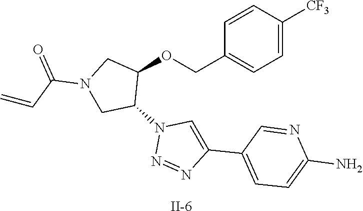 | 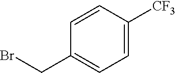 [SMILES: FC(F)(F)c1ccc(CBr)cc1] | 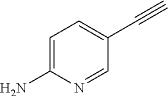 | 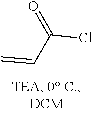 |
| 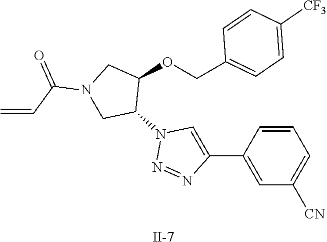 [SMILES: C=CC(=O)N1C[C@@H](n2cc(-c3cccc(C#N)c3)nn2)[C@H](OCc2ccc(C(F)(F)F)cc2)C1 ; "II-7"] | 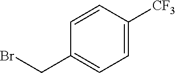 [SMILES: FC(F)(F)c1ccc(CBr)cc1] | 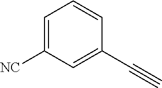 | 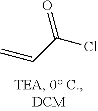 |
| 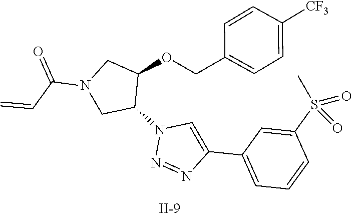 [SMILES: C=CC(=O)N1C[C@@H](n2cc(-c3cccc(S(C)(=O)=O)c3)nn2)[C@H](OCc2ccc(C(F)(F)F)cc2)C1 ; "II-9"] | 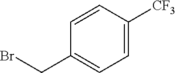 [SMILES: FC(F)(F)c1ccc(CBr)cc1] | 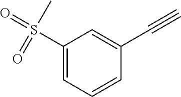 | 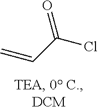 |
| 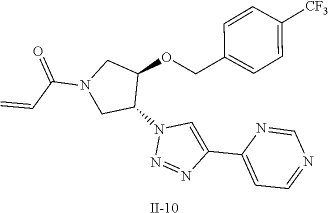 [SMILES: C=CC(=O)N1C[C@@H](n2cc(-c3ccncn3)nn2)[C@H](OCc2ccc(C(F)(F)F)cc2)C1 ; "II-10"] | 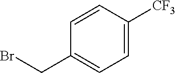 [SMILES: FC(F)(F)c1ccc(CBr)cc1] | 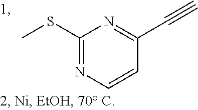 | 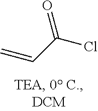 |
| 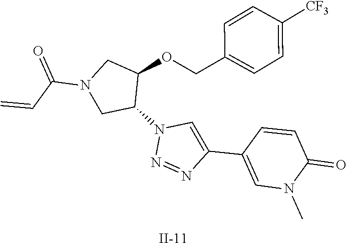 [SMILES: C=CC(=O)N1C[C@@H](n2cc(-c3ccc(=O)n(C)c3)nn2)[C@H](OCc2ccc(C(F)(F)F)cc2)C1 ; "II-11"] | 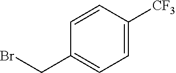 [SMILES: FC(F)(F)c1ccc(CBr)cc1] | 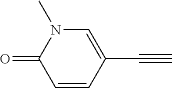 | 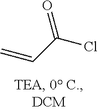 |
| 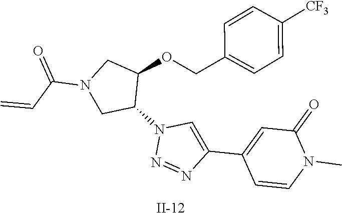 [SMILES: C=CC(=O)N1C[C@@H](n2cc(-c3ccn(C)c(=O)c3)nn2)[C@H](OCc2ccc(C(F)(F)F)cc2)C1 ; "II-12"] | 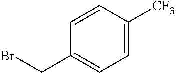 [SMILES: FC(F)(F)c1ccc(CBr)cc1] | 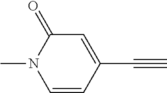 | 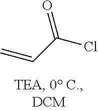 |
| 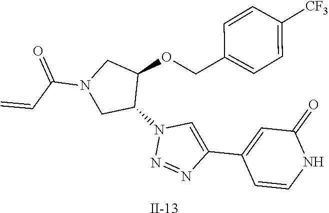 [SMILES: C=CC(=O)N1C[C@@H](n2cc(-c3cc[nH]c(=O)c3)nn2)[C@H](OCc2ccc(C(F)(F)F)cc2)C1 ; "II-13"] | 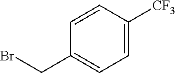 [SMILES: FC(F)(F)c1ccc(CBr)cc1] | 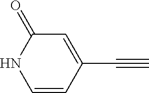 | 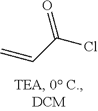 |
| 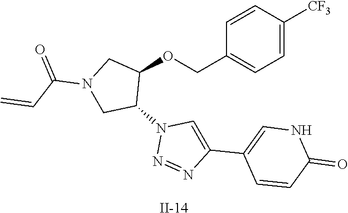 [SMILES: C=CC(=O)N1C[C@@H](n2cc(-c3ccc(=O)[nH]c3)nn2)[C@H](OCc2ccc(C(F)(F)F)cc2)C1 ; "II-14"] | 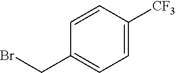 [SMILES: FC(F)(F)c1ccc(CBr)cc1] | 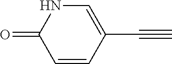 | 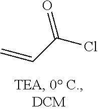 |
| 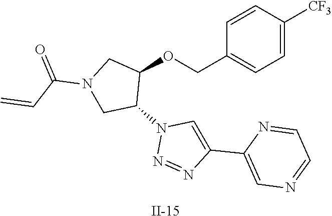 [SMILES: C=CC(=O)N1C[C@@H](n2cc(-c3cnccn3)nn2)[C@H](OCc2ccc(C(F)(F)F)cc2)C1 ; "II-15"] | 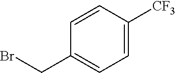 [SMILES: FC(F)(F)c1ccc(CBr)cc1] | 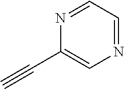 | 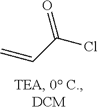 |
| 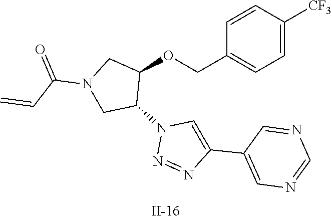 [SMILES: C=CC(=O)N1C[C@@H](n2cc(-c3cncnc3)nn2)[C@H](OCc2ccc(C(F)(F)F)cc2)C1 ; "II-16"] | 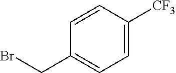 [SMILES: FC(F)(F)c1ccc(CBr)cc1] | 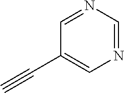 | 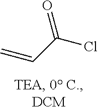 |
| 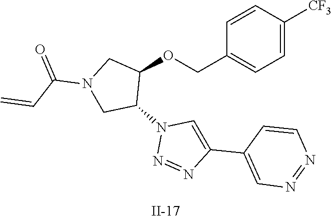 [SMILES: C=CC(=O)N1C[C@@H](n2cc(-c3ccnnc3)nn2)[C@H](OCc2ccc(C(F)(F)F)cc2)C1 ; "II-17"] | 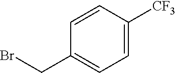 [SMILES: FC(F)(F)c1ccc(CBr)cc1] | 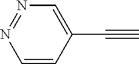 | 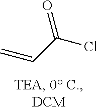 |
| 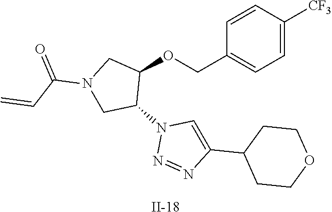 [SMILES: C=CC(=O)N1C[C@@H](n2cc(C3CCOCC3)nn2)[C@H](OCc2ccc(C(F)(F)F)cc2)C1 ; "II-18"] | 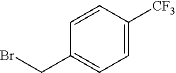 [SMILES: FC(F)(F)c1ccc(CBr)cc1] | 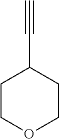 | 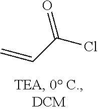 |
| 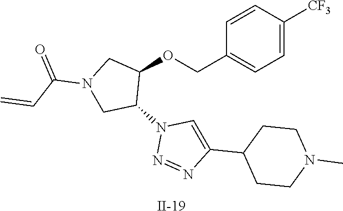 [SMILES: C=CC(=O)N1C[C@@H](n2cc(C3CCN(C)CC3)nn2)[C@H](OCc2ccc(C(F)(F)F)cc2)C1 ; "II-19"] | 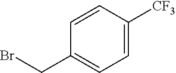 [SMILES: FC(F)(F)c1ccc(CBr)cc1] | 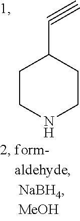 | 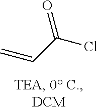 |
| 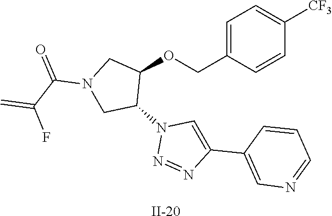 [SMILES: C=C(F)C(=O)N1C[C@@H](n2cc(-c3cccnc3)nn2)[C@H](OCc2ccc(C(F)(F)F)cc2)C1 ; "II-20"] | 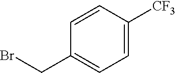 [SMILES: FC(F)(F)c1ccc(CBr)cc1] | 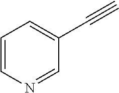 | 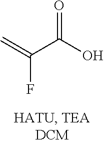 |
| 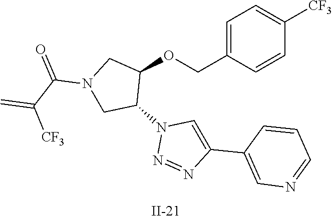 [SMILES: C=C(C(=O)N1C[C@@H](n2cc(-c3cccnc3)nn2)[C@H](OCc2ccc(C(F)(F)F)cc2)C1)C(F)(F)F ; "II-21"] | 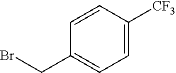 [SMILES: FC(F)(F)c1ccc(CBr)cc1] | 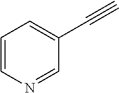 | 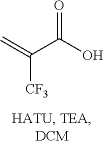 |
| 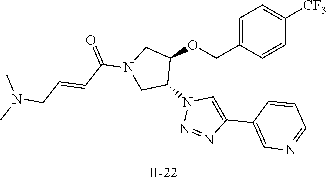 [SMILES: CN(C)C/C=C/C(=O)N1C[C@@H](n2cc(-c3cccnc3)nn2)[C@H](OCc2ccc(C(F)(F)F)cc2)C1 ; "II-22"] | 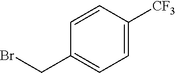 [SMILES: FC(F)(F)c1ccc(CBr)cc1] | 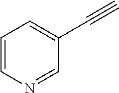 | 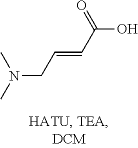 |
| 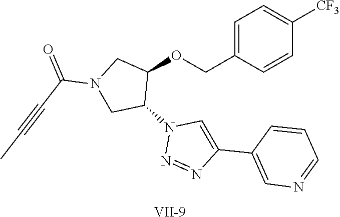 [SMILES: CC#CC(=O)N1C[C@@H](n2cc(-c3cccnc3)nn2)[C@H](OCc2ccc(C(F)(F)F)cc2)C1 ; "VII-9"] | 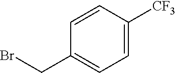 [SMILES: FC(F)(F)c1ccc(CBr)cc1] | 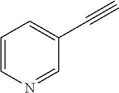 | 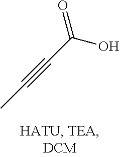 |
| 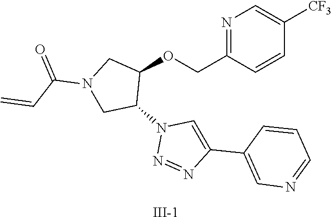 [SMILES: C=CC(=O)N1C[C@@H](n2cc(-c3cccnc3)nn2)[C@H](OCc2ccc(C(F)(F)F)cn2)C1 ; "III-1"] | 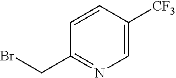 [SMILES: FC(F)(F)c1ccc(CBr)nc1] | 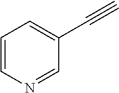 | 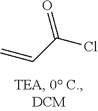 |
| 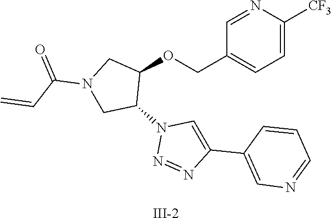 [SMILES: C=CC(=O)N1C[C@@H](n2cc(-c3cccnc3)nn2)[C@H](OCc2ccc(C(F)(F)F)nc2)C1 ; "III-2"] | 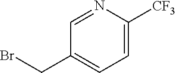 [SMILES: FC(F)(F)c1ccc(CBr)cn1] | 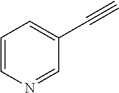 | 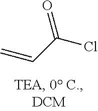 |
| 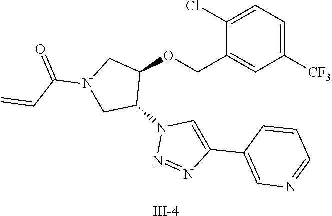 [SMILES: C=CC(=O)N1C[C@@H](n2cc(-c3cccnc3)nn2)[C@H](OCc2cc(C(F)(F)F)ccc2Cl)C1 ; "III-4"] | 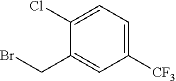 [SMILES: FC(F)(F)c1ccc(Cl)c(CBr)c1] | 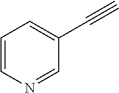 | 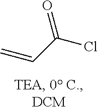 |
| 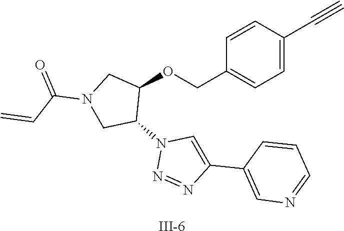 [SMILES: C#Cc1ccc(CO[C@@H]2CN(C(=O)C=C)C[C@H]2n2cc(-c3cccnc3)nn2)cc1 ; "III-6"] | 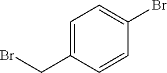 | 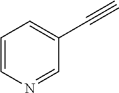 | 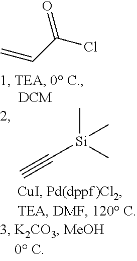 |
| 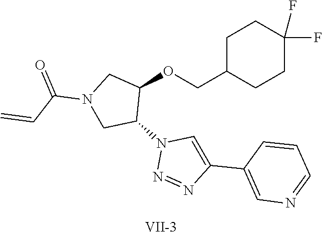 [SMILES: C=CC(=O)N1C[C@@H](n2cc(-c3cccnc3)nn2)[C@H](OCC2CCC(F)(F)CC2)C1 ; "VII-3"] | 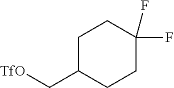 [SMILES: O=S(=O)(OCC1CCC(F)(F)CC1)C(F)(F)F] | 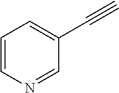 | 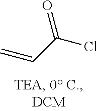 |
| 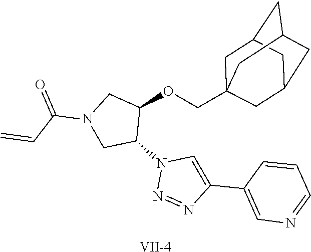 [SMILES: C=CC(=O)N1C[C@@H](n2cc(-c3cccnc3)nn2)[C@H](OCC23CC4CC(CC(C4)C2)C3)C1 ; "VII-4"] | 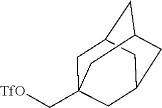 [SMILES: O=S(=O)(OCC12CC3CC(CC(C3)C1)C2)C(F)(F)F] | 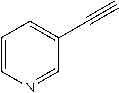 | 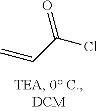 |

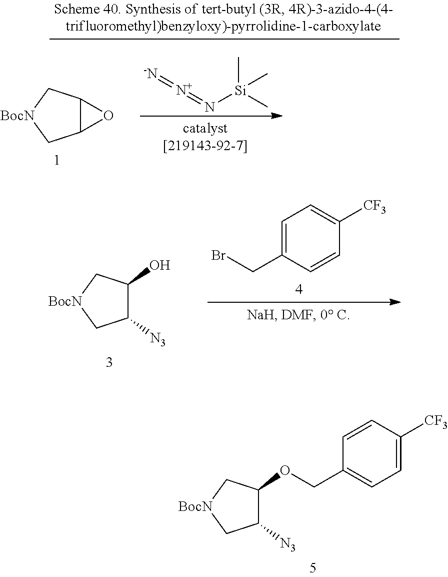

### Step 1: Synthesis of tert-butyl (3R, 4R)-3-azido-4-hydroxypyrrolidine-1-carboxylate (3)

A mixture of tert-butyl 6-oxa-3-azabicyclo[3.1.0]hexane-3-carboxylate (4 g, 21.6 mmol), TMSN₃ (2.664 g, 23.2 mmol) and chiral catalyst (1S,2S)-(−)-[1,2-cyclohexanediamino-N,N′-bis (3,5-di-t-butylsalicylidene)]chromium (III) chloride (328 mg, 0.42 mmol) was stirred at rt under N₂ overnight. The reaction mixture was treated with MeOH (60 mL) and K₂CO₃ (1.788 g, 12.8 mmol) and continued to stir at rt for 5 hours. The reaction mixture was diluted with ethyl acetate (300 mL), washed with water (300 mL×2), dried over anhydrous Na₂SO₄, concentrated and purified by flash column chromatography on silica gel (ethyl acetate in petroleum ether=30% v/v) to obtain the compound 3 as clear oil (3.5 g, 96% e.e., yield 71%). LC-MS (ESI) m/z: 129 [M+H−100]⁺.

### Step 2: Synthesis of tert-butyl (3R, 4R)-3-azido-4-(4-(trifluoromethyl)benzyloxy)-pyrrolidine-1-carboxylate (5)

A mixture of compound 3 (3 g, 13.1 mmol), 1-(bromomethyl)-4-(trifluoromethyl)benzene (3.1 g, 13.1 mmol) and 60% NaH (0.6 g, 15.7 mmol) in DMF (20 mL) was stirred at 0° C. under N₂ for 6 hours. The reaction mixture was diluted with water (200 mL) and extracted with ethyl acetate (200 mL), the organic was washed with water (100 mL), dried over anhydrous Na₂SO₄, concentrated and purified by flash column chromatography on silica gel (ethyl acetate in petroleum ether=20% v/v) to obtain compound 5 as oil (3.8 g, yield 75%). LC-MS (ESI) m/z: 287 [M+H−100]⁺.

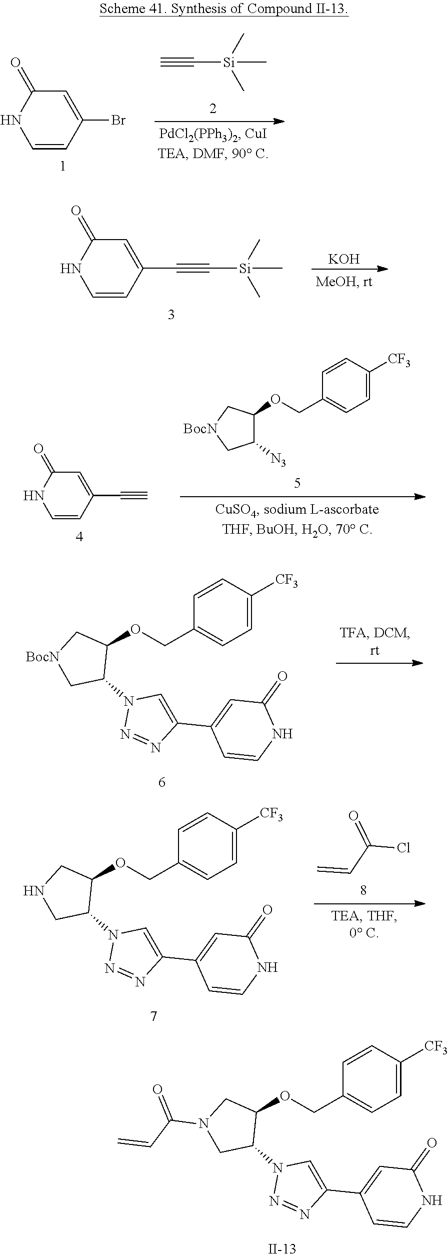

### Step 1: Synthesis of 4-((trimethylsilyl)ethynyl)pyridin-2(1H)-one (Compound 3)

To the solution of ethynyltrimethylsilane (0.595 g. 6.07 mmol) in DMF (40 mL) was added 4-bromopyridin-2(1H)-one (1 g. 5.8 mmol), PdCl₂(PPh₃)₂ (0.204 g. 0.29 mmol), CuI (55 mg, 0.29 mmol) and Et₃N (1.17 g. 11.6 mmol). The mixture was stirred at 90° C. under N₂ for 2 hours. After cooled down to rt the mixture was diluted with water (200 mL) and extracted with EtOAc (50 mL×2), the combined organic was dried over anhydrous Na₂SO₄, concentrated and purified by flash column chromatography on silica gel (ethyl acetate in petroleum ether=50% v/v) to obtain compound 3 as oil (500 mg, yield 45.4%). LC-MS (ESI) m/z: 192 [M+H]⁺.

### Step 2: Synthesis of 4-ethynylpyridin-2(1H)-one (Compound 4)

To the solution of compound 3 (450 mg, 2.35 mmol) in MeOH (20 mL) was added KOH (263 mg, 4.70 mmol). The mixture was stirred at rt under N₂ for 2 hours. The resulted mixture was concentrated and purified by flash column chromatography on silica gel (ethyl acetate in petroleum ether=90% v/v) to obtain compound 4 as oil (200 mg, yield 71.4%). LC-MS (ESI) m/z: 120 [M+H]⁺.

### Step 3: Synthesis of tert-butyl trans-3-(4-(2-oxo-1,2-dihydropyridin-4-yl)-1H-1,2,3-triazol-1-yl)-4-(4-(trifluoromethyl)benzyloxy)pyrrolidine-1-carboxylate (Compound 6)

To the solution of compound 5 (racemic, 250 mg, 0.65 mmol) in THF (10 mL), H₂O (10 mL) and n-BuOH (10 mL) was added compound 4 (116 mg, 0.97 mmol), CuSO₄ (15 mg, 0.065 mmol) and sodium L-ascorbate (26 mg, 0.13 mmol). The mixture was stirred at 70° C. under N₂ for 16 hours. The resulting mixture was concentrated and purified by flash column chromatography on silica gel (methanol in dichloromethane=20% v/v) to obtain compound 6 as solid (200 mg, yield 60%). LC-MS (ESI) m/z: 506 [M+H]⁺.

### Step 4: Synthesis of 4-(1-(trans-4-(4-(trifluoromethyl)benzyloxy)pyrrolidin-3-yl)-1H-1,2,3-triazol-4-yl)pyridin-2(1H)-one (Compound 7)

To the solution of compound 6 (180 mg, 0.36 mmol) in DCM (10 mL) was added TFA (2 mL). The mixture was stirred at rt for 2 hours and concentrated in vacuum, the residue was adjusted to pH˜8 with NaHCO₃ solution and extracted with EtOAc (50 mL×3), the combined organics were washed with brine (100 mL), dried over anhydrous Na₂SO₄, filtered and concentrated to leave crude compound 7 as oil (150 mg, crude). LC-MS (ESI) m/z: 406 [M+H]⁺.

### Step 5: Synthesis of 4-(1-(trans-1-acryloyl-4-(4-(trifluoromethyl)benzyloxy)pyrrolidin-3-yl)-1H-1,2,3-triazol-4-yl)pyridin-2(1H)-one (Compound II-13)

To the solution of compound 7 (130 mg, 0.32 mmol) in THF (10 mL) was added acryloyl chloride (29 mg, 0.32 mmol) and Et₃N (65 mg, 0.64 mmol). The mixture was stirred at 0° C. for 1 hour, and then concentrated and purified by prep-HPLC to obtain compound II-13 as solid (44 mg, yield 30.0%). LC-MS (ESI) m/z: 460 [M+H]⁺. ¹H-NMR (400 MHz, DMSO-d₆) δ (ppm) 11.58 (br, 1H), 8.89 (d, J=5.5 Hz, 1H), 7.70 (d, J=8.0 Hz, 2H), 7.54 (d, J=8.0 Hz, 2H), 7.46 (d, J=6.6 Hz, 1H), 6.79 (s, 1H), 6.70-6.56 (m, 2H), 6.24-6.14 (m, 1H), 5.79-5.71 (m, 1H), 5.53-5.39 (m, 1H), 4.81-4.71 (m, 2H), 4.62-4.50 (m, 1H), 4.30-4.13 (m, 1H), 4.12-3.95 (m, 1H), 3.91-3.79 (m, 1H), 3.68-3.58 (m, 1H).

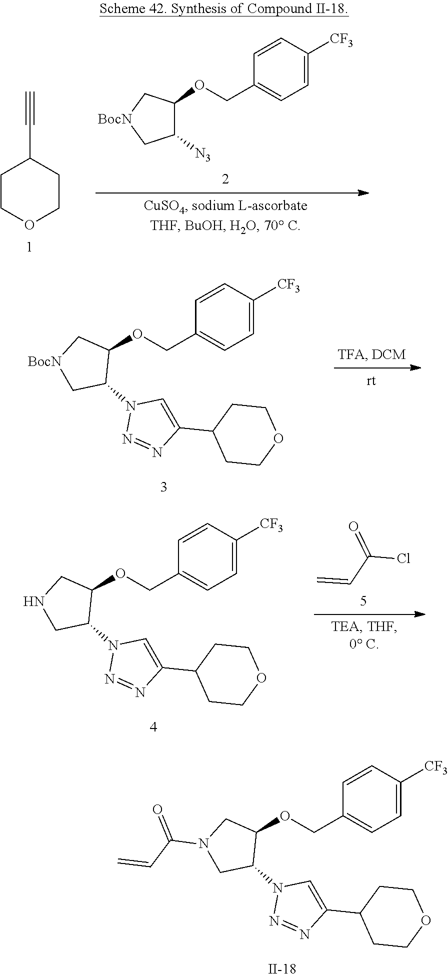

### Step 1: Synthesis of tert-butyl trans-3-(4-(tetrahydro-2H-pyran-4-yl)-1H-1,2,3-triazol-1-yl)-4-(4-(trifluoromethyl)benzyloxy)pyrrolidine-1-carboxylate (Compound 3)

To the solution of compound 2 (racemic, 300 mg, 0.78 mmol) in THF (10 mL), H₂O (10 mL) and ⁿBuOH (10 mL) was added 4-ethynyltetrahydro-2H-pyran (128 mg, 1.16 mmol), CuSO₄ (20 mg, 0.078 mmol) and sodium L-ascorbate (31 mg, 0.156 mmol). The mixture was stirred at 70° C. under N₂ for 16 hours. The resulting mixture was concentrated and purified by flash column chromatography on silica gel (Methanol in dichloromethane=20% v/v) to obtain compound 3 as solid (300 mg, yield 77.9%). LC-MS (ESI) m/z: 497 [M+H]⁺.

### Step 2: Synthesis of 4-(tetrahydro-2H-pyran-4-yl)-1-(trans-4-(4-(trifluoromethyl)-benzyloxy)pyrrolidin-3-yl)-1H-1,2,3-triazole (Compound 4)

To the solution of compound 3 (280 mg, 0.56 mmol) in DCM (10 mL) was added TFA (2 mL). The mixture was stirred at room temperature for 2 hours and concentrated in vacuum, the residue was adjusted to pH˜8 with NaHCO₃ solution and extracted with EtOAc (50 mL×3), the combined organics were washed with brine (100 mL), dried over anhydrous Na₂SO₄, filtered and concentrated to leave the crude compound 4 as oil (250 mg, crude). LC-MS (ESI) m/z: 397 [M+H]⁺.

### Step 3: Synthesis of 1-(trans-3-(4-(tetrahydro-2H-pyran-4-yl)-1H-1,2,3-triazol-1-yl)-4-(4-(trifluoromethyl)benzyloxy)pyrrolidin-1-yl)prop-2-en-1-one (Compound II-18)

To the solution of compound 4 (230 mg, 0.58 mmol) in THF (10 mL) was added acryloyl chloride (52 mg, 0.58 mmol) and Et₃N (117 mg, 1.16 mmol). The mixture was stirred at 0° C. under N₂ for 1 hour, and then concentrated and purified by prep-HPLC to obtain compound II-18 as solid (180 mg, yield 69.0%). LC-MS (ESI) m/z: 451 [M+H]⁺. ¹H-NMR (400 MHz, DMSO-d₆): δ (ppm) 8.05 (d, J=6.6 Hz, 1H), 7.70 (d, J=7.6 Hz, 2H), 7.51 (d, J=7.8 Hz, 2H), 6.66-6.57 (m, 1H), 6.18 (d, J=16.8 Hz, 1H), 5.77-5.67 (m, 1H), 5.43-5.29 (m, 1H), 4.72 (d, J=5.2 Hz, 2H), 4.50 (dd, J=31.4, 5.0 Hz, 1H), 4.24-3.96 (m, 2H), 3.89 (d, J=13.1 Hz, 2H), 3.82 (td, J=13.1, 4.4 Hz, 1H), 3.58 (dd, J=13.1, 3.3 Hz, 1H), 3.44 (t, J=11.3 Hz, 2H), 2.93 (t, J=10.6 Hz, 1H), 1.85 (d, J=12.9 Hz, 2H), 1.61 (q, J=11.4 Hz, 2H).

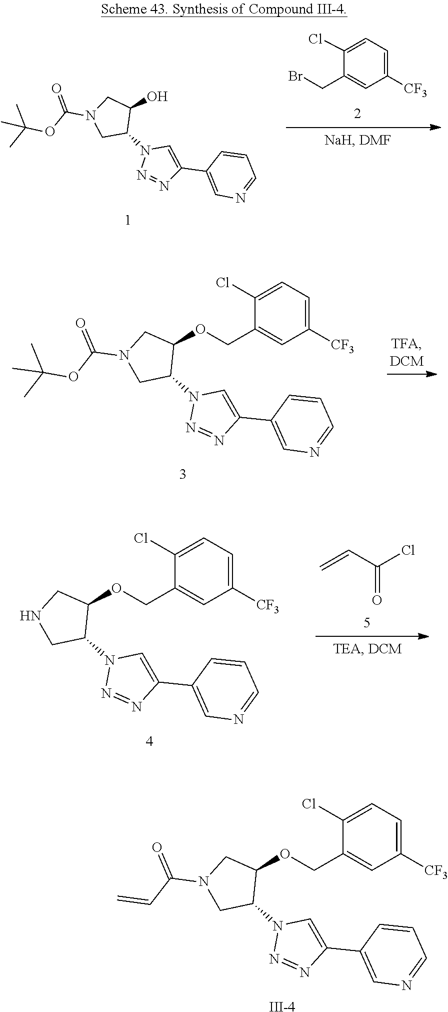

### Step 1: Synthesis of tert-butyl trans-3-(2-chloro-5-(trifluoromethyl)benzyloxy)-4-(4-(pyridin-3-yl)-1H-1,2,3-triazol-1-yl)pyrrolidine-1-carboxylate (Compound 3)

To a mixture of racemic compound 1 (racemic, 100 mg, 0.3 mmol) in DMF (5 mL) was added NaH (60% suspension, 20 mg, 0.33 mmol), the mixture was stirred at rt for 10 minutes, and then 2-(bromomethyl)-1-chloro-4-(trifluoromethyl)benzene (80 mg, 0.3 mmol) was added. The mixture was stirred at rt under N₂ overnight, diluted with water (50 mL) and extracted with ethyl acetate (60 mL), the organic was washed with water (60 mL), dried over anhydrous Na₂SO₄, concentrated and purified by flash column chromatography on silica gel (ethyl acetate in petroleum ether=40% v/v) to obtain compound 3 as yellow solid (130 mg, yield 83%). LC-MS (ESI) m/z: 524 [M+H]⁺.

### Step 2: Synthesis of 3-(1-(trans-4-(2-chloro-5-(trifluoromethyl)benzyloxy)pyrrolidin-3-yl)-1H-1,2,3-triazol-4-yl)pyridine (Compound 4)

A mixture of compound 3 (racemic, 100 mg, 0.19 mmol) and TFA (1 mL) in DCM (3 mL) was stirred at rt under N₂ for 2 hours. The mixture was concentrated to leave crude compound 4 as yellow oil (80 mg, yield 99%). LC-MS (ESI) m/z: 424 [M+H]⁺.

### Step 3: Synthesis of 1-(trans-3-(2-chloro-5-(trifluoromethyl)benzyloxy)-4-(4-(pyridin-3-yl)-1H-1,2,3-triazol-1-yl)pyrrolidin-1-yl)prop-2-en-1-one (III-4)

To a mixture of compound 4 (100 mg, 0.23 mmol) and TEA (46 mg, 0.46 mmol) in DCM (5 mL) was added acryloyl chloride (21 mg, 0.23 mmol), the mixture was stirred at rt for 2 hours, concentrated and purified by prep-HPLC to obtain compound III-4 as white solid (40 mg, yield 36%). LC-MS (ESI) m/z: 478 [M+H]⁺. ¹H NMR (400 MHz, CD₃OD) δ (ppm) 9.03-8.98 (m, 1H), 8.64 (d, J=5.2 Hz, 1H), 8.55-8.50 (m, 1H), 8.31-8.26 (m, 1H), 7.80 (s, 1H), 7.61 (s, 2H), 7.55-7.51 (m, 1H), 6.70-6.61 (m, 1H), 6.37-6.30 (m, 1H), 5.83-5.77 (m, 1H), 5.57-5.45 (m, 1H), 4.86 (s, 2H), 4.79-4.68 (m, 1H), 4.42-3.80 (m, 4H).

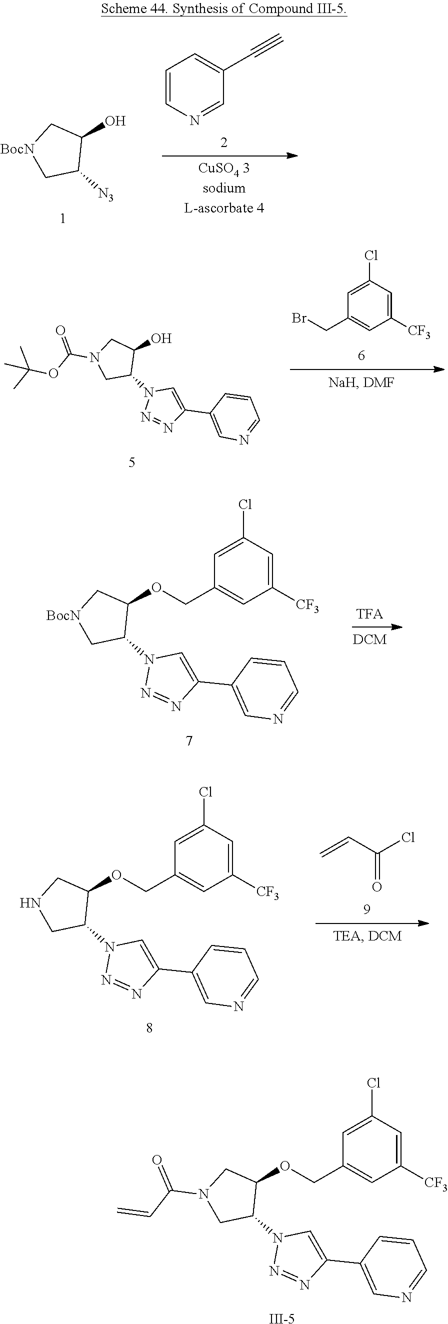

### Step 1: Synthesis of tert-butyl trans-3-hydroxy-4-(4-(pyridin-3-yl)-1H-1,2,3-triazol-1-yl)pyrrolidine-1-carboxylate (Compound 5)

A mixture of tert-butyl trans-3-azido-4-hydroxypyrrolidine-1-carboxylate (1000 mg. 4.38 mmol), 3-ethynylpyridine (451 mg, 4.38 mmol), CuSO₄ (654 mg, 2.6 mmol) and sodium L-ascorbate (257 mg, 1.3 mmol) in THF (3 mL), H₂O (3 mL) and nBuOH (3 mL) was stirred at 70° C. overnight. The mixture was diluted with water (50 mL) and extracted with ethyl acetate (60 mL), the extract was washed with water (40 mL), dried over anhydrous Na₂SO₄, concentrated and purified by flash column chromatography on silica gel (ethyl acetate in petroleum ether=80% v/v) to obtain compound 5 as yellow solid (650 mg, yield 45%). LC-MS (ESI) m/z: 332 [M+H]⁺.

### Step 2: Synthesis of tert-butyl trans-3-(3-chloro-5-(trifluoromethyl)benzyloxy)-4-(4-(pyridin-3-yl)-1H-1,2,3-triazol-1-yl)pyrrolidine-1-carboxylate (Compound 7)

To a mixture of compound 5 (100 mg, 0.3 mmol) in DMF (10 mL) was added NaH (14 mg, 0.33 mmol), the mixture was stirred at rt for 10 minutes, and then 1-(bromomethyl)-3-chloro-5-(trifluoromethyl)benzene (82 mg, 0.3 mmol) was added. The mixture was stirred at rt under N₂ overnight. The mixture was diluted with water (50 mL) and extracted with ethyl acetate (60 mL), the extract was washed with water (40 mL), dried over anhydrous Na₂SO₄, concentrated and purified by flash column chromatography on silica gel (ethyl acetate in petroleum ether=30% v/v) to obtain the compound 7 as yellow solid (80 mg, yield 51%). LC-MS (ESI) m/z: 524 [M+H]⁺.

### Step 3: Synthesis of 3-(1-(trans-4-(3-chloro-5-(trifluoromethyl)benzyloxy)pyrrolidin-3-yl)-1H-1,2,3-triazol-4-yl)pyridine (Compound 8)

A mixture of compound 7 (50 mg, 0.09 mmol) and TFA (1 mL) in DCM (3 mL) was stirred at rt for 2 hours. The mixture was concentrated to leave crude compound 8 as yellow oil (50 mg, yield 100%). LC-MS (ESI) m/z: 424 [M+H]⁺.

### Step 4: Synthesis of 1-(trans-3-(3-chloro-5-(trifluoromethyl)benzyloxy)-4-(4-(pyridin-3-yl)-1H-1,2,3-triazol-1-yl)pyrrolidin-1-yl)prop-2-en-1-one (III-5)

To a mixture of compound 8 (50 mg, 0.1 mmol) and TEA (20 mg, 0.2 mmol) in DCM (5 mL) was added acryloyl chloride (20 mg, 0.1 mmol), the mixture was stirred at rt for 2 hours, and then concentrated and purified by prep-HPLC to obtain compound III-5 as white solid (18 mg, yield 38%). LC-MS (ESI) m/z: 478 [M+H]⁺. ¹H NMR (400 MHz, CD₃OD) δ (ppm) 9.03 (d, J=1.1 Hz, 1H), 8.62 (d, J=4.9 Hz, 1H), 8.55 (d, J=4.9 Hz, 1H), 8.31 (dt, J=8.0, 1.8 Hz, 1H), 7.68-7.51 (m, 4H), 6.67 (ddd, J=16.7, 10.4, 4.4 Hz, 1H), 6.36 (d, J=16.8 Hz, 1H), 5.83 (ddd, J=10.4, 5.6, 1.9 Hz, 1H), 5.56-5.40 (m, 1H), 4.82-4.63 (m, 3H), 4.40-4.05 (m, 3H), 3.97-3.76 (m, 1H).

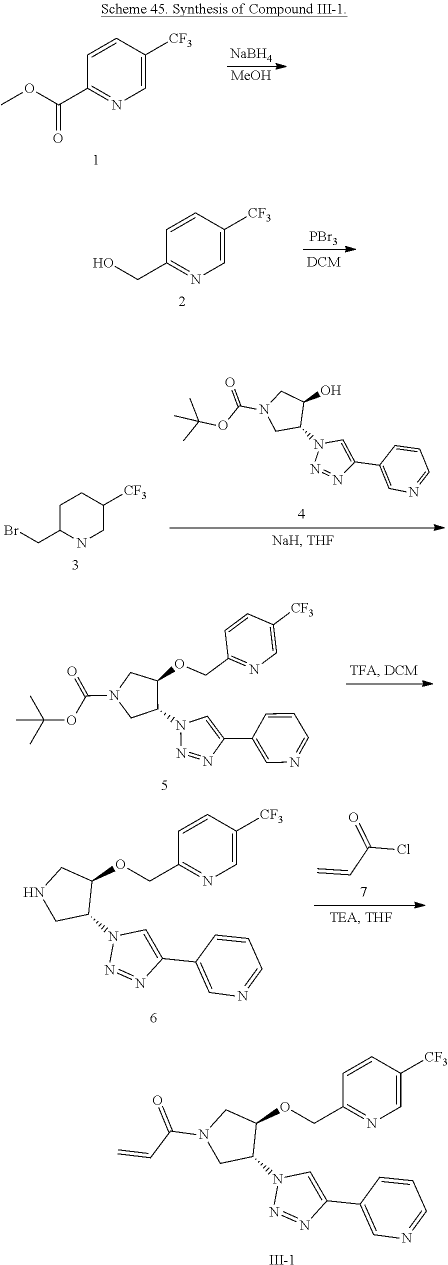

### Step 1: Synthesis of (5-(trifluoromethyl)pyridin-2-yl)methanol (Compound 2)

To the solution of methyl 5-(trifluoromethyl) picolinate (2 g, 9.75 mmol) in MeOH (30 mL) was added NaBH₄ (0.738 g, 19.5 mmol) portionwise. The mixture was stirred at rt for 2 hours, and then concentrated and purified by flash column chromatography on silica gel (ethyl acetate in petroleum ether=50% v/v) to obtain the target compound 2 as oil (1 g, yield 57.9%). LC-MS (ESI) m/z: 178 [M+H]⁺.

### Step 2: Synthesis of 2-(bromomethyl)-5-(trifluoromethyl)pyridine (Compound 3)

To the solution of (5-(trifluoromethyl)pyridin-2-yl)methanol (1 g, 5.6 mmol) in DCM (30 mL) was added PBr₃ (0.9 g, 3.4 mmol) dropwise. The mixture was stirred at rt for 16 hours, concentrated and purified by flash column chromatography on silica gel (ethyl acetate in petroleum ether=90% v/v) to obtain the target compound 3 as oil (1 g, yield 74.1%). LC-MS (ESI) m/z: 240 [M+H]⁺.

### Step 3: Synthesis of tert-butyl trans-3-(4-(pyridin-3-yl)-1H-1,2,3-triazol-1-yl)-4-((5-(trifluoromethyl)pyridin-2-yl)methoxy)pyrrolidine-1-carboxylate (Compound 5)

To the solution of tert-butyl trans-3-hydroxy-4-(4-(pyridin-3-yl)-1H-1,2,3-triazol-1-yl)pyrrolidine-1-carboxylate (100 mg, 0.30 mmol) in THF (10 mL) was added NaH (60%, 12 mg, 0.60 mmol), the mixture was stirred at 0° C. under N₂ for 0.5 hour, and then 2-(bromomethyl)-5-(trifluoromethyl)pyridine (70 mg, 0.30 mmol) was added, the mixture was stirred at rt for 1 hour, concentrated and purified by flash column chromatography on silica gel (methyl alcohol in dichloromethane=20% v/v) to obtain the target compound 5 as solid (100 mg, yield 70.0%). LC-MS (ESI) m/z: 491 [M+H]⁺.

### Step 4: Synthesis of 2-((trans-4-(4-(pyridin-3-yl)-1H-1,2,3-triazol-1-yl)pyrrolidin-3-yloxy)methyl)-5-(trifluoromethyl)pyridine (Compound 6)

To a solution of tert-butyl trans-3-(4-(pyridin-3-yl)-1H-1,2,3-triazol-1-yl)-4-((5-(trifluoromethyl)pyridin-2-yl)methoxy)pyrrolidine-1-carboxylate (80 mg, 0.16 mmol) in DCM (10 mL) was added TFA (2 mL). The mixture was stirred at rt for 2 hours and concentrated to leave crude compound 6 as oil (80 mg, crude). LC-MS (ESI) m/z: 391 [M+H]⁺.

### Step 5: Synthesis of 1-(trans-3-(4-(pyridin-3-yl)-1H-1,2,3-triazol-1-yl)-4-((5-(trifluoromethyl)pyridin-2-yl)methoxy)pyrrolidin-1-yl)prop-2-en-1-one (Compound III-1)

To a solution of 2-((trans-4-(4-(pyridin-3-yl)-1H-1,2,3-triazol-1-yl)pyrrolidin-3-yloxy)methyl)-5-(trifluoromethyl)pyridine (60 mg, 0.15 mmol) and Et₃N (30 mg, 0.30 mmol) in THF (10 mL) was added acryloyl chloride (15 mg, 0.15 mmol), the mixture was stirred at 0° C. for 1 hour, concentrated and purified by prep-HPLC (MeCN/H₂O/TFA) to obtain the target compound III-1 as solid (18 mg, yield 26.3%). LC-MS (ESI) m/z: 445 [M+H]⁺. ¹H-NMR (400 MHz, CD₃OD) δ (ppm) 9.03 (d, J=1.8 Hz, 1H), 8.82 (s, 1H), 8.65 (d, J=6.2 Hz, 1H), 8.58-8.51 (m, 1H), 8.30 (d, J=8.0 Hz, 1H), 8.13 (dd, J=8.2, 1.9 Hz, 1H), 7.70 (d, J=8.2 Hz, 1H), 7.54 (dd, J=7.9, 4.9 Hz, 1H), 6.68 (dd, J=16.8, 10.4 Hz, 1H), 6.36 (dd, J=16.8, 1.4 Hz, 1H), 5.82 (ddd, J=10.5, 3.7, 1.9 Hz, 1H), 5.58-5.47 (m, 1H), 4.91 (d, J=3.5 Hz, 2H), 4.82-4.72 (m, 1H), 4.48-3.82 (m, 4H).

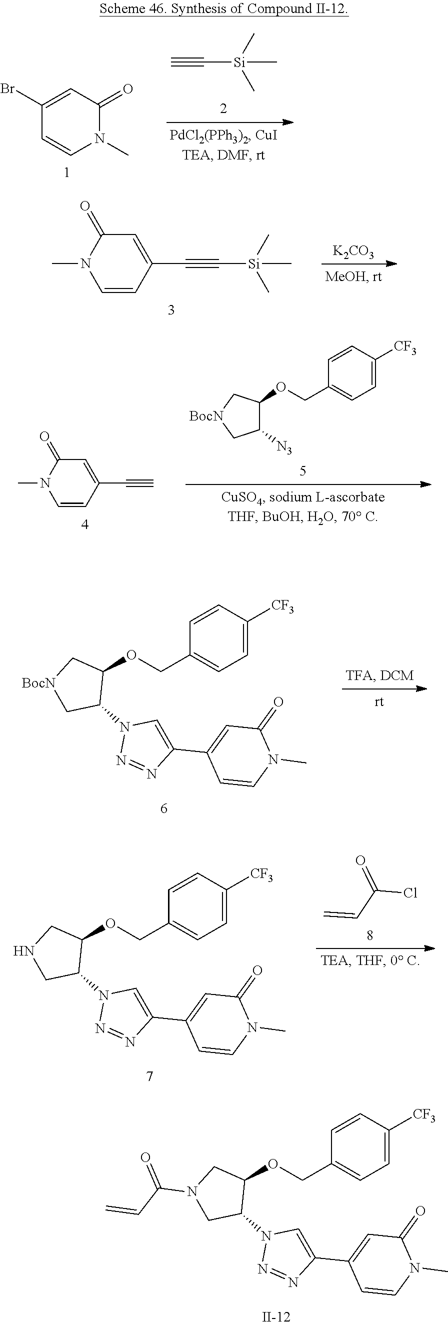

### Step 1: Synthesis of 1-methyl-4-((trimethylsilyl)ethynyl)pyridin-2(1H)-one (Compound 3)

To the solution of 4-bromo-1-methylpyridin-2(1H)-one (1 g, 5.3 mmol) in DMF (20 mL) was added ethynyltrimethylsilane (0.55 g, 5.6 mmol), PdCl₂(PPh₃)₂ (0.21 g, 0.3 mmol), CuI (0.06 g, 0.3 mmol) and Et₃N (1.07 g, 10.6 mmol). The mixture was stirred at room temperature under N₂ for 2 hours. The resulting mixture was concentrated and purified by flash column chromatography on silica gel (ethyl acetate in petroleum ether=50% v/v) to obtain the target compound 3 as oil (1.1 g, yield 99.9%). LC-MS (ESI) m/z: 206 [M+H]⁺.

### Step 2: Synthesis of 4-ethynyl-1-methylpyridin-2(1H)-one (Compound 4)

To the solution of 1-methyl-4-((trimethylsilyl)ethynyl)pyridin-2(1H)-one (1 g, 4.9 mmol) in MeOH (20 mL) was added K₂CO₃ (1.35 g, 9.8 mmol). The mixture was stirred at room temperature under N₂ for 2 hours. The resulting mixture was concentrated and purified by flash column chromatography on silica gel (ethyl acetate in petroleum ether=90% v/v) to obtain the target compound 4 as solid (400 mg, yield 56.1%). LC-MS (ESI) m/z: 134 [M+H]⁺.

### Step 3: Synthesis of tert-butyl trans-3-(4-(1-methyl-2-oxo-1,2-dihydropyridin-4-yl)-1H-1,2,3-triazol-1-yl)-4-(4-(trifluoromethyl)benzyloxy)pyrrolidine-1-carboxylate (Compound 6)

To the solution of tert-butyl trans-3-azido-4-(4-(trifluoromethyl)benzyloxy) pyrrolidine-1-carboxylate (350 mg, 0.91 mmol) in THF (5 mL), H₂O (5 mL) and BuOH (5 mL) was added 4-ethynyl-1-methylpyridin-2(1H)-one (181 mg, 1.36 mmol), CuSO₄ (23 mg, 0.09 mmol) and sodium L-ascorbate (36 mg, 0.18 mmol). The mixture was stirred at 70° C. under N₂ for 16 hours. The resulting mixture was concentrated and purified by flash column chromatography on silica gel (methyl alcohol in dichloromethane=20% v/v) to obtain the target compound 6 as solid (300 mg, yield 27.4%). LC-MS (ESI) m/z: 520 [M+H]⁺.

### Step 4: Synthesis of 1-methyl-4-(1-(trans-4-(4-(trifluoromethyl)benzyloxy)pyrrolidin-3-yl)-1H-1,2,3-triazol-4-yl)pyridin-2(1H)-one (Compound 7)

To the solution of tert-butyl trans-3-(4-(1-methyl-2-oxo-1,2-dihydropyridin-4-yl)-1H-1,2,3-triazol-1-yl)-4-(4-(trifluoromethyl)benzyloxy)pyrrolidine-1-carboxylate (280 mg, 0.54 mmol) in DCM (10 mL) was added TFA (2 mL). The mixture was stirred at room temperature for 2 hours and concentrated in vacuum, the residue was adjusted to pH˜8 with NaHCO₃ solution and extracted with EtOAc (50 mL×3), the combined organics were washed with brine (100 mL), dried over anhydrous Na₂SO₄, filtered and concentrated to leave the crude target compound 7 as oil (200 mg, crude). LC-MS (ESI) m/z: 420 [M+H]⁺.

### Step 5: Synthesis of 4-(1-(trans-1-acryloyl-4-(4-(trifluoromethyl)benzyloxy)pyrrolidin-3-yl)-1H-1,2,3-triazol-4-yl)-1-methylpyridin-2(1H)-one (Compound II-12)

To the solution of 1-methyl-4-(1-(trans-4-(4-(trifluoromethyl)benzyloxy)-pyrrolidin-3-yl)-1H-1,2,3-triazol-4-yl)pyridin-2(1H)-one (180 mg, 0.43 mmol) in THF (10 mL) was added acryloyl chloride (39 mg, 0.43 mmol) and Et₃N (87 mg, 0.86 mmol). The mixture was stirred at 0° C. under N₂ for 1 hour, and then concentrated and purified by prep-HPLC to obtain the target compound II-12 as solid (215 mg, yield 95.5%). LC-MS (ESI) m/z: 474 [M+H]⁺. ¹H-NMR (400 MHz, DMSO-d6): δ (ppm) 8.89 (d, J=5.8 Hz, 1H), 7.78 (d, J=7.1 Hz, 1H), 7.70 (d, J=8.2 Hz, 2H), 7.53 (d, J=8.1 Hz, 2H), 6.85 (s, 1H), 6.76-6.53 (m, 2H), 6.19 (dd, J=16.8, 2.3 Hz, 1H), 5.73 (ddd, J=10.2, 5.1, 2.3 Hz, 1H), 5.47 (d, J=25.4 Hz, 1H), 4.75 (s, 2H), 4.57 (d, J=24.5 Hz, 1H), 4.31-3.57 (m, 4H), 3.44 (s, 3H).

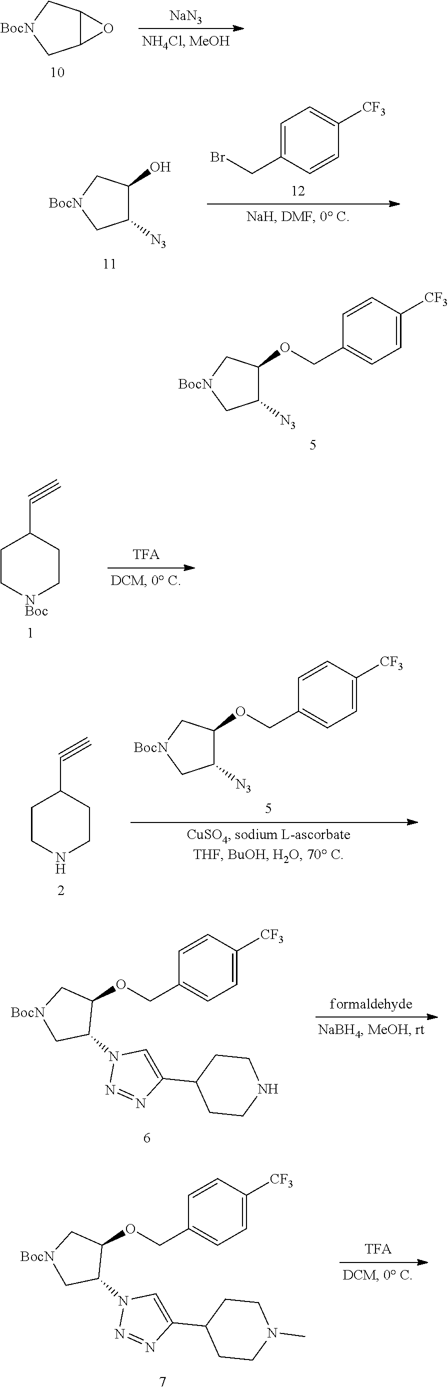

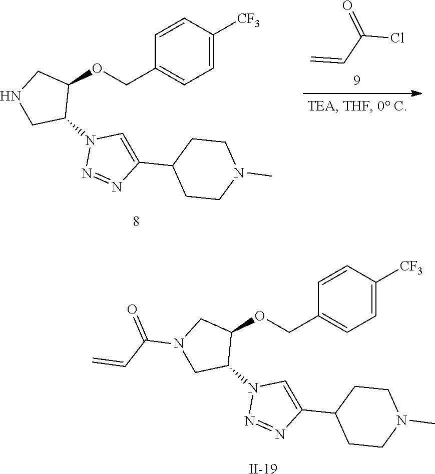

### Step 1: Synthesis of 4-ethynylpiperidine (Compound 11)

A mixture of tert-butyl 6-oxa-3-azabicyclo[3.1.0]hexane-3-carboxylate (10 g, 54 mmol), NaN₃ (7 g, 108 mmol) and NH₄Cl (2.8 g, 54 mmol) in MeOH (120 mL) and H₂O (20 mL) was stirred at 65° C. under N₂ overnight. The reaction mixture was concentrated in vacuum, the residue was extracted with ethyl acetate (300 mL×3), the combined organic was washed with water (200 mL×2), dried over anhydrous Na₂SO₄, concentrated and purified by flash column chromatography on silica gel (ethyl acetate in petroleum ether=20% v/v) to obtain compound 11 (11 g, yield 89%) as oil. LC-MS (ESI) m/z: 129 [M+H−100]⁺.

### Step 2: Synthesis of 4-ethynylpiperidine (Compound 5)

A mixture of compound 11 (3 g, 13.1 mmol), 1-(bromomethyl)-4-(trifluoromethyl)benzene (3.1 g, 13.1 mmol) and NaH (0.6 g, 15.7 mmol) in DMF (20 mL) was stirred at rt under N₂ protection for 6 hours. The reaction mixture was diluted with water (100 mL) and extracted with ethyl acetate (100 mL×2), the combined organic was washed with water (100 mL), dried over anhydrous Na₂SO₄, concentrated and purified by flash column chromatography on silica gel (ethyl acetate in petroleum ether=30% v/v) to obtain compound 5 (3.8 g, yield 75%) as oil. LC-MS (ESI) m/z: 287 [M+H−100]⁺.

### Step 3: Synthesis of 4-ethynylpiperidine (Compound 2)

A mixture of tert-butyl 4-ethynylpiperidine-1-carboxylate (1000 mg, 4.7 mmol) and TFA (0.5 mL) in DCM (1 mL) was stirred at rt under N₂ protection for 2 hours. The mixture was concentrated to leave the crude compound 2 (1.2 g) as white solid, which was used directly in the next step. LC-MS (ESI) m/z: 110 [M+H]⁺.

### Step 4: Synthesis of tert-butyl trans-3-(4-(piperidin-4-yl)-1H-1,2,3-triazol-1-yl)-4-(4-(trifluoromethyl)benzyloxy)pyrrolidine-1-carboxylate (Compound 6)

A mixture of compound 5 (200 mg, 0.52 mmol), compound 2 (113 mg, 1.04 mmol), CuSO₄ (48 mg, 0.3 mmol) and sodium L-ascorbate (28 mg, 0.14 mmol) in THF (1 mL), H₂O (1 mL) and nBuOH (1 mL) was stirred at 70° C. under N₂ overnight. The reaction mixture was concentrated in vacuum, the residue was diluted with water (50 mL) and extracted with ethyl acetate (20 mL×2), the combined organic was washed with brine (20 mL), dried over anhydrous Na₂SO₄, concentrated and purified by flash column chromatography on silica gel (ethyl acetate in petroleum ether=90% v/v) to obtain the target compound 6 as yellow solid (200 mg, yield 78%). LC-MS (ESI) m/z: 496 [M+H]⁺.

### Step 5: Synthesis of tert-butyl trans-3-(4-(1-methylpiperidin-4-yl)-1H-1,2,3-triazol-1-yl)-4-(4-(trifluoromethyl)benzyloxy)pyrrolidine-1-carboxylate (Compound 7)

A mixture of compound 6 (100 mg, 0.2 mmol) and formaldehyde (10 mg, 0.3 mmol) and NaBH₄ (20 mg, 0.4 mmol) in MeOH (10 mL) was stirred at rt for 2 hours. The resulting mixture was concentrated and purified by flash column chromatography on silica gel (ethyl acetate in petroleum ether=80% v/v) to obtain the target compound 7 as oil (70 mg, yield 69%). LC-MS (ESI) m/z: 510 [M+H]⁺.

### Step 6: Synthesis of 1-methyl-4-(1-(trans-4-(4-(trifluoromethyl)benzyloxy)pyrrolidin-3-yl)-1H-1,2,3-triazol-4-yl) piperidine (Compound 8)

A mixture of compound 7 (70 mg, 0.13 mmol) and TFA (0.5 mL) in DCM (1 mL) was stirred at rt under N₂ for 2 hours. The mixture was concentrated to leave the crude target compound 8 (50 mg) as yellow oil, which was used directly in the next step. LC-MS (ESI) m/z: 410 [M+H]⁺.

### Step 7: Synthesis of 1-(trans-3-(4-(1-methylpiperidin-4-yl)-1H-1,2,3-triazol-1-yl)-4-(4-(trifluoromethyl)benzyloxy)pyrrolidin-1-yl)prop-2-en-1-one (Compound II-19)

To the mixture of compound 8 (50 mg, 0.12 mmol) and TEA (30 mg, 0.24 mmol) in DCM (10 mL) was added acryloyl chloride (20 mg, 0.12 mmol), the mixture was stirred at rt under N₂ for 2 hours, and then concentrated and purified by prep-HPLC to obtain the target compound II-19 as white solid (13 mg, yield 23%). LC-MS (ESI) m/z: 464 [M+H]⁺; ¹H NMR (400 MHz, CD₃OD) δ (ppm) 7.89 (d, J=3.6 Hz, 1H), 7.63 (d, J=8.1 Hz, 2H), 7.49 (d, J=8.1 Hz, 2H), 6.61 (dd, J=16.8, 10.4 Hz, 1H), 6.31 (dd, J=16.8, 1.8 Hz, 1H), 5.78 (ddd, J=10.4, 3.6, 1.9 Hz, 1H), 5.39-5.27 (m, 1H), 4.73 (d, J=4.6 Hz, 2H), 4.61-4.50 (m, 1H), 4.34-4.10 (m, 2H), 4.09-3.69 (m, 2H), 2.94 (d, J=11.6 Hz, 2H), 2.80-2.68 (m, 1H), 2.31 (s, 3H), 2.24-2.12 (m, 2H), 2.08-1.98 (m, 2H), 1.80-1.65 (m, 2H)-2.12 (m, 2H), 2.08-1.98 (m, 2H), 2H), 1.80-1.65 (m, 2H), 2H), 1.80-1.65 (m, 2H).

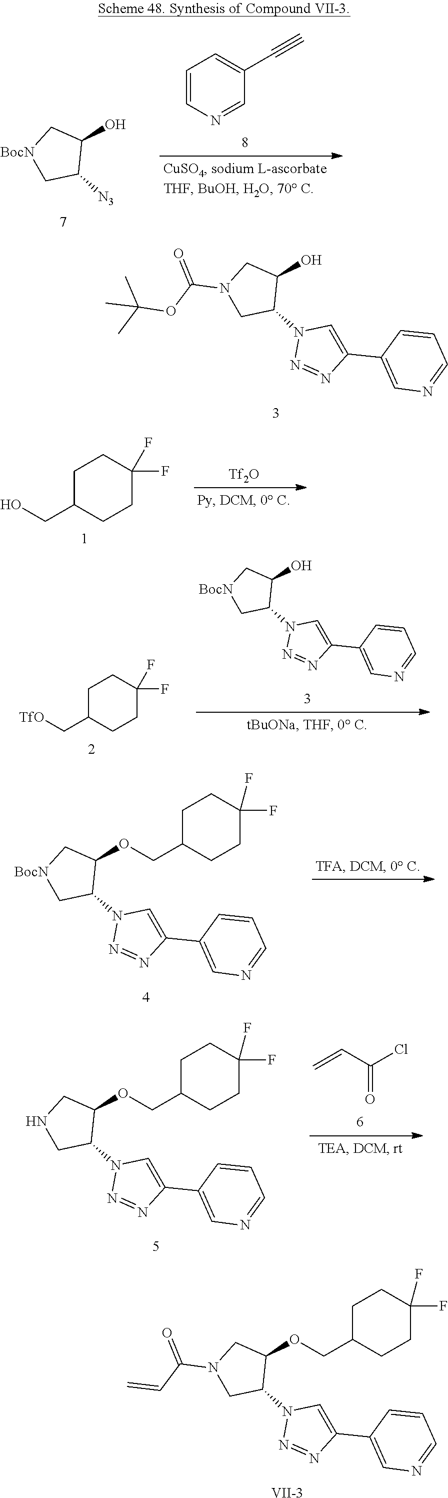

### Step 1: Synthesis of tert-butyl trans-3-hydroxy-4-(4-(pyridin-3-yl)-1H-1,2,3-triazol-1-yl)pyrrolidine-1-carboxylate (Compound 3)

A mixture of tert-butyl trans-3-azido-4-hydroxypyrrolidine-1-carboxylate (1 g, 4.38 mmol), 3-ethynylpyridine (451 mg, 4.38 mmol), CuSO₄ (654 mg, 2.6 mmol) and sodium L-ascorbate (257 mg, 1.3 mmol) in THF (3 mL), H₂O (3 mL) and BuOH (3 mL) was stirred at 70° C. under N₂ protection overnight. The reaction mixture was diluted with ethyl acetate (60 mL), washed with water (40 mL), dried over anhydrous Na₂SO₄, concentrated and purified by flash column chromatography on silica gel (ethyl acetate in petroleum ether=80% v/v) to obtain compound 3 (650 mg, yield 45%) as yellow solid. LC-MS (ESI) m/z: 332 [M+H]⁺.

### Step 2: Synthesis of (4,4-difluorocyclohexyl)methyl Trifluoromethanesulfonate (Compound 2)

A mixture of (4,4-difluorocyclohexyl)methanol (1 g, 6.6 mmol), Tf₂O (2.81 g, 9.9 mmol) and pyridine (1 mL) in DCM (20 mL) was stirred at rt under N₂ protection for 3 hours. The reaction mixture was diluted with ethyl acetate (100 mL), washed with water (100 mL×2), dried over anhydrous Na₂SO₄, filtered and concentrated to leave the crude compound 2 (500 mg) as yellow oil, which was used directly in the next step. LC-MS (ESI) m/z: no MS.

### Step 3: Synthesis of tert-butyl trans-3-((4,4-difluorocyclohexyl)methoxy)-4-(4-(pyridin-3-yl)-1H-1,2,3-triazol-1-yl)pyrrolidine-1-carboxylate (Compound 4)

A mixture of compound 2 (200 mg, 0.7 mmol), compound 3 (58 mg, 0.175 mmol) and tBuONa (25 mg, 0.26 mmol) in THF (5 mL) was stirred at 0° C. under N₂ for 2 hours. The reaction mixture was diluted with ethyl acetate (50 mL), washed with water (50 mL×2), dried over anhydrous Na₂SO₄, concentrated and purified by flash column chromatography on silica gel (ethyl acetate in petroleum ether=50% v/v) to obtain the target compound 4 as oil (100 mg, yield 30%). LC-MS (ESI) m/z: 464 [M+H]⁺.

### Step 4: Synthesis of 3-(1-(trans-4-((4,4-difluorocyclohexyl)methoxy)pyrrolidin-3-yl)-1H-1,2,3-triazol-4-yl)pyridine (Compound 5)

A mixture of compound 4 (50 mg, 0.1 mmol) and TFA (1 mL) in DCM (3 mL) was stirred at rt for 2 hours. The mixture was concentrated to leave the crude compound 5 (50 mg) as yellow oil, which was used directly in the next step. LC-MS (ESI) m/z: 364 [M+H]⁺.

### Step 5: Synthesis of 1-(trans-3-((4,4-difluorocyclohexyl)methoxy)-4-(4-(pyridin-3-yl)-1H-1,2,3-triazol-1-yl)pyrrolidin-1-yl)prop-2-en-1-one (Compound VII-3)

A mixture of compound 5 (50 mg, 0.1 mmol), acryloyl chloride (10 mg, 0.1 mmol), and TEA (20 mg, 0.2 mmol) in DCM (5 mL) was stirred at rt under N₂ for 2 hours. The mixture was diluted with DCM (50 mL), washed with water (50 mL×2), dried over anhydrous Na₂SO₄, concentrated and purified by prep-HPLC to obtain VII-3 as white solid (16 mg, yield 38%). LC-MS (ESI) m/z: 418 [M+H]⁺; ¹H-NMR (400 MHz, CD₃OD) δ (ppm) 8.91 (d, J=1.2 Hz, 1H), 8.55-8.40 (m, 2H), 8.18 (dt, J=8.0, 1.9 Hz, 1H), 7.43 (dd, J=8.0, 4.9 Hz, 1H), 6.55 (ddd, J=16.8, 10.4, 2.5 Hz, 1H), 6.28-6.20 (m, 1H), 5.70 (ddd, J=10.5, 4.7, 1.9 Hz, 1H), 5.29-5.18 (m, 1H), 4.43-4.32 (m, 1H), 4.27-4.16 (m, 1H), 4.07-3.37 (m, 5H), 1.91 (ddd, J=13.9, 7.0, 3.5 Hz, 2H), 1.76-1.54 (m, 5H), 1.26-1.13 (m, 2H).

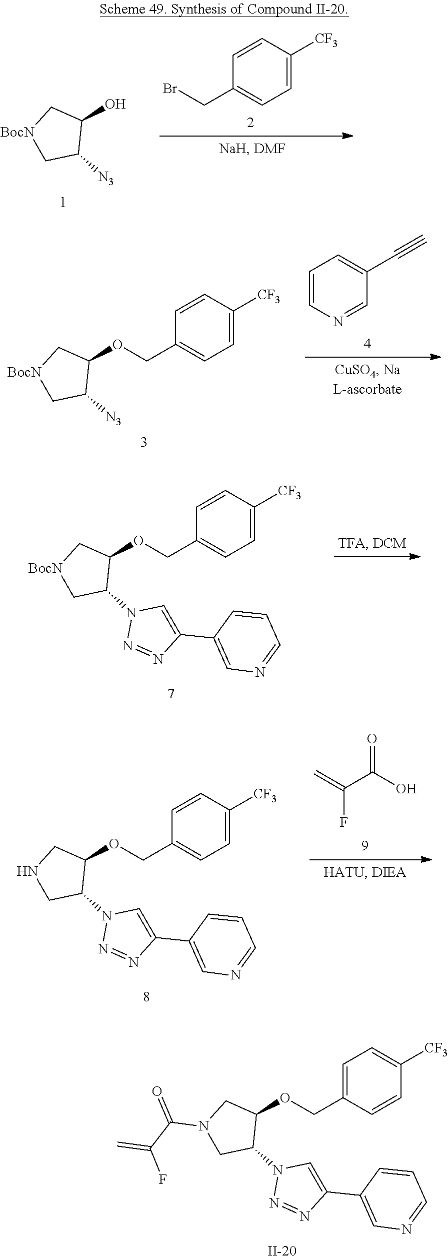

### Step 1: Synthesis of tert-butyl (3R, 4R)-3-azido-4-(4-(trifluoromethyl)benzyloxy)pyrrolidine-1-carboxylate (Compound 3)

To a mixture of tert-butyl (3R, 4R)-3-azido-4-hydroxypyrrolidine-1-carboxylate (500 mg, 2.19 mmol) in DMF (5 mL) was added NaH (175 mg, 4.38 mmol), the mixture was stirred at rt for 10 minutes, and then 1-(bromomethyl)-4-(trifluoromethyl)benzene (524 mg, 2.19 mmol) was added, the mixture was stirred at rt under N₂ overnight. The mixture was diluted with water (100 mL) and extracted with ethyl acetate (60 mL), the organic was washed with water (40 mL), dried over anhydrous Na₂SO₄, concentrated and purified by flash column chromatography on silica gel (ethyl acetate in petroleum ether=20% v/v) to obtain compound 3 as clear oil (700 mg, yield 83%). LC-MS (ESI) m/z: 287 [M+H−100]⁺.

### Step 2: Synthesis of tert-butyl (3R, 4R)-3-(4-(pyridin-3-yl)-1H-1,2,3-triazol-1-yl)-4-(4-(trifluoromethyl)benzyloxy)pyrrolidine-1-carboxylate (Compound 7)

A mixture of compound 3 (700 mg, 3.0 mmol), 3-ethynylpyridine (316 mg, 3 mmol), CuSO₄ (224 mg, 0.9 mmol) and sodium L-ascorbate (356 mg, 0.6 mmol) in THF (3 mL), H₂O (3 mL) and nBuOH (3 mL) was stirred at 70° C. overnight. The mixture was diluted with water (100 mL) and extracted with ethyl acetate (60 mL), the organic was washed with water (40 mL), dried over anhydrous Na₂SO₄, concentrated and purified by flash column chromatography on silica gel (ethyl acetate in petroleum ether=80% v/v) to obtain compound 7 as yellow solid (800 mg, yield 54%). LC-MS (ESI) m/z: 490 [M+H]⁺.

### Step 3: Synthesis of 3-(1-(3R, 4R)-4-(4-(trifluoromethyl)benzyloxy)pyrrolidin-3-yl)-1H-1,2,3-triazol-4-yl)pyridine (Compound 8)

A mixture of compound 7 (800 mg, 1.63 mmol) and TFA (3 mL) in DCM (6 mL) was stirred at rt for 2 hours. The mixture was concentrated to leave the crude compound 8 as white solid (600 mg, yield 94%), which was used directly for next step. LC-MS (ESI) m/z: 390 [M+H]⁺.

### Step 4: Synthesis of 2-fluoro-1-((3R, 4R)-3-(4-(pyridin-3-yl)-1H-1,2,3-triazol-1-yl)-4-(4-(trifluoromethyl)benzyloxy)pyrrolidin-1-yl)prop-2-en-1-one (Compound II-20)

A mixture of compound 8 (100 mg, 0.25 mmol), 2-fluoroacrylic acid (30 mg, 0.25 mmol), HATU (140 mg, 0.37 mmol) and TEA (50 mg, 0.5 mmol) in DCM (10 mL) was stirred at rt for 2 hours. The mixture was concentrated and purified by prep-HPLC to obtain compound II-20 as yellow oil (44 mg, yield 38%). LC-MS (ESI) m/z: 462 [M+H]⁺. ¹H NMR (400 MHz, CD₃OD) δ (ppm) 9.03 (s, 1H), 8.61 (s, 1H), 8.55 (dd, J=5.2, 1.2 Hz, 1H), 8.30 (d, J=7.8 Hz, 1H), 7.70-7.50 (m, 5H), 5.56 (dd, J=47.2, 3.2 Hz, 1H), 5.51-5.42 (m, 1H), 5.31 (dd, J=16.5, 3.5 Hz, 1H), 4.81 (s, 2H), 4.70-4.60 (m, 1H), 4.52-3.79 (m, 4H).

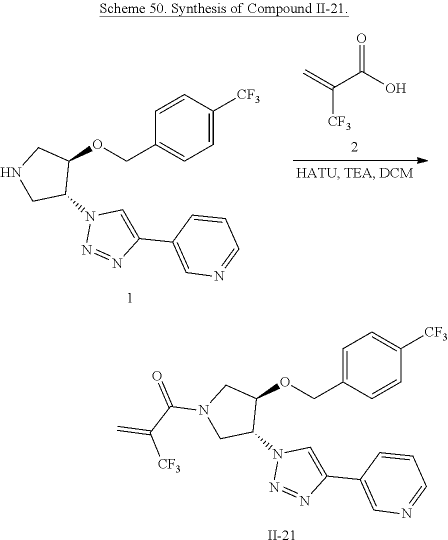

### Step 1: Synthesis of 1-((3R, 4R)-3-(4-(pyridin-3-yl)-1H-1,2,3-triazol-1-yl)-4-(4-(trifluoromethyl)benzyloxy)pyrrolidin-1-yl)-2-(trifluoromethyl) prop-2-en-1-one (Compound II-21)

A mixture of compound 1 (50 mg, 0.12 mmol), 2-(trifluoromethyl) acrylic acid (20 mg, 0.12 mmol), HATU (70 mg, 0.18 mmol) and TEA (25 mg, 0.25 mmol) in DCM (6 mL) was stirred at rt for 2 hours. The mixture was concentrated and purified by prep-HPLC to obtain compound II-21 as white solid (5 mg, 8% yield). LC-MS (ESI) m/z: 512.2 [M+H]⁺. 1H NMR (400 MHz, CD₃OD) δ (ppm) 9.03 (s, 1H), 8.60 (d, J=10.4 Hz, 1H), 8.56 (d, J=4.4 Hz, 1H), 8.30 (d, J=7.5 Hz, 1H), 7.71-7.49 (m, 5H), 6.33 (d, J=10.3 Hz, 1H), 6.12 (s, 1H), 5.52-5.40 (m, 1H), 4.84-4.58 (m, 3H), 4.38-4.03 (m, 3H), 3.88-3.76 (m, 1H).

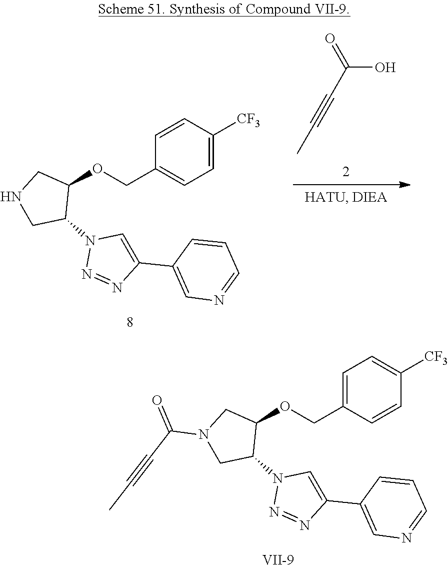

### Synthesis of 1-((3R, 4R)-3-(4-(pyridin-3-yl)-1H-1,2,3-triazol-1-yl)-4-(4-(trifluoromethyl)benzyloxy)pyrrolidin-1-yl)but-2-yn-1-one (VII-9)

A mixture of compound 8 (100 mg, 0.25 mmol), but-2-ynoic acid (30 mg, 0.25 mmol), HATU (140 mg, 0.37 mmol) and TEA (50 mg, 0.5 mmol) in DCM (10 mL) was stirred at rt for 2 hours. The mixture was concentrate and purified by prep-HPLC to obtain compound VII-9 as white solid (30 mg yield 26%). LC-MS (ESI) m/z: 456 [M+H]⁺. ¹H NMR (400 MHz, CD₃OD) δ (ppm) 9.09-8.98 (m, 1H), 8.60 (d, J=4.4 Hz, 1H), 8.57-8.53 (m, 1H), 8.33-8.27 (m, 1H), 7.70-7.50 (m, 5H), 5.52-5.40 (m, 1H), 4.79 (d, J=8.3 Hz, 2H), 4.67-4.62 (m, 1H), 4.44-3.69 (m, 4H), 2.06 (d, J=7.2 Hz, 3H).

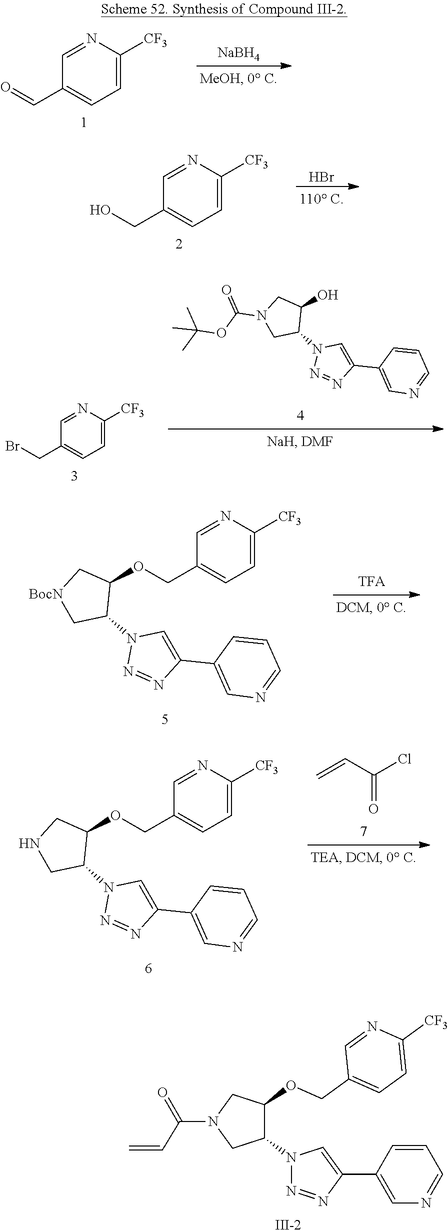

### Step 1: Synthesis of (6-(trifluoromethyl)pyridin-3-yl)methanol (Compound 2)

A mixture of 6-(trifluoromethyl) nicotinaldehyde (170 mg, 1 mmol) and NaBH₄ (76 mg, 2 mmol) in MeOH (3 mL) was stirred at 0° C. for 3 hours. The reaction mixture was concentrated in vacuum, the residue was extracted with ethyl acetate (60 mL), washed with water (40 mL), dried over anhydrous Na₂SO₄, filtered and concentrated to leave the crude product (150 mg, yield 84%) as oil, which was used directly in the next step. LC-MS (ESI) m/z: 178 [M+H]⁺.

### Step 2: Synthesis of 5-(bromomethyl)-2-(trifluoromethyl)pyridine (Compound 3)

A mixture of compound 2 (400 mg, 2.2 mmol) and aqueous HBr solution (48%, 6 mL) was stirred at 110° C. overnight. The reaction mixture was concentrated in vacuum, the residue was diluted with ethyl acetate (60 mL), washed with water (40 mL), dried over anhydrous Na₂SO₄, concentrated and purified by flash column chromatography on silica gel (ethyl acetate in petroleum ether=20% v/v) to obtain the target compound 3 as oil (200 mg, yield 38%). LC-MS (ESI) m/z: 240 [M+H]⁺.

### Step 3: Synthesis of tert-butyl trans-3-(4-(pyridin-3-yl)-1H-1,2,3-triazol-1-yl)-4-((6-(trifluoromethyl)pyridin-3-yl)methoxy)pyrrolidine-1-carboxylate (Compound 5)

A mixture of compound 3 (100 mg, 0.4 mmol), compound 4 (140 mg, 0.4 mmol) and NaH (40 mg, 0.8 mmol) in DMF (5 mL) was stirred at rt under N₂ overnight. The reaction mixture was diluted with water (100 mL) and extracted with ethyl acetate (20 mL×2), the combined organic was washed with water (50 mL), dried over anhydrous Na₂SO₄, concentrated and purified by flash column chromatography on silica gel (ethyl acetate in petroleum ether=90% v/v) to obtain the target compound 5 as solid (100 mg, yield 51%). LC-MS (ESI) m/z: 491 [M+H]⁺.

### Step 4: Synthesis of 5-(trans-4-(4-(pyridin-3-yl)-1H-1,2,3-triazol-1-yl)pyrrolidin-3-yloxy)methyl)-2-(trifluoromethyl)pyridine (Compound 6)

A mixture of compound 5 (100 mg, 0.2 mmol) and TFA (1 mL) in DCM (3 mL) was stirred at rt for 2 hours. The mixture was concentrated to leave the crude compound 6 (100 mg, crude) as yellow oil, which was used directly in the next step. LC-MS (ESI) m/z: 391

### Step 5: Synthesis of 1-(trans-3-(4-(pyridin-3-yl)-1H-1,2,3-triazol-1-yl)-4-((6-(trifluoromethyl)pyridin-3-yl)methoxy)pyrrolidin-1-yl)prop-2-en-1-one (Compound III-2)

A mixture of compound 6 (50 mg, 0.125 mmol), acryloyl chloride (15 mg, 0.125 mmol) and TEA (25 mg, 0.25 mmol) in DCM (3 mL) was stirred at 0° C. for 2 hours. The mixture was concentrated and purified by prep-HPLC to obtain III-2 as white solid (5 mg, yield 9%). LC-MS (ESI) m/z: 445. ¹H NMR (400 MHz, CD₃OD) δ (ppm) 9.04 (d, J=2.2 Hz, 1H), 8.76-8.50 (m, 3H), 8.31 (dt, J=7.9, 1.9 Hz, 1H), 8.03 (d, J=8.2 Hz, 1H), 7.80 (d, J=8.1 Hz, 1H), 7.56 (dd, J=8.0, 4.9 Hz, 1H), 6.67 (ddd, J=16.8, 10.4, 3.5 Hz, 1H), 6.36 (dd, J=16.8, 1.7 Hz, 1H), 5.83 (ddd, J=10.5, 5.1, 1.9 Hz, 1H), 5.58-5.44 (m, 1H), 4.97 (s, 2H), 4.81-4.68 (m, 1H), 4.48-4.03 (m, 3H), 4.02-3.80 (m, 1H).

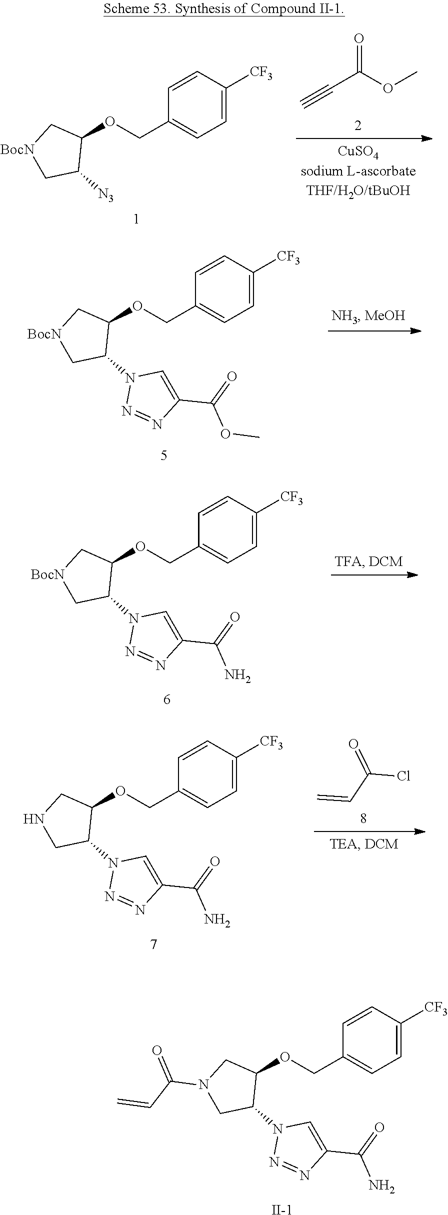

### Step 1: Synthesis of methyl 1-(trans-1-(tert-butoxycarbonyl)-4-(4-(trifluoromethyl)benzyloxy)pyrrolidin-3-yl)-1H-1,2,3-triazole-4-carboxylate (Compound 5)

A mixture of tert-butyl trans-3-azido-4-((4-(trifluoromethyl)benzyl)oxy)pyrrolidine-1-carboxylate (400 mg, 1.04 mmol), methyl propiolate (169 mg, 2.08 mmol), CuSO₄ (174 mg. 0.7 mmol) and sodium L-ascorbate (64 mg, 0.28 mmol) in THF (2 mL), H₂O (2 mL) and nBuOH (2 mL) was stirred at 70° C. under N₂ overnight. The mixture was diluted with water (50 mL) and extracted with ethyl acetate (50 mL), the organic was washed with water (50 mL), dried over anhydrous Na₂SO₄, concentrated and purified by flash column chromatography on silica gel (ethyl acetate in petroleum ether=70% v/v) to afford the target compound 5 as yellow solid (400 mg, yield 82%). LC-MS (ESI) m/z: 471 [M+H]⁺.

### Step 2: Synthesis of tert-butyl trans-3-(4-carbamoyl-1H-1,2,3-triazol-1-yl)-4-(4-(trifluoromethyl)benzyloxy)pyrrolidine-1-carboxylate (Compound 6)

A mixture of compound 5 (60 mg, 0.12 mmol) and ammonia solution in MeOH (7 M. 3 mL) was stirred at 80° C. overnight. The mixture was concentrated to leave the crude compound 6 as white solid (50 mg, yield 91%). LC-MS (ESI) m/z: 400 [M+H−56]⁺.

### Step 3: Synthesis of 1-(trans-4-(4-(trifluoromethyl)benzyloxy)pyrrolidin-3-yl)-1H-1,2,3-triazole-4-carboxamide (Compound 7)

A mixture of compound 6 (60 mg, 0.13 mmol) and TFA (0.5 mL) in DCM (1 mL) was stirred at rt for 2 h. The mixture was concentrated to leave the crude compound 7 as white solid (50 mg, yield 86%). LC-MS (ESI) m/z: 356 [M+H]⁺.

### Step 4: Synthesis of 1-(trans-1-acryloyl-4-(4-(trifluoromethyl)benzyloxy)pyrrolidin-3-yl)-1H-1,2,3-triazole-4-carboxamide (II-1)

A mixture of compound 7 (50 mg, 0.14 mmol), acryloyl chloride (20 mg, 0.14 mmol) and TEA (30 mg, 0.28 mmol) in DCM (5 mL) was stirred at rt for 2 h. The mixture was concentrated and purified by prep-HPLC to obtain compound II-1 as yellow solid (10 mg, yield 17%). LC-MS (ESI) m/z: 410 [M+H]⁺. ¹H NMR (400 MHz, CD₃OD) δ 8.49 (d, J=4.8 Hz, 1H), 7.63 (d, J=8.2 Hz, 2H), 7.51 (d, J=8.1 Hz, 2H), 6.62 (dd, J=16.8, 10.4 Hz, 1H), 6.32 (dd, J=16.8, 1.5 Hz, 1H), 5.79 (ddd, J=10.4, 4.1, 1.9 Hz, 1H), 5.42 (ddd, J=14.7, 7.0, 3.7 Hz, 1H), 4.75 (d, J=4.6 Hz, 2H), 4.64-4.54 (m, 1H), 4.38-3.95 (m, 3H), 3.81 (ddd, J=16.7, 12.5, 3.3 Hz, 1H).

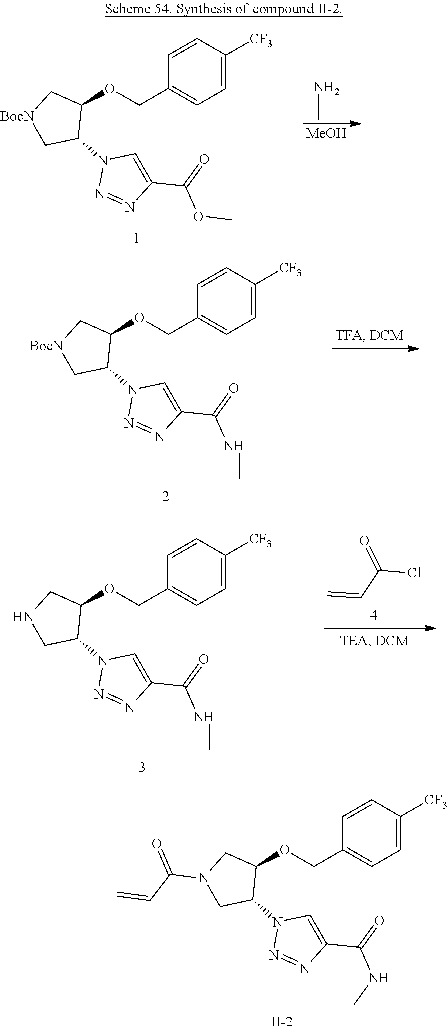

### Step 1: Synthesis of tert-butyl trans-3-(4-(methylcarbamoyl)-1H-1,2,3-triazol-1-yl)-4-(4-(trifluoromethyl)benzyloxy)pyrrolidine-1-carboxylate (Compound 2)

A mixture of methyl 1-(trans-1-(tert-butoxycarbonyl)-4-((4-(trifluoromethyl)benzyl)oxy)pyrrolidin-3-yl)-1H-1,2,3-triazole-4-carboxylate (60 mg, 0.12 mmol) and methylamine (37 mg, 1.2 mmol) in MeOH (3 mL) was stirred at 80° C. overnight. The mixture was concentrated to leave the crude compound 2 as white solid (55 mg, yield 100%). LC-MS (ESI) m/z: 414 [M+H−56]⁺.

### Step 2: Synthesis of N-methyl-1-(trans-4-(4-(trifluoromethyl)benzyloxy)pyrrolidin-3-yl)-1H-1,2,3-triazole-4-carboxamide (Compound 3)

A mixture of compound 2 (60 mg, 0.12 mmol) and TFA (0.5 mL) in DCM (1 mL) was stirred at rt for 2 h. The mixture was concentrated to leave the crude compound 3 as white solid (45 mg, yield 100%). LC-MS (ESI) m/z: 370 [M+H]⁺.

### Step 3: Synthesis of 1-(trans-1-acryloyl-4-(4-(trifluoromethyl)benzyloxy)pyrrolidin-3-yl)-N-methyl-1H-1,2,3-triazole-4-carboxamide (II-2)

A mixture of compound 3 (50 mg, 0.13 mmol), acryloyl chloride (20 mg, 0.13 mmol) and TEA (30 mg, 0.26 mmol) in DCM (5 mL) was stirred at rt for 2 h. The mixture was concentrated and purified by prep-HPLC to obtain compound II-2 as yellow solid (18 mg, yield 33%). LC-MS (ESI) m/z: 424 [M+H]⁺. ¹H NMR (400 MHz, CD₃OD) δ 8.47 (d, J=5.2 Hz, 1H), 7.58 (dd, J=49.5, 8.1 Hz, 4H), 6.63 (dd, J=16.8, 10.4 Hz, 1H), 6.34 (d, J=16.8 Hz, 1H), 5.80 (ddd, J=10.4, 4.7, 1.7 Hz, 1H), 5.50-5.38 (m, 1H), 4.76 (d, J=4.5 Hz, 2H), 4.66-4.55 (m, 1H), 4.37-3.97 (m, 3H), 3.83 (ddd, J=16.6, 12.5, 3.2 Hz, 1H), 2.94 (s, 3H).

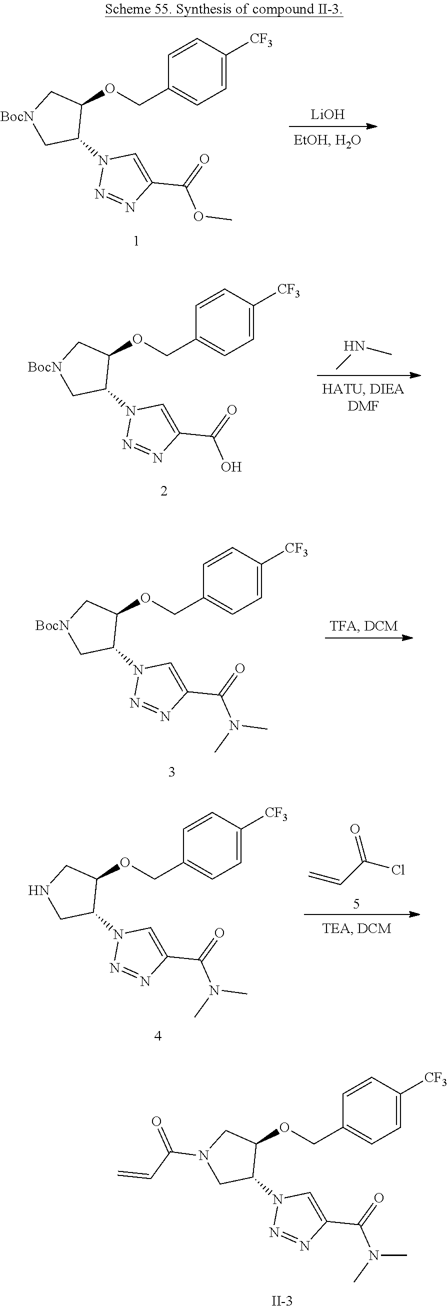

### Step 1: Synthesis of 1-(trans-1-(tert-butoxycarbonyl)-4-(4-(trifluoromethyl)benzyloxy)pyrrolidin-3-yl)-1H-1,2,3-triazole-4-carboxylic Acid (Compound 2)

A mixture of methyl 1-(trans-1-(tert-butoxycarbonyl)-4-((4-(trifluoromethyl)benzyl)oxy)pyrrolidin-3-yl)-1H-1,2,3-triazole-4-carboxylate (150 mg, 0.319 mmol) and LiOH (147 mg, 3.5 mmol) in EtOH (5 mL) and H₂O (2 mL) was stirred at RT overnight. The mixture was adjusted to pH 6-7 with diluted HCl solution, the resulting solid was collected to obtain compound 2 as white solid (100 mg, yield 71%). LC-MS (ESI) m/z: 457 [M+H]⁺.

### Step 2: Synthesis of tert-butyl trans-3-(4-(dimethylcarbamoyl)-1H-1,2,3-triazol-1-yl)-4-(4-(trifluoromethyl)benzyloxy)pyrrolidine-1-carboxylate (Compound 3)

A mixture of compound 2 (70 mg, 0.15 mmol), dimethylamine (7 mg, 0.15 mmol), HATU (85 mg, 0.2 mmol) and DIEA (39 mg, 0.3 mmol) in DMF (6 mL) was stirred at rt for 2 h. The mixture was concentrated and purified by prep-HPLC to obtain compound 3 as white solid (60 mg, yield 83%). LC-MS (ESI) m/z: 484 [M+H]⁺.

### Step 3: Synthesis of N,N-dimethyl-1-(trans-4-(4-(trifluoromethyl)benzyloxy)pyrrolidin-3-yl)-1H-1,2,3-triazole-4-carboxamide (Compound 4)

A mixture of compound 3 (60 mg, 0.12 mmol) and TFA (0.5 mL) in DCM (1 mL) was stirred at rt for 2 h. The mixture was concentrated to leave the crude compound 4 as white solid (46 mg, yield 100%). LC-MS (ESI) m/z: 384 [M+H]⁺.

### Step 4: Synthesis of 1-(trans-1-acryloyl-4-(4-(trifluoromethyl)benzyloxy)pyrrolidin-3-yl)-N,N-dimethyl-1H-1,2,3-triazole-4-carboxamide (II-3)

A mixture of compound 4 (50 mg, 0.13 mmol), acryloyl chloride (20 mg, 0.13 mmol) and TEA (30 mg, 0.26 mmol) in DCM (5 mL) was stirred at rt for 2 h. The mixture was concentrated and purified by prep-HPLC to obtain compound II-3 as yellow solid (10 mg, yield 18%). LC-MS (ESI) m/z: 438 [M+H]⁺. ¹H NMR (400 MHz, CD₃OD) δ 8.44 (d, J=5.1 Hz, 1H), 7.57 (dd, J=47.9, 8.1 Hz, 4H), 6.62 (dd, J=16.8, 10.4 Hz, 1H), 6.32 (d, J=16.8 Hz, 1H), 5.79 (ddd, J=10.4, 4.1, 1.7 Hz, 1H), 5.50-5.34 (m, 1H), 4.75 (d, J=4.5 Hz, 2H), 4.60 (ddd, J=18.5, 3.6, 1.8 Hz, 1H), 4.38-3.96 (m, 3H), 3.92-3.72 (m, 1H), 3.41 (s. 3H), 3.11 (s, 3H).

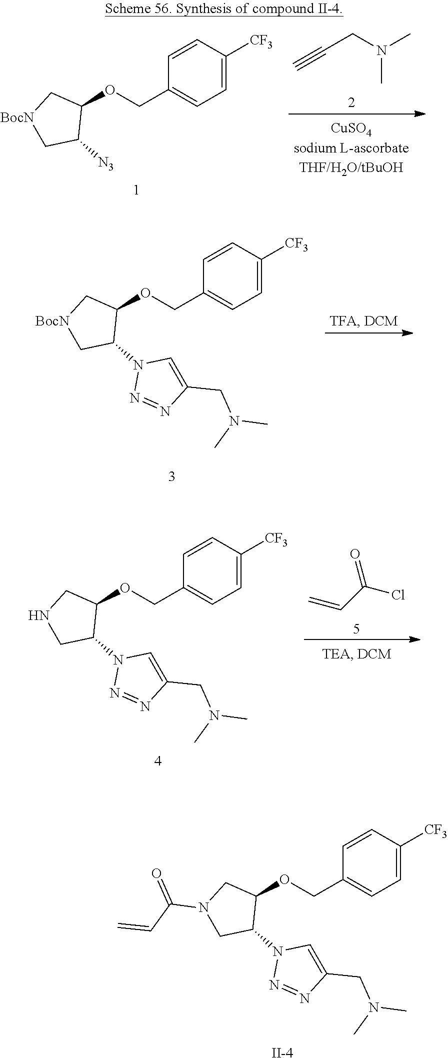

### Step 1: Synthesis of tert-butyl trans-3-(4-((dimethylamino)methyl)-1H-1,2,3-triazol-1-yl)-4-(4-(trifluoromethyl)benzyloxy)pyrrolidine-1-carboxylate (Compound 3)

A mixture of tert-butyl trans-3-azido-4-((4-(trifluoromethyl)benzyl)oxy)pyrrolidine-1-carboxylate (100 mg, 0.26 mmol), N,N-dimethylprop-2-yn-1-amine (40 mg, 0.52 mmol), CuSO₄ (40 mg, 0.15 mmol) and sodium L-ascorbate (20 mg, 0.07 mmol) in THF (1 mL), H₂O (1 mL) and nBuOH (1 mL) was stirred at 70° C. under N₂ overnight. The mixture was concentrated and purified by flash column chromatography on silica gel (ethyl acetate in petroleum ether=80% v/v) to obtain compound 3 as oil (100 mg, yield 79%). LC-MS (ESI) m/z: 470 [M+H]⁺.

### Step 2: Synthesis of N,N-dimethyl-1-(1-(trans-4-(4-(trifluoromethyl)benzyloxy)pyrrolidin-3-yl)-1H-1,2,3-triazol-4-yl)methanamine (Compound 4)

A mixture of compound 3 (60 mg, 0.12 mmol) and TFA (0.5 mL) in DCM (1 mL) was stirred at rt for 2 h. The mixture was concentrated to leave the crude compound 4 as white solid (45 mg, yield 100%). LC-MS (ESI) m/z: 370 [M+H]⁺.

### Step 3: Synthesis of 1-(trans-3-(4-((dimethylamino)methyl)-1H-1,2,3-triazol-1-yl)-4-(4-(trifluoromethyl)benzyloxy)pyrrolidin-1-yl)prop-2-en-1-one (II-4)

A mixture of compound 4 (50 mg, 0.13 mmol), acryloyl chloride (20 mg, 0.13 mmol) and TEA (30 mg, 0.26 mmol) in DCM (5 mL) was stirred at rt for 2 h. The mixture was concentrated and purified by prep-HPLC to obtain compound II-4 as white solid (12 mg, yield 22%). LC-MS (ESI) m/z: 424 [M+H]⁺. ¹H NMR (400 MHz, CD₃OD) δ 8.00 (d, J=3.2 Hz, 1H), 7.57 (dd, J=49.1, 8.1 Hz, 4H), 6.62 (ddd, J=16.8, 10.4, 2.0 Hz, 1H), 6.31 (dd, J=16.8, 1.8 Hz, 1H), 5.79 (ddd, J=10.4, 3.0, 2.0 Hz, 1H), 5.48-5.26 (m, 1H), 4.74 (d, J=4.7 Hz, 2H), 4.57 (ddd, J=20.8, 3.6, 1.8 Hz, 1H), 4.37-3.94 (m, 3H), 3.90-3.70 (m, 1H), 3.62 (s, 2H), 2.26 (s, 6H).

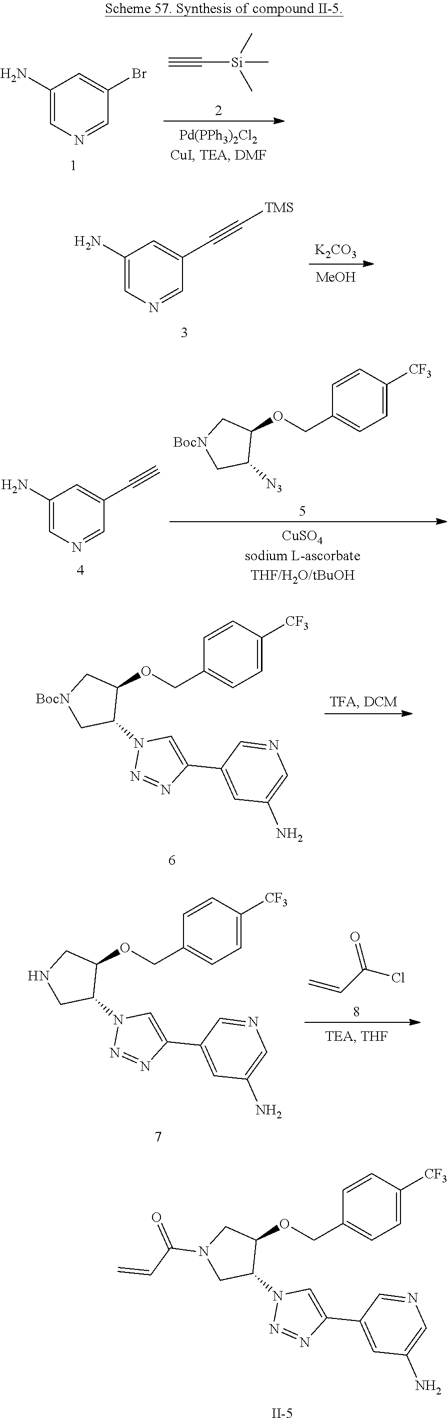

### Step 1: Synthesis of 5-((trimethylsilyl)ethynyl)pyridin-3-amine (Compound 3)

To a solution of 5-bromopyridin-3-amine (1 g, 5.85 mmol) in DMF (20 mL) was added ethynyltrimethylsilane (0.6 g, 6.14 mmol), Pd(PPh₃)₂Cl₂ (0.24 g, 0.29 mmol), CuI (0.056 g, 0.29 mmol) and Et₃N (1.18 g, 11.7 mmol). The mixture was stirred at RT under N₂ for 2 h., concentrated and purified by flash column chromatography on silica gel (ethyl acetate in petroleum ether=50% v/v) to obtain the target compound 3 as oil (800 mg, yield 72.7%). LC-MS (ESI) m/z: 190 [M+H]⁺.

### Step 2: Synthesis of 5-ethynylpyridin-3-amine (Compound 4)

To a solution of 5-((trimethylsilyl)ethynyl)pyridin-3-amine (800 mg, 4.2 mmol) in MeOH (20 mL) was added K₂CO₃ (1159.2 mg, 8.4 mmol). The mixture was stirred at RT for 2 h., concentrated and purified by flash column chromatography on silica gel (ethyl acetate in petroleum ether=50% v/v) to obtain the target compound 4 as oil (500 mg, yield 99%). LC-MS (ESI) m/z: 118 [M+H]⁺.

### Step 3: Synthesis of tert-butyl trans-3-(4-(5-aminopyridin-3-yl)-1H-1,2,3-triazol-1-yl)-4-(4-(trifluoromethyl)benzyloxy)pyrrolidine-1-carboxylate (Compound 6)

To a solution of tert-butyl trans-3-azido-4-(4-(trifluoromethyl)benzyloxy)pyrrolidine-1-carboxylate (450 mg, 1.17 mmol) in THF (10 mL), H₂O (10 mL) and BuOH (10 mL) was added 5-ethynylpyridin-3-amine (180 mg, 1.74 mmol), CuSO₄ (30 mg, 0.117 mmol) and sodium L-ascorbate (47 mg, 0.234 mmol). The mixture was stirred at 70° C. under N₂ for 16 h., concentrated and purified by flash column chromatography on silica gel (ethyl acetate in petroleum ether=50% v/v) to obtain the target compound 6 as solid (400 mg, yield 52.1%). LC-MS (ESI) m/z: 504 [M+H]⁺.

### Step 4: Synthesis of 5-(1-(trans-4-(4-(trifluoromethyl)benzyloxy)pyrrolidin-3-yl)-1H-1,2,3-triazol-4-yl)pyridin-3-amine (Compound 7)

A mixture of tert-butyl trans-3-(4-(5-aminopyridin-3-yl)-1H-1,2,3-triazol-1-yl)-4-(4-(trifluoromethyl)benzyloxy)pyrrolidine-1-carboxylate (350 mg, 0.7 mmol) and TFA (2 mL) in DCM (10 mL) was stirred at RT for 2 h. The mixture was concentrated to leave crude compound 7 as oil (300 mg, crude). LC-MS (ESI) m/z: 404 [M+H]⁺.

### Step 5: Synthesis of 1-(trans-3-(4-(5-aminopyridin-3-yl)-1H-1,2,3-triazol-1-yl)-4-(4-(trifluoromethyl)benzyloxy)pyrrolidin-1-yl)prop-2-en-1-one (Compound II-5)

To a solution of 5-(1-(trans-4-(4-(trifluoromethyl)benzyloxy)pyrrolidin-3-yl)-1H-1,2,3-triazol-4-yl)pyridin-3-amine (280 mg, 0.7 mmol) in THF (10 mL) was added acryloyl chloride (63 mg, 0.7 mmol) and Et₃N (141 mg, 1.4 mmol). The mixture was stirred at −78° C. for 1 h., concentrated and purified by prep-HPLC to obtain the target compound II-5 as solid (37 mg, yield 11.7%). LC-MS (ESI) m/z: 459 [M+H]. ¹H NMR (400 MHz, DMSO) δ 8.71 (d, J=5.0 Hz, 1H), 8.17 (s, 1H), 7.90 (d, J=2.5 Hz, 1H), 7.70 (d, J=8.0 Hz, 2H), 7.54 (d, J=8.0 Hz, 2H), 7.43-7.37 (m, 1H), 6.63 (dd, J=16.7, 10.3 Hz, 1H), 6.19 (d, J=16.7 Hz, 1H), 5.79-5.68 (m, 1H), 5.45 (d, J=14.3 Hz, 2H), 4.76 (s, 2H), 4.58 (d, J=24.3 Hz, 1H), 4.30-4.15 (m, 1H), 4.11-3.94 (m, 2H), 3.86 (dd, J=13.6, 5.5 Hz, 1H), 3.62 (d, J=16.3 Hz, 1H).

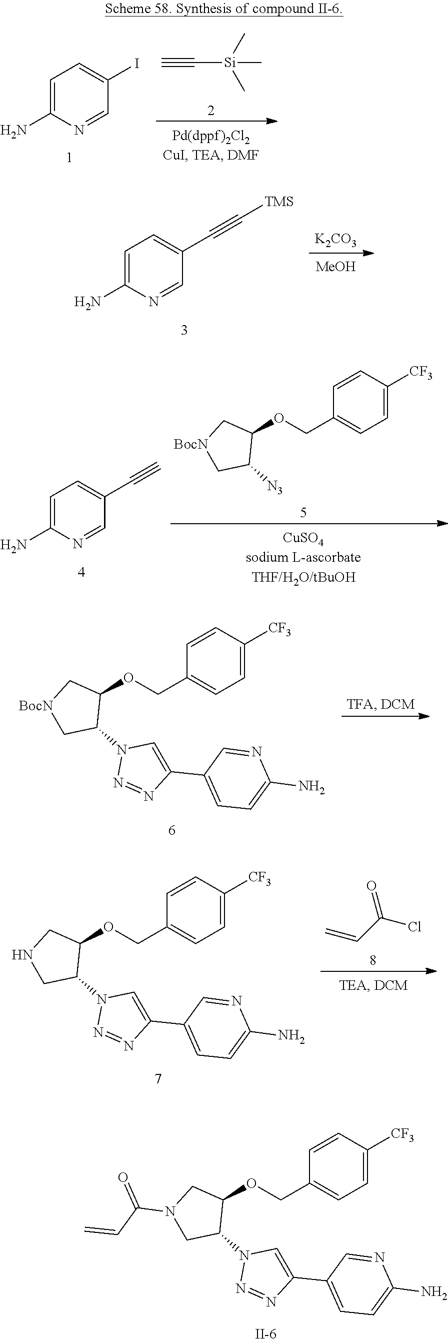

### Step 1: Synthesis of 5-((trimethylsilyl)ethynyl)pyridin-2-amine (Compound 3)

A mixture of 5-iodopyridin-2-amine (460 mg, 2.0 mmol), ethynyltrimethylsilane (305 mg, 3.15 mmol), CuI (80 mg, 0.4 mmol), Pd(dppf)Cl₂ (145 mg, 0.2 mmol) and TEA (404 mg, 101 mmol) in DMF (5 mL) was stirred at 70° C. under N₂ overnight. The mixture was diluted with water (100 mL) and extracted with ethyl acetate (50 mL×2), the combined organic was washed with brine (100 mL×2), dried over anhydrous Na₂SO₄, concentrated and purified by flash column chromatography on silica gel (ethyl acetate in petroleum ether=20% v/v) to obtain the target compound 3 as oil (300 mg, yield 79%). LC-MS (ESI) m/z: 191 [M+H]⁺.

### Step 2: Synthesis of 5-ethynylpyridin-2-amine (Compound 4)

A mixture of compound 3 (100 mg, 0.52 mmol) and K₂CO₃ (79 mg, 0.57 mmol) in MeOH (15 mL) was stirred at 0° C. under N₂ for 3 h. The mixture was concentrated to leave the crude compound 4 as yellow solid (50 mg, yield 81%). LC-MS (ESI) m/z: 119 [M+H]⁺.

### Step 3: Synthesis of tert-butyl trans-3-(4-(6-aminopyridin-3-yl)-1H-1,2,3-triazol-1-yl)-4-(4-(trifluoromethyl)benzyloxy)pyrrolidine-1-carboxylate (Compound 6)

A mixture of compound 4 (101 mg, 0.26 mmol), tert-butyl trans-3-azido-4-((4-(trifluoromethyl)benzyl)oxy)pyrrolidine-1-carboxylate (60 mg, 0.52 mmol), CuSO₄ (40 mg, 0.15 mmol) and sodium L-ascorbate (20 mg, 0.07 mmol) in THF (2 mL), H₂O (2 mL) and nBuOH (2 mL) was stirred at 70° C. under N₂ overnight. The mixture was diluted with water (100 mL) and extracted with ethyl acetate (50 mL×2), the combined organic was washed with brine (100 mL×2), dried over anhydrous Na₂SO₄, concentrated and purified by flash column chromatography on silica gel (ethyl acetate in petroleum ether=90% v/v) to obtain compound 6 as yellow solid (100 mg, yield 76%). LC-MS (ESI) m/z: 505 [M+H]⁺.

### Step 4: Synthesis of 5-(1-(trans-4-(4-(trifluoromethyl)benzyloxy)pyrrolidin-3-yl)-1H-1,2,3-triazol-4-yl)pyridin-2-amine (Compound 7)

A mixture of compound 6 (50 mg, 0.1 mmol) and TFA (0.5 mL) in DCM (1 mL) was stirred at rt for 2 h. The mixture was concentrated to leave the crude compound 7 as white solid (50 mg, yield 98%). LC-MS (ESI) m/z: 405 [M+H]⁺.

### Step 5: Synthesis of 1-(trans-3-(4-(6-aminopyridin-3-yl)-1H-1,2,3-triazol-1-yl)-4-(4-(trifluoromethyl)benzyloxy)pyrrolidin-1-yl)prop-2-en-1-one (II-6)

A mixture of compound 7 (50 mg, 0.12 mmol), acryloyl chloride (20 mg, 0.12 mmol) and TEA (30 mg, 0.24 mmol) in DCM (1 mL) was stirred at rt for 2 h. The mixture was concentrated and purified by prep-HPLC to obtain compound II-6 as white solid (15 mg, yield 27%). LC-MS (ESI) m/z: 459 [M+H]⁺. ¹H NMR (400 MHz, CD₃OD) δ (ppm) 8.40-8.26 (m, 2H), 7.87 (dd, J=8.7, 2.4 Hz, 1H), 7.62 (d, J=8.1 Hz, 2H), 7.51 (d, J=8.1 Hz, 2H), 6.73-6.57 (m, 2H), 6.32 (dd, J=16.8, 1.9 Hz, 1H), 5.86-5.74 (m, 1H), 5.39 (ddd, J=14.8, 7.0, 3.9 Hz, 1H), 4.76 (d, J=4.7 Hz, 2H), 4.68-4.56 (m, 1H), 4.39-3.96 (m, 3H), 3.90-3.70 (m, 1H).

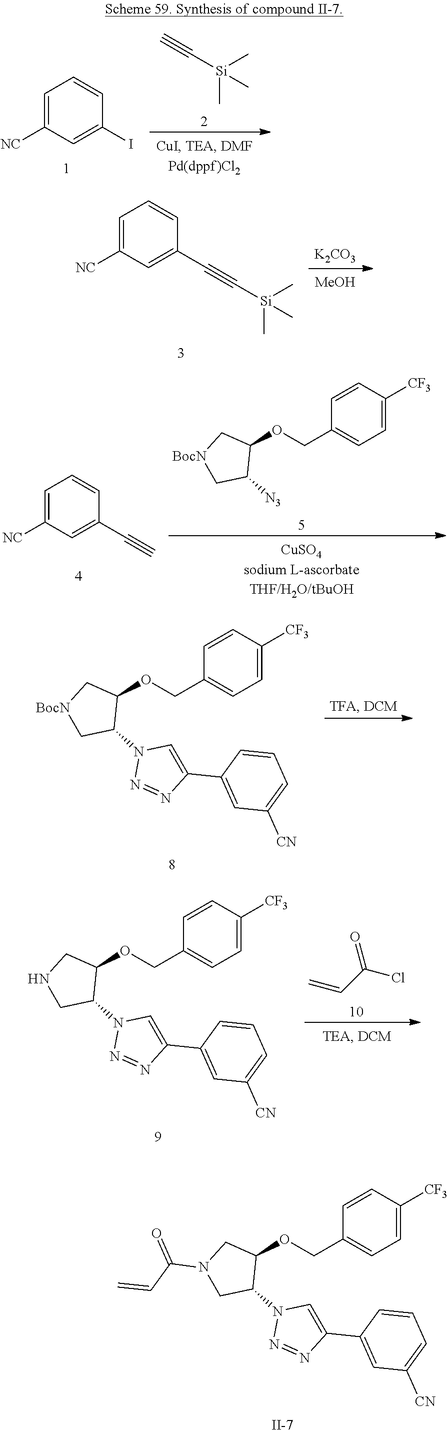

### Step 1: Synthesis of 3-((trimethylsilyl)ethynyl)benzonitrile (Compound 3)

A mixture of 3-iodobenzonitrile (960 mg, 4.2 mmol), ethynyltrimethylsilane (610 mg, 6.3 mmol), CuI (160 mg, 0.8 mmol), Pd(dppf)Cl₂ (290 mg, 0.4 mmol) and TEA (200 mg, 21 mmol) in DMF (25 mL) was stirred at 70° C. under N₂ overnight. The mixture was diluted with water (100 mL) and extracted with ethyl acetate (50 mL×2), the combined organic was washed with brine (100 mL×2), dried over anhydrous Na₂SO₄, concentrated and purified by flash column chromatography on silica gel (ethyl acetate in petroleum ether=20% v/v) to obtain compound 3 as white solid (600 mg, yield 72%). LC-MS (ESI) m/z: 200 [M+H]⁺.

### Step 2: Synthesis of 3-ethynylbenzonitrile (Compound 4)

A mixture of compound 3 (150 mg, 0.75 mmol) and K₂CO₃ (114 mg, 0.82 mmol) in MeOH (15 mL) was stirred at 0° C. for 3 h. The mixture was concentrated, the residue was diluted with water (100 mL) and extracted with ethyl acetate (60 mL), the organic was washed with water (50 mL), dried over anhydrous Na₂SO₄, filtered and concentrated to leave the crude compound 4 as yellow solid (80 mg, yield 83%). LC-MS (ESI) m/z: 128 [M+H]⁺.

### Step 3: Synthesis of tert-butyl trans-3-(4-(3-cyanophenyl)-1H-1,2,3-triazol-1-yl)-4-(4-(trifluoromethyl)benzyloxy)pyrrolidine-1-carboxylate (Compound 8)

A mixture of compound 4 (100 mg, 0.26 mmol), tert-butyl trans-3-azido-4-((4-(trifluoromethyl)benzyl)oxy)pyrrolidine-1-carboxylate (70 mg, 0.52 mmol), CuSO₄ (40 mg, 0.15 mmol) and sodium L-ascorbate (20 mg, 0.07 mmol) in THF (2 mL), H₂O (2 mL) and nBuOH (2 mL) was stirred at 70° C. under N₂ overnight. The mixture was diluted with water (100 mL) and extracted with ethyl acetate (50 mL×2), the combined organic was washed with brine (100 mL×2), dried over anhydrous Na₂SO₄, concentrated and purified by prep-HPLC to obtain compound 8 as yellow solid (110 mg, yield 82%). LC-MS (ESI) m/z: 514 [M+H]⁺.

### Step 4: Synthesis of 3-(1-(trans-4-(4-(trifluoromethyl)benzyloxy)pyrrolidin-3-yl)-1H-1,2,3-triazol-4-yl)benzonitrile (Compound 9)

A mixture of compound 8 (50 mg, 0.01 mmol) and TFA (1 mL) in DCM (3 mL) was stirred at rt for 2 h. The mixture was concentrated to leave the crude compound 9 as yellow oil (40 mg, yield 96%). LC-MS (ESI) m/z: 414 [M+H]⁺.

### Step 5: Synthesis of 3-(1-(trans-1-acryloyl-4-(4-(trifluoromethyl)benzyloxy)pyrrolidin-3-yl)-1H-1,2,3-triazol-4-yl)benzonitrile (II-7)

A mixture of compound 9 (50 mg, 0.12 mmol), acryloyl chloride (11 mg, 0.12 mmol) and TEA (24 mg, 0.24 mmol) in DCM (5 mL) was stirred at rt for 2 h. The mixture was concentrated and purified by prep-HPLC to obtain compound II-7 as white solid (12 mg, yield 21%). LC-MS (ESI) m/z: 468 [M+H]⁺. ¹H NMR (400 MHz, CD₃OD) δ 8.55 (d, J=5.3 Hz, 1H), 8.22-8.10 (m, 2H), 7.62 (ddd, J=48.0, 34.1, 7.9 Hz, 6H), 6.64 (dd, J=16.8, 10.4 Hz, 1H), 6.33 (dd, J=16.8, 1.7 Hz, 1H), 5.87-5.74 (m, 1H), 5.49-5.36 (m, 1H), 4.77 (d, J=4.0 Hz, 2H), 4.64 (dd, J=21.9, 5.2 Hz, 1H), 4.42-3.98 (m, 3H), 3.93-3.73 (m, 1H).

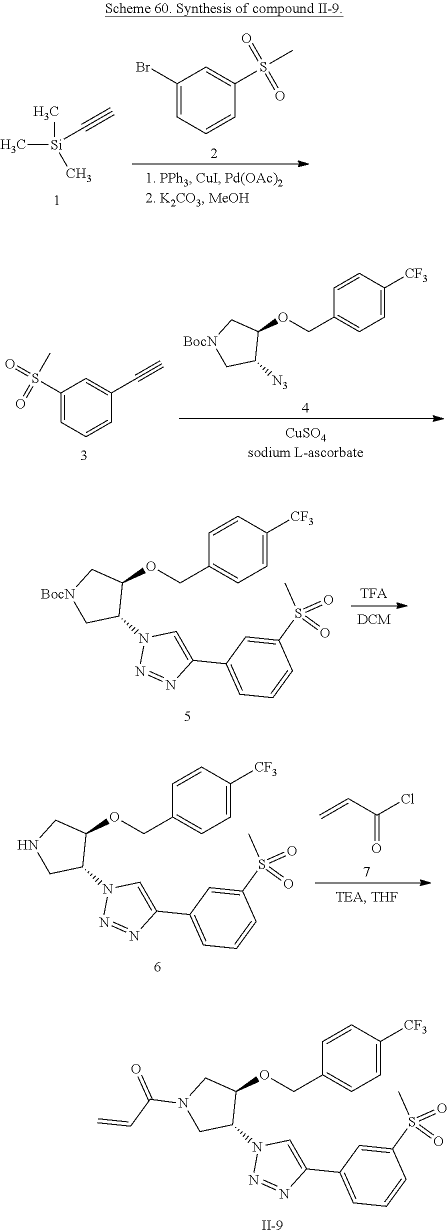

### Step 1: Synthesis of 1-ethynyl-3-(methylsulfonyl)benzene (Compound 3)

To the solution of 1-bromo-3-(methylsulfonyl)benzene (2.06 g, 8.76 mmol) in Et₃N (10 mL) was added ethynyltrimethylsilane (2 mL), PPh₃ (0.122 g, 1.2 mmol), CuI (0.079 g, 0.42 mmol) and Pd(OAc)₂ (0.093 g, 0.42 mmol). The mixture was stirred at 80° C. under N₂ for 1 h. The mixture was diluted with water (100 mL) and extracted with EtOAc (3×50 mL), the combined organic was dried over anhydrous Na₂SO₄, concentrated and purified by flash column chromatography on silica gel (ethyl acetate in petroleum ether=30% v/v) to obtain the target compound (3-methanesulfonyl-phenylethynyl)-trimethylsilane (2.1 g). To a solution of (3-methanesulfonyl-phenylethynyl)-trimethylsilane (2.1 g, 8.01 mmol) in MeOH (20 mL) was added K₂CO₃ (1.45 g, 10.5 mmol), the mixture was stirred at RT for 1 h. The mixture was diluted with water (100 mL) and extracted with EtOAc (3×50 mL), the combined organic was washed with ammonium chloride (50 mL) and brine (50 mL), dried over anhydrous Na₂SO₄, concentrated and purified by flash column chromatography on silica gel (ethyl acetate in petroleum ether=50% v/v) to obtain the target compound 3 as solid (1.2 g, yield 65.6%). LC-MS (ESI) m/z: 181 [M+H]⁺.

### Step 2: Synthesis of tert-butyl trans-3-(4-(3-(methylsulfonyl)phenyl)-1H-1,2,3-triazol-1-yl)-4-(4-(trifluoromethyl)benzyloxy)pyrrolidine-1-carboxylate (Compound 5)

To the solution of tert-butyl trans-3-azido-4-(4-(trifluoromethyl)benzyloxy)pyrrolidine-1-carboxylate (250 mg, 0.65 mmol) in THF (5 mL), H₂O (5 mL) and BuOH (5 mL) was added 1-ethynyl-3-(methylsulfonyl)benzene (174 mg, 0.97 mmol), CuSO₄ (16 mg, 0.065 mmol) and sodium L-ascorbate (25 mg, 0.13 mmol). The mixture was stirred at 70° C. under N₂ for 5 h. The mixture was concentrated and purified by flash column chromatography on silica gel (methanol in methylene chloride=10% v/v) to obtain the target compound 5 as solid (350 mg, yield 44.5%). LC-MS (ESI) m/z: 567 [M+H]⁺.

### Step 3: Synthesis of 4-(3-(methylsulfonyl)phenyl)-1-(trans-4-(4-(trifluoromethyl)benzyloxy)pyrrolidin-3-yl)-1H-1,2,3-triazole (Compound 6)

The mixture of tert-butyl trans-3-azido-4-(4-(trifluoromethyl)benzyloxy)pyrrolidine-1-carboxylate (300 mg, 0.53 mmol) and TFA (2 mL) in DCM (10 mL) was stirred at RT for 2 h. The mixture was concentrated and to leave crude compound 6 as oil (300 mg, crude). LC-MS (ESI) m/z: 467 [M+H]⁺.

### Step 4: Synthesis of 1-(trans-3-(4-(3-(methylsulfonyl)phenyl)-1H-1,2,3-triazol-1-yl)-4-(4-(trifluoromethyl)benzyloxy)pyrrolidin-1-yl)prop-2-en-1-one (Compound II-9)

To a solution of 4-(3-(methylsulfonyl)phenyl)-1-(trans-4-(4-(trifluoromethyl)benzyloxy)pyrrolidin-3-yl)-1H-1,2,3-triazole (250 mg, 0.54 mmol) in THF (10 mL) was added acryloyl chloride (49 mg, 0.54 mmol) and Et₃N (108 mg, 1.07 mmol). The mixture was stirred at 0° C. for 1 h. The resulting mixture was concentrated and purified by prep-HPLC to obtain the target compound II-9 as solid (246 mg, yield 88.5%). LC-MS (ESI) m/z: 521 [M+H]⁺. ¹H-NMR (400 MHz, DMSO-d6) δ (ppm) 8.94 (d, J=1.7 Hz, 1H), 8.37 (s, 1H), 8.20 (d, J=7.7 Hz, 1H), 7.91 (d, J=7.8 Hz, 1H), 7.80-7.65 (m, 3H), 7.54 (d, J=7.9 Hz, 2H), 6.64 (dd, J=16.8, 10.3 Hz, 1H), 6.20 (d, J=16.7 Hz, 1H), 5.74 (d, J=10.1 Hz, 1H), 5.56-5.40 (m, 1H), 4.82-4.71 (m, 2H), 4.60 (d, J=26.5 Hz, 1H), 4.25 (dt, J=11.7, 9.6 Hz, 1H), 4.10-3.58 (m, 3H), 3.27 (s, 3H).

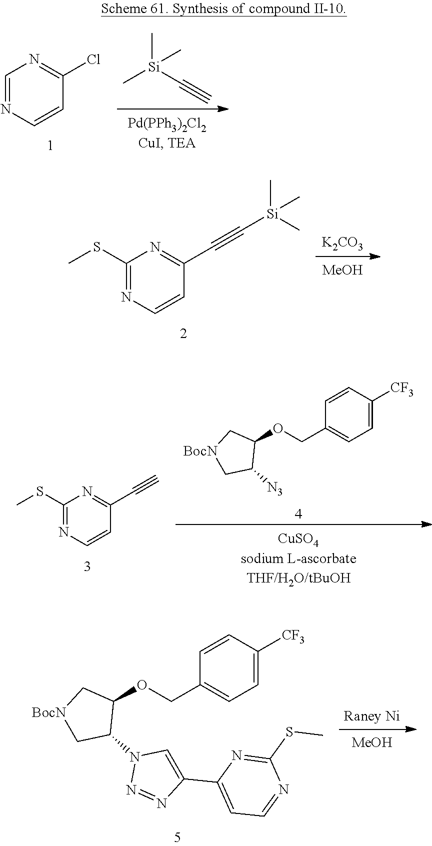

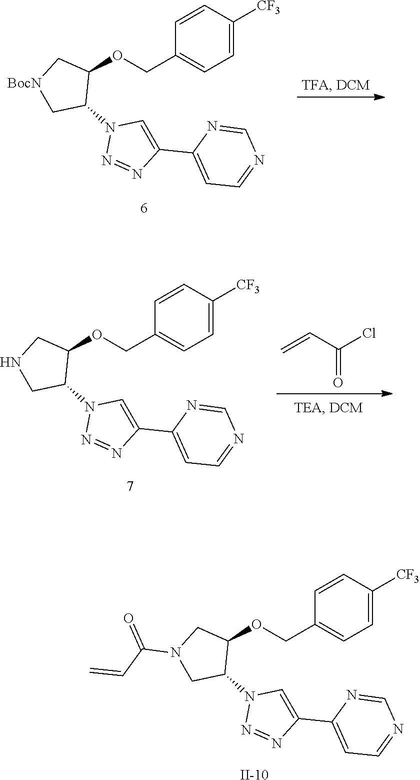

### Step 1: Synthesis of 2-(methylthio)-4-((trimethylsilyl)ethynyl)pyrimidine (Compound 2)

A mixture of 4-chloropyrimidine (900 mg, 5.6 mmol), ethynyltrimethylsilane (815 mg, 8.3 mmol), CuI (212 mg, 1.1 mmol), Pd(PPh₃)₂Cl₂ (196 mg, 0.28 mmol) and TEA (2828 mg, 28 mmol) in DMF (10 mL) was stirred at 70° C. under N₂ overnight. The mixture was concentrated and purified by flash column chromatography on silica gel (ethyl acetate in petroleum ether=20% v/v) to obtain compound 2 as yellow solid (900 mg, yield 72%). LC-MS (ESI) m/z: 223 [M+H]⁺.

### Step 2: Synthesis of 4-ethynyl-2-(methylthio)pyrimidine (Compound 3)

A mixture of compound 2 (500 mg, 2.25 mmol) and K₂CO₃ (340 mg, 2.45 mmol) in MeOH (20 mL) was stirred at 0° C. for 3 hours. The mixture was concentrated in vacuum, the residue was diluted with water (100 mL) and extracted with ethyl acetate (60 mL), the organic was washed with water (40 mL), dried over anhydrous Na₂SO₄, filtered and concentrated to leave crude compound 3 as yellow solid (200 mg, yield 59%). LC-MS (ESI) m/z: 151 [M+H]⁺.

### Step 3: Synthesis of tert-butyl trans-3-(4-(2-(methylthio)pyrimidin-4-yl)-1H-1,2,3-triazol-1-yl)-4-(4-(trifluoromethyl)benzyloxy)pyrrolidine-1-carboxylate (Compound 5)

A mixture of compound 3 (180 mg, 1.2 mmol), tert-butyl trans-3-azido-4-((4-(trifluoromethyl)benzyl)oxy)pyrrolidine-1-carboxylate (463 mg, 1.2 mmol), CuSO₄ (179 mg, 0.72 mmol) and sodium L-ascorbate (140 mg, 0.36 mmol) in THF (2 mL), H₂O (2 mL) and nBuOH (2 mL) was stirred at 60° C. under N₂ overnight. The mixture was diluted with water (100 mL) and extracted with ethyl acetate (60 mL), the organic was washed with water (40 mL), dried over anhydrous Na₂SO₄, concentrated and purified by flash column chromatography on silica gel (ethyl acetate in petroleum ether=60% v/v) to obtain compound 5 as yellow solid (500 mg, yield 78%). LC-MS (ESI) m/z: 537 [M+H]⁺.

### Step 4: Synthesis of tert-butyl trans-3-(4-(pyrimidin-4-yl)-1H-1,2,3-triazol-1-yl)-4-(4-(trifluoromethyl)benzyloxy)pyrrolidine-1-carboxylate (Compound 6)

A mixture of compound 5 (250 mg, 0.46 mmol) and Raney Ni (250 mg, 4.3 mmol) in EtOH (10 mL) was stirred at 70° C. under N₂ overnight. The mixture was filtered, the filtrate was concentrated to leave crude compound 6 as yellow solid (180 mg, yield 79%). LC-MS (ESI) m/z: 491 [M+H]⁺.

### Step 5: Synthesis of 4-(1-(trans-4-(4-(trifluoromethyl)benzyloxy)pyrrolidin-3-yl)-1H-1,2,3-triazol-4-yl)pyrimidine (Compound 7)

A mixture of compound 6 (60 mg, 0.1 mmol) and TFA (1 mL) in DCM (3 mL) was stirred at rt for 2 hours. The mixture was concentrated to leave crude compound 7 as oil (50 mg, yield 100%). LC-MS (ESI) m/z: 391 [M+H]⁺.

### Step 6: Synthesis of 1-(trans-3-(4-(pyrimidin-4-yl)-1H-1,2,3-triazol-1-yl)-4-(4-(trifluoromethyl)benzyloxy)pyrrolidin-1-yl)prop-2-en-1-one (II-10)

A mixture of compound 7 (45 mg, 0.09 mmol), acryloyl chloride (9 mg, 0.09 mmol) and TEA (20 mg, 0.18 mmol) in DCM (5 mL) was stirred at rt for 2 hours. The mixture was concentrated and purified by prep-HPLC to obtain compound II-10 as white solid (15 mg, yield 37%). LC-MS (ESI) m/z: 445 [M+H]⁺. ¹H NMR (400 MHz, CD₃OD) δ (ppm) 9.17 (d, J=1.2 Hz, 1H), 8.85 (d, J=5.0 Hz, 1H), 8.76 (d, J=5.0 Hz, 1H), 8.12 (dd, J=4.4, 1.2 Hz, 1H), 7.63 (d, J=8.1 Hz, 2H), 7.53 (d, J=8.1 Hz, 2H), 6.66 (dd, J=16.8, 10.4 Hz, 1H), 6.35 (d, J=16.8 Hz, 1H), 5.82 (ddd, J=10.4, 5.4, 1.9 Hz, 1H), 5.60-5.44 (m, 1H), 4.79 (d, J=3.4 Hz, 2H), 4.73-4.62 (m, 1H), 4.46-3.74 (m, 4H).

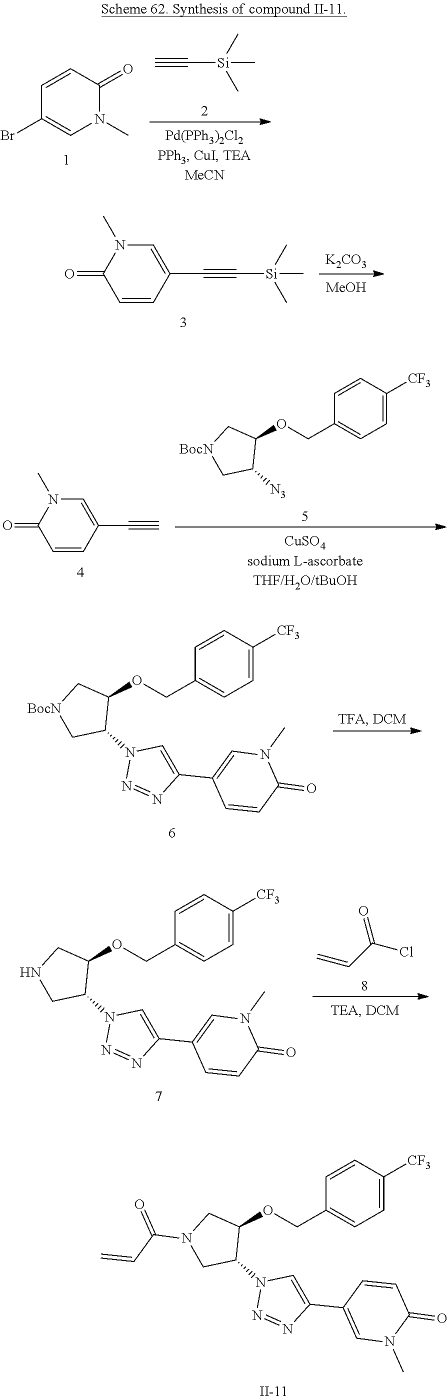

### Step 1: Synthesis of 1-methyl-5-((trimethylsilyl)ethynyl)pyridin-2(1H)-one (Compound 3)

A mixture of 5-bromo-1-methylpyridin-2(1H)-one (500 mg, 2.65 mmol), ethynyltrimethylsilane (275 mg, 2.78 mmol), Pd(PPh₃)₂Cl₂ (105 mg, 0.15 mmol), PPh₃ (140 mg, 0.53 mmol), CuI (30 mg, 0.15 mmol) and Et₃N (535 mg, 5.30 mmol) in MeCN (20 mL) was stirred at 120° C. under N₂ for 1 hour. The mixture was concentrated and purified by flash column chromatography on silica gel (ethyl acetate in petroleum ether=50% v/v) to obtain the target compound 3 as oil (500 mg, yield 90.7%). LC-MS (ESI) m/z: 206 [M+H]⁺.

### Step 2: Synthesis of 5-ethynyl-1-methylpyridin-2(1H)-one (Compound 4)

A mixture of 1-methyl-5-((trimethylsilyl)ethynyl)pyridin-2(1H)-one (400 mg, 1.96 mmol) and K₂CO₃ (539 mg, 3.90 mmol) in MeOH (20 mL) was stirred at rt for 2 hours. The mixture diluted with water (100 mL) and extracted with ethyl acetate (100 mL), the organic was washed with water (50 mL), dried over anhydrous Na₂SO₄, concentrated and purified by flash column chromatography on silica gel (ethyl acetate in petroleum ether=50% v/v) to obtain the target compound 4 as solid (250 mg, yield 96.5%). LC-MS (ESI) m/z: 134 [M+H]⁺.

### Step 3: Synthesis of tert-butyl trans-3-(4-(1-methyl-6-oxo-1,6-dihydropyridin-3-yl)-1H-1,2,3-triazol-1-yl)-4-(4-(trifluoromethyl)benzyloxy)pyrrolidine-1-carboxylate (Compound 6)

To a solution of tert-butyl trans-3-azido-4-(4-(trifluoromethyl)benzyloxy)pyrrolidine-1-carboxylate (200 mg, 0.52 mmol) in THF (10 mL), H₂O (10 mL) and BuOH (10 mL) was added 5-ethynyl-1-methylpyridin-2(1H)-one (100 mg, 0.78 mmol), CuSO₄ (16 mg, 0.052 mmol) and sodium L-ascorbate (20 mg, 0.104 mmol). The mixture was stirred at 70° C. for 16 hours. After cooled down to rt the mixture was diluted with water (100 mL) and extracted with EtOAc (3×50 mL), the combined organics were washed with brine (100 mL), dried over anhydrous Na₂SO₄, filtered and concentrated to leave crude compound 6 as oil (200 mg, yield 74.3%). LC-MS (ESI) m/z: 520 [M+H]⁺.

### Step 4: Synthesis of 1-methyl-5-(1-(trans-4-(4-(trifluoromethyl)benzyloxy)pyrrolidin-3-yl)-1H-1,2,3-triazol-4-yl)pyridin-2(1H)-one (Compound 7)

A mixture of tert-butyl trans-3-(4-(1-methyl-6-oxo-1,6-dihydropyridin-3-yl)-1H-1,2,3-triazol-1-yl)-4-(4-(trifluoromethyl)benzyloxy)pyrrolidine-1-carboxylate (180 mg, 0.25 mmol) and TFA (2 mL) in DCM (10 mL) was stirred at rt for 2 hours. The mixture was basified to pH˜8 with NaHCO₃ solution and extracted with EtOAc (3×50 mL), the combined organics were washed with brine (100 mL), dried over anhydrous Na₂SO₄, filtered and concentrated to leave crude compound 7 as oil (150 mg, yield 93.1%). LC-MS (ESI) m/z: 420 [M+H]⁺.

### Step 5: Synthesis of 5-(1-(trans-1-acryloyl-4-(4-(trifluoromethyl)benzyloxy)pyrrolidin-3-yl)-1H-1,2,3-triazol-4-yl)-1-methylpyridin-2(1H)-one (II-11)

To the solution of 1-methyl-5-(1-(trans-4-(4-(trifluoromethyl)benzyloxy)pyrrolidin-3-yl)-1H-1,2,3-triazol-4-yl)pyridin-2(1H)-one (130 mg, 0.31 mmol) in THF (10 mL) was added acryloyl chloride (28 mg, 0.31 mmol) and Et₃N (64 mg, 0.62 mmol). The mixture was stirred at 0° C. for 1 hour, and then concentrated and purified by prep-HPLC to obtain the target compound II-11 as solid (148 mg, yield 87.5%). LC-MS (ESI) m/z: 474 [M+H]⁺. 1H ¹H NMR (400 MHz, DMSO-d6) δ (ppm) 8.50 (d, J=3.5 Hz, 1H), 8.25 (d, J=2.3 Hz, 1H), 7.85 (dd, J=9.4, 1.6 Hz, 1H), 7.70 (d, J=8.0 Hz, 2H), 7.54 (d, J=8.0 Hz, 2H), 6.64 (dd, J=16.7, 10.3 Hz, 1H), 6.51 (d, J=9.4 Hz, 1H), 6.20 (dt, J=16.8, 2.0 Hz, 1H), 5.74 (ddd, J=10.3, 5.2, 2.3 Hz, 1H), 5.55-5.38 (m, 1H), 4.84-4.70 (m, 2H), 4.54 (ddd, J=12.7, 8.7, 3.6 Hz, 1H), 4.21 (ddd, J=15.8, 11.6, 5.6 Hz, 1H), 4.11-4.03 (m, 1H), 4.01-3.60 (m, 2H), 3.51 (s, 3H).

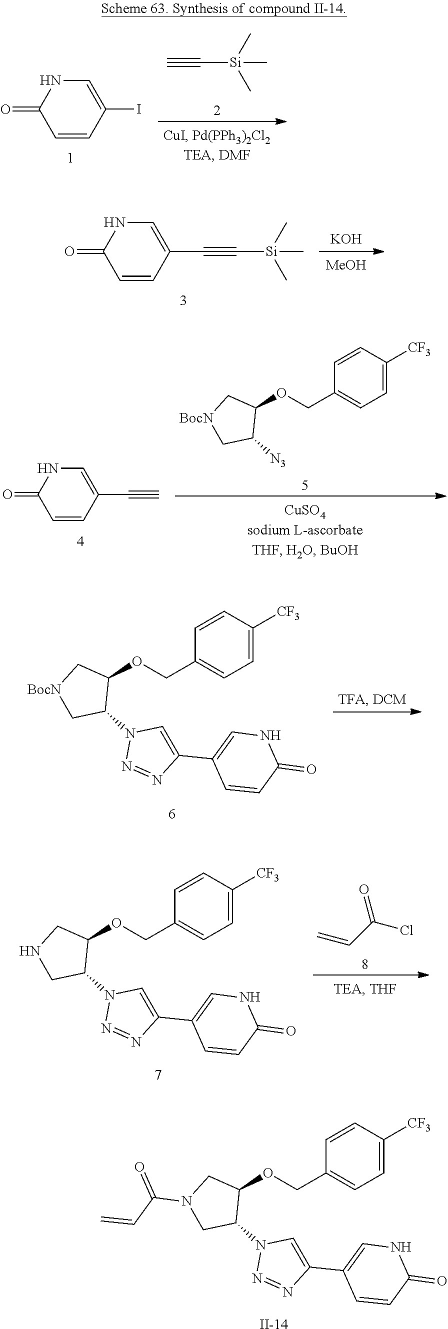

### Step 1: Synthesis of 5-((trimethylsilyl)ethynyl)pyridin-2(1H)-one (Compound 3)

The mixture of 5-iodopyridin-2(1H)-one (1 g, 4.5 mmol), ethynyltrimethylsilane (0.465 g, 4.75 mmol), Pd(PPh₃)₂Cl₂ (0.158 mg, 0.225 mmol), CuI (0.043 g, 0.225 mmol) and Et₃N (0.909 g, 9.0 mmol) in DMF (10 mL) was stirred at 90° C. under N₂ for 2 h. The resulting mixture was diluted with water (100 mL) and extracted with EtOAc (3×50 mL), the combined organics were washed with brine (100 mL), dried over anhydrous Na₂SO₄, filtered and concentrated to leave crude compound 3 as oil (1 g, crude). LC-MS (ESI) m/z: 192 [M+H]⁺.

### Step 2: Synthesis of 5-ethynylpyridin-2(1H)-one (Compound 4)

To a solution of 5-((trimethylsilyl)ethynyl)pyridin-2(1H)-one (900 mg, 4.71 mmol) in MeOH (20 mL) was added KOH (528 mg, 9.42 mmol). The mixture was stirred at RT for 2 h., concentrated and purified by flash column chromatography on silica gel (ethyl acetate in petroleum ether=80% v/v) to obtain compound 4 as oil (400 mg, yield 64.2%). LC-MS (ESI) m/z: 120 [M+H]⁺.

### Step 3: Synthesis of tert-butyl trans-3-(4-(6-oxo-1,6-dihydropyridin-3-yl)-1H-1,2,3-triazol-1-yl)-4-(4-(trifluoromethyl)benzyloxy)pyrrolidine-1-carboxylate (Compound 6)

To a solution of 5-ethynylpyridin-2(1H)-one (350 mg, 0.913 mmol) in THF (5 mL), H₂O (5 mL) and BuOH (5 mL) was added tert-butyl trans-3-azido-4-((4-(trifluoromethyl)benzyl)oxy)pyrrolidine-1-carboxylate (162 mg, 1.36 mmol), CuSO₄ (23 mg, 0.09 mmol) and sodium L-ascorbate (36 mg, 0.18 mmol). The mixture was stirred at 70° C. for 16 h., concentrated and purified by flash column chromatography on silica gel (Dichloromethane in methanol=20% v/v) to obtain the target compound 6 as solid (100 mg, yield 6.7%). LC-MS (ESI) m/z: 505 [M+H]⁺.

### Step 4: Synthesis of 5-(1-(trans-4-(4-(trifluoromethyl)benzyloxy)pyrrolidin-3-yl)-1H-1,2,3-triazol-4-yl)pyridin-2(1H)-one (Compound 7)

A mixture of tert-butyl trans-3-(4-(6-oxo-1,6-dihydropyridin-3-yl)-1H-1,2,3-triazol-1-yl)-4-(4-(trifluoromethyl)benzyloxy)pyrrolidine-1-carboxylate (80 mg, 0.16 mmol) and TFA (2 mL) in DCM (10 mL) was stirred at RT for 2 h. The resulting mixture was concentrated to leave crude compound 7 as oil (70 mg, crude). LC-MS (ESI) m/z: 406 [M+H]⁺.

### Step 5: Synthesis of 5-(1-(trans-1-acryloyl-4-(4-(trifluoromethyl)benzyloxy)pyrrolidin-3-yl)-1H-1,2,3-triazol-4-yl)pyridin-2(1H)-one (II-14)

To a solution of 5-(1-(trans-4-(4-(trifluoromethyl)benzyloxy)pyrrolidin-3-yl)-1H-1,2,3-triazol-4-yl)pyridin-2(1H)-one (50 mg, 0.12 mmol) in THF (10 mL) was added acryloyl chloride (11 mg, 0.12 mmol) and Et₃N (25 mg, 0.25 mmol). The mixture was stirred at 0° C. for 1 h., concentrated and purified by prep-HPLC to obtain the target compound II-14 as solid (7 mg, yield 12.3%). LC-MS (ESI) m/z: 460 [M+H]⁺. ¹H NMR (400 MHz, CD₃OD) δ 8.21 (d, J=4.5 Hz, 1H), 7.91-7.81 (m, 2H), 7.52 (d, J=8.1 Hz, 2H), 7.41 (d, J=8.1 Hz, 2H), 6.60-6.48 (m, 2H), 6.23 (dd, J=16.8, 1.9 Hz, 1H), 5.76-5.67 (m, 1H), 5.35-5.24 (m, 1H), 4.66 (d, J=3.6 Hz, 2H), 4.60-4.43 (m, 2H), 4.31-3.59 (m, 4H).

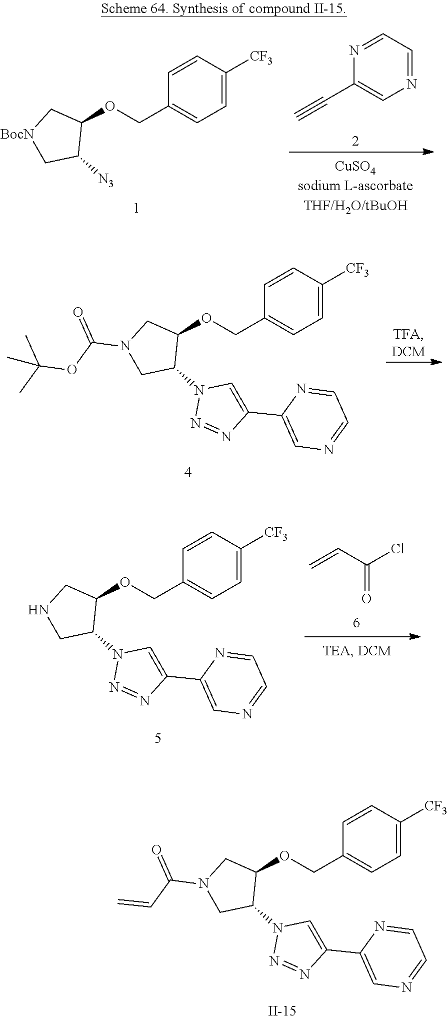

### Step 1: Synthesis of tert-butyl trans-3-(4-(pyrazin-2-yl)-1H-1,2,3-triazol-1-yl)-4-(4-(trifluoromethyl)benzyloxy)pyrrolidine-1-carboxylate (Compound 4)

A mixture of tert-butyl trans-3-azido-4-((4-(trifluoromethyl)benzyl)oxy)pyrrolidine-1-carboxylate (200 mg, 0.42 mmol), 2-ethynylpyrazine (64 mg, 0.6 mmol), CuSO₄ (12 mg, 0.042 mmol) and sodium L-ascorbate (20 mg, 0.104 mmol) in THF (1 mL), H₂O (1 mL) and nBuOH (1 mL) was stirred at 60° C. under N₂ overnight. The mixture was diluted with water (100 mL) and extracted with ethyl acetate (60 mL), the organic was washed with water (40 mL), dried over anhydrous Na₂SO₄, concentrated and purified by flash column chromatography on silica gel (ethyl acetate in petroleum ether=80% v/v) to obtain compound 4 as oil (200 mg, yield 97%). LC-MS (ESI) m/z: 491 [M+H]⁺.

### Step 2: Synthesis of 2-(1-(trans-4-(4-(trifluoromethyl)benzyloxy)pyrrolidin-3-yl)-1H-1,2,3-triazol-4-yl) pyrazine (Compound 5)

A mixture of compound 4 (100 mg, 0.2 mmol) and TFA (1 mL) in DCM (3 mL) was stirred at rt for 2 hours. The mixture was concentrated to leave crude compound 5 as yellow oil (80 mg, yield 100%). LC-MS (ESI) m/z: 391 [M+H]⁺.

### Step 3: Synthesis of 1-(trans-3-(4-(pyrazin-2-yl)-1H-1,2,3-triazol-1-yl)-4-(4-(trifluoromethyl)benzyloxy)pyrrolidin-1-yl)prop-2-en-1-one (II-15)

A mixture of compound 5 (70 mg, 0.21 mmol), acryloyl chloride (21 mg, 0.21 mmol) and TEA (42 mg, 0.42 mmol) in DCM (5 mL) was stirred at rt under N₂ for 2 hours. The mixture was concentrated and purified by prep-HPLC to obtain compound II-15 as white solid (30 mg, yield 32%). LC-MS (ESI) m/z: 445 [M+H]⁺. ¹H NMR (400 MHz, CD₃OD) δ (ppm) 9.27 (s, 1H), 8.74-8.55 (m, 3H), 7.62 (d, J=8.1 Hz, 4H), 7.55 (d, J=8.1 Hz, 4H), 6.67 (dd, J=16.8, 10.4 Hz, 1H), 6.40-6.30 (m, 1H), 5.82 (ddd, J=10.5, 5.0, 1.9 Hz, 1H), 5.56-5.43 (m, 1H), 4.82-4.64 (m, 3H), 4.43-4.04 (m, 3H), 3.94-3.76 (m, 1H).

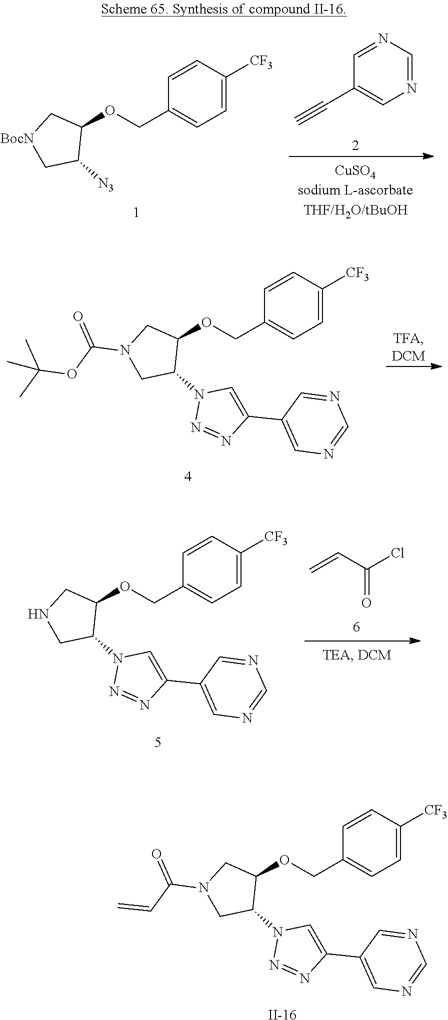

### Step 1: Synthesis of tert-butyl trans-3-(4-(pyrimidin-5-yl)-1H-1,2,3-triazol-1-yl)-4-(4-(trifluoromethyl)benzyloxy)pyrrolidine-1-carboxylate (Compound 4)

A mixture of tert-butyl trans-3-azido-4-((4-(trifluoromethyl)benzyl)oxy)pyrrolidine-1-carboxylate (300 mg, 0.78 mmol), 5-ethynylpyrimidine (96 mg, 0.9 mmol), CuSO₄ (18 mg, 0.078 mmol) and sodium L-ascorbate (30 mg, 0.156 mmol) in THF (1 mL), H₂O (1 mL) and nBuOH (1 mL) was stirred at 60° C. under N₂ overnight. The mixture was diluted with water (100 mL) and extracted with ethyl acetate (60 mL), the organic was washed with water (40 mL), dried over anhydrous Na₂SO₄, concentrated and purified by flash column chromatography on silica gel (ethyl acetate in petroleum ether=80% v/v) to obtain compound 4 as oil (270 mg, yield 71%). LC-MS (ESI) m/z: 491 [M+H]⁺.

### Step 2: Synthesis of 5-(1-(trans-4-(4-(trifluoromethyl)benzyloxy)pyrrolidin-3-yl)-1H-1,2,3-triazol-4-yl)pyrimidine (Compound 5)

A mixture of compound 4 (100 mg, 0.2 mmol) and TFA (1 mL) in DCM (3 mL) was stirred at rt for 2 hours. The mixture was concentrated to leave crude compound 5 as yellow oil (80 mg, yield 100%). LC-MS (ESI) m/z: 391 [M+H]⁺.

### Step 3: Synthesis of 1-(trans-3-(4-(pyrimidin-5-yl)-1H-1,2,3-triazol-1-yl)-4-(4-(trifluoromethyl)benzyloxy)pyrrolidin-1-yl)prop-2-en-1-one (II-16)

A mixture of compound 5 (70 mg, 0.21 mmol), acryloyl chloride (21 mg, 0.21 mmol) and TEA (42 mg, 0.42 mmol) in DCM (5 mL) was stirred at rt under N₂ for 2 hours. The mixture was concentrated and purified by prep-HPLC to obtain compound II-16 as white solid (35 mg, yield 37%). LC-MS (ESI) m/z: 445 [M+H]⁺. ¹H NMR (400 MHz, CD₃OD) δ (ppm) 9.25 (s, 2H), 9.16 (s, 1H), 8.69 (d, J=5.4 Hz, 1H), 7.64 (d, J=8.1 Hz, 4H), 7.54 (d, J=8.1 Hz, 4H), 6.67 (dd, J=16.8, 10.4 Hz, 1H), 6.36 (dd, J=16.8, 1.9 Hz, 1H), 5.83 (ddd, J=10.4, 3.3, 1.9 Hz, 1H), 5.55-5.43 (m, 1H), 4.80 (d, J=3.5 Hz, 2H), 4.72-4.62 (m, 1H), 4.42-4.00 (m, 3H), 3.95-3.76 (m, 1H).

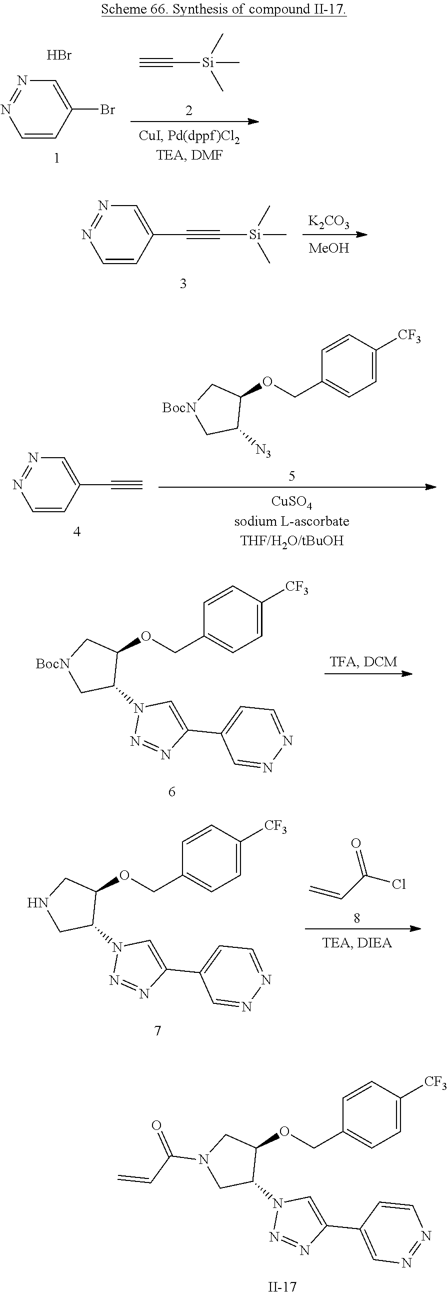

### Step 1: Synthesis of 4-((trimethylsilyl)ethynyl)pyridazine (Compound 3)

A mixture of 4-bromopyridazine hydrogen bromide (500 mg, 2.1 mmol), ethynyltrimethylsilane (305 mg, 3.15 mmol), CuI (80 mg, 0.4 mmol), Pd(dppf)Cl₂ (145 mg, 0.2 mmol) and TEA (100 mg, 10.5 mmol) in DMF (5 mL) was stirred at 70° C. under N₂ overnight. The mixture was diluted with water (100 mL) and extracted with ethyl acetate (50 mL×2), the combined organic was washed with brine (50 mL×2), dried over anhydrous Na₂SO₄, concentrated and purified by flash column chromatography on silica gel (ethyl acetate in petroleum ether=20% v/v) to obtain compound 3 as brine oil (100 mg, yield 27%). LC-MS (ESI) m/z: 177 [M+H]⁺.

### Step 2: Synthesis of 4-ethynylpyridazine (Compound 4)

A mixture of compound 3 (90 mg, 0.5 mmol) and K₂CO₃ (77 mg, 0.56 mmol) in MeOH (20 mL) was stirred at 0° C. for 3 h. The mixture was concentrated in vacuum, the residue was diluted with ethyl acetate (60 mL), washed with water (50 mL), dried over anhydrous Na₂SO₄, filtered and concentrated to obtain crude compound 4 as yellow solid (40 mg, yield 76%). LC-MS (ESI) m/z: 105 [M+H]⁺.

### Step 3: Synthesis of tert-butyl trans-3-(4-(pyridazin-4-yl)-1H-1,2,3-triazol-1-yl)-4-(4-(trifluoromethyl)benzyloxy)pyrrolidine-1-carboxylate (Compound 6)

A mixture of compound 4 (35 mg, 0.14 mmol), tert-butyl trans-3-azido-4-((4-(trifluoromethyl)benzyl)oxy)pyrrolidine-1-carboxylate (21 mg, 0.14 mmol), CuSO₄ (21 mg, 0.084 mmol) and sodium L-ascorbate (7 mg, 0.042 mmol) in THF (1 mL), H₂O (1 mL) and nBuOH (1 mL) was stirred at 70° C. under N₂ overnight. The mixture was diluted with water (100 mL) and extracted with ethyl acetate (50 mL), the organic was washed with water (50 mL), dried over anhydrous Na₂SO₄, concentrated and purified by flash column chromatography on silica gel (ethyl acetate in petroleum ether=70% v/v) to obtain compound 6 as brine oil (8 mg, yield 16%). LC-MS (ESI) m/z: 491 [M+H]⁺.

### Step 4: Synthesis of 4-(1-(trans-4-(4-(trifluoromethyl)benzyloxy)pyrrolidin-3-yl)-1H-1,2,3-triazol-4-yl)pyridazine (Compound 7)

A mixture of compound 6 (8 mg, 0.016 mmol) and TFA (0.5 mL) in DCM (1 mL) was stirred at rt for 2 h. The mixture was concentrated to leave crude compound 7 as oil (10 mg, yield 96%). LC-MS (ESI) m/z: 391 [M+H]⁺.

### Step 5: Synthesis of 1-(trans-3-(4-(pyridazin-4-yl)-1H-1,2,3-triazol-1-yl)-4-(4-(trifluoromethyl)benzyloxy)pyrrolidin-1-yl)prop-2-en-1-one (II-17)

A mixture of compound 7 (10 mg, 0.02 mmol), acryloyl chloride (2 mg, 0.02 mmol) and TEA (4 mg, 0.04 mmol) in DCM (1 mL) was stirred at rt for 2 h. The mixture was concentrated and purified by prep-HPLC to obtain compound II-17 as yellow solid (2 mg, yield 22%). LC-MS (ESI) m/z: 445 [M+H]⁺. ¹H NMR (400 MHz, CD₃OD) δ (ppm) 9.68 (s, 1H), 9.25 (d, J=5.4 Hz, 1H), 8.84 (d, J=5.4 Hz, 1H), 8.14 (dd, J=5.4, 2.2 Hz, 1H), 7.59 (dd, J=38.3, 8.1 Hz, 4H), 6.66 (dd, J=16.8, 10.4 Hz, 1H), 6.36 (dd, J=16.8, 1.9 Hz, 1H), 5.83 (ddd, J=10.5, 3.6, 1.9 Hz, 1H), 5.55-5.43 (m, 1H), 4.83-3.72 (m, 7H).

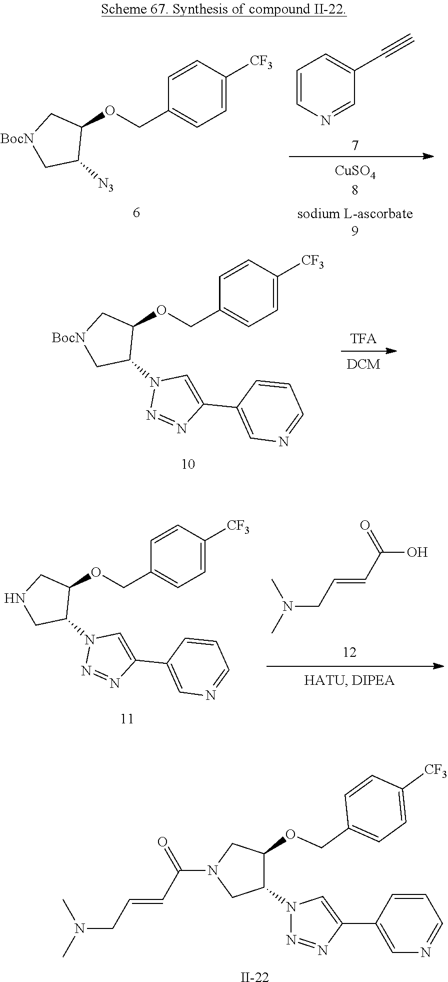

### Step 1: Synthesis of tert-butyl (3R,4R)-3-(4-(pyridin-3-yl)-1H-1,2,3-triazol-1-yl)-4-(4-(trifluoromethyl)benzyloxy)pyrrolidine-1-carboxylate (Compound 10)

A mixture of tert-butyl (3R,4R)-3-azido-4-((4-(trifluoromethyl)benzyl)oxy)pyrrolidine-1-carboxylate (200 mg, 0.877 mmol), 3-ethynylpyridine (90 mg, 0.877 mmol), CuSO₄ (131 mg, 0.5 mmol) and sodium L-ascorbate (50 mg, 0.25 mmol) in THF (2 mL), H₂O (2 mL) and n-BuOH (2 mL) was stirred at 70° C. under N₂ overnight. The reaction mixture was diluted with water (100 mL) and extracted with ethyl acetate (50 mL), the organic was washed with water (50 mL), dried over anhydrous Na₂SO₄, concentrated and purified by flash column chromatography on silica gel (ethyl acetate in petroleum ether=80% v/v) to obtain compound 10 as white solid (180 mg, yield 42%). LC-MS (ESI) m/z: 490 [M+H]⁺.

### Step 2: Synthesis of 3-(1-((3R,4R)-4-(4-(trifluoromethyl)benzyloxy)pyrrolidin-3-yl)-1H-1,2,3-triazol-4-yl)pyridine (Compound 11)

A mixture of compound 10 (170 mg, 0.34 mmol) and TFA (3 mL) in DCM (6 mL) was stirred at rt for 2 h. The mixture was concentrated to leave crude compound 11 as white solid (130 mg, yield 98%). LC-MS (ESI) m/z: 390 [M+H]⁺.

### Step 3: Synthesis of (E)-4-(dimethylamino)-1-((3R,4R)-3-(4-(pyridin-3-yl)-1H-1,2,3-triazol-1-yl)-4-(4-(trifluoromethyl)benzyloxy)pyrrolidin-1-yl)but-2-en-1-one (II-22)

A mixture of compound 11 (100 mg, 0.25 mmol), (E)-4-(dimethylamino) but-2-enoic acid (385 mg, 0.25 mmol), HATU (142 mg, 0.37 mmol) and TEA (50 mg, 0.5 mmol) in DCM (5 mL) was stirred at rt for 2 h. The mixture was concentrated and purified by prep-HPLC to obtain compound II-22 as white solid (60 mg, yield 48%). LC-MS (ESI) m/z: 501 [M+H]⁺. ¹H NMR (400 MHz, CD₃OD) δ (ppm) 9.03 (d, J=2.1 Hz, 1H), 8.65-8.51 (m, 2H), 8.35-8.25 (m, 1H), 7.66-7.50 (m, 5H), 6.91 (dt, J=15.2, 6.5 Hz, 1H), 6.51 (d, J=15.3 Hz, 1H), 5.53-5.38 (m, 1H), 4.80 (d, J=6.1 Hz, 2H), 4.67 (dd, J=24.5, 5.4 Hz, 1H), 4.44-3.99 (m, 3H), 3.96-3.75 (m, 1H), 3.21 (dd, J=8.7, 3.3 Hz, 2H), 2.30 (d, J=5.5 Hz, 6H).

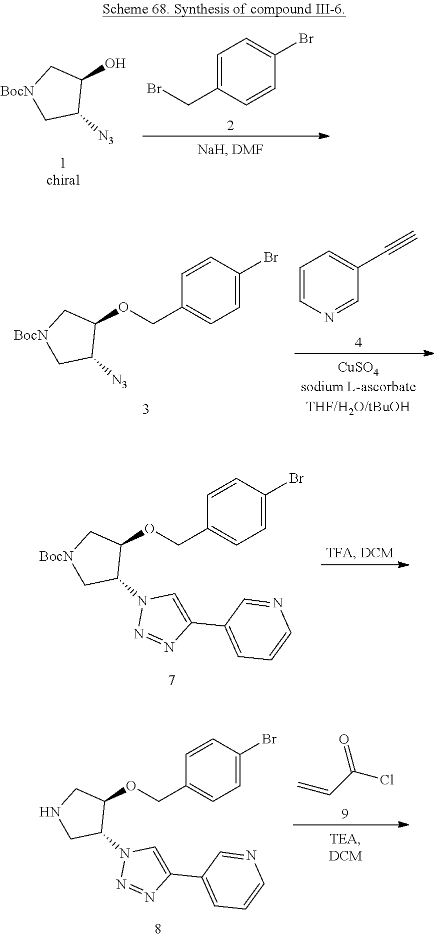

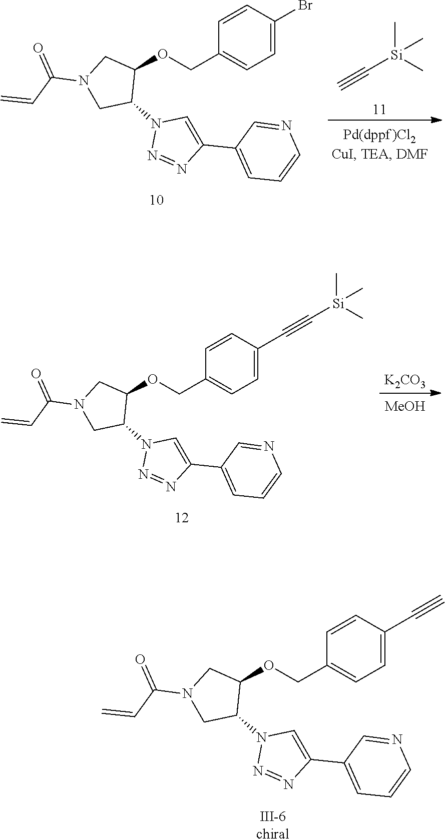

### Step 1: Synthesis of tert-butyl (3R,4R)-3-azido-4-(4-bromobenzyloxy)pyrrolidine-1-carboxylate (Compound 3)

To a solution of tert-butyl (3R,4R)-3-azido-4-hydroxypyrrolidine-1-carboxylate (350 mg, 1.5 mmol) in DMF (10 mL) was added NaH (60%, 72 mg, 1.8 mmol), the mixture was stirred at rt for 10 minutes, and then 1-bromo-4-(bromomethyl)benzene (380 mg, 1.5 mmol) was added. The mixture was stirred at rt under N₂ for 6 h., diluted with water (100 mL) and extracted with EtOAc (3×50 mL), the combined organics were washed with brine (100 mL), dried over anhydrous Na₂SO₄, filtered and concentrated to leave the crude compound 3 as yellow oil (500 mg, yield 83%). LC-MS (ESI) m/z: 297 [M+H−100]⁺.

### Step 2: Synthesis of tert-butyl (3R,4R)-3-(4-bromobenzyloxy)-4-(4-(pyridin-3-yl)-1H-1,2,3-triazol-1-yl)pyrrolidine-1-carboxylate (Compound 7)

A mixture of compound 3 (396 mg, 1.0 mmol), 3-ethynylpyridine (206 mg, 2.0 mmol), CuSO₄ (149 mg, 0.6 mmol) and sodium L-ascorbate (59 mg, 0.3 mmol) in THF (2 mL) and H₂O (2 mL) was stirred at 90° C. under N₂ overnight. The mixture was diluted with water (100 mL) and extracted with EtOAc (3×50 mL), the combined organics were washed with brine (100 mL), dried over anhydrous Na₂SO₄, concentrated and purified by prep-HPLC to obtain compound 7 as yellow solid (400 mg, yield 80%). LC-MS (ESI) m/z: 501 [M+H]⁺.

### Step 3: Synthesis of 3-(1-((3R,4R)-4-(4-bromobenzyloxy)pyrrolidin-3-yl)-1H-1,2,3-triazol-4-yl)pyridine (Compound 8)

A mixture of compound 7 (400 mg, 0.8 mmol) and TFA (2 mL) in DCM (3 mL) was stirred at 0° C. for 1 h. The mixture was concentrated to leave the crude compound 8 as yellow oil (300 mg, yield 94%). LC-MS (ESI) m/z: 401 [M+H]⁺.

### Step 4: Synthesis of 1-((3R,4R)-3-(4-bromobenzyloxy)-4-(4-(pyridin-3-yl)-1H-1,2,3-triazol-1-yl)pyrrolidin-1-yl)prop-2-en-1-one (Compound 10)

A mixture of compound 8 (300 mg, 0.75 mmol), acryloyl chloride (67 mg, 0.75 mmol) and TEA (151 mg, 1.5 mmol) in DCM (10 mL) was stirred at rt for 2 h. The mixture was concentrated to leave the crude compound 10 as yellow solid (300 mg, yield 88%). LC-MS (ESI) m/z: 455 [M+H]⁺.

### Step 5: Synthesis of 1-((3R,4R)-3-(4-(pyridin-3-yl)-1H-1,2,3-triazol-1-yl)-4-(4-((trimethylsilyl)ethynyl)benzyloxy)pyrrolidin-1-yl)prop-2-en-1-one (Compound 12)

A mixture of compound 10 (10 mg, 0.1 mmol), ethynyltrimethylsilane (15 mg, 0.15 mmol), CuI (5 mg, 0.02 mmol), Pd(dppf)Cl₂ (10 mg, 0.01 mmol) and TEA (50 mg, 0.5 mmol) in DMF (2 mL) was stirred at 120° C. under N₂ overnight. The mixture was concentrated and purified by flash column chromatography on silica gel (ethyl acetate in petroleum ether=90% v/v) to obtain compound 12 as brown solid (35 mg, yield 74%). LC-MS (ESI) m/z: 472 [M+H]⁺.

### Step 6: Synthesis of 1-((3R,4R)-3-(4-ethynylbenzyloxy)-4-(4-(pyridin-3-yl)-1H-1,2,3-triazol-1-yl)pyrrolidin-1-yl)prop-2-en-1-one (III-6)

A mixture of compound 12 (10 mg, 0.02 mmol) and K₂CO₃ (3 mg, 0.023 mmol) in MeOH (5 mL) was stirred at 0° C. for 3 h. The mixture was concentrated, the residue was diluted with water (20 mL) and extracted with ethyl acetate (20 mL), the organic was washed with water (15 mL), dried over anhydrous Na₂SO₄, concentrated and purified by prep-HPLC to obtain compound III-6 as white solid (1.5 mg, yield 19%). LC-MS (ESI) m/z: 400 [M+H]⁺. ¹H NMR (400 MHz, CD₃OD) δ (ppm): 9.00 (d, J=1.8 Hz, 1H), 8.53 (t, J=3.8 Hz, 2H), 8.27 (d, J=8.0 Hz, 1H), 7.53 (dd, J=7.9, 4.9 Hz, 1H), 7.40 (d, J=8.1 Hz, 2H), 7.30 (d, J=8.1 Hz, 2H), 6.63 (dd, J=16.8, 10.4 Hz, 1H), 6.33 (dd, J=16.8, 1.8 Hz, 1H), 5.80 (ddd, J=10.4, 5.1, 1.8 Hz, 1H), 5.48-5.31 (m, 1H), 4.72-3.98 (m, 6H), 3.90-3.69 (m, 1H), 3.45 (s, 1H).

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I′): 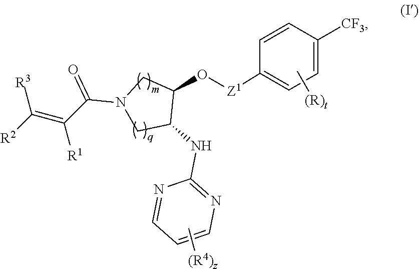
or a pharmaceutically acceptable salt, solvate, hydrate, polymorph, co-crystal, tautomer, stereoisomer, isotopically labeled derivative, prodrug, composition, or mixture thereof, wherein:
R, and R⁴ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{a}, —N(R^{a})₂, —SR^{a}, —C(═O)R^{a}, —C(═O)OR^{a}, —C(═O)N(R^{a})₂, —OC(═O)R^{a}, —N(R^{a})C(═O)R^{a}, —CN, and —NO₂;
Z¹ is optionally substituted alkylene, optionally substituted alkenylene, optionally substituted alkynylene, optionally substituted carbocyclylene, optionally substituted heterocyclylene, optionally substituted arylene, or optionally substituted heteroarylene; each occurrence of R^{a} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group, a sulfur protecting group, or a nitrogen protecting group, or two instances of R^{a}, when present, can be joined together with the heteroatom to which they are attached to form an optionally substituted heterocyclic ring
m is 1 or 2;
q is 1 or 2;
tis 0, 1, 2, 3, or 4;
z is 0, 1, 2, or 3.

2. (canceled)

3. The compound of claim 1, wherein Z¹ is of the formula: 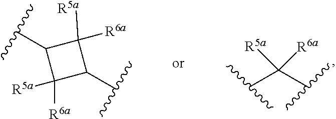
wherein R^{5a} and R^{6a} are each independently selected from hydrogen, halogen, optionally substituted alkyl; or optionally, R^{5a} and R^{6a} are joined together to form a carbocyclic or heterocyclic ring; and
R is halogen or H.

4-6. (canceled)

7. The compound of claim 1, wherein the compound of Formula I′ is a compound of the formula: 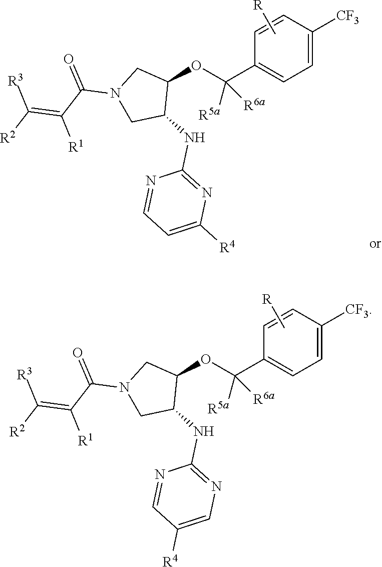
or a pharmaceutically acceptable salt, solvate, hydrate, polymorph, co-crystal, tautomer, stereoisomer, isotopically labeled derivative, or prodrug thereof.

8-31. (canceled)

32. The compound of claim 1, wherein R⁴ is hydrogen, halogen, —OR^{a}, or optionally substituted heterocyclyl; and
R^{a} is optionally substituted alkyl.

33-44. (canceled)

45. The compound of claim 1, wherein the compound of Formula I′ is 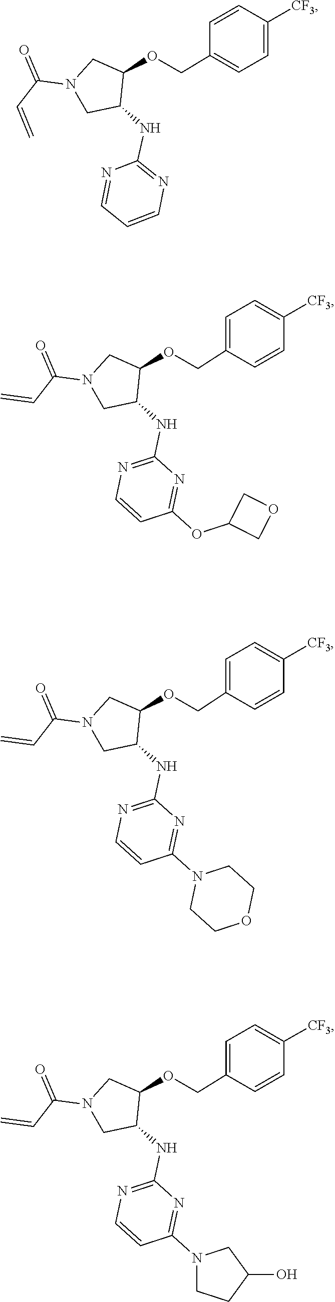 [SMILES: C=CC(=O)N1C[C@@H](Nc2ncccn2)[C@H](OCc2ccc(C(F)(F)F)cc2)C1 ; C=CC(=O)N1C[C@@H](Nc2nccc(OC3COC3)n2)[C@H](OCc2ccc(C(F)(F)F)cc2)C1 ; C=CC(=O)N1C[C@@H](Nc2nccc(N3CCOCC3)n2)[C@H](OCc2ccc(C(F)(F)F)cc2)C1 ; C=CC(=O)N1C[C@@H](Nc2nccc(N3CCC(O)C3)n2)[C@H](OCc2ccc(C(F)(F)F)cc2)C1] 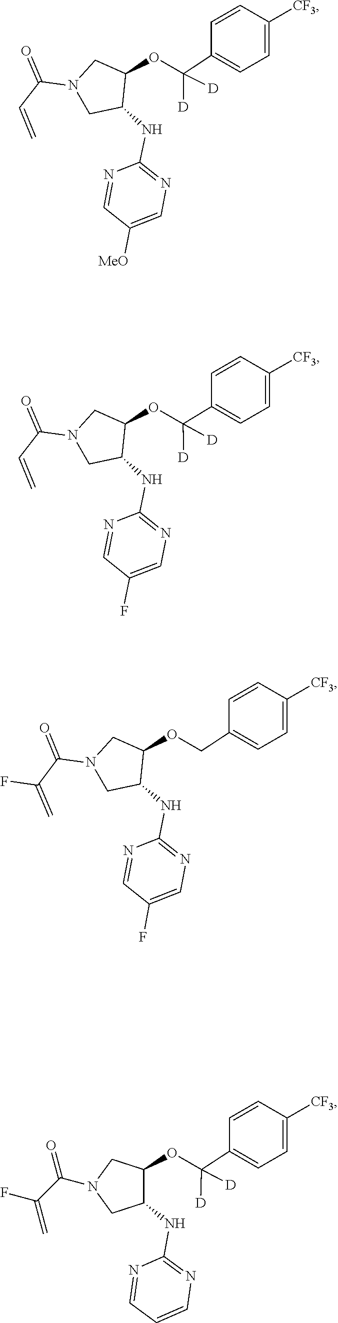 [SMILES: [2H]C([2H])(O[C@@H]1CN(C(=O)C=C)C[C@H]1Nc1ncc(OC)cn1)c1ccc(C(F)(F)F)cc1 ; [2H]C([2H])(O[C@@H]1CN(C(=O)C=C)C[C@H]1Nc1ncc(F)cn1)c1ccc(C(F)(F)F)cc1 ; C=C(F)C(=O)N1C[C@@H](Nc2ncc(F)cn2)[C@H](OCc2ccc(C(F)(F)F)cc2)C1 ; [2H]C([2H])(O[C@@H]1CN(C(=O)C(=C)F)C[C@H]1Nc1ncccn1)c1ccc(C(F)(F)F)cc1] 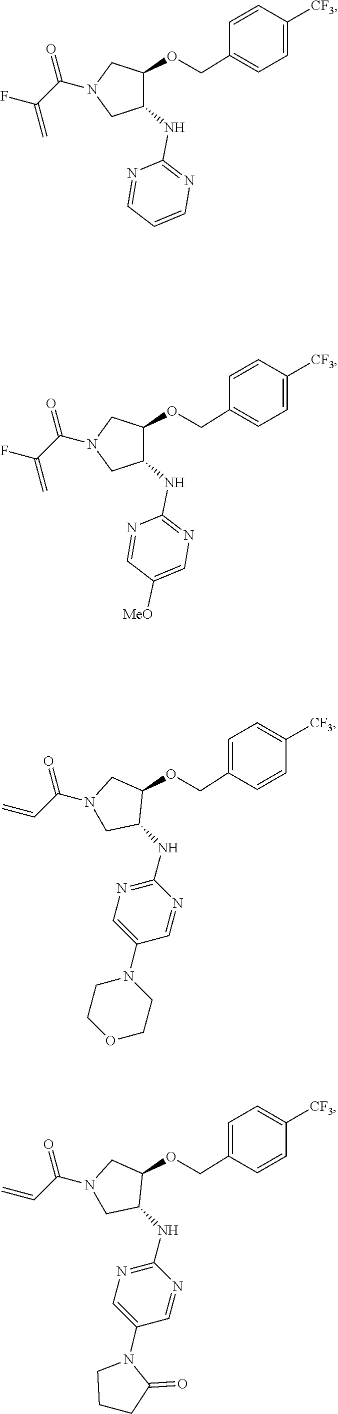 [SMILES: C=C(F)C(=O)N1C[C@@H](Nc2ncccn2)[C@H](OCc2ccc(C(F)(F)F)cc2)C1 ; C=C(F)C(=O)N1C[C@@H](Nc2ncc(OC)cn2)[C@H](OCc2ccc(C(F)(F)F)cc2)C1 ; C=CC(=O)N1C[C@@H](Nc2ncc(N3CCOCC3)cn2)[C@H](OCc2ccc(C(F)(F)F)cc2)C1 ; C=CC(=O)N1C[C@@H](Nc2ncc(N3CCCC3=O)cn2)[C@H](OCc2ccc(C(F)(F)F)cc2)C1] 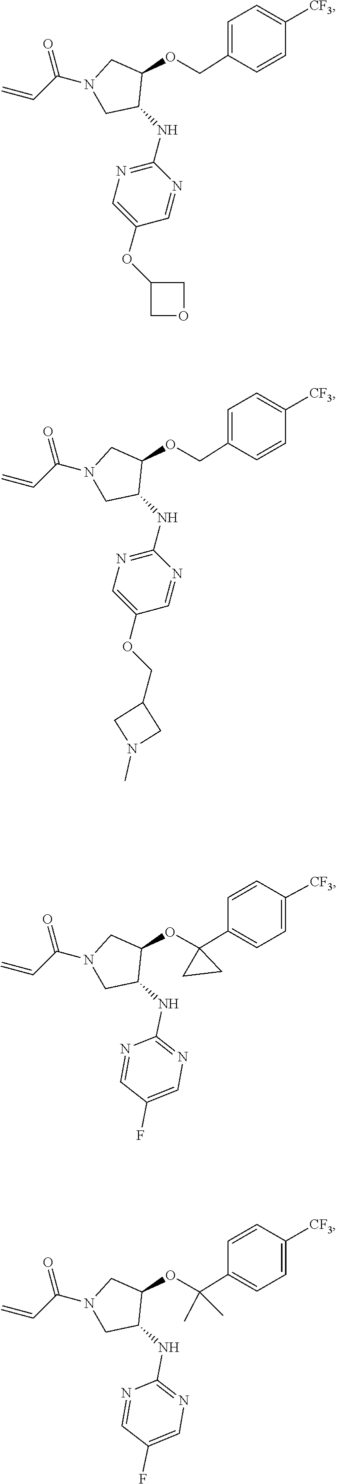 [SMILES: C=CC(=O)N1C[C@@H](Nc2ncc(OC3COC3)cn2)[C@H](OCc2ccc(C(F)(F)F)cc2)C1 ; C=CC(=O)N1C[C@@H](Nc2ncc(OCC3CN(C)C3)cn2)[C@H](OCc2ccc(C(F)(F)F)cc2)C1 ; C=CC(=O)N1C[C@@H](Nc2ncc(F)cn2)[C@H](OC2(c3ccc(C(F)(F)F)cc3)CC2)C1 ; C=CC(=O)N1C[C@@H](Nc2ncc(F)cn2)[C@H](OC(C)(C)c2ccc(C(F)(F)F)cc2)C1] 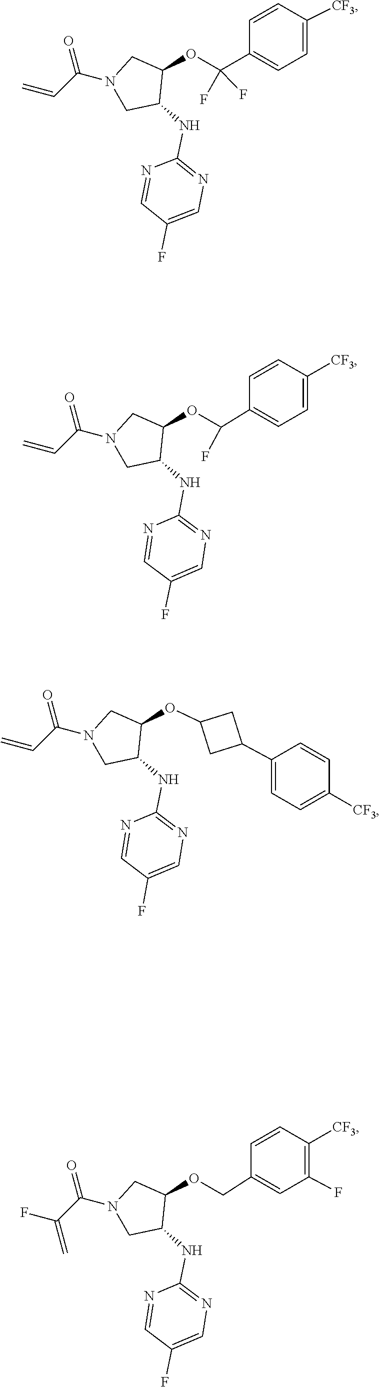 [SMILES: C=CC(=O)N1C[C@@H](Nc2ncc(F)cn2)[C@H](OC(F)(F)c2ccc(C(F)(F)F)cc2)C1 ; C=CC(=O)N1C[C@@H](Nc2ncc(F)cn2)[C@H](OC(F)c2ccc(C(F)(F)F)cc2)C1 ; C=CC(=O)N1C[C@@H](Nc2ncc(F)cn2)[C@H](OC2CC(c3ccc(C(F)(F)F)cc3)C2)C1 ; C=C(F)C(=O)N1C[C@@H](Nc2ncc(F)cn2)[C@H](OCc2ccc(C(F)(F)F)c(F)c2)C1] 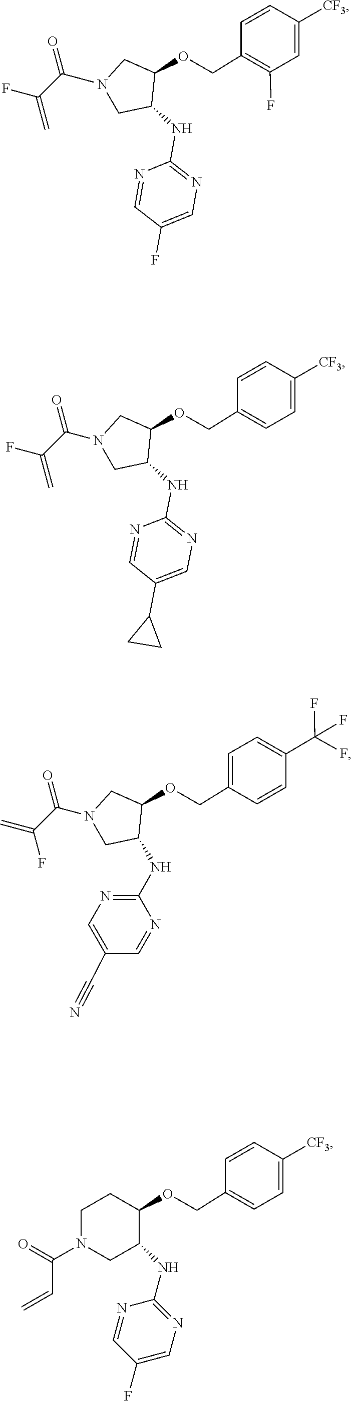 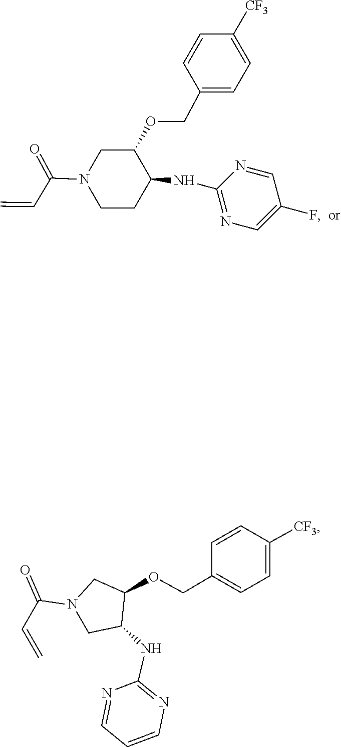
or a pharmaceutically acceptable salt, solvate, hydrate, polymorph, co-crystal, tautomer, stereoisomer, isotopically labeled derivative, or prodrug thereof.

46-48. (canceled)

49. A compound of Formula (II): 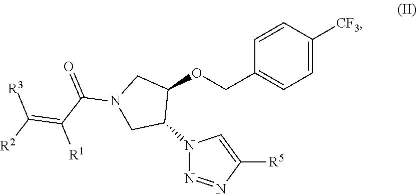
or a pharmaceutically acceptable salt, solvate, hydrate, polymorph, co-crystal, tautomer, stereoisomer, isotopically labeled derivative, or prodrug thereof, wherein:
R¹, R², and R³ are each independently selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{b}, —N(R^{b})₂, —SR^{b}, —C(═O)R^{b}, —C(═O)OR^{b}, —C(═O)N(R^{b})₂, —OC(═O)R^{b}, —N(R^{b})C(═O)R^{b}, —CN, and —NO₂;
R⁵ is selected from hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —ORD, —N(R^{b})₂, —SRD, —C(═O)RD, —C(═O)OR^{b}, —C(═O)N(R^{b})₂, —OC(═O)R^{b}, —N(R^{b})C(═O)R^{b}, —CN, and —NO₂;
each occurrence of R^{b} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{b}, when present, can be joined together with the heteroatom to which they are attached to form an optionally substituted heterocyclic ring
provided that the compound is not of the formula:

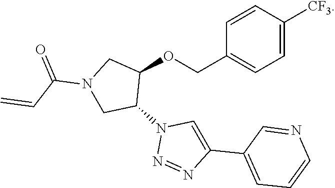 [SMILES: C=CC(=O)N1C[C@@H](n2cc(-c3cccnc3)nn2)[C@H](OCc2ccc(C(F)(F)F)cc2)C1]

50. The compound of claim 49, wherein R⁵ is optionally substituted heteroaryl, —C(═O)N(R^{b})₂, —N(R^{b})₂, optionally substituted C₁-C₆ alkyl, —CH₂N(R^{b})₂, optionally substituted aryl or optionally substituted heterocyclyl.

51-75. (canceled)

76. The compound of claim 49, wherein the compound is of the formula: 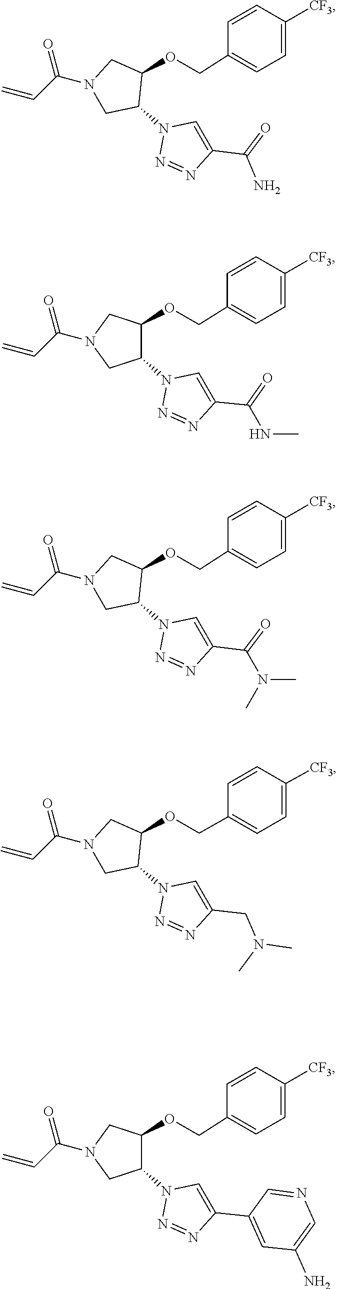 [SMILES: C=CC(=O)N1C[C@@H](n2cc(C(N)=O)nn2)[C@H](OCc2ccc(C(F)(F)F)cc2)C1 ; C=CC(=O)N1C[C@@H](n2cc(C(=O)NC)nn2)[C@H](OCc2ccc(C(F)(F)F)cc2)C1 ; C=CC(=O)N1C[C@@H](n2cc(C(=O)N(C)C)nn2)[C@H](OCc2ccc(C(F)(F)F)cc2)C1 ; C=CC(=O)N1C[C@@H](n2cc(CN(C)C)nn2)[C@H](OCc2ccc(C(F)(F)F)cc2)C1 ; C=CC(=O)N1C[C@@H](n2cc(-c3cncc(N)c3)nn2)[C@H](OCc2ccc(C(F)(F)F)cc2)C1] 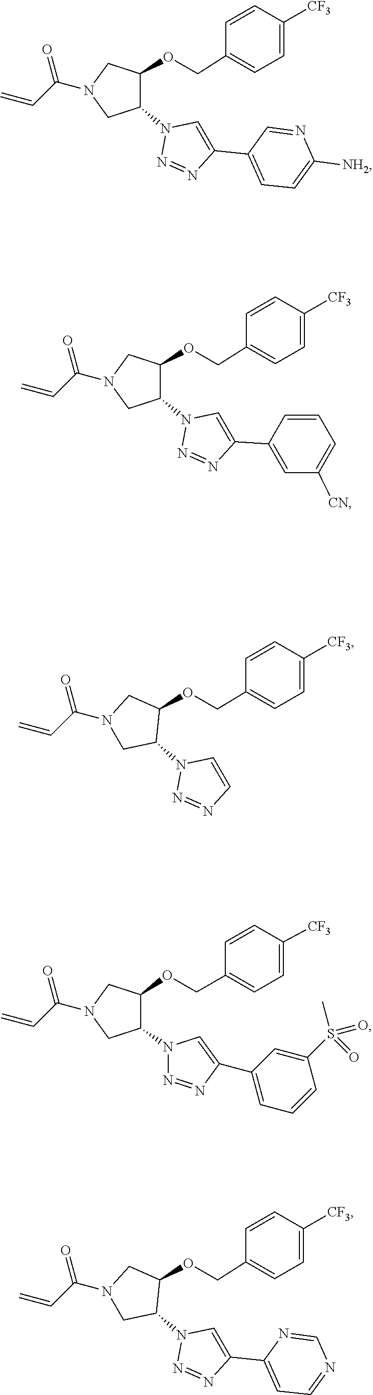 [SMILES: C=CC(=O)N1C[C@@H](n2cc(-c3ccc(N)nc3)nn2)[C@H](OCc2ccc(C(F)(F)F)cc2)C1 ; C=CC(=O)N1C[C@@H](n2cc(-c3cccc(C#N)c3)nn2)[C@H](OCc2ccc(C(F)(F)F)cc2)C1 ; C=CC(=O)N1C[C@@H](n2ccnn2)[C@H](OCc2ccc(C(F)(F)F)cc2)C1 ; C=CC(=O)N1C[C@@H](n2cc(-c3cccc(S(C)(=O)=O)c3)nn2)[C@H](OCc2ccc(C(F)(F)F)cc2)C1 ; C=CC(=O)N1C[C@@H](n2cc(-c3ccncn3)nn2)[C@H](OCc2ccc(C(F)(F)F)cc2)C1] 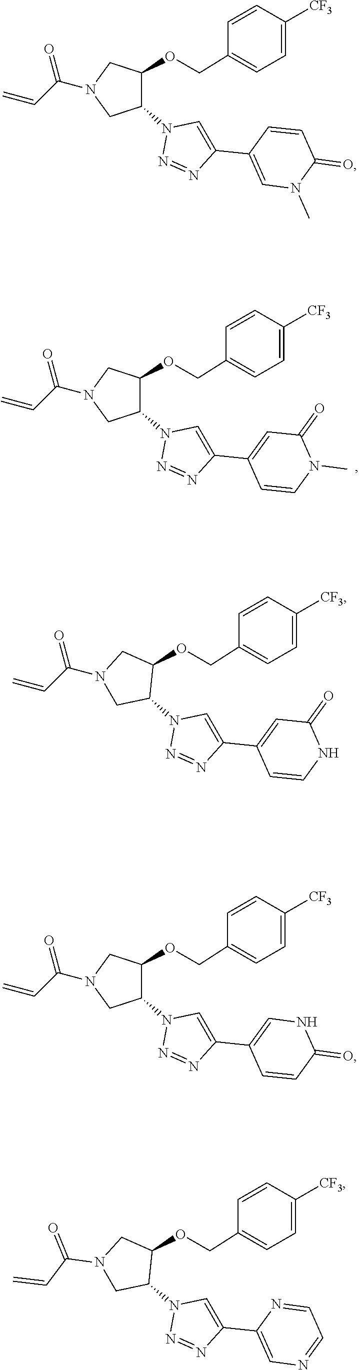 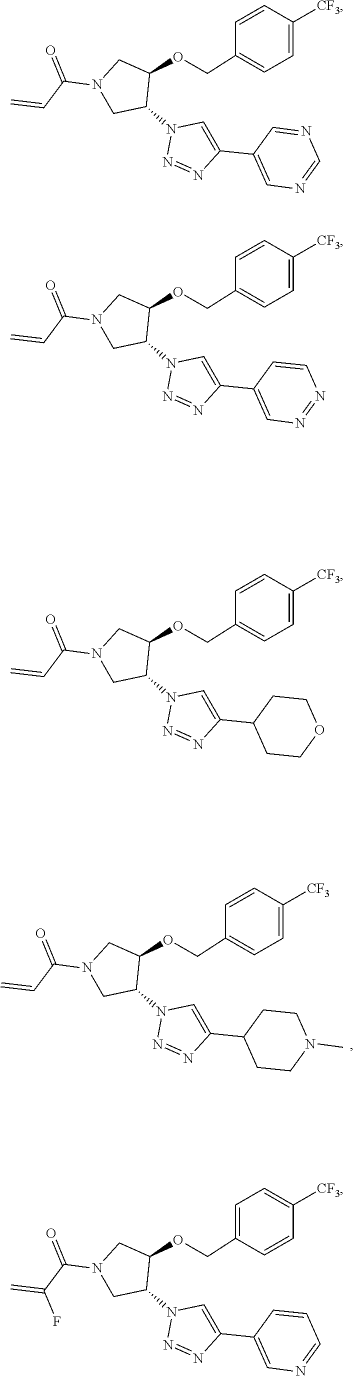 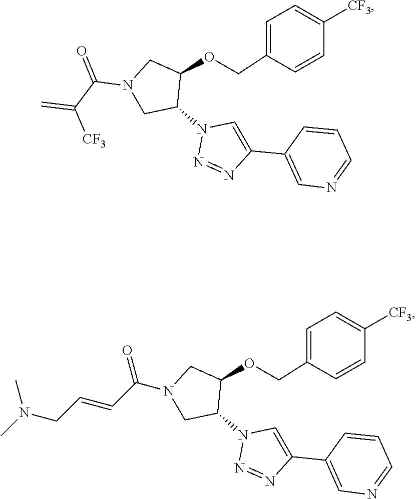 [SMILES: C=C(C(=O)N1C[C@@H](n2cc(-c3cccnc3)nn2)[C@H](OCc2ccc(C(F)(F)F)cc2)C1)C(F)(F)F ; CN(C)C/C=C/C(=O)N1C[C@@H](n2cc(-c3cccnc3)nn2)[C@H](OCc2ccc(C(F)(F)F)cc2)C1] 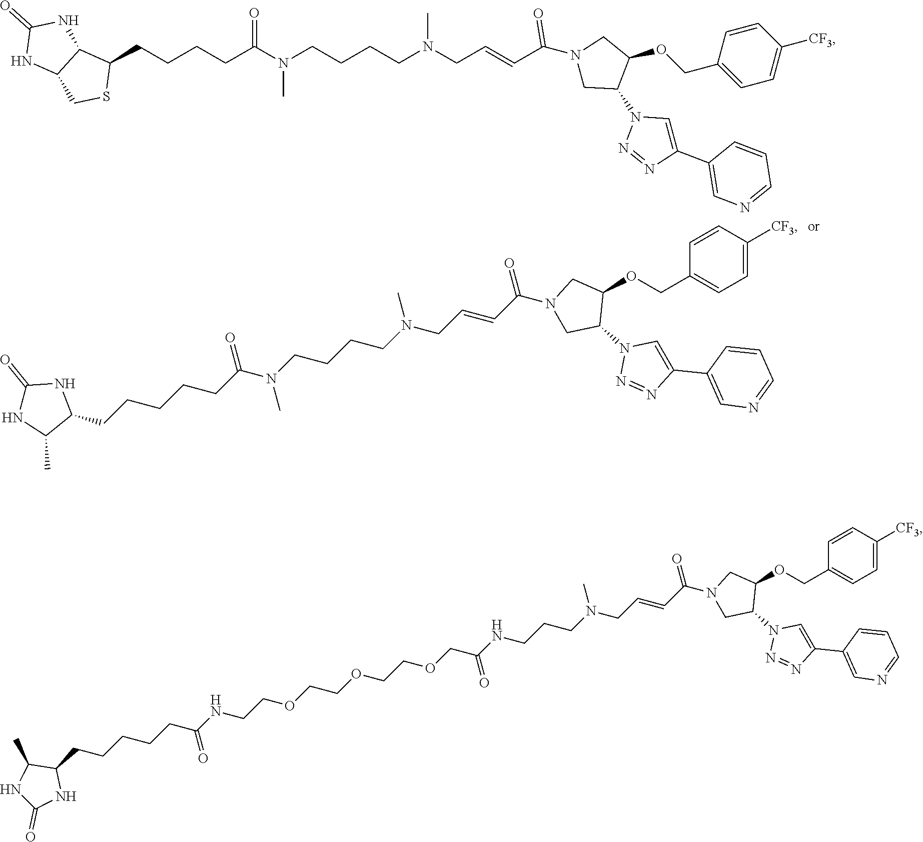
or a pharmaceutically acceptable salt, solvate, hydrate, polymorph, co-crystal, tautomer, stereoisomer, isotopically labeled derivative, or prodrug thereof.

77. The compound of claim 49, wherein the compound of Formula II is of the Formula (II-a): 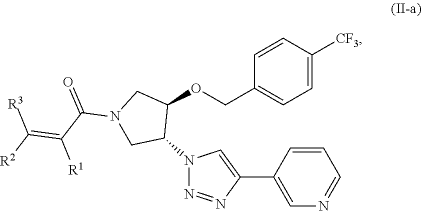
wherein at least one of R¹, R², or R³ is not hydrogen.

78. The compound of claim 1, wherein
R¹ is halogen, optionally substituted alkyl, or —H;
R² is optionally substituted alkyl, or —H; and
R³ is —H.

79-88. (canceled)

89. A compound of Formula (III): 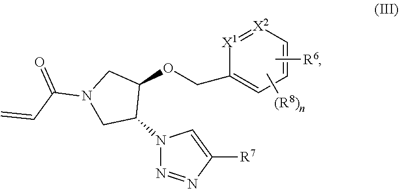
or a pharmaceutically acceptable salt, solvate, hydrate, polymorph, co-crystal, tautomer, stereoisomer, isotopically labeled derivative, or prodrug thereof, wherein:
R⁶ is —CF₃, or —C≡C—H;
R⁷ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
each instance of R⁸ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{c}, —N(R^{c})₂, —SR^{c}, —C(═O)R^{c}, —C(═O)OR^{c}, —C(═O)N(R^{c})₂, —OC(═O)R^{c}, —N(R^{c})C(═O)R^{c}, —CN, or —NO₂;
each occurrence of R^{c} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{c}, when present, can be joined together with the heteroatom to which they are attached to form an optionally substituted heterocyclic ring;
X¹ and X² are each independently —N═ or —C(H)═; and
n is 0, 1, or 2.

90. The compound of claim 89, wherein R⁷ is —H or optionally substituted heteroaryl; and
R⁸ is —H or halo.

91-95. (canceled)

96. The compound of claim 89, wherein the compound of Formula III is of Formula (III-a) or Formula (III-b):

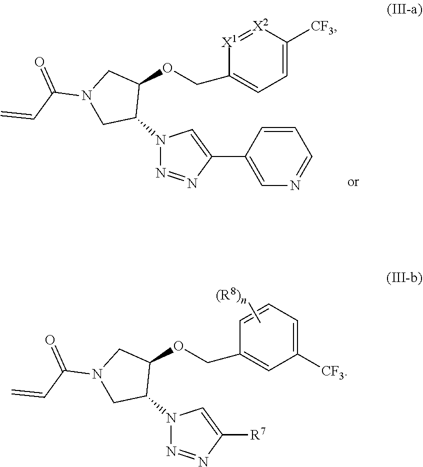

97-103. (canceled)

104. The compound of claim 89 of the formula: 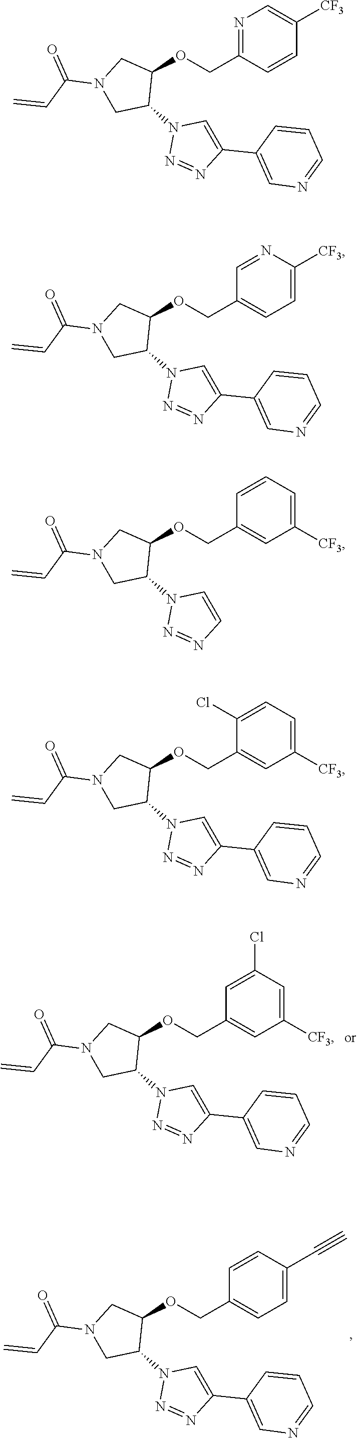 [SMILES: C=CC(=O)N1C[C@@H](n2cc(-c3cccnc3)nn2)[C@H](OCc2ccc(C(F)(F)F)cn2)C1 ; C=CC(=O)N1C[C@@H](n2cc(-c3cccnc3)nn2)[C@H](OCc2ccc(C(F)(F)F)nc2)C1 ; C=CC(=O)N1C[C@@H](n2ccnn2)[C@H](OCc2cccc(C(F)(F)F)c2)C1 ; C=CC(=O)N1C[C@@H](n2cc(-c3cccnc3)nn2)[C@H](OCc2cc(C(F)(F)F)ccc2Cl)C1 ; C=CC(=O)N1C[C@@H](n2cc(-c3cccnc3)nn2)[C@H](OCc2cc(Cl)cc(C(F)(F)F)c2)C1 ; C#Cc1ccc(CO[C@@H]2CN(C(=O)C=C)C[C@H]2n2cc(-c3cccnc3)nn2)cc1]
or a pharmaceutically acceptable salt, solvate, hydrate, polymorph, co-crystal, tautomer, stereoisomer, isotopically labeled derivative, or prodrug thereof.

105-109. (canceled)

110. A compound of Formula (IV): 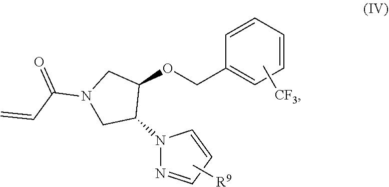
or a pharmaceutically acceptable salt, solvate, hydrate, polymorph, co-crystal, tautomer, stereoisomer, isotopically labeled derivative, or prodrug thereof, wherein:
R⁹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{d}, —N(R^{d})₂, —SR^{d}, —C(═O)R^{d}, —C(═O)OR^{d}, —C(═O)N(R^{d})₂, —OC(═O)R^{d}, —N(Rd)C(═O)R^{d}, —CN, or —NO₂;
each occurrence of R^{d} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{d}, when present, can be joined together with the heteroatom to which they are attached to form an optionally substituted heterocyclic ring.

111. The compound of claim 110, wherein the compound of Formula IV is a compound of Formula (IV-a): 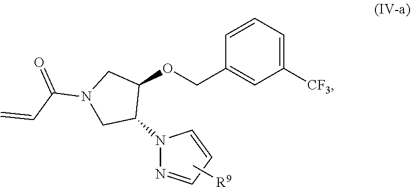
of Formulae: 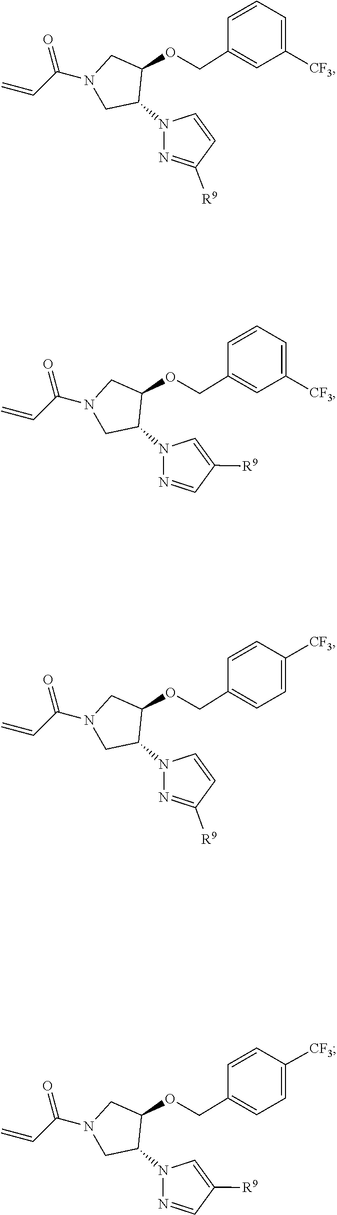
or of Formula IV-b: 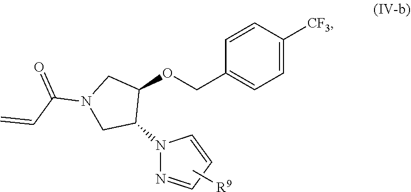
or a pharmaceutically acceptable salt, solvate, hydrate, polymorph, co-crystal, tautomer, stereoisomer, isotopically labeled derivative, or prodrug thereof.

112-116. (canceled)

117. The compound of claim 110, wherein R⁹ is —C(═O)N(R^{d})₂ or —CN; and
at least one instance of R^{d} is optionally substituted alkyl.

118-121. (canceled)

122. The compound of claim 110, of the formula: 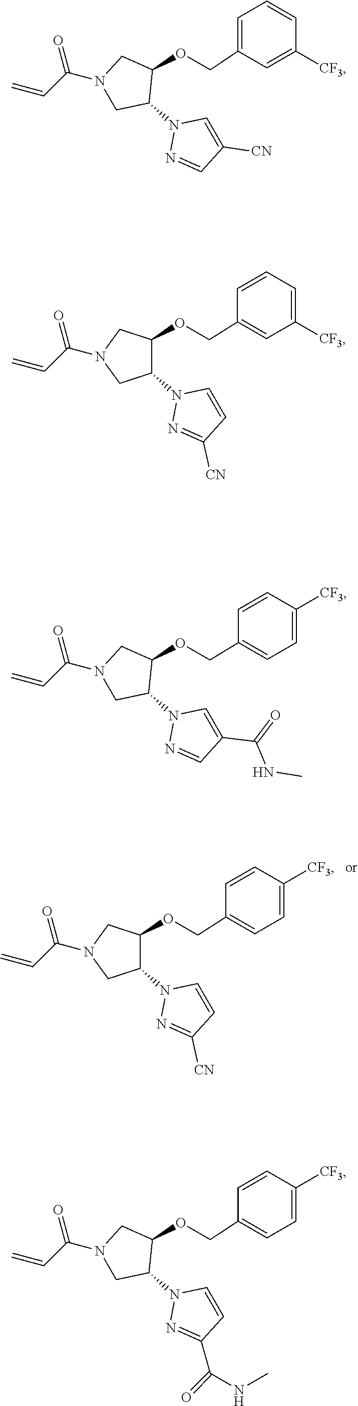
or a pharmaceutically acceptable salt, solvate, hydrate, polymorph, co-crystal, tautomer, stereoisomer, isotopically labeled derivative, or prodrug thereof.

123. (canceled)

124. A compound of Formula (V): 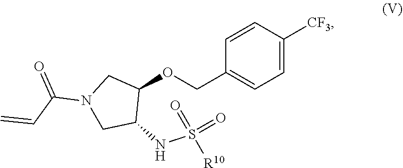
or a pharmaceutically acceptable salt, solvate, hydrate, polymorph, co-crystal, tautomer, stereoisomer, isotopically labeled derivative, or prodrug thereof, wherein:
R¹⁰ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{e}, —N(R^{e})₂, —SR^{e}, —C(═O)R^{e}, —C(═O)OR^{e}, —C(═O)N(R^{e})₂, —OC(═O)R^{e}, —N(R^{e})C(═O)R^{e}, —CN, or —NO₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{e}, when present, can be joined together with the heteroatom to which they are attached to form an optionally substituted heterocyclic ring.

125. The compound of claim 124, wherein R¹⁰ is optionally substituted heteroaryl or optionally substituted aryl.

126-127. (canceled)

128. The compound of claim 124, of the formula: 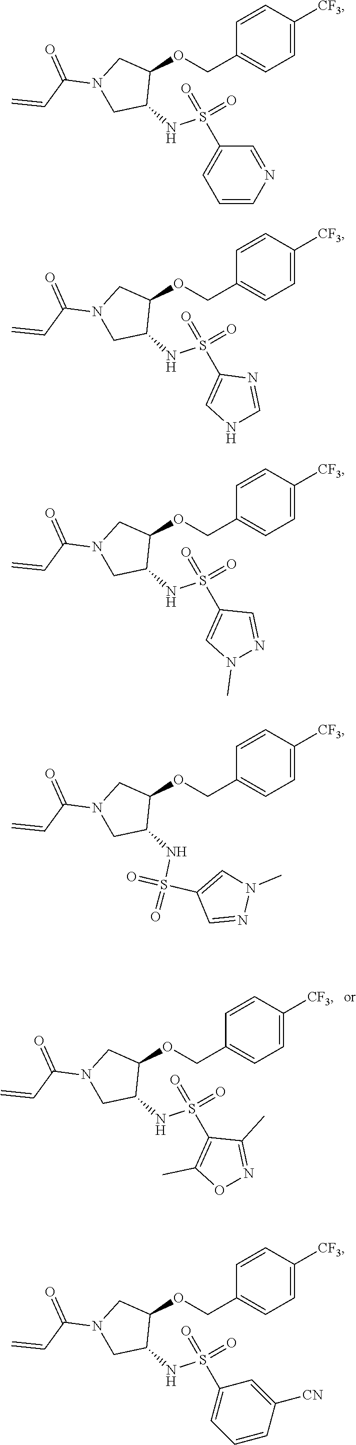
or a pharmaceutically acceptable salt, solvate, hydrate, polymorph, co-crystal, tautomer, stereoisomer, isotopically labeled derivative, or prodrug thereof.

129-132. (canceled)

133. A compound of Formula (VI): 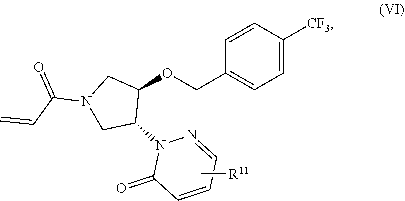
or a pharmaceutically acceptable salt, solvate, hydrate, polymorph, co-crystal, tautomer, stereoisomer, isotopically labeled derivative, or prodrug thereof, wherein:
R¹¹ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{f}, —N(R^{f})₂, —SR^{f}, —C(═O)R^{f}, —C(═O)OR^{f}, —C(═O)N(R^{f})₂, —OC(═O)R^{f}, —N(R^{f})C(═O)R^{f}, —CN, or —NO₂;
each occurrence of R^{f} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group, or two instances of R^{f}, when present, can be joined together with the heteroatom to which they are attached to form an optionally substituted heterocyclic ring.

134-135. (canceled)

136. The compound of claim 133, wherein R¹¹ is optionally substituted heteroaryl or —H.

137-139. (canceled)

140. The compound of claim 133, of the formula: 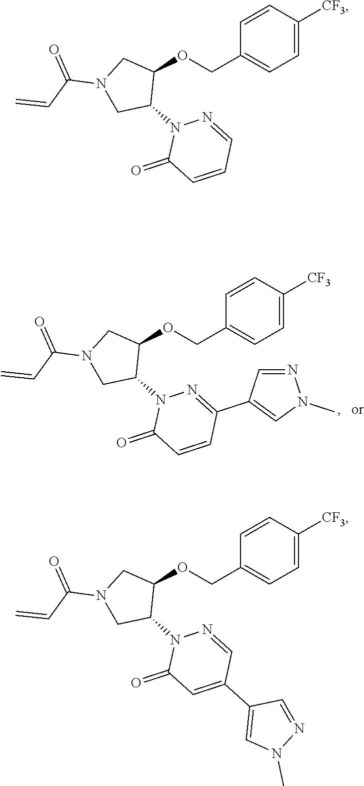
or a pharmaceutically acceptable salt, solvate, hydrate, polymorph, co-crystal, tautomer, stereoisomer, isotopically labeled derivative, or prodrug thereof.

141. A compound selected from the group consisting of 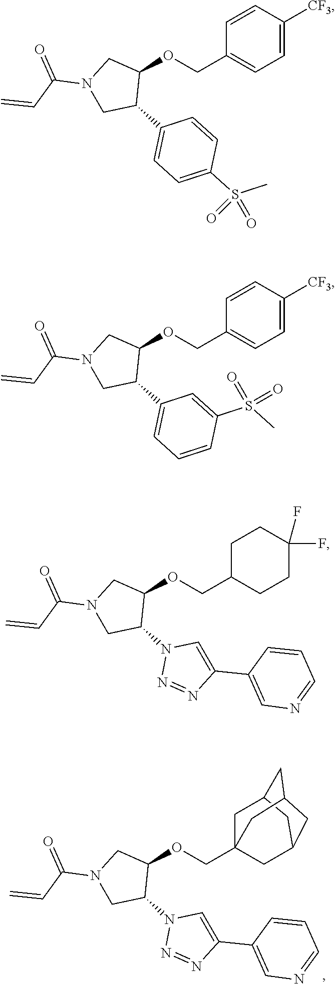 [SMILES: C=CC(=O)N1C[C@@H](OCc2ccc(C(F)(F)F)cc2)[C@H](c2ccc(S(C)(=O)=O)cc2)C1 ; C=CC(=O)N1C[C@@H](OCc2ccc(C(F)(F)F)cc2)[C@H](c2cccc(S(C)(=O)=O)c2)C1 ; C=CC(=O)N1C[C@@H](n2cc(-c3cccnc3)nn2)[C@H](OCC2CCC(F)(F)CC2)C1 ; C=CC(=O)N1C[C@@H](n2cc(-c3cccnc3)nn2)[C@H](OCC23CC4CC(CC(C4)C2)C3)C1] 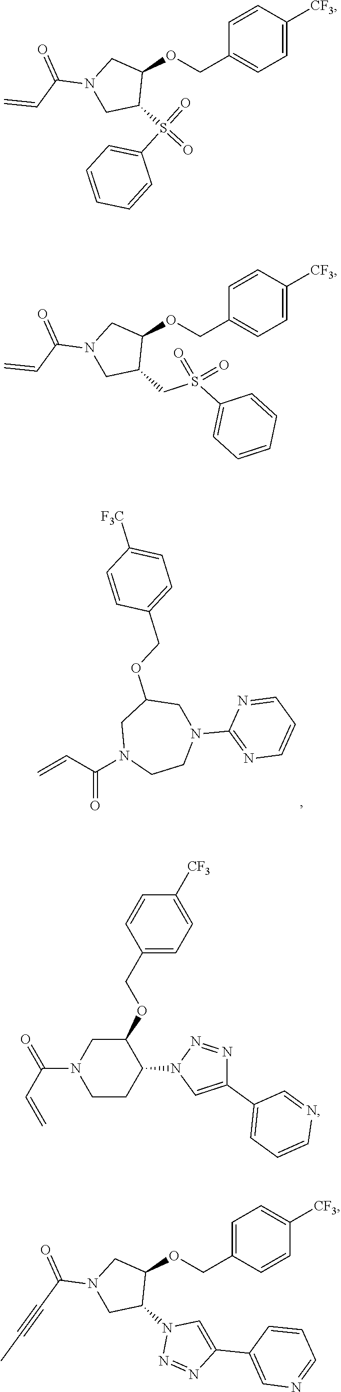 [SMILES: C=CC(=O)N1C[C@@H](S(=O)(=O)c2ccccc2)[C@H](OCc2ccc(C(F)(F)F)cc2)C1 ; C=CC(=O)N1C[C@@H](CS(=O)(=O)c2ccccc2)[C@H](OCc2ccc(C(F)(F)F)cc2)C1 ; C=CC(=O)N1CCN(c2ncccn2)CC(OCc2ccc(C(F)(F)F)cc2)C1 ; C=CC(=O)N1CC[C@@H](n2cc(-c3cccnc3)nn2)[C@H](OCc2ccc(C(F)(F)F)cc2)C1 ; CC#CC(=O)N1C[C@@H](n2cc(-c3cccnc3)nn2)[C@H](OCc2ccc(C(F)(F)F)cc2)C1] 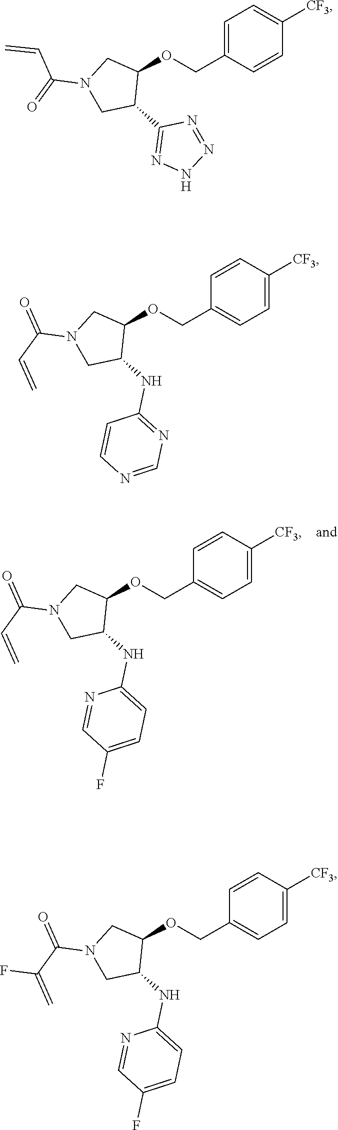 [SMILES: C=CC(=O)N1C[C@@H](OCc2ccc(C(F)(F)F)cc2)[C@H](c2nn[nH]n2)C1 ; C=CC(=O)N1C[C@@H](Nc2ccncn2)[C@H](OCc2ccc(C(F)(F)F)cc2)C1 ; C=CC(=O)N1C[C@@H](Nc2ccc(F)cn2)[C@H](OCc2ccc(C(F)(F)F)cc2)C1 ; C=C(F)C(=O)N1C[C@@H](Nc2ccc(F)cn2)[C@H](OCc2ccc(C(F)(F)F)cc2)C1]
or a pharmaceutically acceptable salt, solvate, hydrate, polymorph, co-crystal, tautomer, stereoisomer, isotopically labeled derivative, or prodrug thereof.

142-147. (canceled)

148. A pharmaceutical composition comprising a compound of claim 1, or a pharmaceutically acceptable salt, solvate, hydrate, polymorph, co-crystal, tautomer, stereoisomer, isotopically labeled derivative, or prodrug thereof, and optionally a pharmaceutically acceptable excipient.

149. (canceled)

150. A method of treating cancer in a subject in need thereof, the method comprising administering to the subject a therapeutically effective amount of a compound of claim 1, or a pharmaceutically acceptable salt, solvate, hydrate, polymorph, co-crystal, tautomer, stereoisomer, isotopically labeled derivative, or prodrug thereof.

151. (canceled)

152. The method of claim 150, wherein the cancer is a sarcoma, lung cancer, thyroid cancer, breast cancer, liver cancer, prostate cancer, pancreatic cancer, colorectal cancer, ovarian cancer, skin cancer, esophageal cancer, or carcinoma.

153-165. (canceled)

166. A method of inhibiting a transcription factor in a subject in need thereof, the method comprising:
administering to the subject a therapeutically effective amount of a compound of claim 1, or a pharmaceutically acceptable salt, solvate, hydrate, polymorph, co-crystal, tautomer, stereoisomer, isotopically labeled derivative, or prodrug thereof, wherein the transcription factor is TEAD1, TEAD2, TEAD3, or TEAD4.

167-175. (canceled)
